(19) United States
(12) Patent Application Publication
(10) Pub. No.: US 2005/0287546 A1
(43) Pub. Date: Dec. 29, 2005

(54) Novel proteases

(75) Inventors: Gregory Plowman, San Carlos, CA (US); David Whyte, Belmont, CA (US); Sean Caenepeel, Woodland Hills, CA (US); Glen Charydczak, Princeton Jct., NJ (US); Gerard Manning, La Jolla, CA (US); Sucha Sudarsanam, Greenbrae, CA (US)

Correspondence Address: FOLEY AND LARDNER LLP;SUITE 500, 3000 K STREET NW, WASHINGTON, DC, 20007, US

(73) Assignee: SUGEN, INC.

(21) Appl. No.: 11/037,243

(22) Filed: Jan. 19, 2005

Related U.S. Application Data
(63) Continuation of application No. 09/888,615, filed on Jun. 26, 2001.
(60) Provisional application No. 60/214,047, filed on Jun. 26, 2000.

(51) Int. Cl. C12Q 1/68; C07H 21/04; C12P 21/06; C12N 9/48; C12N 9/64

(52) U.S. Cl. 435/6; 435/69.1; 435/320.1; 435/325; 435/226; 536/23.2

(57) ABSTRACT

The present invention relates to protease polypeptides, nucleotide sequences encoding the protease polypeptides, as well as various products and methods useful for the diagnosis and treatment of various protease-related diseases and conditions.

The present invention claims priority to provisional application Ser. No. 60/214,047 filed Jun. 26, 2000, which is hereby incorporated by reference in its entirety.

### FIELD OF THE INVENTION

The present invention relates to protease polypeptides, nucleotide sequences encoding the protease polypeptides, as well as various products and methods useful for the diagnosis and treatment of various protease-related diseases and conditions.

### BACKGROUND OF THE INVENTION

Proteases and Human Disease

“Protease,” “proteinase,” and “peptidase” are synonymous terms applying to all enzymes that hydrolyse peptide bonds, i.e. proteolytic enzymes. Proteases are an exceptionally important group of enzymes in medical research and biotechnology. They are necessary for the survival of all living creatures, and are encoded by 1-2% of all mammalian genes. Rawlings and Barrett (MEROPS: the peptidase database. Nucleic Acids Res., 1999, 27:325-331) (http://www.babraham.co.uk/Merops/Merops.htm (Which is incorporated herein by reference in its entirety including any figures, tables, or drawings.) have classified peptidases into 157 families based on structural similarity at the catalytic core sequence. These families are further classed into 26 clans, based on indications of common evolutionary relationship. Peptidases play key roles in both the normal physiology and disease-related pathways in mammalian cells. Examples include the modulation of apoptosis (caspases), control of blood pressure (renin, angiotensin-converting enzymes), tissue remodeling and tumor invasion (collagenase), the development of Alzheimer's Disease (β-secretase), protein turnover and cell-cycle regulation (proteosome), and inflammation (TNF-α convertase). (Barrett et al., Handbook of Proteolytic Enzymes, 1998, Academic Press, San Diego which is incorporated herein by reference in its entirety including any figures, tables, or drawings.)

Peptidases are classed as either exopeptidases or endopeptidases. The exopeptidases act only near the ends of polypeptide chains: aminopeptidases act at the free N-terminus and carboxypeptidases at the free C-terminus. The endopeptidases are divided, on the basis of their mechanism of action, into six sub-subclasses: aspartyl endopeptidases (3.4.23), cysteine endopeptidases (3.4.22), metalloendopeptidases (3.4.24), serine endopeptidases (3.4.21), threonine endopeptidases (3.4.25), and a final group that could not be assigned to any of the above classes (3.4.99). (Enzyme nomenclature and numbering are based on “Recommendations of the Nomenclature Committee of the International Union of Biochemistry and Molecular Biology (NC-IUBMB) 1992, (http://www.chem.qmw.ac.uk/iubmb/enzyme/EC34/intro.html).)

In serine-, threonine- and cysteine-type peptidases, the catalytic nucleophile is the reactive group of an amino acid side chain, either a hydroxyl group (serine- and threonine-type peptidases) or a sulfhydryl group (cysteine-type peptidases). In aspartic-type and metallopeptidases, the nucleophile is commonly an activated water molecule. In aspartic-type peptidases, the water molecule is directly bound by the side chains of aspartate residues. In metallopeptidases, one or two metal ions hold the water molecule in place, and charged amino acid side chains are ligands for the metal ions. The metal may be zinc, cobalt or manganese. One metal ion is usually attached to three amino acid ligands. Families of peptidases are referred to by use of the numbering system of Rawlings & Barrett (Rawlings, N. D. & Barrett, A. J. MEROPS: the peptidase database. Nucleic Acids Research 27 (1999) 325-331, which is incorporated herein by reference in its entirety including any figures, tables, or drawings). ). Enzyme nomenclature and numbering are based on “Recommendations of the Nomenclature Committee of the International Union of Biochemistry and Molecular Biology (NC-IUBMB) 1992, (http://www.chem.gmw.ac.uk/iubmb/enzyme/EC34/intro.html).

Protease Families

1. Aspartyl Proteases (Prosite Number PS00141)

Aspartyl proteases, also known as acid proteases, are a widely distributed family of proteolytic enzymes in vertebrates, fungi, plants, retroviruses and some plant viruses. Aspartate proteases of eukaryotes are monomeric enzymes which consist of two domains. Each domain contains an active site centered on a catalytic aspartyl residue. The two domains most probably evolved from the duplication of an ancestral gene encoding a primordial domain. Enzymes in this class include cathepsin E, renin, presenilin (PS1), and the APP secretases.

2. Cysteine Proteases (Prosite PDOC00126)

Eukaryotic cysteine proteases are a family of proteolytic enzymes which contain an active site cysteine. Catalysis proceeds through a thioester intermediate and is facilitated by a nearby histidine side chain; an asparagine completes the essential catalytic triad. Peptidases in this family with important roles in disease include the caspases, calpain, hedgehog, ubiquitin hydrolases, and papain.

3. Metalloproteases (Prosite PDOC00129)

The metalloproteases are a class which includes matrix metalloproteases (MMPs), collagenase, stromelysin, gelatinase, neprylisin, carboxypeptidase, dipeptidase, and membrane-associated metalloproteases, such as those of the ADAM family. They require a metal co-factor for activity; frequently the required metal ion is zinc but some metalloproteases utilize cobalt and manganese.

Proteins of the extracellular matrix interact directly with cell surface receptors thereby initiating signal transduction pathways and modulating those triggered by growth factors, some of which may require binding to the extracellular matrix for optimal activity. Therefore-the extracellular matrix has a profound effect on the cells encased by it and adjacent to-it. Remodeling of the extracellular matrix requires protease of several families, including metalloproteases (MMPs).

4. Serine Proteases (S1) (Prosite PS00134 Trypsin-His, PS00135 Trypsin-Ser)

The catalytic activity of the serine proteases from the trypsin family is provided by a charge relay system involving an aspartic acid residue hydrogen-bonded to a histidine, which itself is hydrogen-bonded to a serine. The sequences in the vicinity of the active site serine and histidine residues are well conserved in this family of proteases. A partial list of proteases known to belong to this large and important family include: blood coagulation factors VII, IX, X, XI and XII; thrombin; plasminogen; complement components C1r, C1s, C2; complement factors B, D and I; complement-activating component of RA-reactive factor; elastases 1, 2, 3A, 3B (protease E); hepatocyte growth factor activator; glandular (tissue) kallikreins including EGF-binding protein types A, B, and C; NGF-γ hain, γ-renin, and prostate specific antigen (PSA); plasma kallikrein; mast cell proteases; myeloblastin (proteinase 3) (Wegener's autoantigen); plasminogen activators (urokinase-type, and tissue-type); and the trypsins I, II, III, and IV. These peptidases play key roles in coagulation, tumorigenesis, control of blood pressure, release of growth factors, and other roles.

5. Threonine Peptidases (T1)—(Prosite PDOC00326/PDOC00668)

Threonine proteases are characterized by their use of a hydroxyl group of a threonine residue in the catalytic site of these enyzmes. Only a few of these enzymes have been characterized thus far, such as the 20S proteasome from the archaebacterium Thermoplasma acidophilum (Seemuller et al., 1995, Science, 268:579-82, and chapter 167 of Barrett et al., Handbook of Proteolytic Enzymes, 1998, Academic Press, San Diego).

### SUMMARY OF THE INVENTION

This invention concerns the isolation and characterization of novel sequences of human proteases. These sequences are obtained via bioinformatics searching strategies on the predicted amino acid translations of new human genetic sequences. These sequences, now identified as proteases, are translated into polypeptides which are further characterized. Additionally, the nucleic acid sequences of these proteases are used to obtain full-length cDNA clones of the proteases. The partial or complete sequences of these proteases are presented here, together with their classification, predicted or deduced protein structure.

Modulation of the activities of these proteases will prove useful therapeutically. Additionally, the presence or absence of these proteases or the DNA sequence encoding them will prove useful in diagnosis or prognosis of a variety of diseases. In this regard, Example 8 describes the chromosomal localization of proteases of the present invention, and describes diseases mapping to the chromosomal locations of the proteases of the invention.

A first aspect of the invention features an identified, isolated, enriched, or purified nucleic acid molecule having an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118 and biological domains thereof.

The term “identified” in reference to a nucleic acid is meant that a sequence was selected from a genomic, EST, or cDNA sequence database based on being predicted to encode a portion of a previously unknown or novel protease.

By “isolated” in reference to nucleic acid is meant a polymer of 10 (preferably 21, more preferably 39, most preferably 75) or more nucleotides conjugated to each other, including DNA and RNA that is isolated from a natural source or that is synthesized as the sense or complementary antisense strand. In certain embodiments of the invention, longer nucleic acids are preferred, for example those of 300, 600, 900, 1200, 1500, or more nucleotides and/or those having at least 50%, 60%, 75%, 80%, 85%, 90%, 91%, 92%, 93%, 94%, 95%, 96%, 97%, 98% or 99% identity to a sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, and SEQ ID NO:59.

It is understood that by nucleic acid it is meant, without limitation, DNA, RNA or cDNA, and where the nucleic acid is RNA, the thymine (T) will be uracil (U).

The isolated nucleic acid of the present invention is unique in the sense that it is not found in a pure or separated state in nature. Use of the term “isolated” indicates that a naturally occurring sequence has been removed from its normal cellular (i.e., chromosomal) environment. Thus, the sequence may be in a cell-free solution or placed in a different cellular environment. The term does not imply that the sequence is the only nucleotide chain present, but that it is essentially free (preferably about 90% pure, more preferably at least about 95% pure) of non-nucleotide material naturally associated with it, and thus is distinguished from isolated chromosomes.

By the use of the term “enriched” in reference to nucleic acid is meant that the specific DNA or RNA sequence constitutes a significantly higher fraction (2- to 5-fold) of the total DNA or RNA present in the cells or solution of interest than in normal or diseased cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that enriched does not imply that there are no other DNA or RNA sequences present, just that the relative amount of the sequence of interest has been significantly increased. The term “significant” is used to indicate that the level of increase is useful to the person making such an increase, and generally means an increase relative to other nucleic acids of about at least 2-fold, more preferably at least 5-fold, more preferably at least 10-fold or even more. The term also does not imply that there is no DNA or RNA from other sources. The DNA from other sources may, for example, comprise DNA from a yeast or bacterial genome, or a cloning vector such as pUC19. This term distinguishes from naturally occurring events, such as viral infection, or tumor-type growths, in which the level of one mRNA may be naturally increased relative to other species of mRNA. That is, the term is meant to cover only those situations in which a person has intervened to elevate the proportion of the desired nucleic acid.

It is also advantageous for some purposes that a nucleotide sequence be in purified form. The term “purified” in reference to nucleic acid does not require absolute purity (such as a homogeneous preparation). Instead, it represents an indication that the sequence is relatively more pure than in the natural environment (compared to the natural level this level should be at least 2- to 5-fold greater, e.g., in terms of mg/mL). Individual clones isolated from a cDNA library may be purified to electrophoretic homogeneity. The claimed DNA molecules obtained from these clones could be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁶-fold purification of the native message. Thus, purification of at least one order of magnitude, preferably two or three orders, and more preferably four or five orders of magnitude is expressly contemplated.

By a “protease polypeptide” is meant 32 (preferably 40, more preferably 45, most preferably 55) or more contiguous amino acids in a polypeptide having an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118 and biological domains thereof. In certain aspects, polypeptides of 100, 200, 300, 400, 450, 500, 550, 600, 700, 800, 900 or more amino acids are preferred. The protease polypeptide can be encoded by a full-length nucleic acid sequence or any portion of the full-length nucleic acid sequence, so long as a functional activity of the polypeptide is retained. It is well known in the art that due to the degeneracy of the genetic code numerous different nucleic acid sequences can code for the same amino acid sequence. Equally, it is also well known in the art that conservative changes in amino acid can be made to arrive at a protein or polypeptide which retains the functionality of the original. Such substitutions may include the replacement of an amino acid by a residue having similar physicochemical properties, such as substituting one aliphatic residue (Ile, Val, Leu or Ala) for another, or substitution between basic residues Lys and Arg, acidic residues Glu and Asp, amide residues Gln and Asn, hydroxyl residues Ser and Tyr, or aromatic residues Phe and Tyr. Further information regarding making amino acid exchanges which have only slight, if any, effects on the overall protein can be found in Bowie et al., Science, 1990, 247:1306-1310, which is incorporated herein by reference in its entirety including any figures, tables, or drawings. In all cases, all permutations are intended to be covered by this disclosure.

The amino acid sequence of the protease peptide of the invention will be substantially similar to a sequence having an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118, or the corresponding full-length amino acid sequence, or fragments thereof.

A sequence that is substantially similar to a sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118 will preferably have at least 50%, 60%, 75%, 80%, 85%, 90%, 91%, 92%, 93%, 94%, 95%, 96%, 97%, 98% or 99% identity to a sequence selected from the group consisting of SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118. Preferably the protease polypeptide will have at least about 90%, 91%, 92%, 93%, 94%, 95%, 96%, 97%, 98% or 99% identity to one of the aforementioned sequences.

By “identity” is meant a property of sequences that measures their similarity or relationship. Identity is measured by dividing the number of identical residues by the total number of residues and gaps and multiplying the product by 100. “Gaps” are spaces in an alignment that are the result of additions or deletions of amino acids. Thus, two copies of exactly the same sequence have 100% identity, but sequences that are less highly conserved, and have deletions, additions, or replacements, may have a lower degree of identity. Those skilled in the art will recognize that several computer programs are available for determining sequence identity using standard parameters, for example Gapped BLAST or PSI-BLAST (Altschul, et al. (1997) Nucleic Acids Res. 25:3389-3402), BLAST (Altschul, et al. (1990) J. Mol. Biol. 215:403-410), and Smith-Waterman (Smith, et al. (1981) J. Mol. Biol. 147:195-197). Preferably, the default settings of these programs will be employed, but those skilled in the art recognize whether these settings need to be changed and know how to make the changes.

“Similarity” is measured by dividing the number of identical residues plus the number of conservatively substituted residues (see Bowie, et al. Science, 1999), 247:1306-1310, which is incorporated herein by reference in its entirety, including any drawings, figures, or tables) by the total number of residues and gaps and multiplying the product by 100.

In preferred embodiments, the invention features isolated, enriched, or purified nucleic acid molecules encoding a protease polypeptide comprising a nucleotide sequence that: (a) encodes a polypeptide having an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118 and biological domains thereof; (b) is the complement of the nucleotide sequence of (a); or (c) hybridizes under highly stringent conditions to the nucleotide molecule of (a) and encodes a naturally occurring protease polypeptide.

In preferred embodiments, the invention features isolated, enriched or purified nucleic acid molecules comprising a nucleotide sequence substantially identical to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, and SEQ ID NO:59. Preferably the sequence has at least 50%, 60%, 75%, 80%, 85%, 90%, 91%, 92%, 93%, 94%, 95%, 96%, 97%, 98%, 99% or 100% identity to the above listed sequences.

The term “complement” refers to two nucleotides that can form multiple favorable interactions with one another. For example, adenine is complementary to thymine as they can form two hydrogen bonds. Similarly, guanine and cytosine are complementary since they can form three hydrogen bonds. A nucleotide sequence is the complement of another nucleotide sequence if all of the nucleotides of the first sequence are complementary to all of the nucleotides of the second sequence.

Various low or high stringency hybridization conditions may be used depending upon the specificity and selectivity desired. These conditions are well known to those skilled in the art. Under stringent hybridization conditions only highly complementary nucleic acid sequences hybridize. Preferably, such conditions prevent hybridization of nucleic acids having more than 1 or 2 mismatches out of 20 contiguous nucleotides, more preferably, such conditions prevent hybridization of nucleic acids having more than 1 or 2 mismatches out of 50 contiguous nucleotides, most preferably, such conditions prevent hybridization of nucleic acids having more than 1 or 2 mismatches out of 100 contiguous nucleotides. In some instances, the conditions may prevent hybridization of nucleic acids having more than 5 mismatches in the full-length sequence.

By stringent hybridization assay conditions is meant hybridization assay conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5× Denhardt's solution at 42° C. overnight; washing with 2×SSC, 0.1% SDS at 45° C.; and washing with 0.2×SSC, 0.1% SDS at 45° C. Under some of the most stringent hybridization assay conditions, the second wash can be done with 0.1×SSC at a temperature up to 70° C. (Berger et al. (1987) Guide to Molecular Cloning Techniques pg 421, hereby incorporated by reference herein in its entirety including any figures, tables, or drawings.). However, other applications may require the use of conditions falling between these sets of conditions. Methods of determining the conditions required to achieve desired hybridizations are well known to those with ordinary skill in the art, and are based on several factors, including but not limited to, the sequences to be hybridized and the samples to be tested. Washing conditions of lower stringency frequently utilize a lower temperature during the washing steps, such as 65° C., 60° C., 55° C., 50° C., or 42° C.

The term “activity” means that the polypeptide hydrolyzes peptide bonds.

The term “catalytic activity”, as used herein, defines the rate at which a protease catalytic domain cleaves a substrate. Catalytic activity can be measured, for example, by determining the amount of a substrate cleaved as a function of time. Catalytic activity can be measured by methods of the invention by holding time constant and determining the concentration of a cleaved substrate after a fixed period of time. Cleavage of a substrate occurs at the active site of the protease. The active site is normally a cavity in which the substrate binds to the protease and is cleaved.

The term “biological domain” means a domain or region of the protease polypeptide which has catalytic activity or which binds to the substrate of the protease.

The term “substrate” as used herein refers to a polypeptide or protein which is cleaved by a protease of the invention. The term “cleaved” refers to the severing of a covalent bond between amino acid residues of the backbone of the polypeptide or protein.

The term “insert” as used herein refers to a portion of a protease that is absent from a close homolog. Inserts may or may not be the product alternative splicing of exons. Inserts can be identified by using a Smith-Waterman sequence alignment of the protein sequence against the non-redundant protein database, or by means of a multiple sequence alignment of homologous sequences using the DNAStar program Megalign (Preferably, the default settings of this program will be used, but those skilled in the art will recognize whether these settings need to be changed and know how to make the changes.). Inserts may play a functional role by presenting a new interface for protein-protein interactions, or by interfering with such interactions.

In other preferred embodiments, the invention features isolated, enriched, or purified nucleic acid molecules encoding protease polypeptides, further comprising a vector or promoter operably linked to the nucleotide sequence. The invention also features recombinant nucleic acid, preferably in a cell or an organism. The recombinant nucleic acid may contain a sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, and SEQ ID NO:59, or a functional derivative thereof and a vector or a promoter operably linked to the nucleotide sequence. The recombinant nucleic acid can alternatively contain a transcriptional initiation region functional in a cell, a sequence complementary to an RNA sequence encoding a protease polypeptide and a transcriptional termination region functional in a cell. Specific vectors and host cell combinations are discussed herein.

The term “vector” relates to a single or double-stranded circular nucleic acid molecule that can be transfected into cells and replicated within or independently of a cell genome. A circular double-stranded nucleic acid molecule can be cut and thereby linearized upon treatment with restriction enzymes. An assortment of nucleic acid vectors, restriction enzymes, and the knowledge of the nucleotide sequences cut by restriction enzymes are readily available to those skilled in the art. A nucleic acid molecule encoding a protease can be inserted into a vector by cutting the vector with restriction enzymes and ligating the two pieces together.

An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation.

The term “transfecting” defines a number of methods to insert a nucleic acid vector or other nucleic acid molecules into a cellular organism. These methods involve a variety of techniques, such as treating the cells with high concentrations of salt, an electric field, detergent, or DMSO to render the outer membrane or wall of the cells permeable to nucleic acid molecules of interest or use of various viral transduction strategies.

The term “promoter” as used herein, refers to nucleic acid sequence needed for gene sequence expression. Promoter regions vary from organism to organism, but are well known to persons skilled in the art for different organisms. For example, in prokaryotes, the promoter region contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

In preferred embodiments, the isolated nucleic acid comprises, consists essentially of, or consists of a nucleic acid sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, and SEQ ID NO:59 which encodes an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118, a functional derivative thereof, or at least 35, 40, 45, 50, 60, 75, 100, 200, or 300 contiguous amino acids selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118. The nucleic acid may be isolated from a natural source by cDNA cloning or by subtractive hybridization. The natural source may be mammalian, preferably human, blood, semen, or tissue, and the nucleic acid may be synthesized by the triester method or by using an automated DNA synthesizer.

The term “mammal” refers preferably to such organisms as mice, rats, rabbits, guinea pigs, sheep, and goats, more preferably to cats, dogs, monkeys, and apes, and most preferably to humans.

In yet other preferred embodiments, the nucleic acid is a conserved or unique region, for example those useful for: the design of hybridization probes to facilitate identification and cloning of additional polypeptides, the design of PCR probes to facilitate cloning of additional polypeptides, obtaining antibodies to polypeptide regions, and designing antisense oligonucleotides.

By “conserved nucleic acid regions”, are meant regions present on two or more nucleic acids encoding a protease polypeptide, to which a particular nucleic acid sequence can hybridize under lower stringency conditions. Examples of lower stringency conditions suitable for screening for nucleic acid encoding protease polypeptides are provided in Wahl et al. Meth. Enzym. 152:399-407 (1987) and in Wahl et al. Meth. Enzym. 152:415-423 (1987), which are hereby incorporated by reference herein in its entirety, including any drawings, figures, or tables. Preferably, conserved regions differ by no more than 5 out of 20 nucleotides, even more preferably 2 out of 20 nucleotides or most preferably 1 out of 20 nucleotides.

By “unique nucleic acid region” is meant a sequence present in a nucleic acid coding for a protease polypeptide that is not present in a sequence coding for any other naturally occurring polypeptide. Such regions preferably encode 32 (preferably 40, more preferably 45, most preferably 55) or more contiguous amino acids set forth in a full-length amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118 in a sample. The nucleic acid probe contains a nucleotide base sequence that will hybridize to the sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, and SEQ ID NO:59, or a functional derivative thereof.

In preferred embodiments, the nucleic acid probe hybridizes to nucleic acid encoding at least 12, 32, 75, 90, 105, 120, 150, 200, 250, 300 or 350 contiguous amino acids of a fill-length sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118, or a functional derivative thereof.

Methods for using the probes include detecting the presence or amount of protease RNA in a sample by contacting the sample with a nucleic acid probe under conditions such that hybridization occurs and detecting the presence or amount of the probe bound to protease RNA. The nucleic acid duplex formed between the probe and a nucleic acid sequence coding for a protease polypeptide may be used in the identification of the sequence of the nucleic acid detected (Nelson et al., in Nonisotopic DNA Probe Techniques, Academic Press, San Diego, Kricka, ed., p. 275, 1992, hereby incorporated by reference herein in its entirety, including any drawings, figures, or tables). Kits for performing such methods may be constructed to include a container means having disposed therein a nucleic acid probe.

Methods for using the probes also include using these probes to find the full-length clone of each of the predicted proteases by techniques known to one skilled in the art. These clones will be useful for screening for small molecule compounds that inhibit the catalytic activity of the encoded protease with potential utility in treating cancers, immune-related diseases and disorders, cardiovascular disease, brain or neuronal-associated diseases, and metabolic disorders. More specifically disorders including cancers of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, multiple sclerosis, and amyotrophic lateral sclerosis; viral or non-viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

In another aspect, the invention describes a recombinant cell or tissue comprising a nucleic acid molecule encoding a protease polypeptide having an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118. In such cells, the nucleic acid may be under the control of the genomic regulatory elements, or may be under the control of exogenous regulatory elements including an exogenous promoter. By “exogenous” it is meant a promoter that is not normally coupled in vivo transcriptionally to the coding sequence for the protease polypeptides.

The polypeptide is preferably a fragment of the protein encoded by a full-length amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118. By “fragment,” is meant an amino acid sequence present in a protease polypeptide. Preferably, such a sequence comprises at least 32, 45, 50, 60, 100, 200, or 300 contiguous amino acids of a full-length sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118.

In another aspect, the invention features an isolated, enriched, or purified protease polypeptide having a sequence substantially identical to an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118 and biological domains thereof. Preferable the polypeptide sequence has at least 50%, 60%, 75%, 80%, 85%, 90%, 91%, 92%, 93%, 94%, 95%, 96%, 97%, 98%, 99% or 100% identity to the above listed sequences.

By “isolated” in reference to a polypeptide is meant a polymer of 6 (preferably 12, more preferably 18, most preferably 25, 32, 40, or 50) or more amino acids conjugated to each other, including polypeptides that are isolated from a natural source or that are synthesized. In certain aspects longer polypeptides are preferred, such as those with 100, 200, 300, 400, 450, 500, 550, 600, 700, 800, 900 or more contiguous amino acids of a full-length sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118, and/or those polypeptides having at least 50%, 60%, 75%, 80%, 85%, 90%, 91%, 92%, 93%, 94%, 95%, 96%, 97%, 98% or 99% identity to a sequence selected from the group consisting of SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118.

The isolated polypeptides of the present invention are unique in the sense that they are not found in a pure or separated state in nature. Use of the term “isolated” indicates that a naturally occurring sequence has been removed from its normal cellular environment. Thus, the sequence may be in a cell-free solution or placed in a different cellular environment. The term does not imply that the sequence is the only amino acid chain present, but that it is essentially free (at least about 90% pure, more preferably at least about 95% pure or more) of non-amino acid-based material naturally associated with it.

By the use of the term “enriched” in reference to a polypeptide is meant that the specific amino acid sequence constitutes a significantly higher fraction (2- to 5-fold) of the total amino acid sequences present in the cells or solution of interest than in normal or diseased cells or in the cells from which the sequence was taken. This could be caused by a person by preferential reduction in the amount of other amino acid sequences present, or by a preferential increase in the amount of the specific amino acid sequence of interest, or by a combination of the two. However, it should be noted that enriched does not imply that there are no other amino acid sequences present, just that the relative amount of the sequence of interest has been significantly increased. The term significant here is used to indicate that the level of increase is useful to the person making such an increase, and generally means an increase relative to other amino acid sequences of about at least 2-fold, more preferably at least 5- to 10-fold or even more. The term also does not imply that there is no amino acid sequence from other sources. The other source of amino acid sequences may, for example, comprise amino acid sequence encoded by a yeast or bacterial genome, or a cloning vector such as pUC19. The term is meant to cover only those situations in which man has intervened to increase the proportion of the desired amino acid sequence.

It is also advantageous for some purposes that an amino acid sequence be in purified form. The term “purified” in reference to a polypeptide does not require absolute purity (such as a homogeneous preparation); instead, it represents an indication that the sequence is relatively purer than in the natural environment. Compared to the natural level this level should be at least 2-to 5-fold greater (e.g., in terms of mg/mL). Purification of at least one order of magnitude, preferably two or three orders, and more preferably four or five orders of magnitude is expressly contemplated. The substance is preferably free of contamination at a functionally significant level, for example 90%, 95%, or 99% pure.

In preferred embodiments, the protease polypeptide is a fragment of the protein encoded by a full-length amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118. Preferably, the protease polypeptide contains at least 32, 45, 50, 60, 100, 200, or 300 contiguous amino acids of a full-length sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118, or a functional derivative thereof.

In preferred embodiments, the protease polypeptide comprises an amino acid sequence having an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118.

The polypeptide can be isolated from a natural source by methods well-known in the art. The natural source may be mammalian, preferably human, blood, semen, or tissue, and the polypeptide may be synthesized using an automated polypeptide synthesizer.

In some embodiments the invention includes a recombinant protease polypeptide having (a) an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118. By “recombinant protease polypeptide” is meant a polypeptide produced by recombinant DNA techniques such that it is distinct from a naturally occurring polypeptide either in its location (e.g., present in a different cell or tissue than found in nature), purity or structure. Generally, such a recombinant polypeptide will be present in a cell in an amount different from that normally observed in nature.

The polypeptides to be expressed in host cells may also be fusion proteins which include regions from heterologous proteins. Such regions may be included to allow, e.g., secretion, improved stability, or facilitated purification of the polypeptide. For example, a sequence encoding an appropriate signal peptide can be incorporated into expression vectors. A DNA sequence for a signal peptide (secretory leader) may be fused in-frame to the polynucleotide sequence so that the polypeptide is translated as a fusion protein comprising the signal peptide. A signal peptide that is functional in the intended host cell promotes extracellular secretion of the polypeptide. Preferably, the signal sequence will be cleaved from the polypeptide upon secretion of the polypeptide from the cell. Thus, preferred fusion proteins can be produced in which the N-terminus of a protease polypeptide is fused to a carrier peptide.

In one embodiment, the polypeptide comprises a fusion protein which includes a heterologous region used to facilitate purification of the polypeptide. Many of the available peptides used for such a function allow selective binding of the fusion protein to a binding partner. A preferred binding partner includes one or more of the IgG binding domains of protein A are easily purified to homogeneity by affinity chromatography on, for example, IgG-coupled Sepharose. Alternatively, many vectors have the advantage of carrying a stretch of histidine residues that can be expressed at the N-terminal or C-terminal end of the target protein, and thus the protein of interest can be recovered by metal chelation chromatography. A nucleotide sequence encoding a recognition site for a proteolytic enzyme such as enterokinase, factor X procollagenase or thrombine may immediately precede the sequence for a protease polypeptide to permit cleavage of the fusion protein to obtain the mature protease polypeptide. Additional examples of fusion-protein binding partners include, but are not limited to, the yeast I-factor, the honeybee melatin leader in sf9 insect cells, 6-His tag, thioredoxin tag, hemaglutinin tag, GST tag, and OmpA signal sequence tag. As will be understood by one of skill in the art, the binding partner which recognizes and binds to the peptide may be any ion, molecule or compound including metal ions (e.g., metal affinity columns), antibodies, or fragments thereof, and any protein or peptide which binds the peptide, such as the FLAG tag.

Antibodies

In another aspect, the invention features an antibody (e.g., a monoclonal or polyclonal antibody) having specific binding affinity to a protease polypeptide or a protease polypeptide domain or fragment where the polypeptide is selected from the group having a sequence at least about 90% identical to an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118. Preferably the polypeptide is has at least about 90%, 91%, 92%, 93%, 94%, 95%, 96%, 97%, 98% 99% or 100% identity with the sequences listed above. By “specific binding affinity” is meant that the antibody binds to the target protease polypeptide with greater affinity than it binds to other polypeptides under specified conditions. Antibodies or antibody fragments are polypeptides that contain regions that can bind other polypeptides. The term “specific binding affinity” describes an antibody that binds to a protease polypeptide with greater affinity than it binds to other polypeptides under specified conditions. Antibodies can be used to identify an endogenous source of protease polypeptides, to monitor cell cycle regulation, and for immuno-localization of protease polypeptides within the cell.

The term “polyclonal” refers to antibodies that are heterogenous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

“Monoclonal antibodies” are substantially homogenous populations of antibodies to a particular antigen. They may be obtained by any technique which provides for the production of antibody molecules by continuous cell lines in culture. Monoclonal antibodies may be obtained by methods known to those skilled in the art (Kohler et al., Nature, 1975, 256:495-497, and U.S. Pat. No. 4,376,110, both of which are hereby incorporated by reference herein in their entirety including any figures, tables, or drawings).

An antibody of the present invention includes “humanized” monoclonal and polyclonal antibodies. Humanized antibodies are recombinant proteins in which non-human (typically murine) complementarity determining regions of an antibody have been transferred from heavy and light variable chains of the non-human (e.g. murine) immunoglobulin into a human variable domain, followed by the replacement of some human residues in the framework regions of their murine counterparts. Humanized antibodies in accordance with this invention are suitable for use in therapeutic methods. General techniques for cloning murine immunoglobulin variable domains are described, for example, by the publication of Orlandi et al., Proc. Nat'l Acad. Sci. USA 86: 3833 (1989). Techniques for producing humanized monoclonal antibodies are described, for example, by Jones et al., Nature 321:522 (1986), Riechmann et al., Nature 332:323 (1988), Verhoeyen et al., Science 239:1534 (1988), Carter et al., Proc. Nat'l Acad. Sci. USA 89:4285 (1992), Sandhu, Crit. Rev. Biotech. 12:437 (1992), and Singer et al., J. Immun. 150:2844 (1993).

The term “antibody fragment” refers to a portion of an antibody, often the hypervariable region and portions of the surrounding heavy and light chains, that displays specific binding affinity for a particular molecule. A hypervariable region is a portion of an antibody that physically binds to the polypeptide target.

An antibody fragment of the present invention includes a “single-chain antibody,” a phrase used in this description to denote a linear polypeptide that binds antigen with specificity and that comprises variable or hypervariable regions from the heavy and light chain chains of an antibody. Such single chain antibodies can be produced by conventional methodology. The Vh and Vl regions of the Fv fragment can be covalently joined and stabilized by the insertion of a disulfide bond. See Glockshuber, et al., Biochemistry 1362 (1990). Alternatively, the Vh and Vl regions can be joined by the insertion of a peptide linker. A gene encoding the Vh, VI and peptide linker sequences can be constructed and expressed using a recombinant expression vector. See Colcher, et al., J. Nat'l Cancer Inst. 82:1191(1990). Amino acid sequences comprising hypervariable regions from the Vh and Vl antibody chains can also be constructed using disulfide bonds or peptide linkers.

Antibodies or antibody fragments having specific binding affinity to a protease polypeptide of the invention may be used in methods for detecting the presence and/or amount of protease polypeptide in a sample by probing the sample with the antibody under conditions suitable for protease-antibody immunocomplex formation and detecting the presence and/or amount of the antibody conjugated to the protease polypeptide. Diagnostic kits for performing such methods may be constructed to include antibodies or antibody fragments specific for the protease as well as a conjugate of a binding partner of the antibodies or the antibodies themselves.

An antibody or antibody fragment with specific binding affinity to a protease polypeptide of the invention can be isolated, enriched, or purified from a prokaryotic or eukaryotic organism. Routine methods known to those skilled in the art enable production of antibodies or antibody fragments, in both prokaryotic and eukaryotic organisms. Purification, enrichment, and isolation of antibodies, which are polypeptide molecules, are described above.

Antibodies having specific binding affinity to a protease polypeptide of the invention may be used in methods for detecting the presence and/or amount of protease polypeptide in a sample by contacting the sample with the antibody under conditions such that an immunocomplex forms and detecting the presence and/or amount of the antibody conjugated to the protease polypeptide. Diagnostic kits for performing such methods may be constructed to include a first container containing the antibody and a second container having a conjugate of a binding partner of the antibody and a label, such as, for example, a radioisotope. The diagnostic kit may also include notification of an FDA approved use and instructions therefor.

In another aspect, the invention features a hybridoma which produces an antibody having specific binding affinity to a protease polypeptide or a protease polypeptide domain, where the polypeptide is selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118. By “hybridoma” is meant an immortalized cell line that is capable of secreting an antibody, for example an antibody to a protease of the invention. In preferred embodiments, the antibody to the protease comprises a sequence of amino acids that is able to specifically bind a protease polypeptide of the invention.

In another aspect, the present invention is also directed to kits comprising antibodies that bind to a polypeptide encoded by any of the nucleic acid molecules described above, and a negative control antibody.

The term “negative control antibody” refers to an antibody derived from similar source as the antibody having specific binding affinity, but where it displays no binding affinity to a polypeptide of the invention.

In another aspect, the invention features a protease polypeptide binding agent able to bind to a protease polypeptide selected from the group having an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118. The binding agent is preferably a purified antibody that recognizes an epitope present on a protease polypeptide of the invention. Other binding agents include molecules that bind to protease polypeptides and analogous molecules that bind to a protease polypeptide. Such binding agents may be identified by using assays that measure protease binding partner activity, or they may be identified using assays that measure protease activity, such as the release of a fluorogenic or radioactive marker attached to a substrate molecule.

Screening Methods to Detect Protease Polypeptides

The invention also features a method for screening for human cells containing a protease polypeptide of the invention or an equivalent sequence. The method involves identifying the novel polypeptide in human cells using techniques that are routine and standard in the art, such as those described herein for identifying the proteases of the invention (e.g., cloning, Southern or Northern blot analysis, in situ hybridization, PCR amplification, etc.).

Screening Methods to Identify Substances that Modulate Protease Activity

In another aspect, the invention features methods for identifying a substance that modulates protease activity comprising the steps of: (a) contacting a protease polypeptide comprising an amino acid sequence substantially identical to a sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118 with a test substance; (b) measuring the activity of said polypeptide; and (c) determining whether said substance modulates the activity of said polypeptide. More preferably the sequence is at least about 90%, 91%, 92%, 93%, 94%, 95%, 96%, 97%, 98% or 99% identical to the listed sequences.

The term “modulates” refers to the ability of a compound to alter the function of a protease of the invention. A modulator preferably activates or inhibits the activity of a protease of the invention depending on the concentration of the compound exposed to the protease.

The term “modulates” also refers to altering the function of proteases of the invention by increasing or decreasing the probability that a complex forms between the protease and a natural binding partner. A modulator preferably increases the probability that such a complex forms between the protease and the natural binding partner, more preferably increases or decreases the probability that a complex forms between the protease and the natural binding partner depending on the concentration of the compound exposed to the protease, and most preferably decreases the probability that a complex forms between the protease and the natural binding partner.

The term “activates” refers to increasing the cellular activity of the protease. The term “inhibits” refers to decreasing the cellular activity of the protease.

The term “complex” refers to an assembly of at least two molecules bound to one another. Signal transduction complexes often contain at least two protein molecules bound to one another. For instance, a protein tyrosine receptor protein kinase, GRB2, SOS, RAF, and RAS assemble to form a signal transduction complex in response to a mitogenic ligand. Similarly, the proteases involved in blood coagulation and their cofactors are known to form macromolecular complexes on cellular membranes. Additionally, proteases involved in modification of the extracellular matrix are known to form complexes with their inhibitors and also with components of the extracellular matrix.

The term “natural binding partner” refers to polypeptides, lipids, small molecules, or nucleic acids that bind to proteases in cells. A change in the interaction between a protease and a natural binding partner can manifest itself as an increased or decreased probability that the interaction forms, or an increased or decreased concentration of protease/natural binding partner complex.

The term “contacting” as used herein refers to mixing a solution comprising the test compound with a liquid medium bathing the cells of the methods. The solution comprising the compound may also comprise another component, such as dimethyl sulfoxide (DMSO), which facilitates the uptake of the test compound or compounds into the cells of the methods. The solution comprising the test compound may be added to the medium bathing the cells by utilizing a delivery apparatus, such as a pipette-based device or syringe-based device.

In another aspect, the invention features methods for identifying a substance that modulates protease activity in a cell comprising the steps of: (a) expressing a protease polypeptide in a cell, wherein said polypeptide has a sequence substantially identical to an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:100, SEQ ID NO:111, SEQ ID NO:112, SEQ ID NO:113, SEQ ID NO:114, SEQ ID NO:115, SEQ ID NO:116, SEQ ID NO:117 and SEQ ID NO:118; (b) adding a test substance to said cell; and (c) monitoring a change in cell phenotype, cell proliferation, cell differentiation or the interaction between said polypeptide and a natural binding partner.

The term “expressing” as used herein refers to the production of proteases of the invention from a nucleic acid vector containing protease genes within a cell. The nucleic acid vector is transfected into cells using well known techniques in the art as described herein.

Another aspect of the instant invention is directed to methods of identifying compounds that bind to protease polypeptides of the present invention, comprising contacting the protease polypeptides with a compound, and determining whether the compound binds the protease polypeptides. Binding can be determined by binding assays which are well known to the skilled artisan, including, but not limited to, gel-shift assays, Western blots, radiolabeled competition assay, phage-based expression cloning, co-fractionation by chromatography, co-precipitation, cross linking, interaction trap/two-hybrid analysis, southwestern analysis, ELISA, and the like, which are described in, for example, Current Protocols in Molecular Biology, 1999, John Wiley & Sons, NY, which is incorporated herein by reference in its entirety. The compounds to be screened include, but are not limited to, compounds of extracellular, intracellular, biological or chemical origin.

The methods of the invention also embrace compounds that are attached to a label, such as a radiolabel (e.g., ¹²⁵I, ³⁵S, ³²P, ³³P, ³H), a fluorescence label, a chemiluminescent label, an enzymic label and an immunogenic label. The protease polypeptides employed in such a test may either be free in solution, attached to a solid support, borne on a cell surface, located intracellularly or associated with a portion of a cell. One skilled in the art can, for example, measure the formation of complexes between a protease polypeptide and the compound being tested. Alternatively, one skilled in the art can examine the diminution in complex formation between a protease polypeptide and its substrate caused by the compound being tested.

Other assays can be used to examine enzymatic activity including, but not limited to, photometric, radiometric, HPLC, electrochemical, and the like, which are described in, for example, Enzyme Assays: A Practical Approach, eds. R. Eisenthal and M. J. Danson, 1992, Oxford University Press, which is incorporated herein by reference in its entirety.

Another aspect of the present invention is directed to methods of identifying compounds which modulate (i.e., increase or decrease) activity of a protease polypeptide comprising contacting the protease polypeptide with a compound, and determining whether the compound modifies activity of the protease polypeptide. These compounds are also referred to as “modulators of proteases.” The activity in the presence of the test compound is measured to the activity in the absence of the test compound. Where the activity of a sample containing the test compound is higher than the activity in a sample lacking the test compound, the compound will have increased the activity. Similarly, where the activity of a sample containing the test compound is lower than the activity in the sample lacking the test compound, the compound will have inhibited the activity.

The present invention is particularly useful for screening compounds by using a protease polypeptide in any of a variety of drug screening techniques. The compounds to be screened include, but are not limited to, extracellular, intracellular, biological or chemical origin. The protease polypeptide employed in such a test may be in any form, preferably, free in solution, attached to a solid support, borne on a cell surface or located intracellularly. One skilled in the art can measure the change in rate that a protease of the invention cleaves a substrate polypeptide. One skilled in the art can also, for example, measure the formation of complexes between a protease polypeptide and the compound being tested. Alternatively, one skilled in the art can examine the diminution in complex formation between a protease polypeptide and its substrate caused by the compound being tested.

The activity of protease polypeptides of the invention can be determined by, for example, examining the ability to bind or be activated by chemically synthesised peptide ligands. Alternatively, the activity of the protease polypeptides can be assayed by examining their ability to bind metal ions such as calcium, hormones, chemokines, neuropeptides, neurotransmitters, nucleotides, lipids, odorants, and photons. Thus, modulators of the protease polypeptide's activity may alter a protease function, such as a binding property of a protease or an activity such as cleaving protein substrates or polypeptide substrates, or membrane localization.

In various embodiments of the method, the assay may take the form of a yeast growth assay, an Aequorin assay, a Luciferase assay, a mitogenesis assay, a MAP Kinase activity assay, as well as other binding or function-based assays of protease activity that are generally known in the art. In several of these embodiments, the invention includes any of the serine proteases, cysteine proteases, aspartyl proteases, metalloproteases, threonine proteases, and other proteases. Biological activities of proteases according to the invention include, but are not limited to, the binding of a natural or a synthetic ligand, as well as any one of the functional activities of proteases known in the art. Non-limiting examples of protease activities include cleavage of polypeptide chains, processing the pro-form of a polypeptide chain to the active product, transmembrane signaling of various forms, and/or the modification of the extraceullar matrix.

The modulators of the invention exhibit a variety of chemical structures, which can be generally grouped into mimetics of natural protease ligands, and peptide and non-peptide allosteric effectors of proteases. The invention does not restrict the sources for suitable modulators, which may be obtained from natural sources such as plant, animal or mineral extracts, or non-natural sources such as small molecule libraries, including the products of combinatorial chemical approaches to library construction, and peptide libraries.

The use of cDNAs encoding proteins in drug discovery programs is well-known; assays capable of testing thousands of unknown compounds per day in high-throughput screens (HTSs) are thoroughly documented. The literature is replete with examples of the use of radiolabelled ligands in HTS binding assays for drug discovery (see, Williams, Medicinal Research Reviews, 1991, 11:147-184; Sweetnam, et al., J. Natural Products, 1993, 56:441-455 for review). Recombinant proteins are preferred for binding assay HTS because they allow for better specificity (higher relative purity), provide the ability to generate large amounts of receptor material, and can be used in a broad variety of formats (see Hodgson, Bio/Technology, 1992, 10:973-980 which is incorporated herein by reference in its entirety). A variety of heterologous systems is available for functional expression of recombinant proteins that are well known to those skilled in the art. Such systems include bacteria (Strosberg, et al., Trends in Pharmacological Sciences, 1992, 13:95-98), yeast (Pausch, Trends in Biotechnology, 1997, 15:487-494), several kinds of insect cells (Vanden Broeck, Int. Rev. Cytology, 1996, 164:189-268), amphibian cells (Jayawickreme et al., Current Opinion in Biotechnology, 1997, 8:629-634) and several mammalian cell lines (CHO, HEK293, COS, etc.; see, Gerhardt, et al., Eur. J. Pharmacology, 1997, 334:1-23). These examples do not preclude the use of other possible cell expression systems, including cell lines obtained from nematodes (PCT application WO 98/37177).

An expressed protease can be used for HTS binding assays in conjunction with its defined ligand, in this case the corresponding peptide that activates it. The identified peptide is labeled with a suitable radioisotope, including, but not limited to, ¹²⁵I, ³H, 35S or ³²P, by methods that are well known to those skilled in the art. Alternatively, the peptides may be labeled by well-known methods with a suitable fluorescent derivative (Baindur, et al, Drug Dev. Res., 1994, 33:373-398; Rogers, Drug Discovery Today, 1997, 2:156-160). Radioactive ligand specifically bound to the receptor in membrane preparations made from the cell line expressing the recombinant protein can be detected in HTS assays in one of several standard ways, including filtration of the receptor-ligand complex to separate bound ligand from unbound ligand (Williams, Med. Res. Rev., 1991, 11:147-184.; Sweetnam, et al, J. Natural Products, 1993, 56:441-455). Alternative methods include a scintillation proximity assay (SPA) or a FlashPlate format in which such separation is unnecessary (Nakayama, Cur. Opinion Drug Disc. Dev., 1998, 1:85-91 Bossé, et al., J. Biomolecular Screening, 1998, 3:285-292.). Binding of fluorescent ligands can be detected in various ways, including fluorescence energy transfer (FRET), direct spectrophotofluorometric analysis of bound ligand, or fluorescence polarization (Rogers, Drug Discovery Today, 1997, 2:156-160; Hill, Cur. Opinion Drug Disc. Dev., 1998, 1:92-97).

The proteases and natural binding partners required for functional expression of heterologous protease polypeptides can be native constituents of the host cell or can be introduced through well-known recombinant technology. The protease polypeptides can be intact or chimeric. The protease activation may result in the stimulation or inhibition of other native proteins, events that can be linked to a measurable response.

Examples of such biological responses include, but are not limited to, the following: the ability to survive in the absence of a limiting nutrient in specifically engineered yeast cells (Pausch, Trends in Biotechnology, 1997, 15:487-494); changes in intracellular Ca²⁺ concentration as measured by fluorescent dyes (Murphy, et al., Cur. Opinion Drug Disc. Dev., 1998, 1:192-199). Fluorescence changes can also be used to monitor ligand-induced changes in membrane potential or intracellular pH; an automated system suitable for HTS has been described for these purposes (Schroeder, et al., J. Biomolecular Screening, 1996, 1:75-80). Assays are also available for the measurement of common second but these are not generally preferred for HTS.

The invention contemplates a multitude of assays to screen and identify inhibitors of ligand binding to protease polypeptides or of substrate cleavage by protease polypeptides. In one example, the protease polypeptide is immobilized and interaction with a binding partner or substrate is assessed in the presence and absence of a candidate modulator such as an inhibitor compound. In another example, interaction between the protease polypeptide and its binding partner or a substrate is assessed in a solution assay, both in the presence and absence of a candidate inhibitor compound. In either assay, an inhibitor is identified as a compound that decreases binding between the protease polypeptide and its natural binding partner or the activity of a protease polypeptide in cleaving a substrate molecule. Another contemplated assay involves a variation of the di-hybrid assay wherein an inhibitor of protein/protein interactions is identified by detection of a positive signal in a transformed or transfected host cell, as described in PCT publication number WO 95/20652, published Aug. 3, 1995 and is included by reference herein including any figures, tables, or drawings.

Candidate modulators contemplated by the invention include compounds selected from libraries of either potential activators or potential inhibitors. There are a number of different libraries used for the identification of small molecule modulators, including: (1) chemical libraries, (2) natural product libraries, and (3) combinatorial libraries comprised of random peptides, oligonucleotides or organic molecules. Chemical libraries consist of random chemical structures, some of which are analogs of known compounds or analogs of compounds that have been identified as “hits” or “leads” in other drug discovery screens, while others are derived from natural products, and still others arise from non-directed synthetic organic chemistry. Natural product libraries are collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see, Science 282:63-68 (1998). Combinatorial libraries are composed of large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see, Myers, Curr. Opin. Biotechnol. 8:701-707 (1997). Identification of modulators through use of the various libraries described herein permits modification of the candidate “hit” (or “lead”) to optimize the capacity of the “hit” to modulate activity.

Still other candidate inhibitors contemplated by the invention can be designed and include soluble forms of binding partners, as well as such binding partners as chimeric, or fusion, proteins. A “binding partner” as used herein broadly encompasses both natural binding partners as described above as well as chimeric polypeptides, peptide modulators other than natural ligands, antibodies, antibody fragments, and modified compounds comprising antibody domains that are immunospecific for the expression product of the identified protease gene.

Other assays may be used to identify specific peptide ligands of a protease polypeptide, including assays that identify ligands of the target protein through measuring direct binding of test ligands to the target protein, as well as assays that identify ligands of target proteins through affinity ultrafiltration with ion spray mass spectroscopy/HPLC methods or other physical and analytical methods. Alternatively, such binding interactions are evaluated indirectly using the yeast two-hybrid system described in Fields et al., Nature, 340:245-246 (1989), and Fields et al., Trends in Genetics, 10:286-292 (1994), both of which are incorporated herein by reference. The two-hybrid system is a genetic assay for detecting interactions between two proteins or polypeptides. It can be used to identify proteins that bind to a known protein of interest, or to delineate domains or residues critical for an interaction. Variations on this methodology have been developed to clone genes that encode DNA binding proteins, to identify peptides that bind to a protein, and to screen for drugs. The two-hybrid system exploits the ability of a pair of interacting proteins to bring a transcription activation domain into close proximity with a DNA binding domain that binds to an upstream activation sequence (UAS) of a reporter gene, and is generally performed in yeast. The assay requires the construction of two hybrid genes encoding (1) a DNA-binding domain that is fused to a first protein and (2) an activation domain fused to a second protein. The DNA-binding domain targets the first hybrid protein to the UAS of the reporter gene; however, because most proteins lack an activation domain, this DNA-binding hybrid protein does not activate transcription of the reporter gene. The second hybrid protein, which contains the activation domain, cannot by itself activate expression of the reporter gene because it does not bind the UAS. However, when both hybrid proteins are present, the noncovalent interaction of the first and second proteins tethers the activation domain to the UAS, activating transcription of the reporter gene. For example, when the first protein is a protease gene product, or fragment thereof, that is known to interact with another protein or nucleic acid, this assay can be used to detect agents that interfere with the binding interaction. Expression of the reporter gene is monitored as different test agents are added to the system. The presence of an inhibitory agent results in lack of a reporter signal.

When the function of the protease polypeptide gene product is unknown and no ligands are known to bind the gene product, the yeast two-hybrid assay can also be used to identify proteins that bind to the gene product. In an assay to identify proteins that bind to a protease polypeptide, or fragment thereof, a fusion polynucleotide encoding both a protease polypeptide (or fragment) and a UAS binding domain (i.e., a first protein) may be used. In addition, a large number of hybrid genes each encoding a different second protein fused to an activation domain are produced and screened in the assay. Typically, the second protein is encoded by one or more members of a total cDNA or genomic DNA fusion library, with each second protein coding region being fused to the activation domain. This system is applicable to a wide variety of proteins, and it is not even necessary to know the identity or function of the second binding protein. The system is highly sensitive and can detect interactions not revealed by other methods; even transient interactions may trigger transcription to produce a stable mRNA that can be repeatedly translated to yield the reporter protein.

Other assays may be used to search for agents that bind to the target protein. One such screening method to identify direct binding of test ligands to a target protein is described in U.S. Pat. No. 5,585,277, incorporated herein by reference. This method relies on the principle that proteins generally exist as a mixture of folded and unfolded states, and continually alternate between the two states. When a test ligand binds to the folded form of a target protein (i e., when the test ligand is a ligand of the target protein), the target protein molecule bound by the ligand remains in its folded state. Thus, the folded target protein is present to a greater extent in the presence of a test ligand which binds the target protein, than in the absence of a ligand. Binding of the ligand to the target protein can be determined by any method which distinguishes between the folded and unfolded states of the target protein. The function of the target protein need not be known in order for this assay to be performed. Virtually any agent can be assessed by this method as a test ligand, including, but not limited to, metals, polypeptides, proteins, lipids, polysaccharides, polynucleotides and small organic molecules.

Another method for identifying ligands of a target protein is described in Wieboldt et al., Anal. Chem., 69:1683-1691 (1997), incorporated herein by reference. This technique screens combinatorial libraries of 20-30 agents at a time in solution phase for binding to the target protein. Agents that bind to the target protein are separated from other library components by simple membrane washing. The specifically selected molecules that are retained on the filter are subsequently liberated from the target protein and analyzed by HPLC and pneumatically assisted electrospray (ion spray) ionization mass spectroscopy. This procedure selects library components with the greatest affinity for the target protein, and is particularly useful for small molecule libraries.

In preferred embodiments of the invention, methods of screening for compounds which modulate protease activity comprise contacting test compounds with protease polypeptides and assaying for the presence of a complex between the compound and the protease polypeptide. In such assays, the ligand is typically labelled. After suitable incubation, free ligand is separated from that present in bound form, and the amount of free or uncomplexed label is a measure of the ability of the particular compound to bind to the protease polypeptide.

In another embodiment of the invention, high throughput screening for compounds having suitable binding affinity to protease polypeptides is employed. Briefly, large numbers of different small peptide test compounds are synthesised on a solid substrate. The peptide test compounds are contacted with the protease polypeptide and washed. Bound protease polypeptide is then detected by methods well known in the art. Purified polypeptides of the invention can also be coated directly onto plates for use in the aforementioned drug screening techniques. In addition, non-neutralizing antibodies can be used to capture the protein and immobilize it on the solid support.

Other embodiments of the invention comprise using competitive screening assays in which neutralizing antibodies capable of binding a polypeptide of the invention specifically compete with a test compound for binding to the polypeptide. In this manner, the antibodies can be used to detect the presence of any peptide that shares one or more antigenic determinants with a protease polypeptide. Radiolabeled competitive binding studies are described in A. H. Lin et al. Antimicrobial Agents and Chemotherapy, 1997, vol. 41, no. 10. pp. 2127-2131, the disclosure of which is incorporated herein by reference in its entirety.

Therapeutic Methods

The invention includes methods for treating a disease or disorder by administering to a patient in need of such treatment a protease polypeptide substantially identical to an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118, and any other protease polypeptide of the present invention. As discussed in the section “Gene Therapy,” a protease polypeptide of the invention may also be administered indirectly by via administration of suitable polynucleotide means for in vivo expression of the protease polypeptide. Preferably the protease polypeptide will have at least about 90%, 91%, 92%, 93%, 94%, 95%, 96%, 97%, 98%, 99% or 100% identity to one of the aforementioned sequences.

In another aspect, the invention provides methods for treating a disease or disorder by administering to a patient in need of such treatment a substance that modulates the activity of a protease substantially identical to a sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118.

Preferably the disease is selected from the group consisting of cancers, immune-related diseases and disorders, cardiovascular disease, brain or neuronal-associated diseases, and metabolic disorders. More specifically these diseases include cancer of tissues, blood, or hematopoietic origin, particularly those involving breast, colon, lung, prostate, cervical, brain, ovarian, bladder, or kidney; central or peripheral nervous system-diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

In preferred embodiments, the invention provides methods for treating or preventing a disease or disorder by administering to a patient in need of such treatment a substance that modulates the activity of a protease polypeptide having an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118.

Preferably the disease is selected from the group consisting of cancers, immune-related diseases and disorders, cardiovascular disease, brain or neuronal-associated diseases, and metabolic disorders. More specifically these diseases include cancer of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

Preferably the disease is selected from the group consisting of immune-related diseases and disorders, cardiovascular disease, and cancer. Most preferably, the immune-related diseases and disorders are selected from the group consisting of rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplantation.

Substances useful for treatment of protease-related disorders or diseases preferably show positive results in one or more in vitro assays for an activity corresponding to treatment of the disease or disorder in question (Examples of such assays are provided herein, including Example 7). Examples of substances that can be screened for favorable activity are provided and referenced throughout the specification, including this section (Screening Methods to Identify Substances that Modulate Protease Activity). The substances that modulate the activity of the proteases preferably include, but are not limited to, antisense oligonucleotides, ribozymes, and other inhibitors of proteases, as determined by methods and screens referenced this section and in Example 7, below, and any other suitable methods. The use of antisense oligonucleotides and ribozymes are discussed more fully in the Section “Gene Therapy,” below.

The term “preventing” refers to decreasing the probability that an organism contracts or develops an abnormal condition.

The term “treating” refers to having a therapeutic effect and at least partially alleviating or abrogating an abnormal condition in the organism.

The term “therapeutic effect” refers to the inhibition or activation factors causing or contributing to the abnormal condition. A therapeutic effect relieves to some extent one or more of the symptoms of the abnormal condition. In reference to the treatment of abnormal conditions, a therapeutic effect can refer to one or more of the following: (a) an increase or decrease in the proliferation, growth, and/or differentiation of cells; (b) activation or inhibition (i.e., slowing or stopping) of cell death; (c) inhibition of degeneration; (d) relieving to some extent one or more of the symptoms associated with the abnormal condition; and (e) enhancing the function of the affected population of cells. Compounds demonstrating efficacy against abnormal conditions can be identified as described herein.

The term “abnormal condition” refers to a function in the cells or tissues of an organism that deviates from their normal functions in that organism. An abnormal condition can relate to cell proliferation, cell differentiation, or cell survival.

Abnormal cell proliferative conditions include cancers such as fibrotic and mesangial disorders, abnormal angiogenesis and vasculogenesis, wound healing, psoriasis, diabetes mellitus, and inflammation.

Abnormal differentiation conditions include, but are not limited to neurodegenerative disorders, slow wound healing rates, and slow tissue grafting healing rates.

Abnormal cell survival conditions relate to conditions in which programmed cell death (apoptosis) pathways are activated or abrogated. A number of proteases are associated with the apoptosis pathways. Aberrations in the function of any one of the proteases could lead to cell immortality or premature cell death.

The term “aberration”, in conjunction with the function of a protease in a signal transduction process, refers to a protease that is over- or under-expressed in an organism, mutated such that its catalytic activity is lower or higher than wild-type protease activity, mutated such that it can no longer interact with a natural binding partner, is no longer modified by another protein, or no longer interacts with a natural binding partner.

The term “administering” relates to a method of incorporating a compound into cells or tissues of an organism. The abnormal condition can be prevented or treated when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral, parenteral, dermal, injection, and aerosol applications. For cells outside of the organism, multiple techniques exist in the art to administer the compounds, including (but not limited to) cell microinjection techniques, transformation techniques, and carrier techniques.

The abnormal condition can also be prevented or treated by administering a compound to a group of cells having an aberration in a signal transduction pathway to an organism. The effect of administering a compound on organism function can then be monitored. The organism is preferably a mouse, rat, rabbit, guinea pig, or goat, more preferably a monkey or ape, and most preferably a human.

In another aspect, the invention features methods for detection of a protease polypeptide in a sample as a diagnostic tool for diseases or disorders, wherein the method comprises the steps of: (a) contacting the sample with a nucleic acid probe which hybridizes under hybridization assay conditions to a nucleic acid target region of a protease polypeptide having an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118, said probe comprising the nucleic acid sequence encoding the polypeptide, fragments thereof, and the complements of the sequences and fragments; and (b) detecting the presence or amount of the probe:target region hybrid as an indication of the disease.

In preferred embodiments of the invention, the disease or disorder is selected from the group consisting of rheumatoid arthritis, arteriosclerosis, autoimmune disorders, organ transplantation, myocardial infarction, cardiomyopathies, stroke, renal failure, oxidative stress-related neurodegenerative disorders, and cancer. Preferably the disease is selected from the group consisting of cancers, immune-related diseases and disorders, cardiovascular disease, brain or neuronal-associated diseases, and metabolic disorders. More specifically these diseases include cancer of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

The protease “target region” is the nucleotide base sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, and SEQ ID NO:59, or the corresponding full-length sequences, a functional derivative thereof, or a fragment thereof or a domain thereof to which the nucleic acid probe will specifically hybridize. Specific hybridization indicates that in the presence of other nucleic acids the probe only hybridizes detectably with the nucleic acid target region of the protease of the invention. Putative target regions can be identified by methods well known in the art consisting of alignment and comparison of the most closely related sequences in the database.

In preferred embodiments the nucleic acid probe hybridizes to a protease target region encoding at least 6, 12, 75, 90, 105, 120, 150, 200, 250, 300 or 350 contiguous amino acids of a sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118, or the corresponding full-length amino acid sequence, or a functional derivative thereof. Hybridization conditions should be such that hybridization occurs only with the protease genes in the presence of other nucleic acid molecules. Under stringent hybridization conditions only highly complementary nucleic acid sequences hybridize. Preferably, such conditions prevent hybridization of nucleic acids having more than 1 or 2 mismatches out of 20 contiguous nucleotides. Such conditions are defined in Berger et al. (1987) (Guide to Molecular Cloning Techniques pg 421, hereby incorporated by reference herein in its entirety including any figures, tables, or drawings.).

The diseases for which detection of protease genes in a sample could be diagnostic include diseases in which protease nucleic acid (DNA and/or RNA) is amplified in comparison to normal cells. By “amplification” is meant increased numbers of protease DNA or RNA in a cell compared with normal cells. In normal cells, proteases may be found as single copy genes. In selected diseases, the chromosomal location of the protease genes may be amplified, resulting in multiple copies of the gene, or amplification. Gene amplification can lead to amplification of protease RNA, or protease RNA can be amplified in the absence of protease DNA amplification.

“Amplification” as it refers to RNA can be the detectable presence of protease RNA in cells, since in some normal cells there is no basal expression of protease RNA. In other normal cells, a basal level of expression of protease exists, therefore in these cases amplification is the detection of at least 1-2-fold, and preferably more, protease RNA, compared to the basal level.

The diseases that could be diagnosed by detection of protease nucleic acid in a sample preferably include cancers. The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample that is compatible with the method utilized.

In a final aspect, the invention features a method for detection of a protease polypeptide in a sample as a diagnostic tool for a disease or disorder, wherein the method comprises: (a) comparing a nucleic acid target region encoding the protease polypeptide in a sample, where the protease polypeptide has an amino acid sequence selected from the group consisting those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118, or one or more fragments thereof, with a control nucleic acid target region encoding the protease polypeptide, or one or more fragments thereof; and (b) detecting differences in sequence or amount between the target region and the control target region, as an indication of the disease or disorder. Preferably the disease is selected from the group consisting of cancers, immune-related diseases and disorders, cardiovascular disease, brain or neuronal-associated diseases, and metabolic disorders.

More specifically these diseases include cancer of tissues, blood, or hematopoietic origin, particularly those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral or non-viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

The term “comparing” as used herein refers to identifying discrepancies between the nucleic acid target region isolated from a sample, and the control nucleic acid target region. The discrepancies can be in the nucleotide sequences, e.g. insertions, deletions, or point mutations, or in the amount of a given nucleotide sequence. Methods to determine these discrepancies in sequences are well-known to one of ordinary skill in the art. The “control” nucleic acid target region refers to the sequence or amount of the sequence found in normal cells, e.g. cells that are not diseased as discussed previously.

The term “domain” refers to a region of a polypeptide which serves a particular function. For instance, N-terminal or C-terminal domains of signal transduction proteins can serve functions including, but not limited to, binding molecules that localize the signal transduction molecule to different regions of the cell or binding other signaling molecules directly responsible for propagating a particular cellular signal. Some domains can be expressed separately from the rest of the protein and function by themselves, while others must remain part of the intact protein to retain function. The latter are termed functional regions of proteins and also relate to domains.

The expression of proteases can be modulated by signal transduction pathways such as the Ras/MAP kinase signaling pathways. Additionally, the activity of proteases can modulate the activity of the MAP kinase signal transduction pathway. Furthermore, proteases can be shown to be instrumental in the communication between disparate signal transduction pathways.

The term “signal transduction pathway” refers to the molecules that propagate an extracellular signal through the cell membrane to become an intracellular signal. This signal can then stimulate a cellular response. The polypeptide molecules involved in signal transduction processes are typically receptor and non-receptor protein tyrosine kinases, receptor and non-receptor protein phosphatases, polypeptides containing SRC homology 2 and 3 domains, phosphotyrosine binding proteins (SRC homology 2 (SH2) and phosphotyrosine binding (PTB and PH) domain containing proteins), proline-rich binding proteins (SH3 domain containing proteins), GTPases, phosphodiesterases, phospholipases, prolyl isomerases, proteases, Ca²⁺ binding proteins, cAMP binding proteins, guanyl cyclases, adenylyl cyclases, NO generating proteins, nucleotide exchange factors, and transcription factors.

The summary of the invention described above is not limiting and other features and advantages of the invention will be apparent from the following detailed description of the invention, and from the claims.

### BRIEF DESCRIPTION OF THE FIGURES

FIGS. 1A-WW shows the nucleotide sequences for human proteases oriented in a 5′ to 3′ direction (SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, and SEQ ID NO:59). In the sequences, N means any nucleotide.

FIG. 2A-S shows the amino acid sequences for the human proteases encoded by SEQ ID No. 1-59 in the direction of translation (SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70 SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118). In the sequences, X means any amino acid.

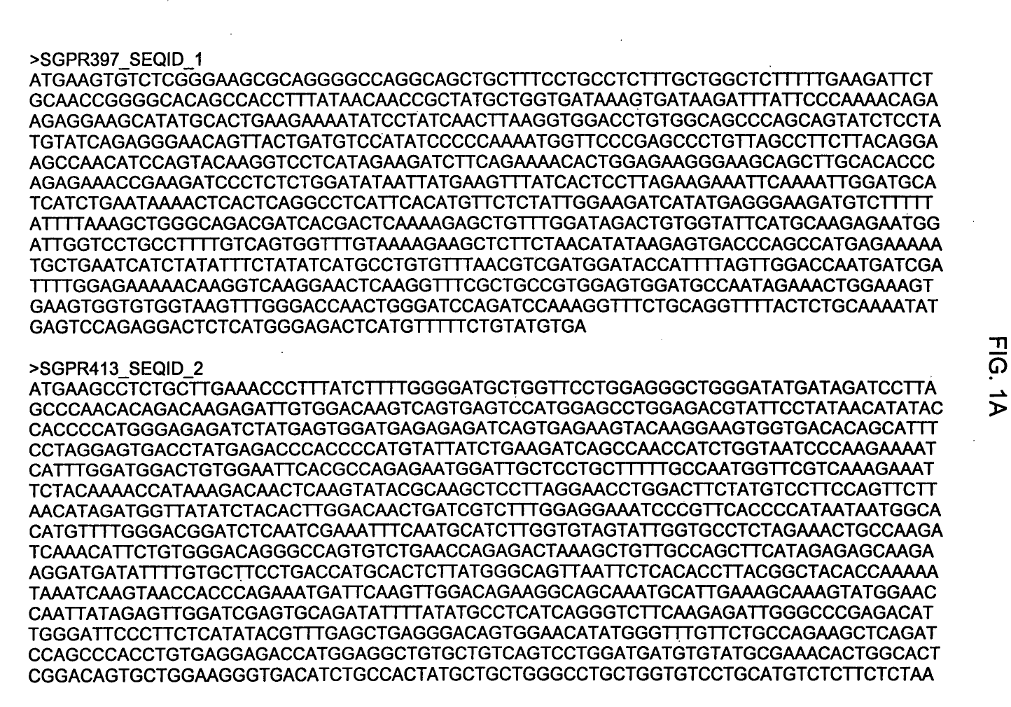

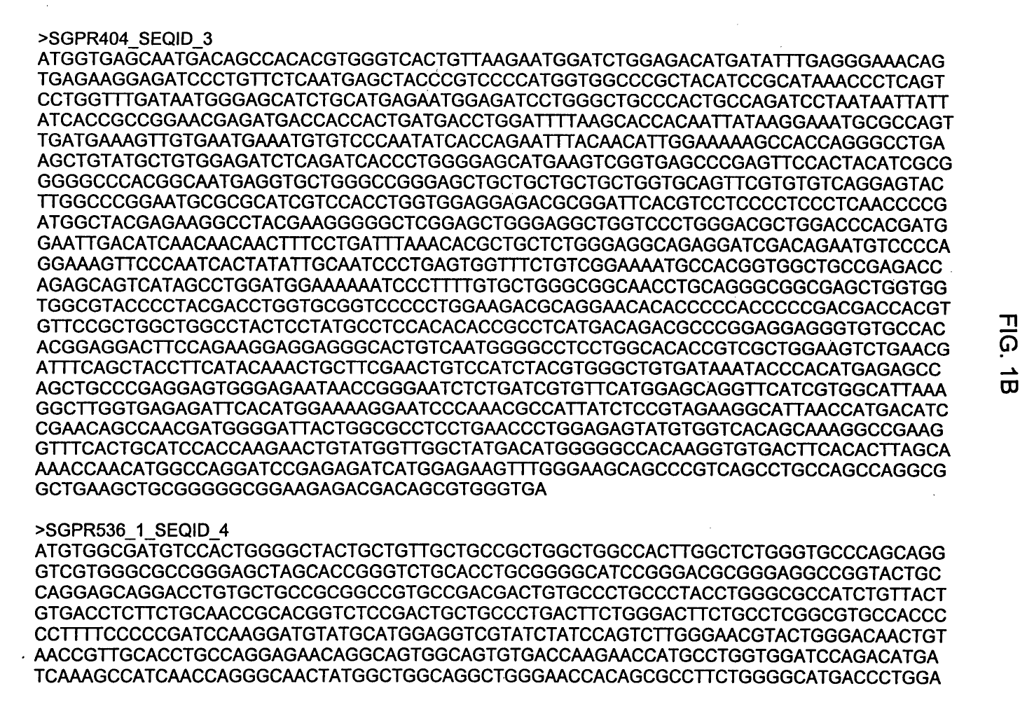

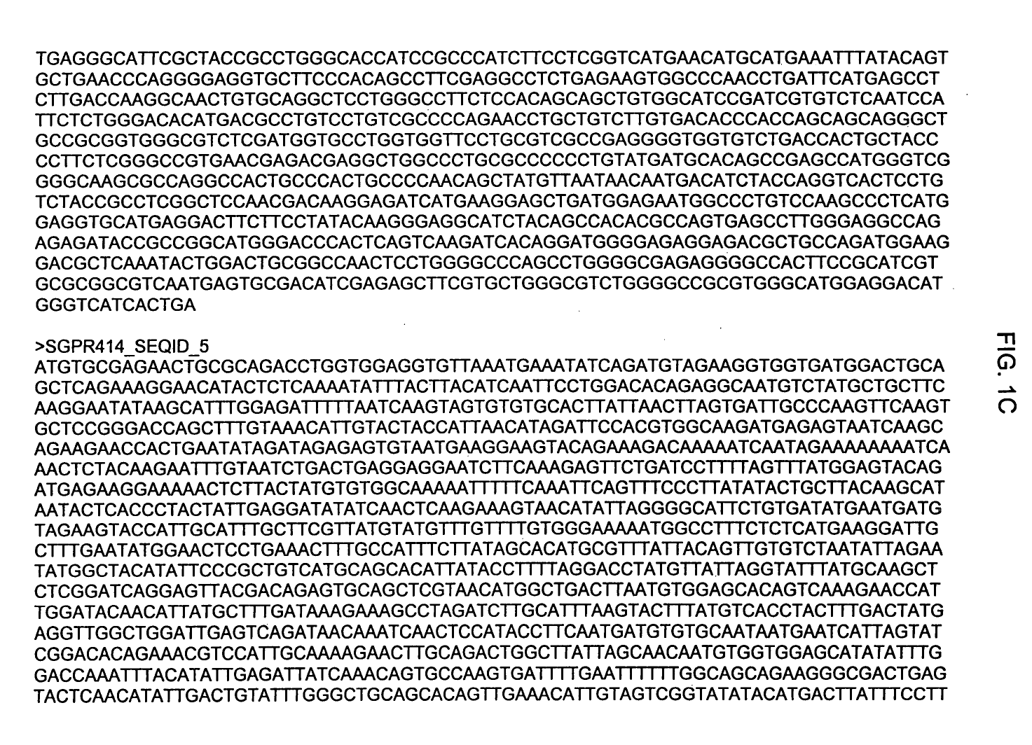

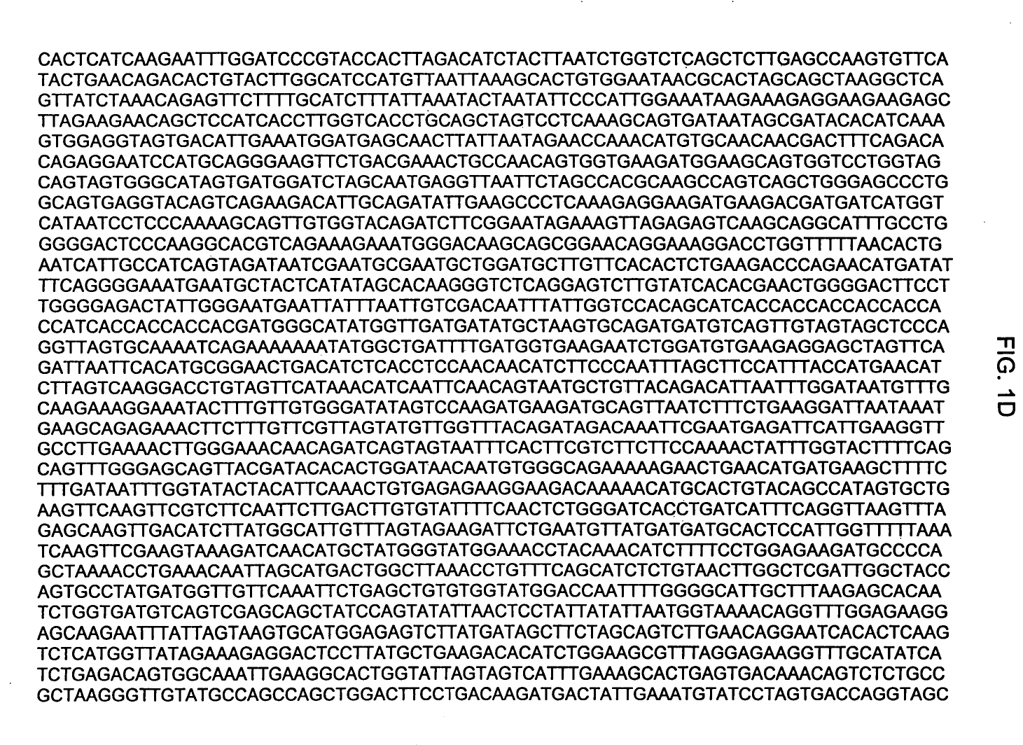

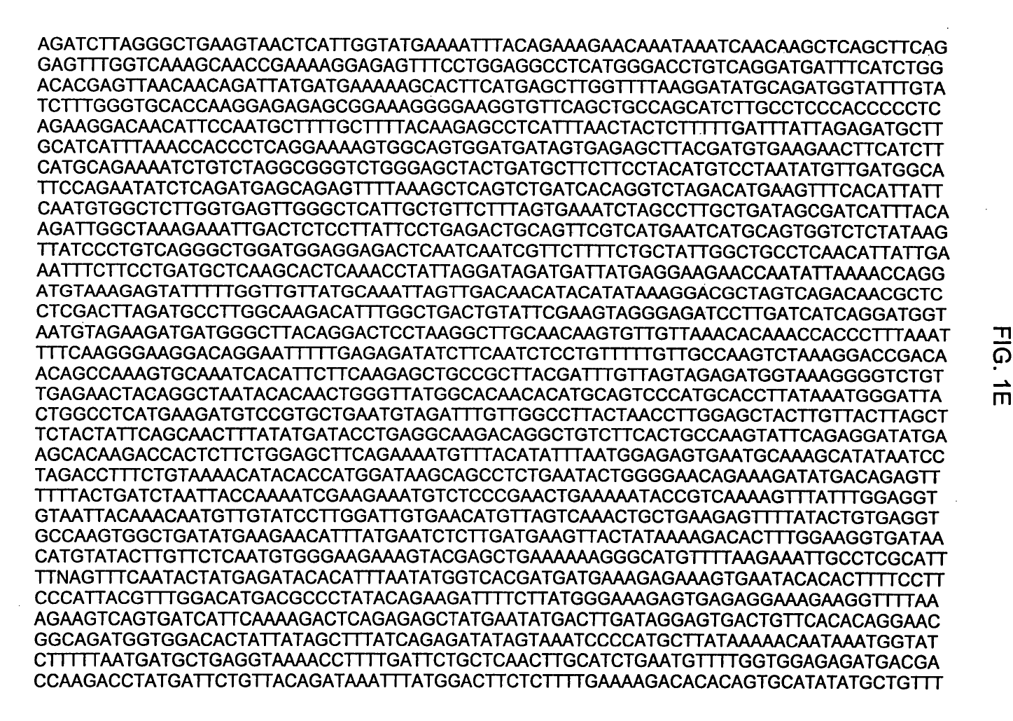

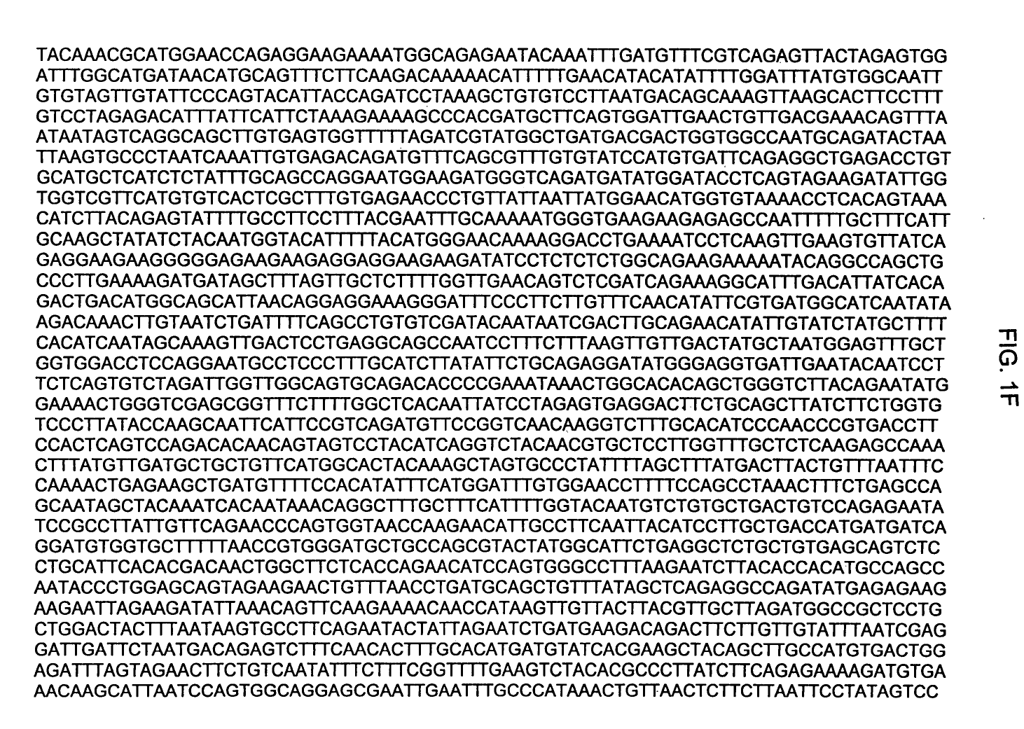

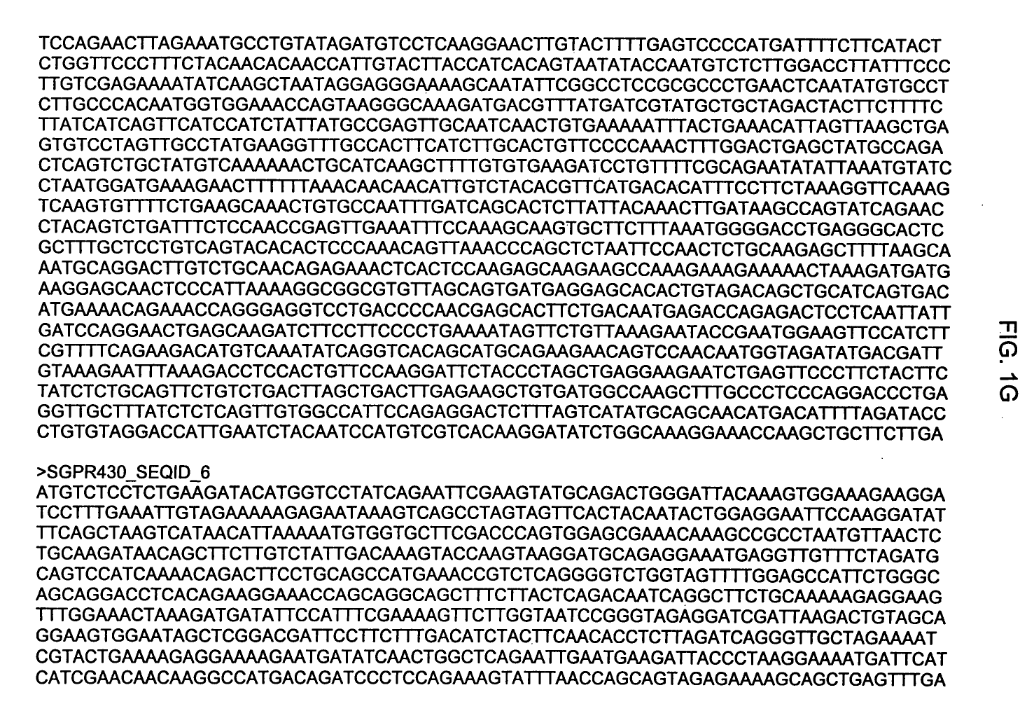

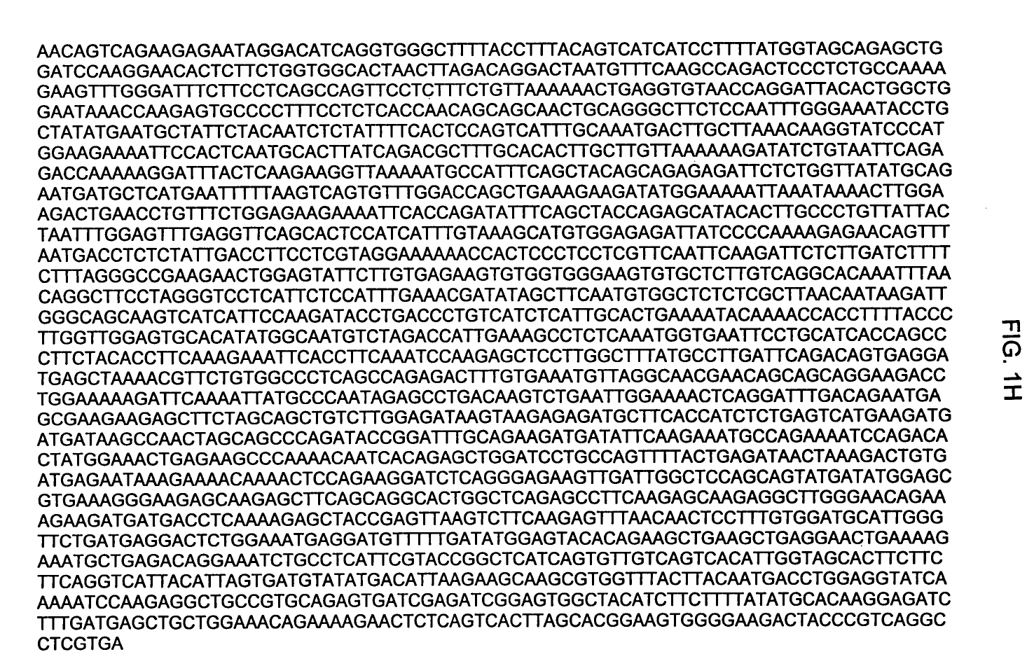

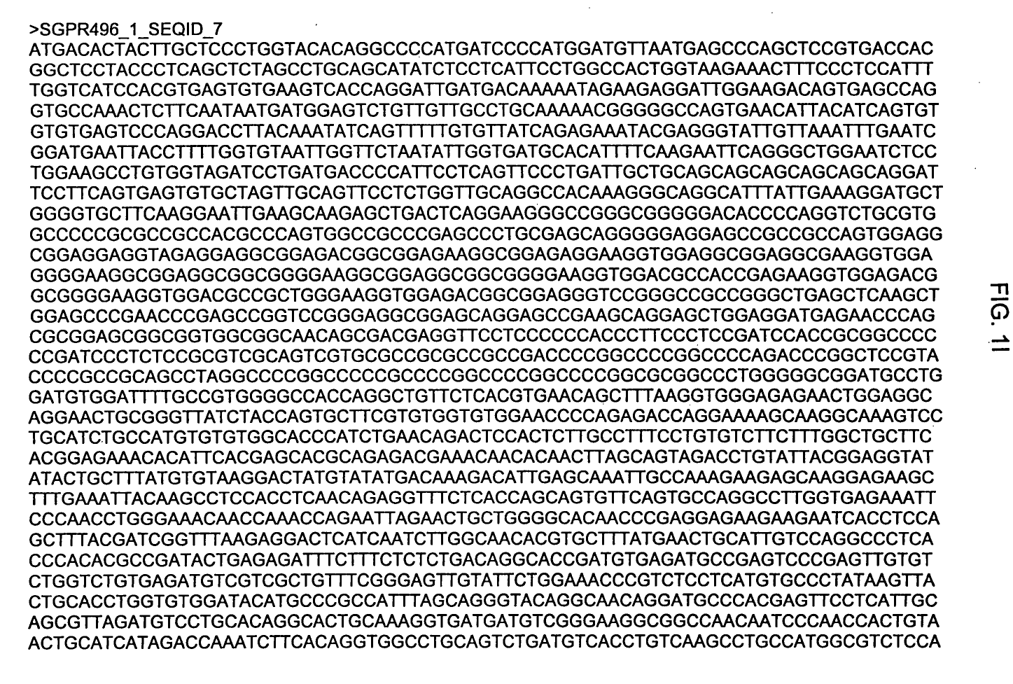

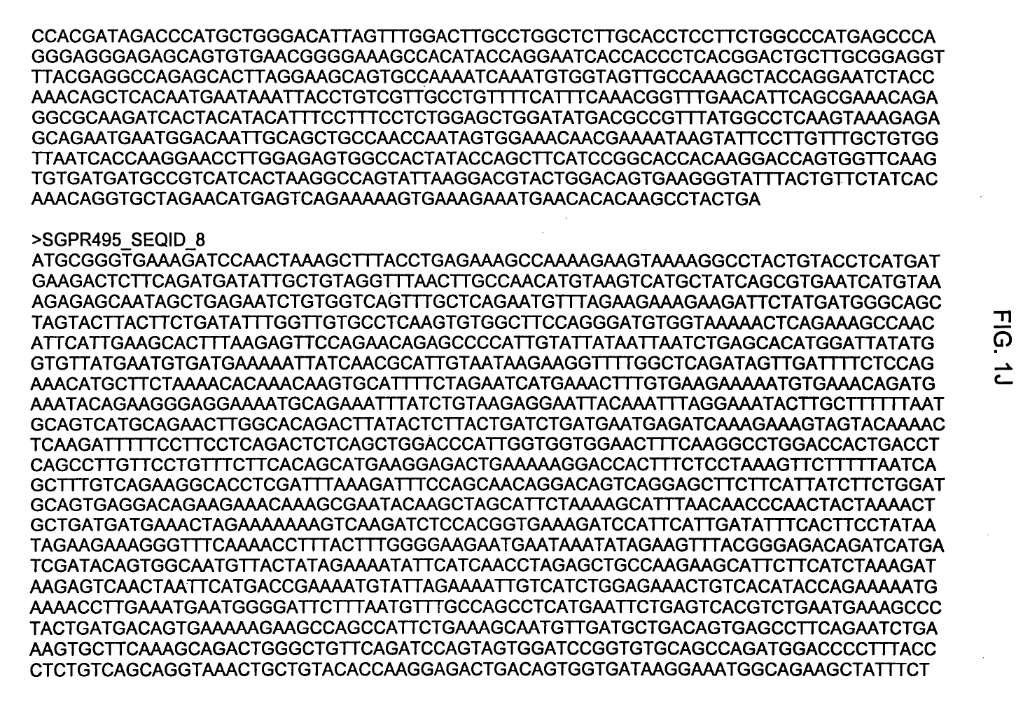

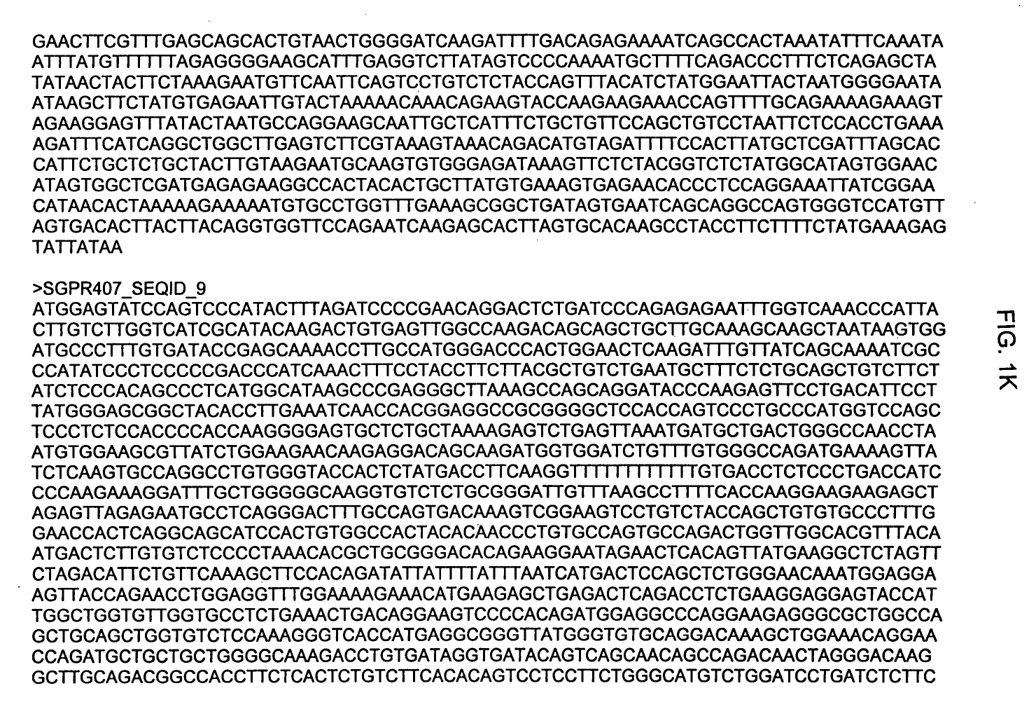

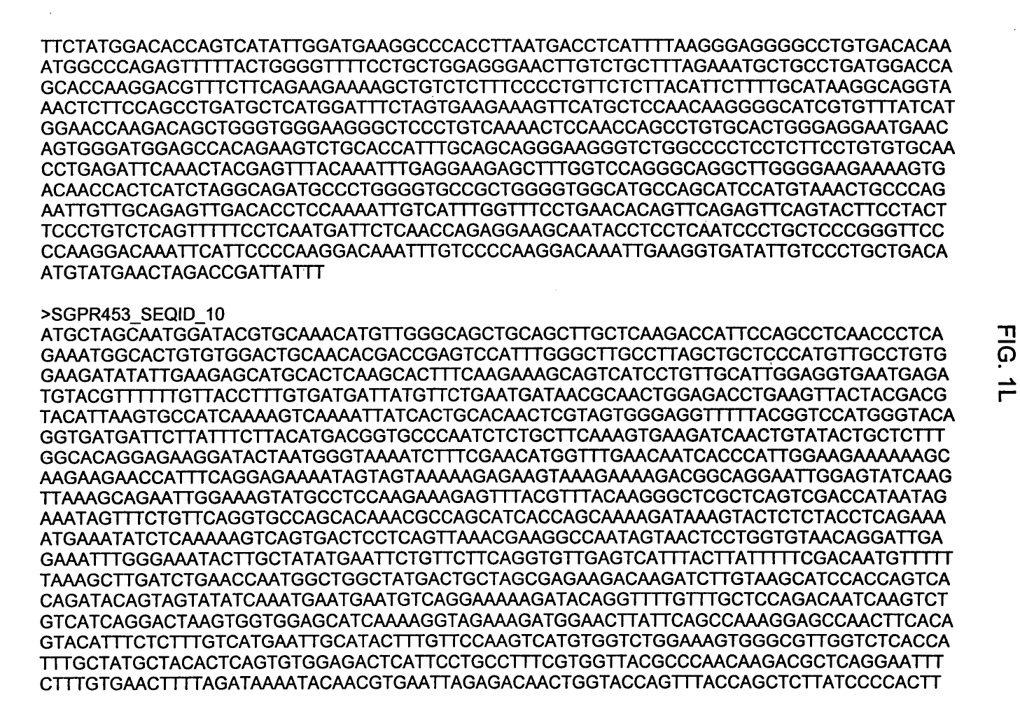

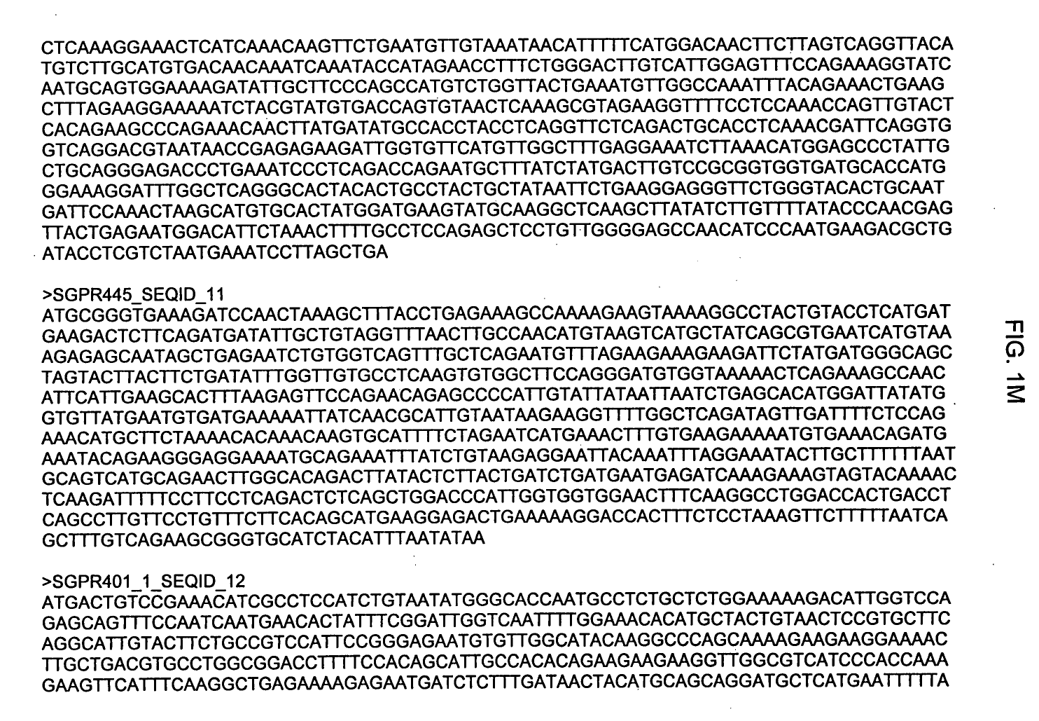

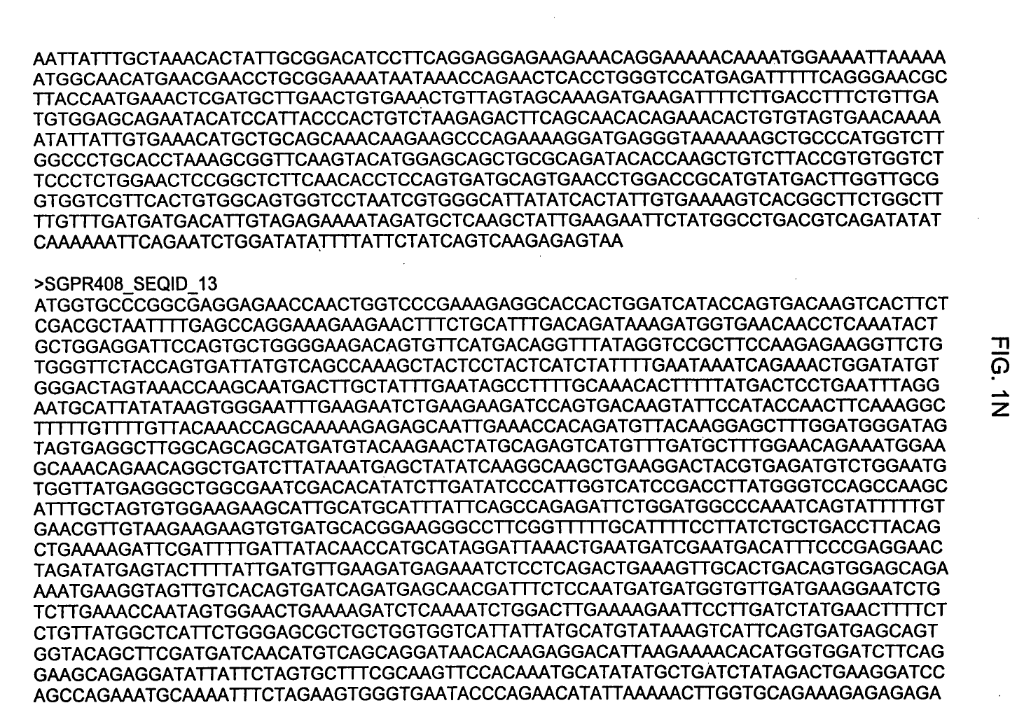

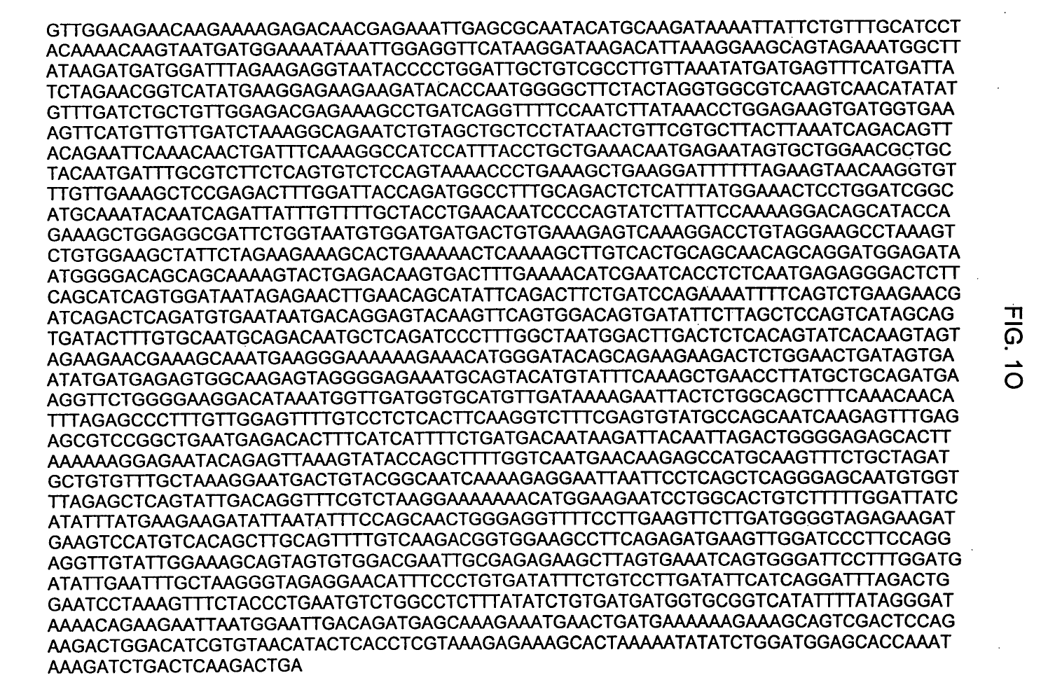

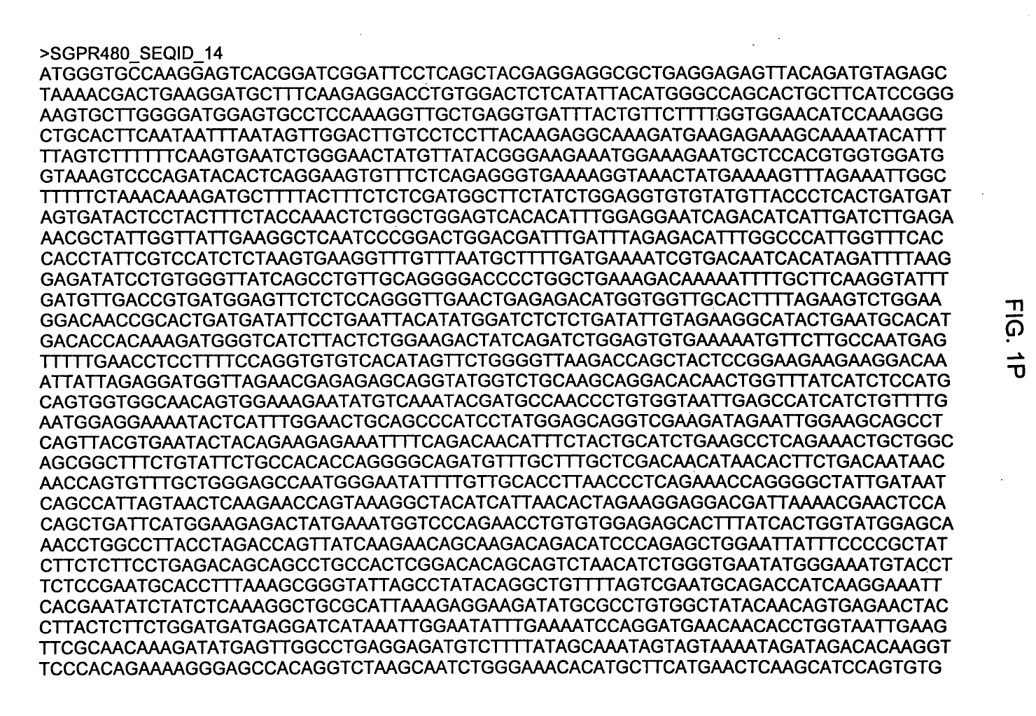

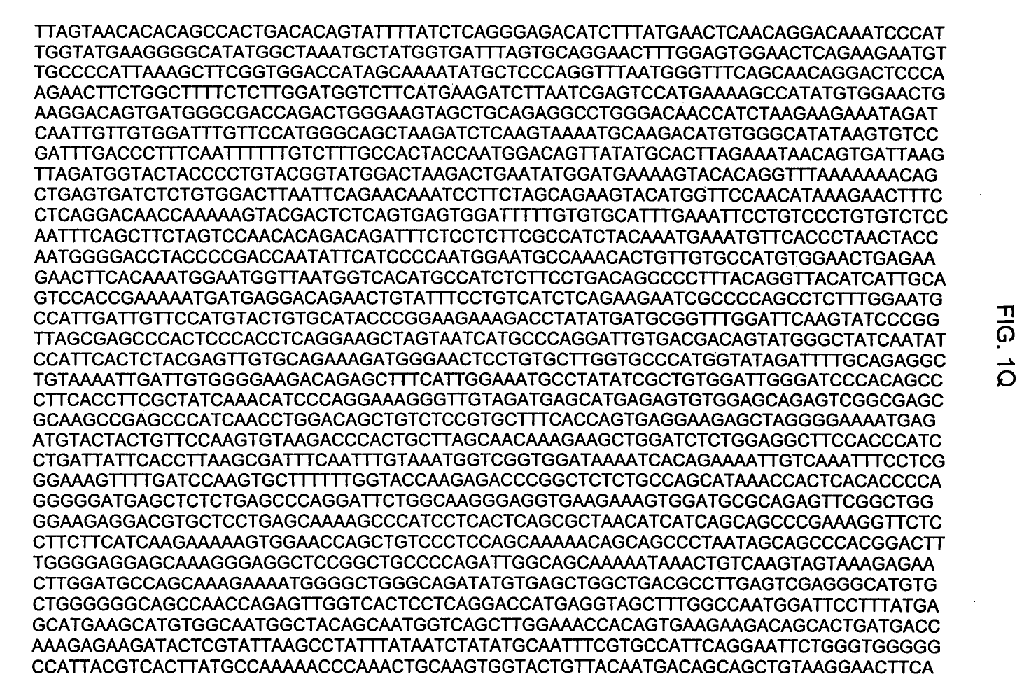

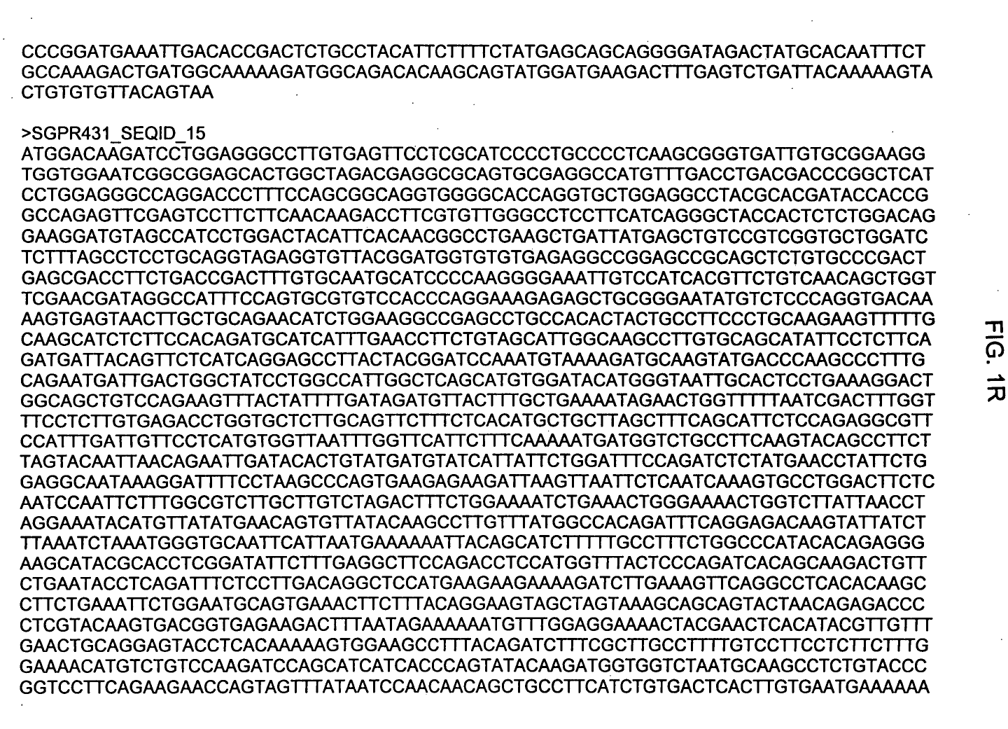

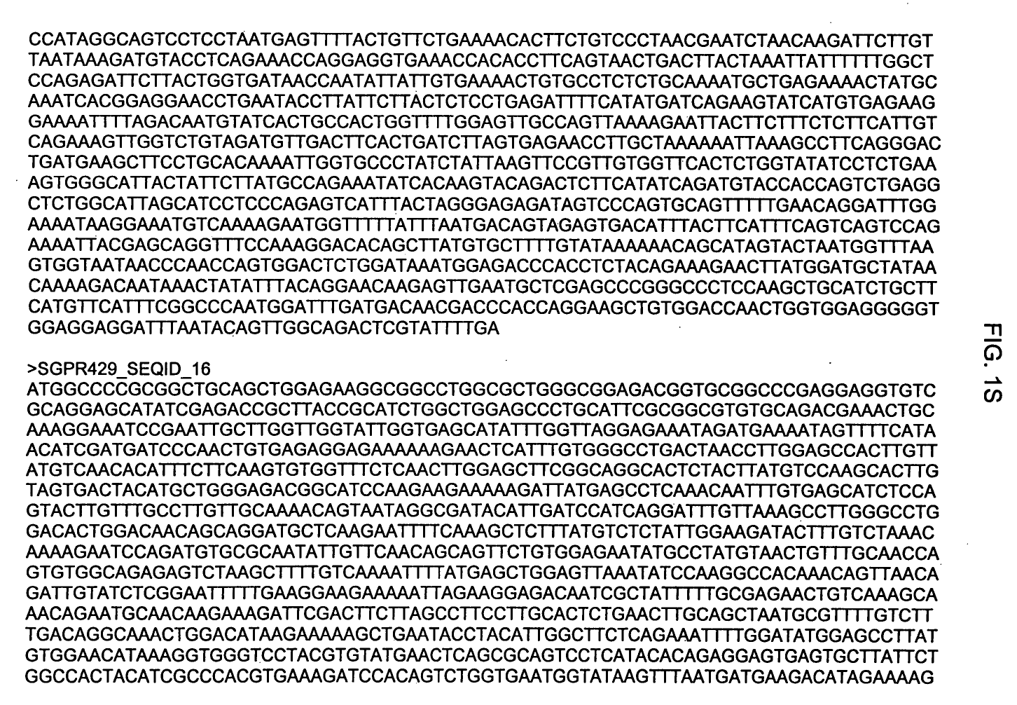

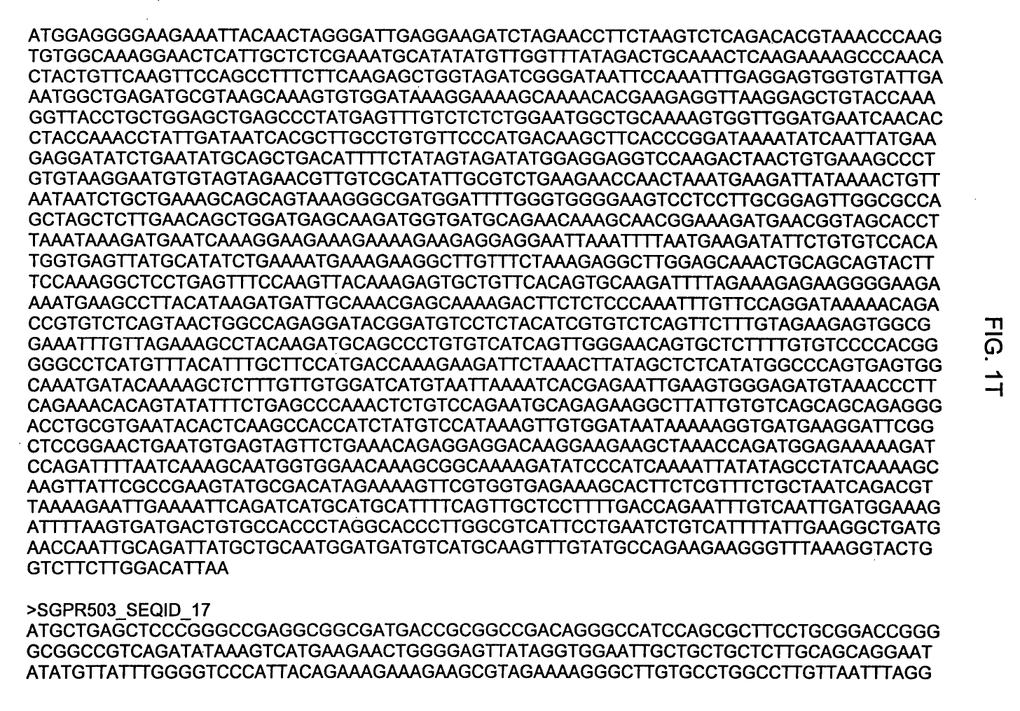

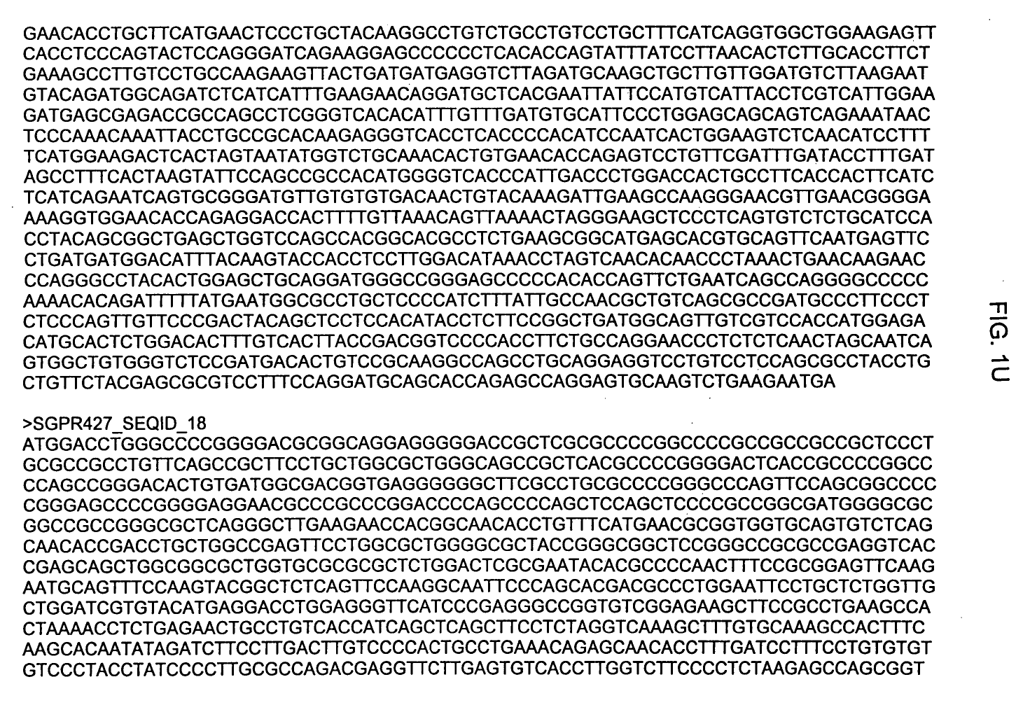

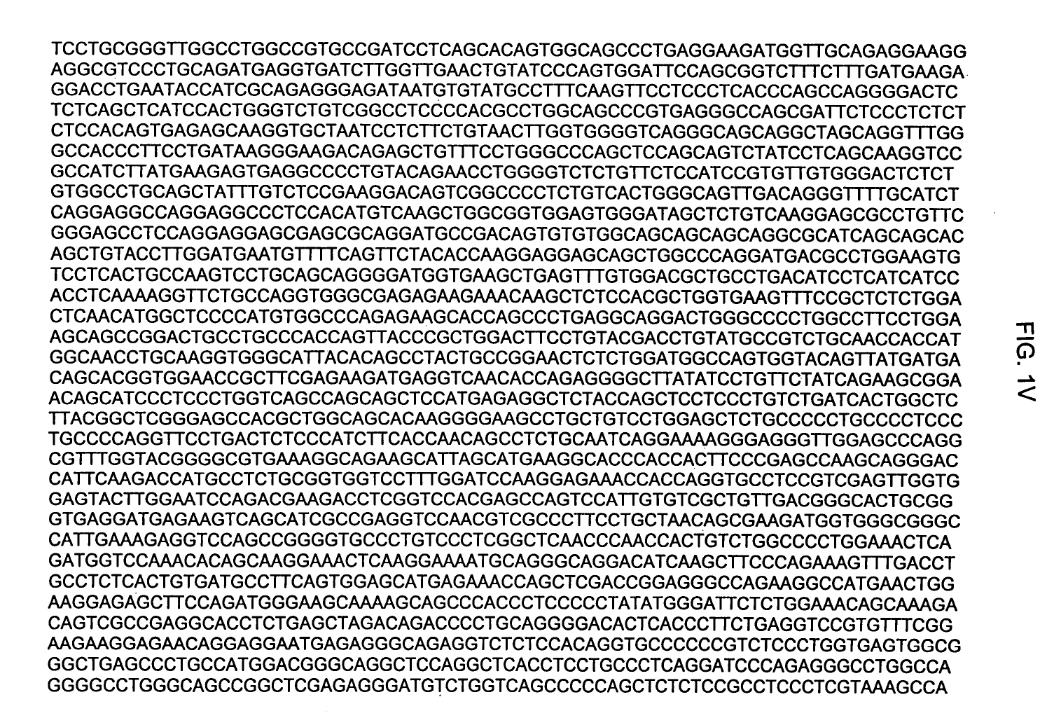

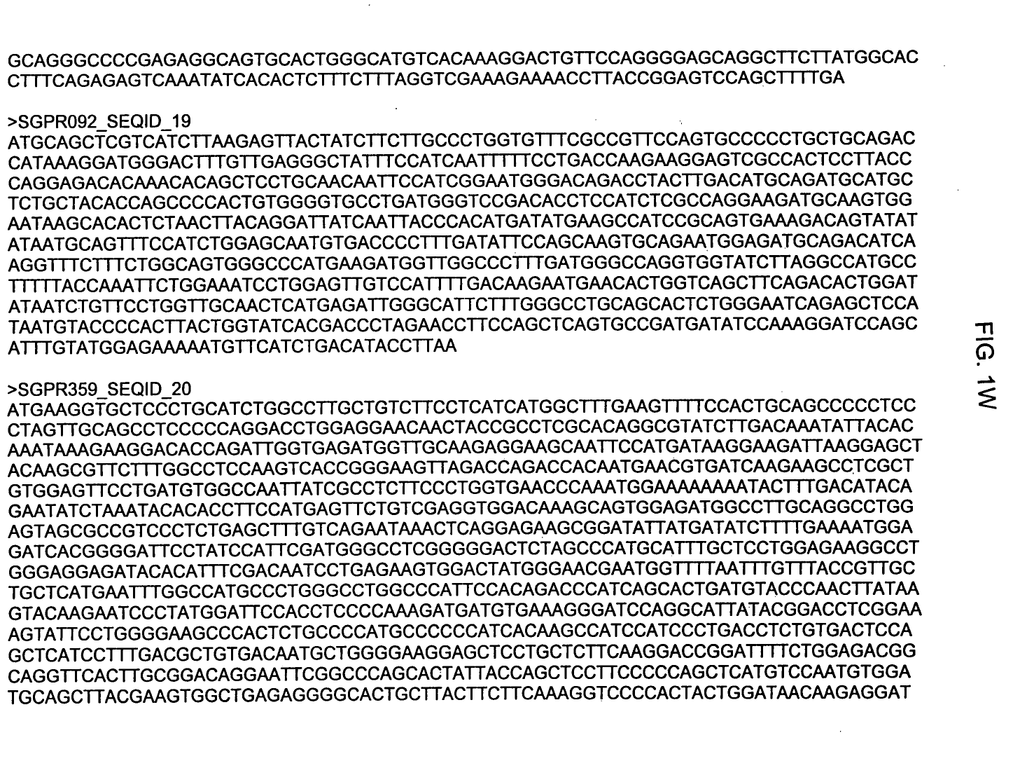

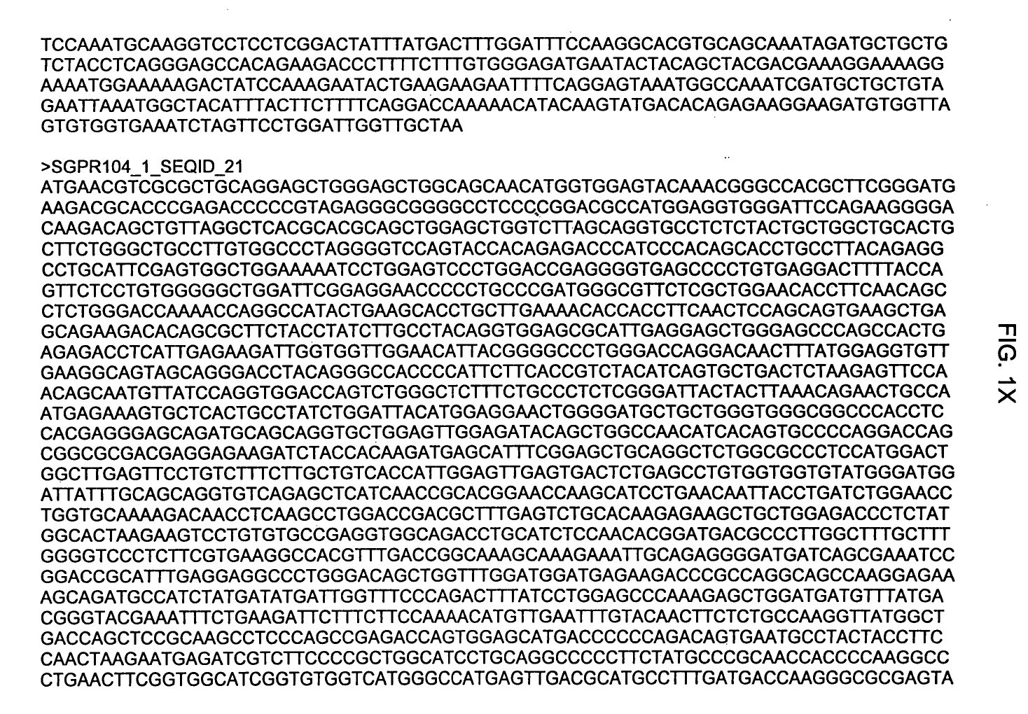

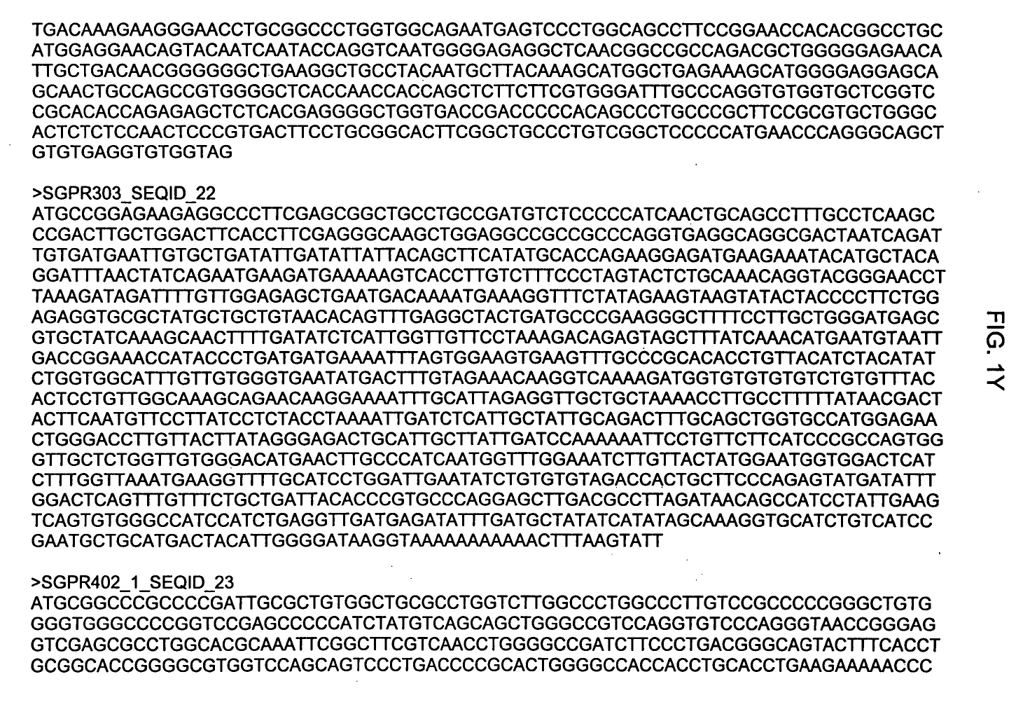

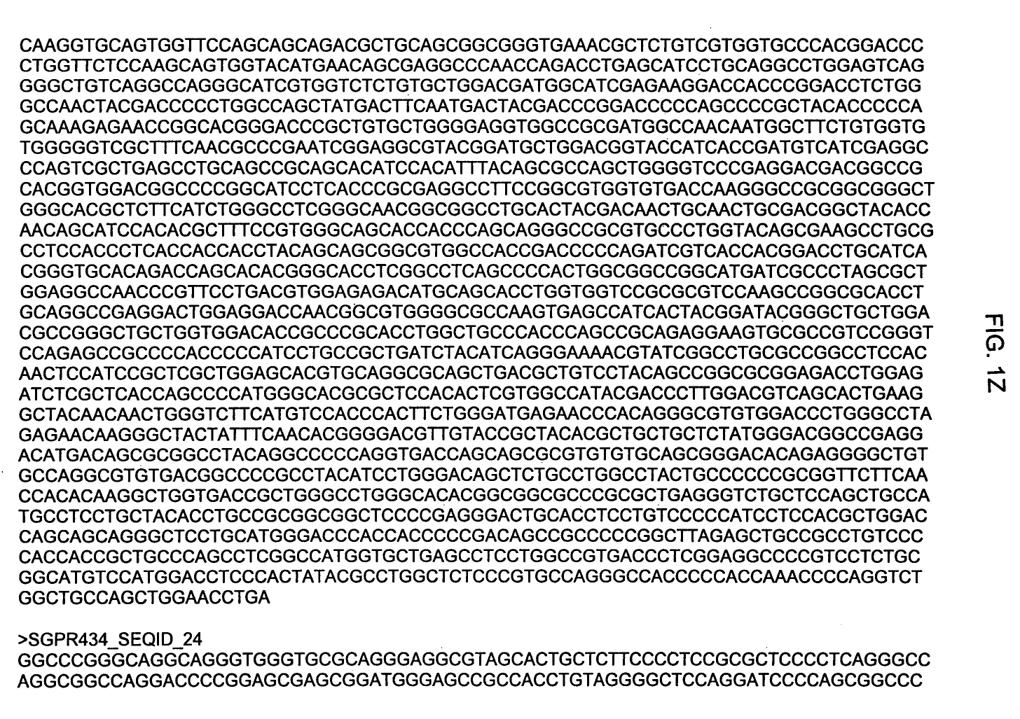

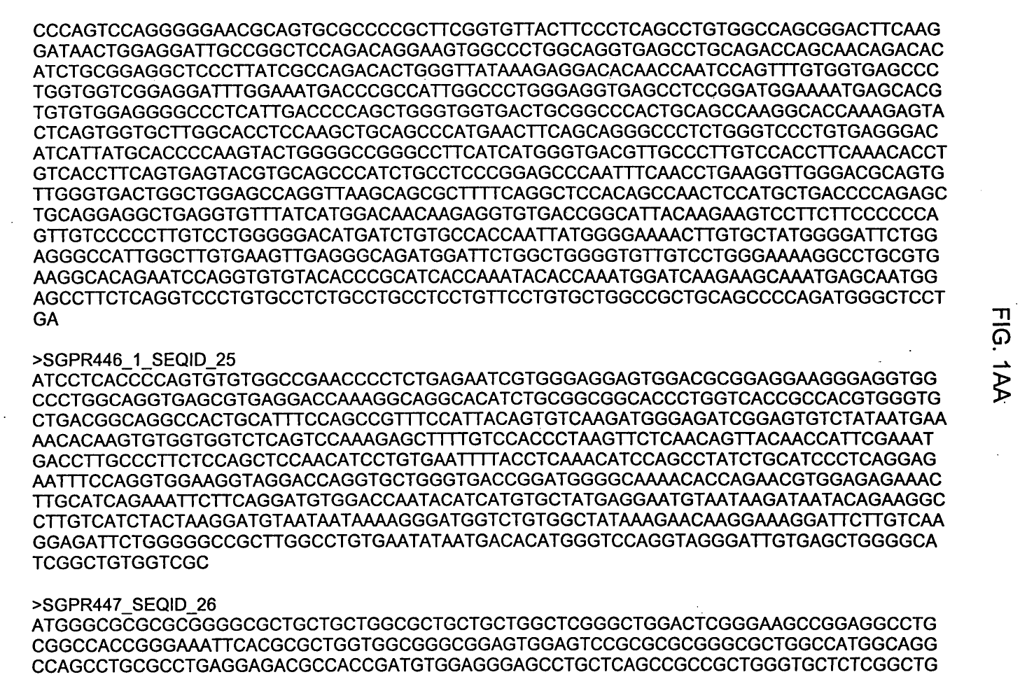

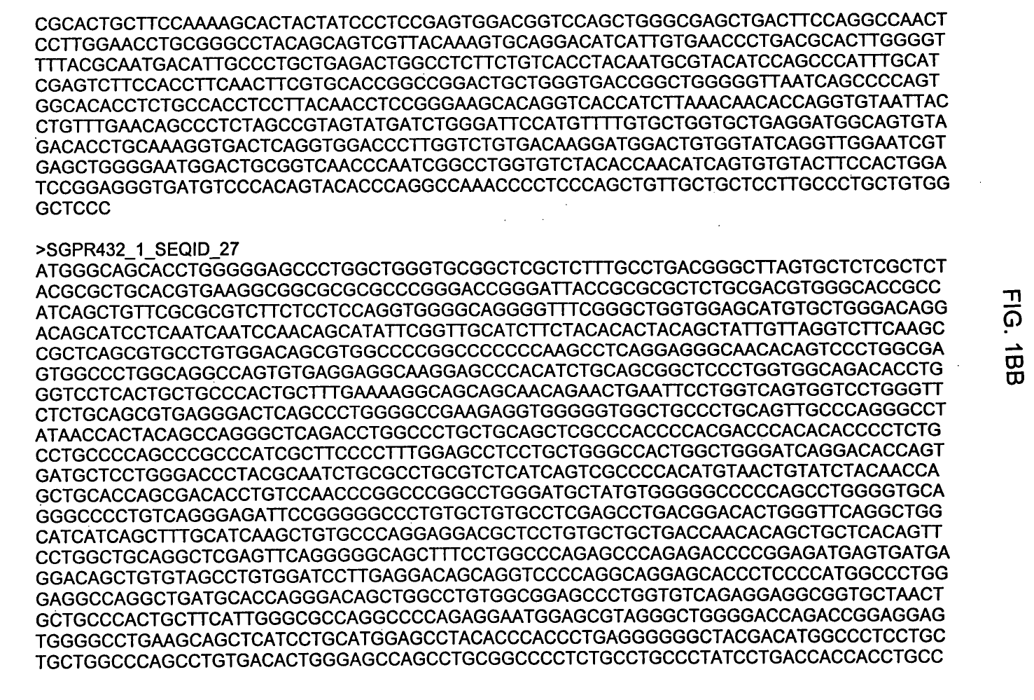

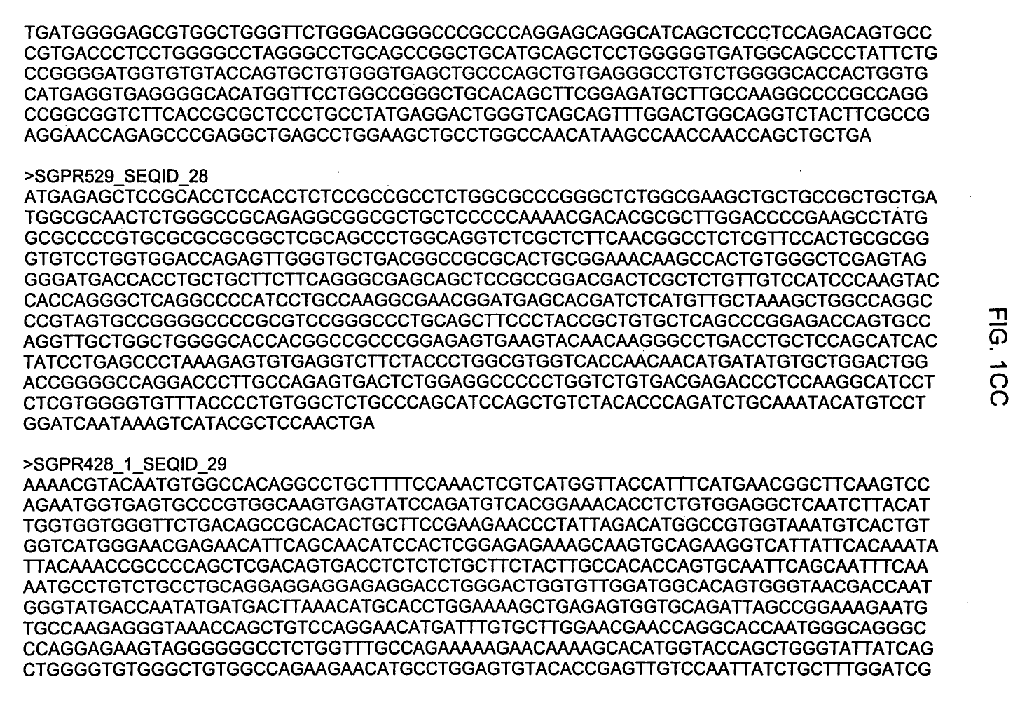

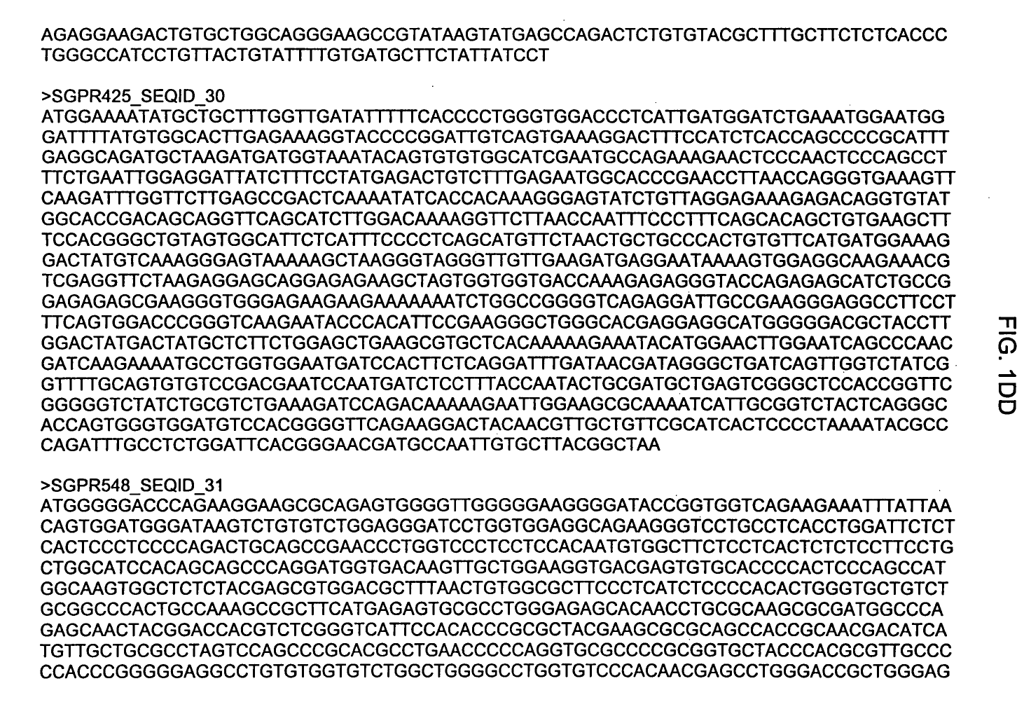

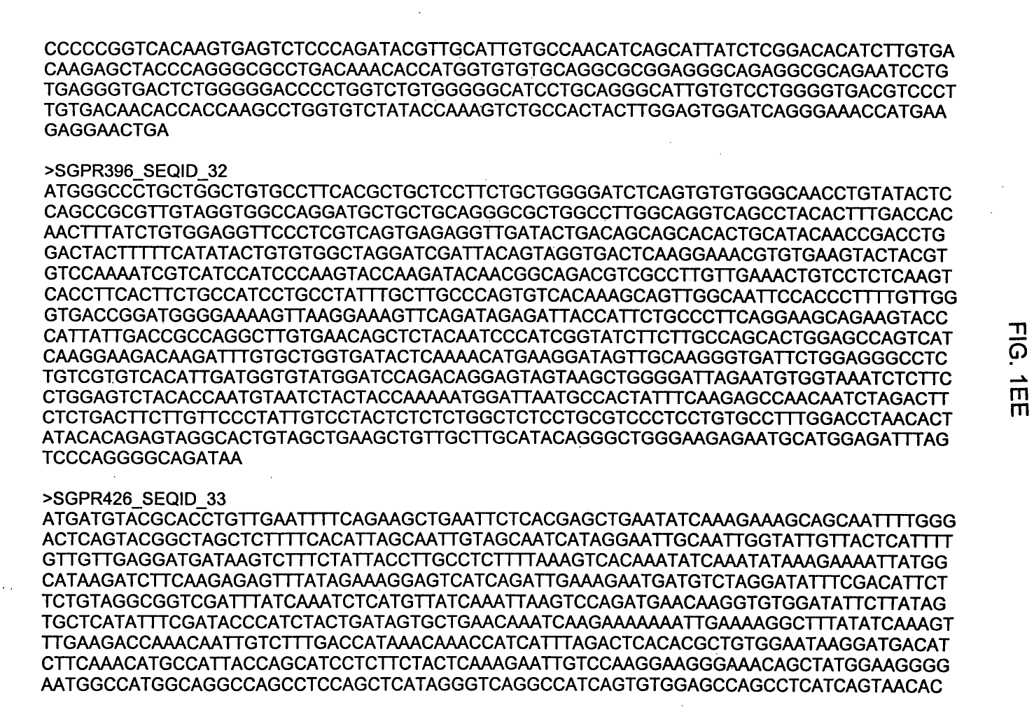

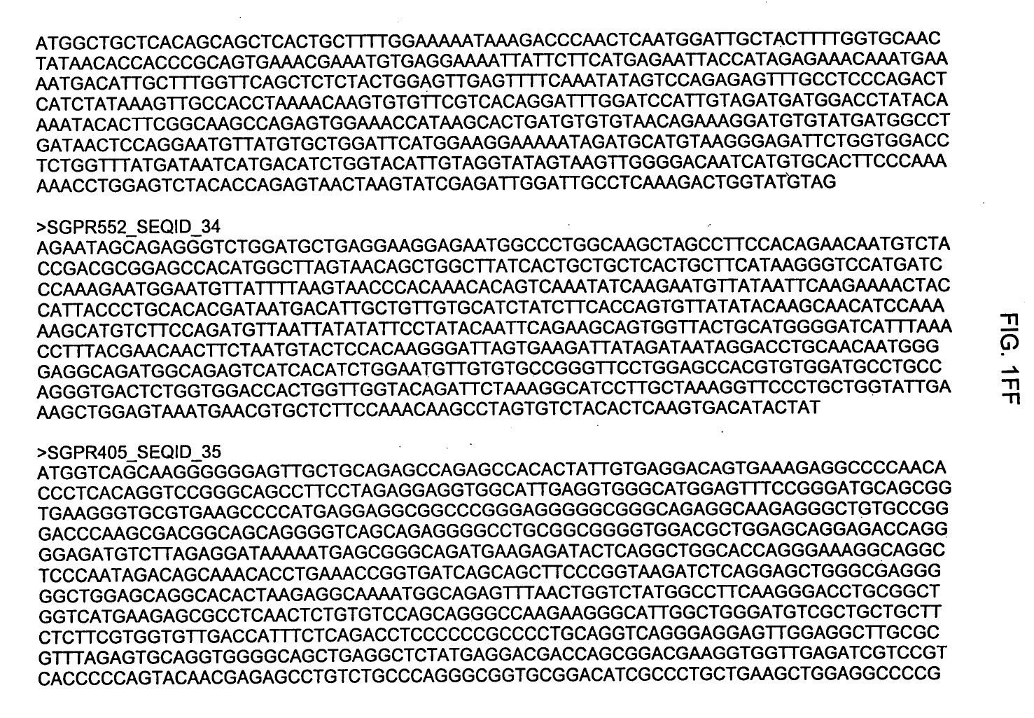

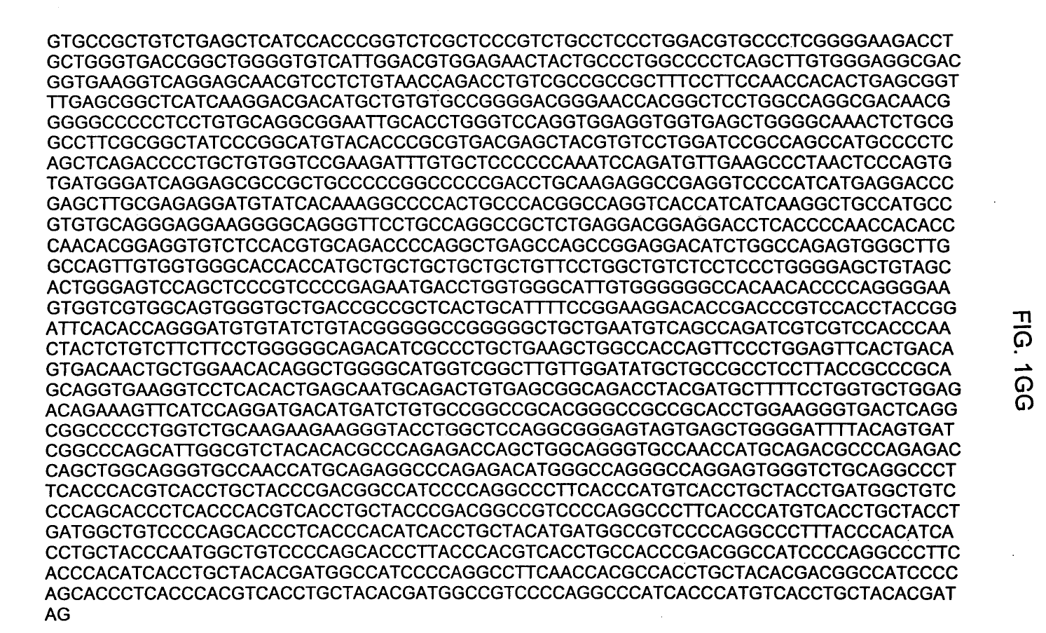

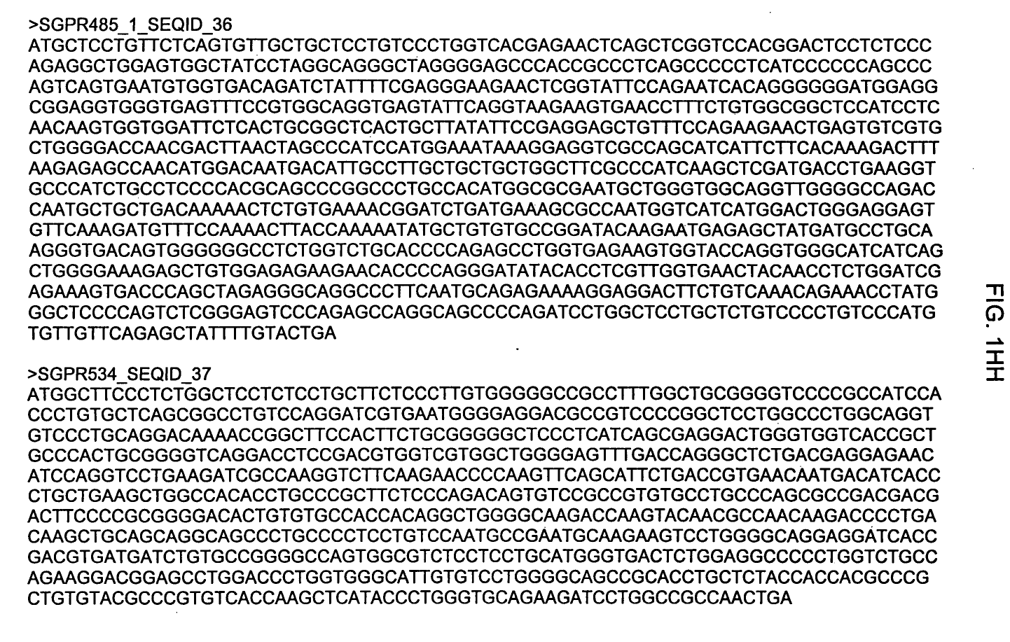

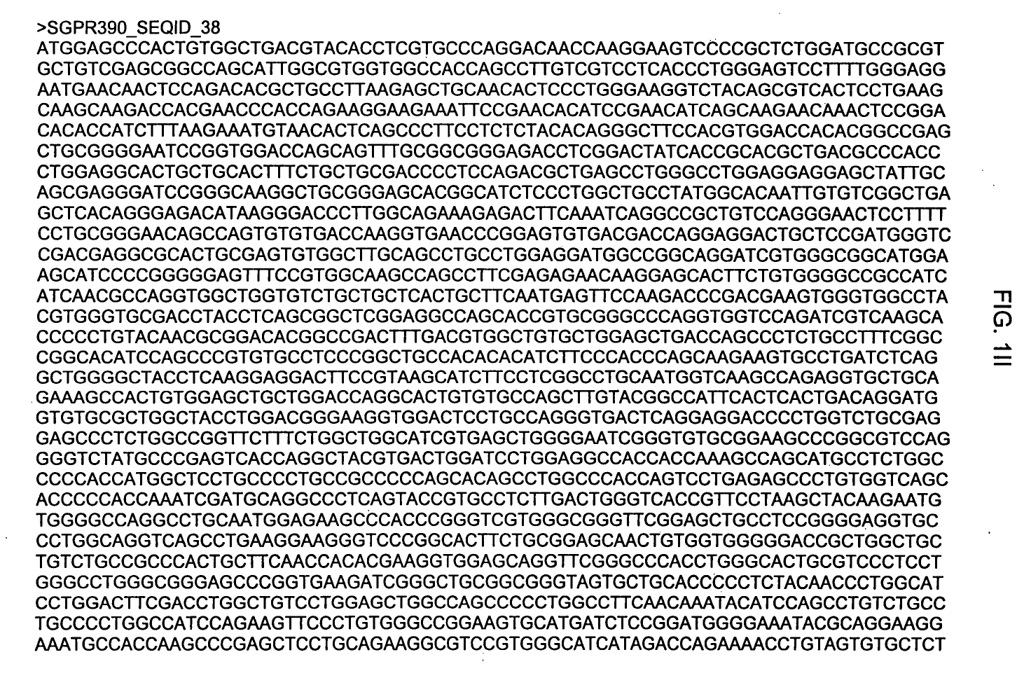

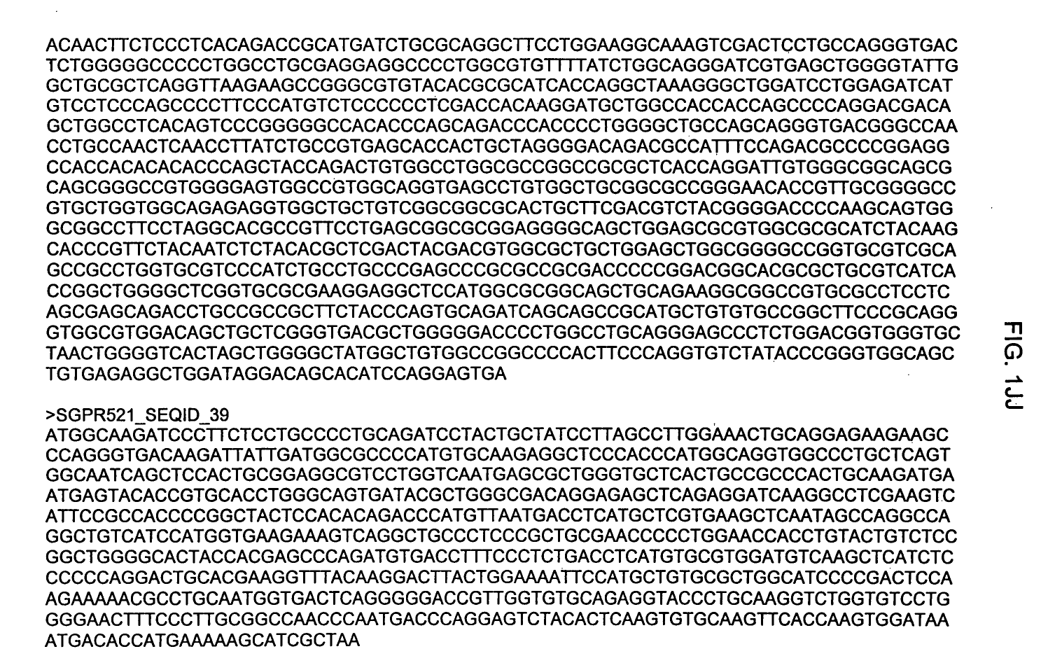

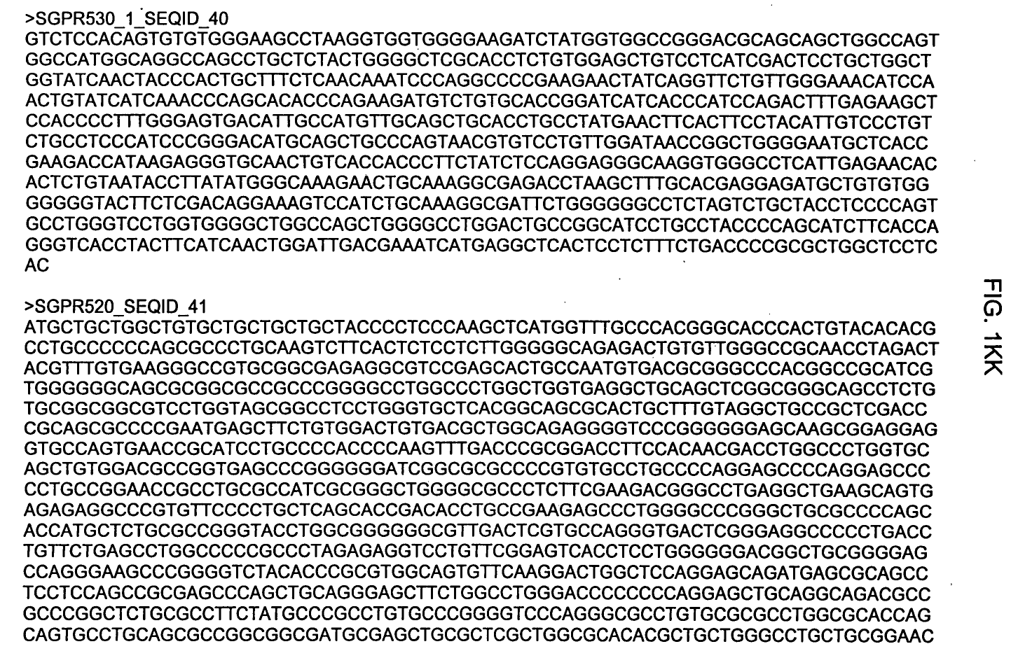

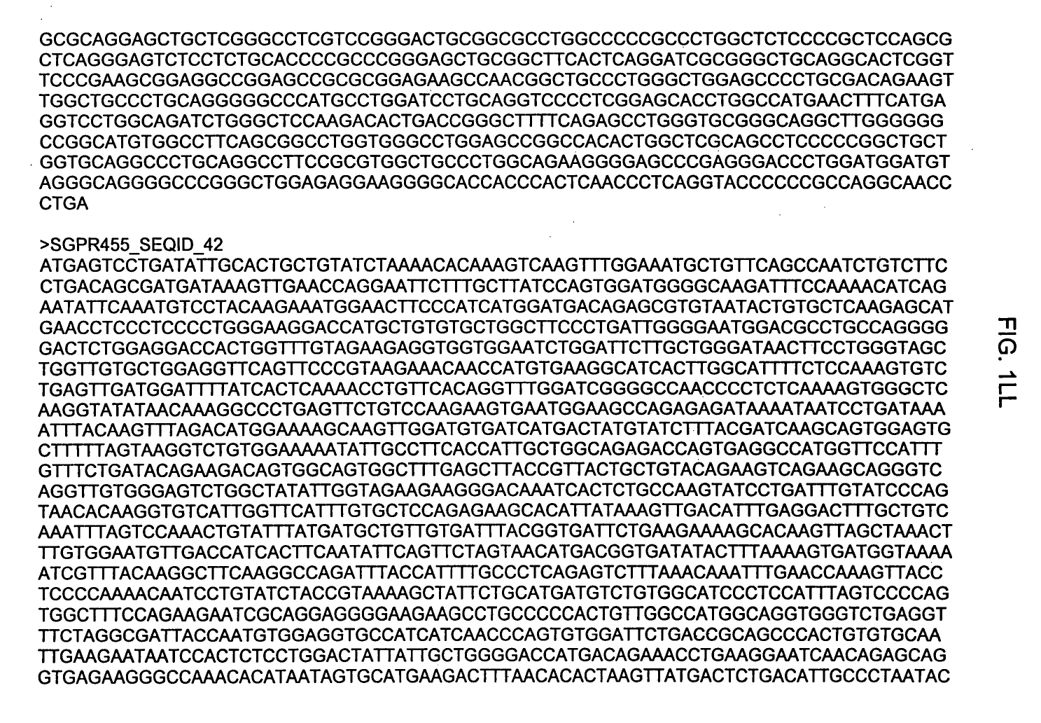

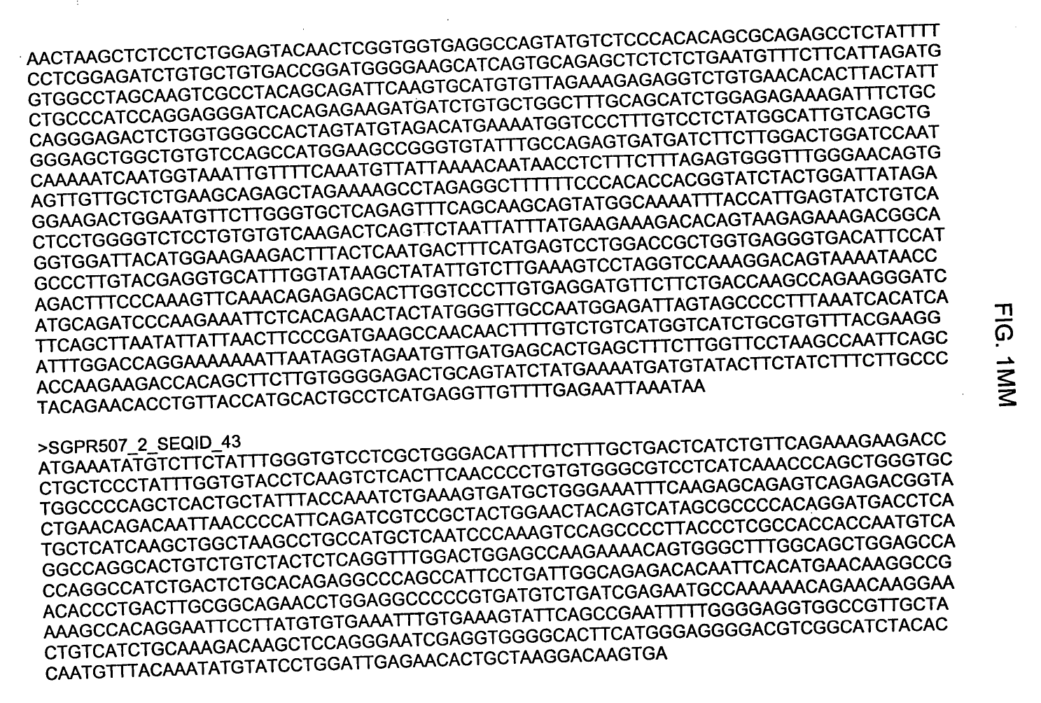

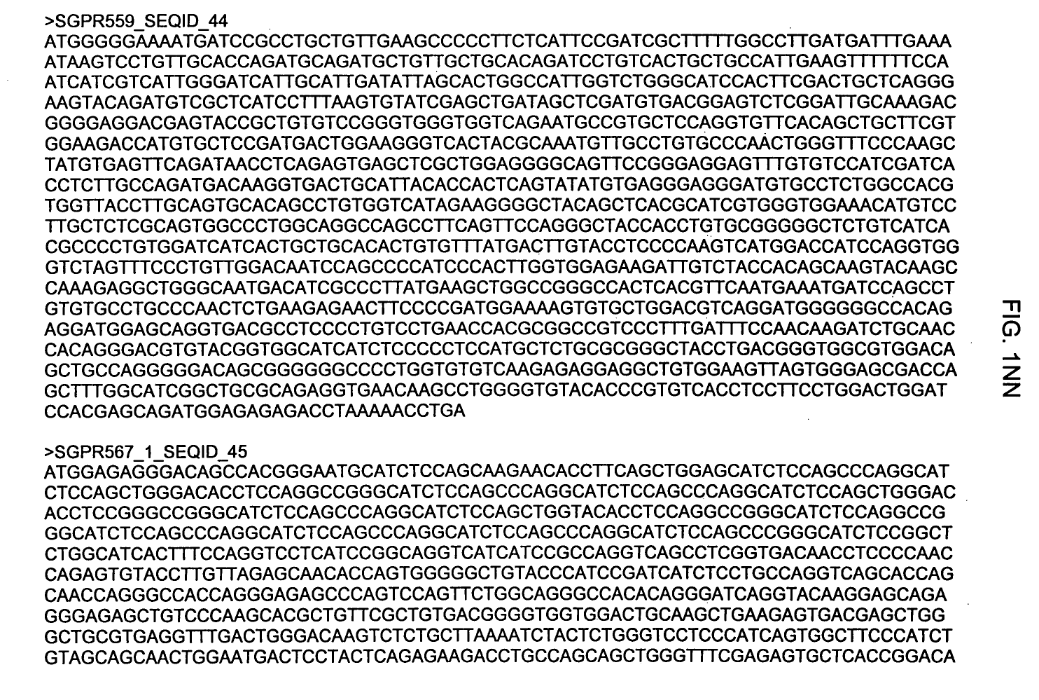

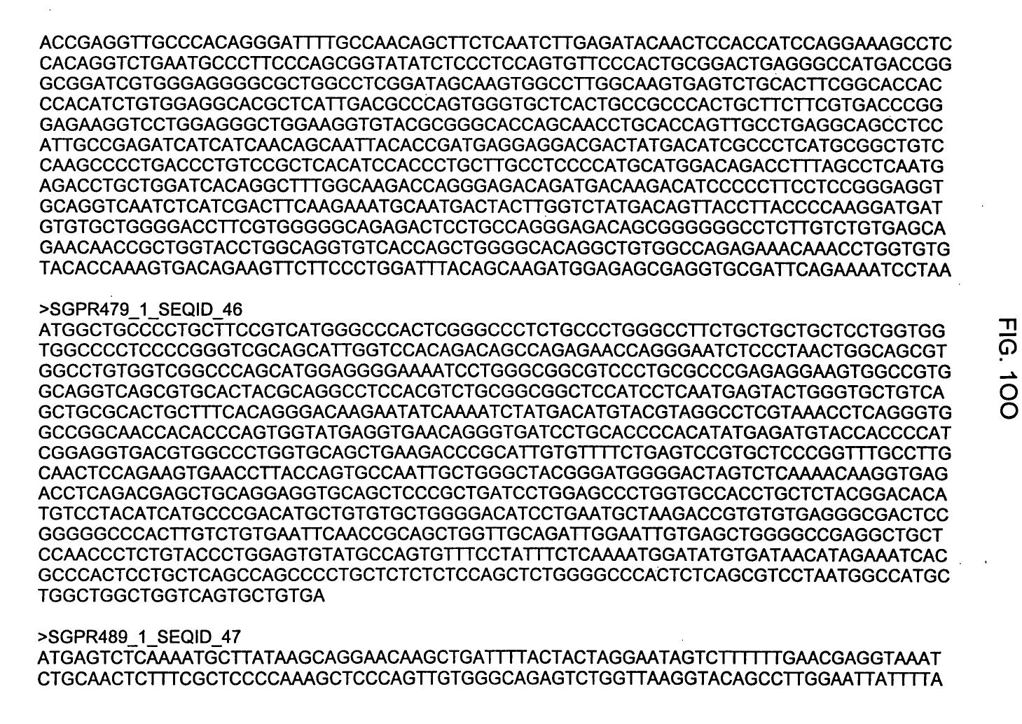

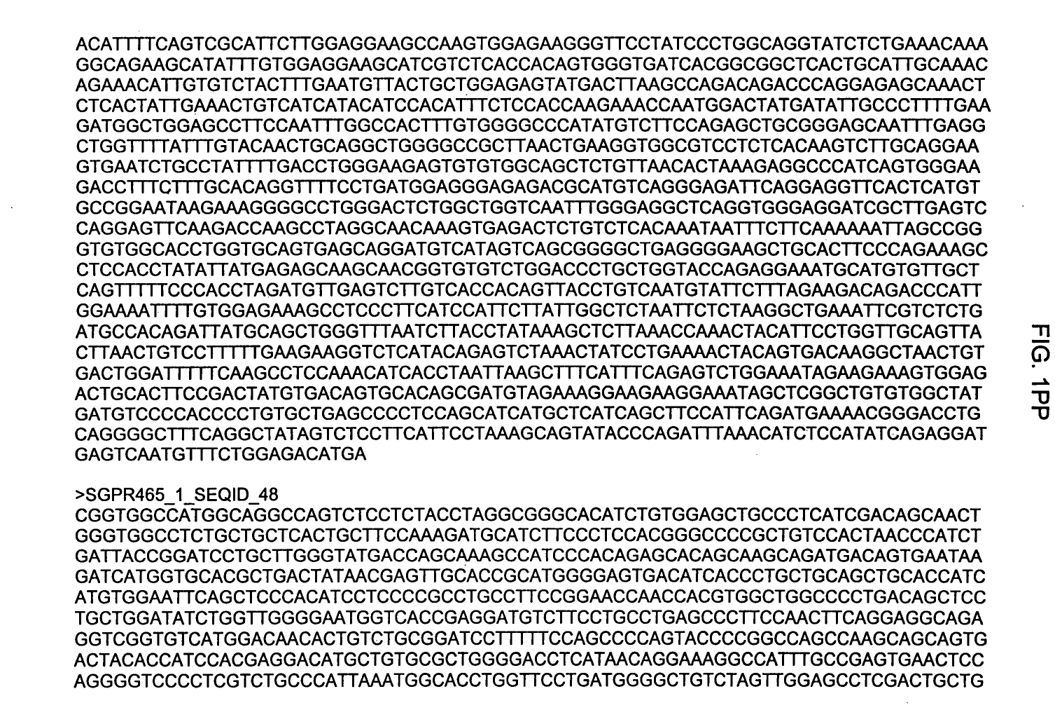

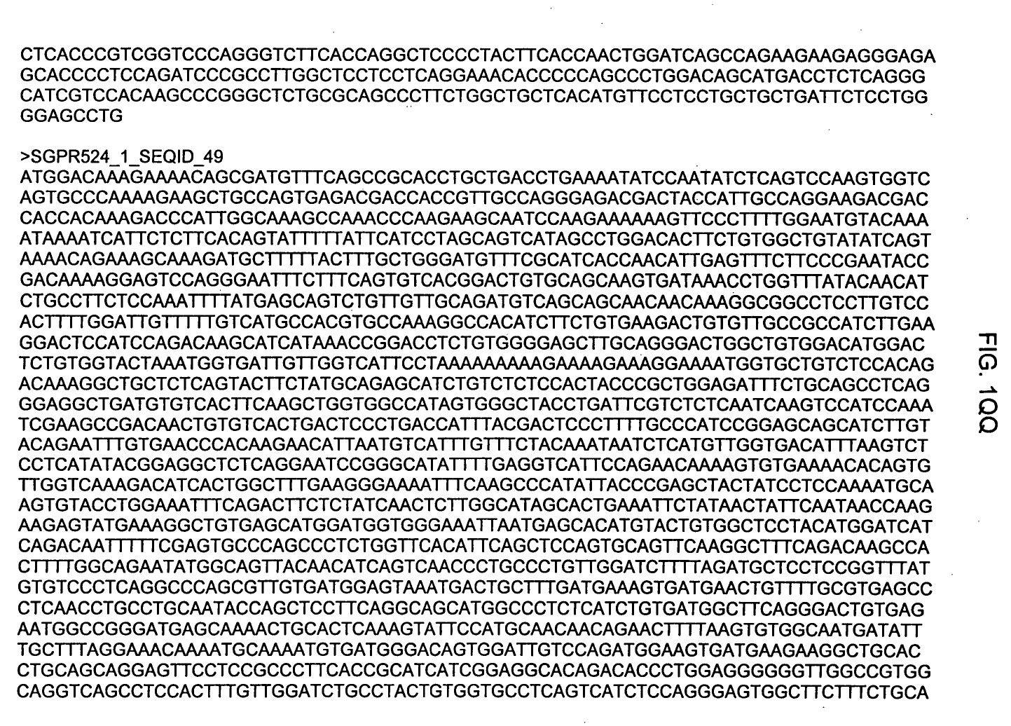

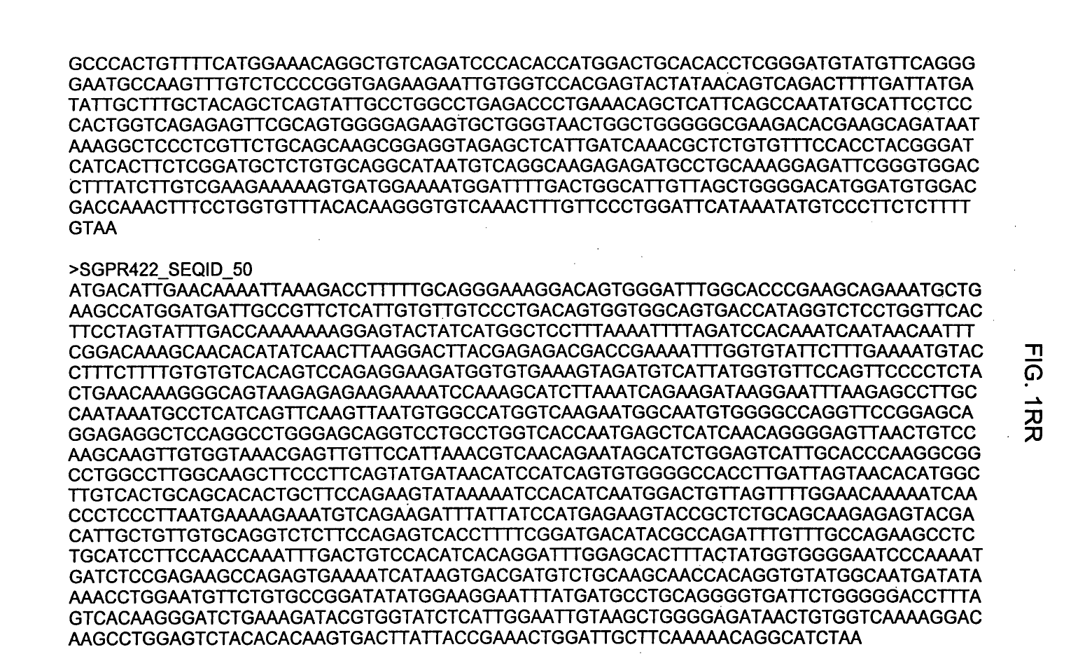

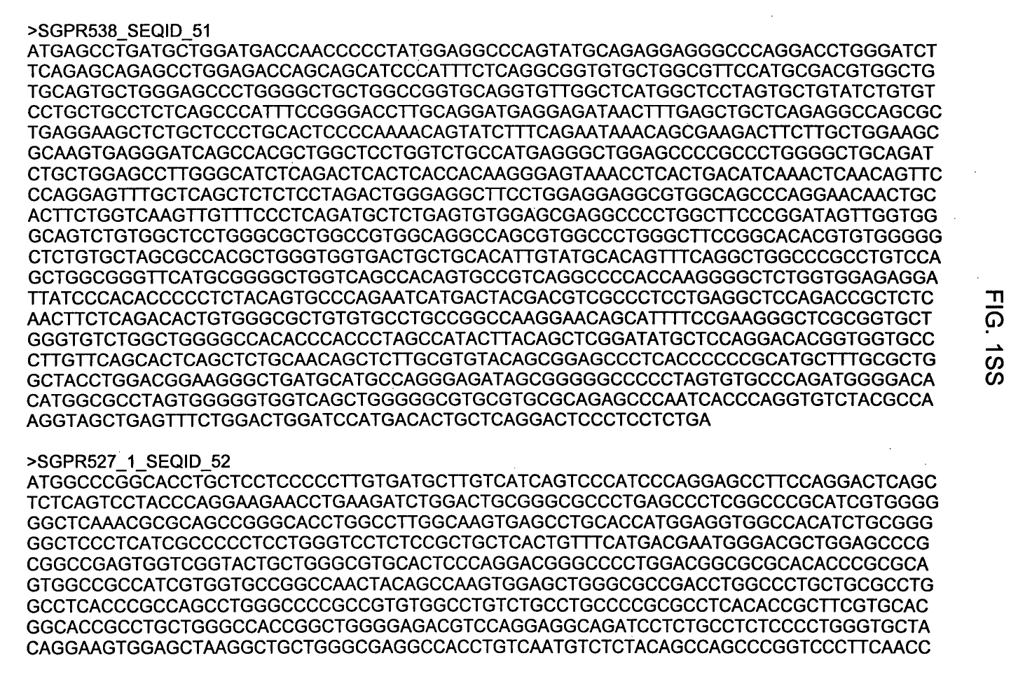

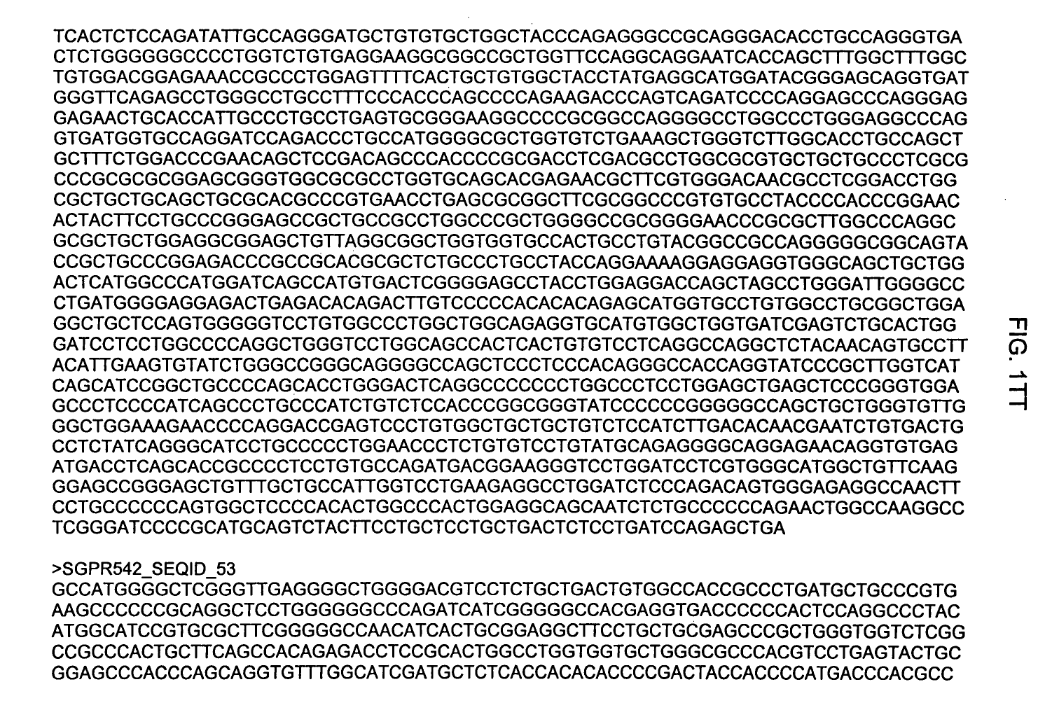

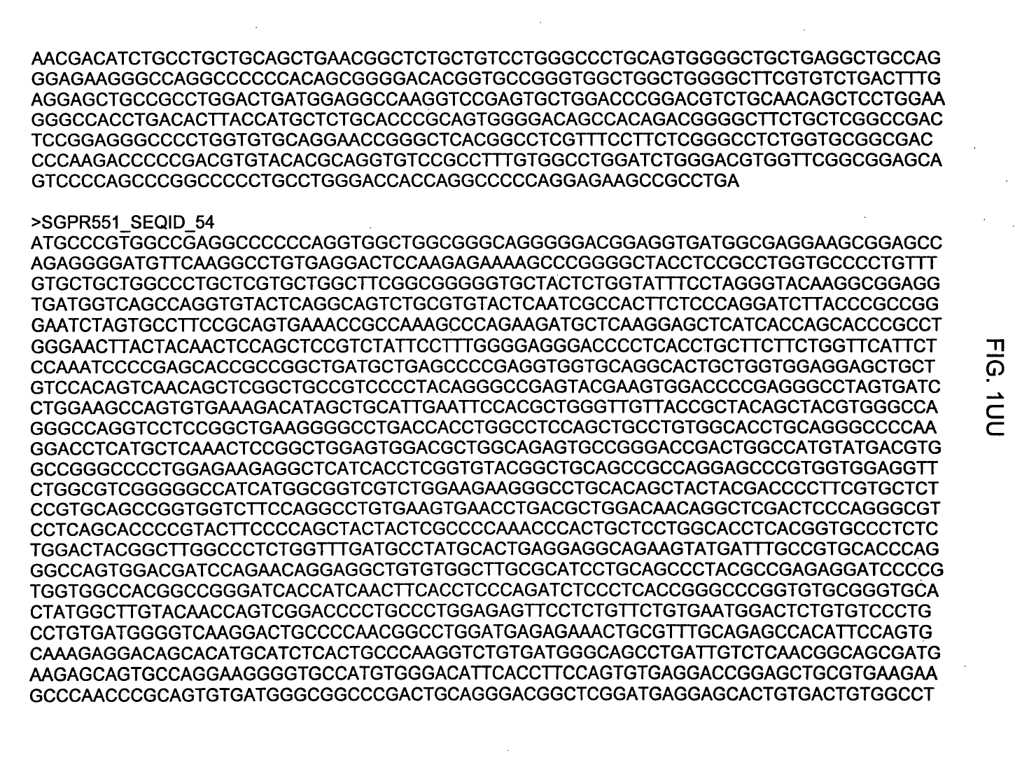

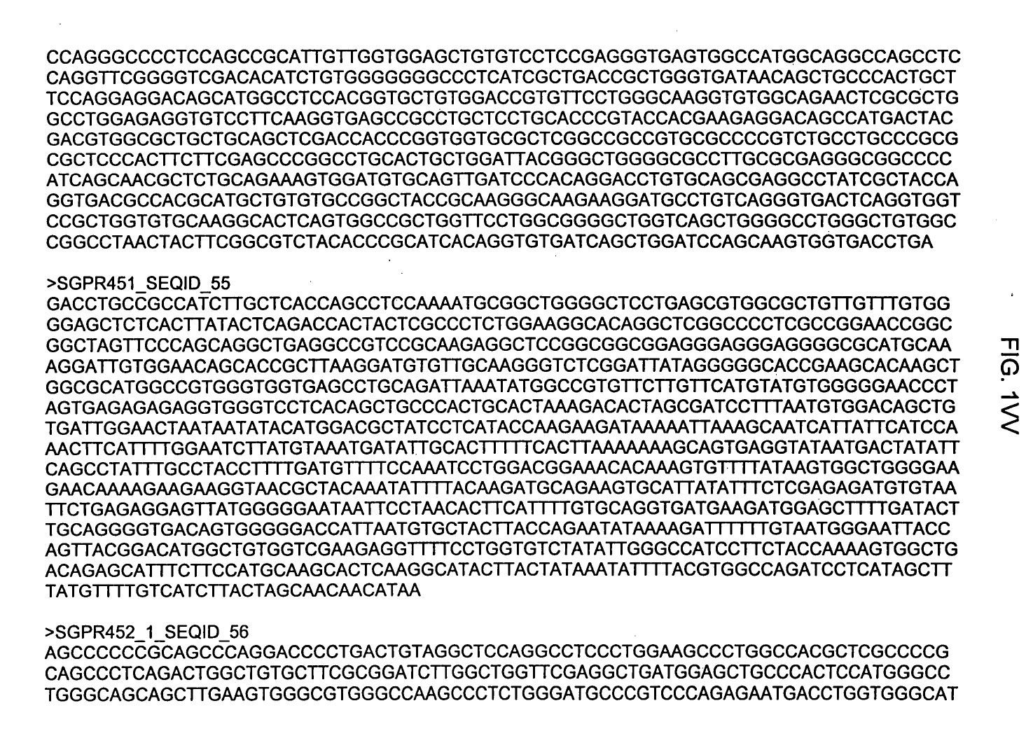

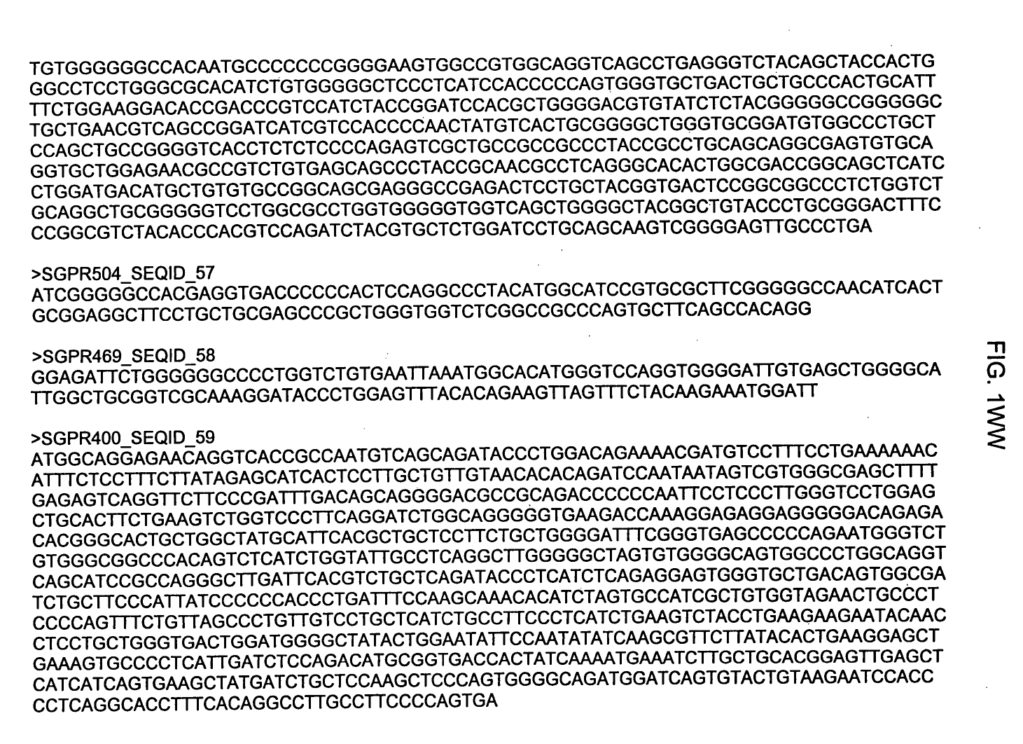

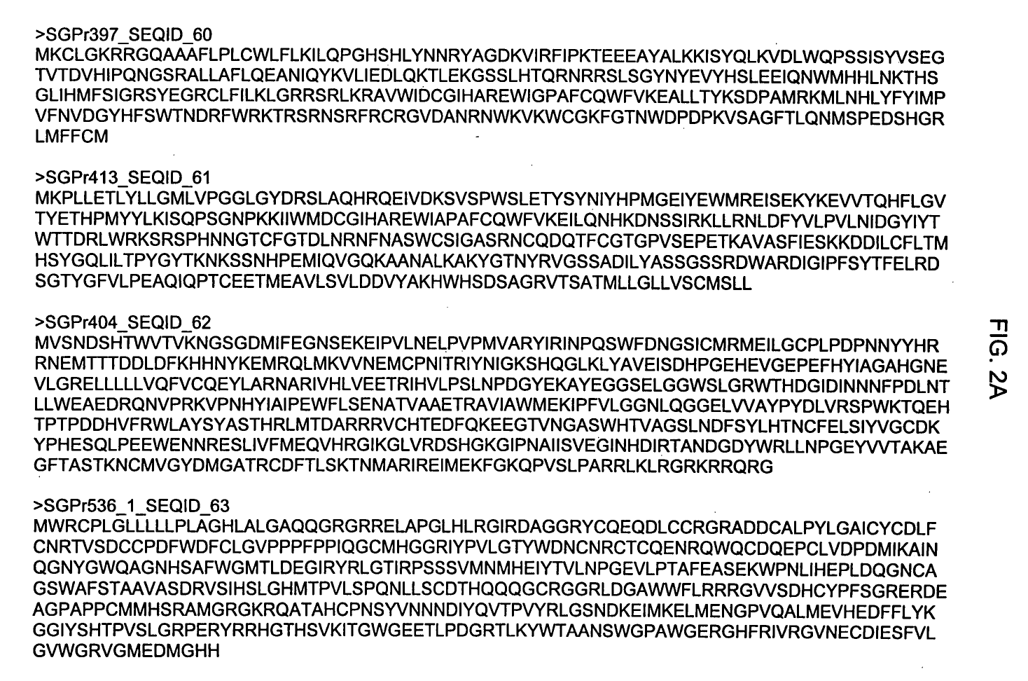

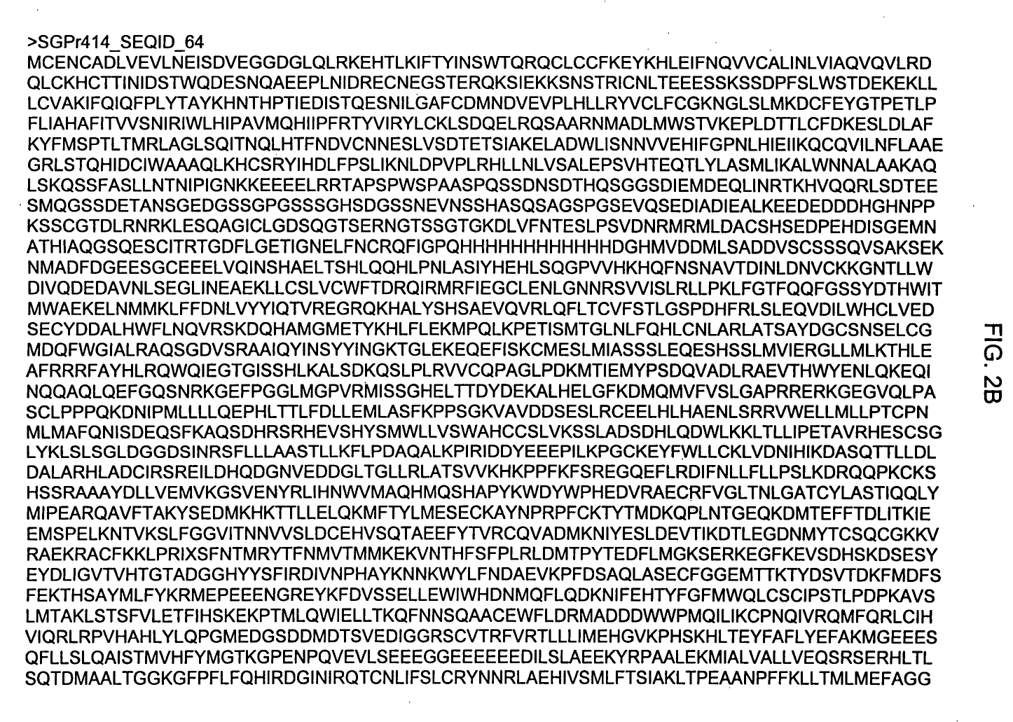

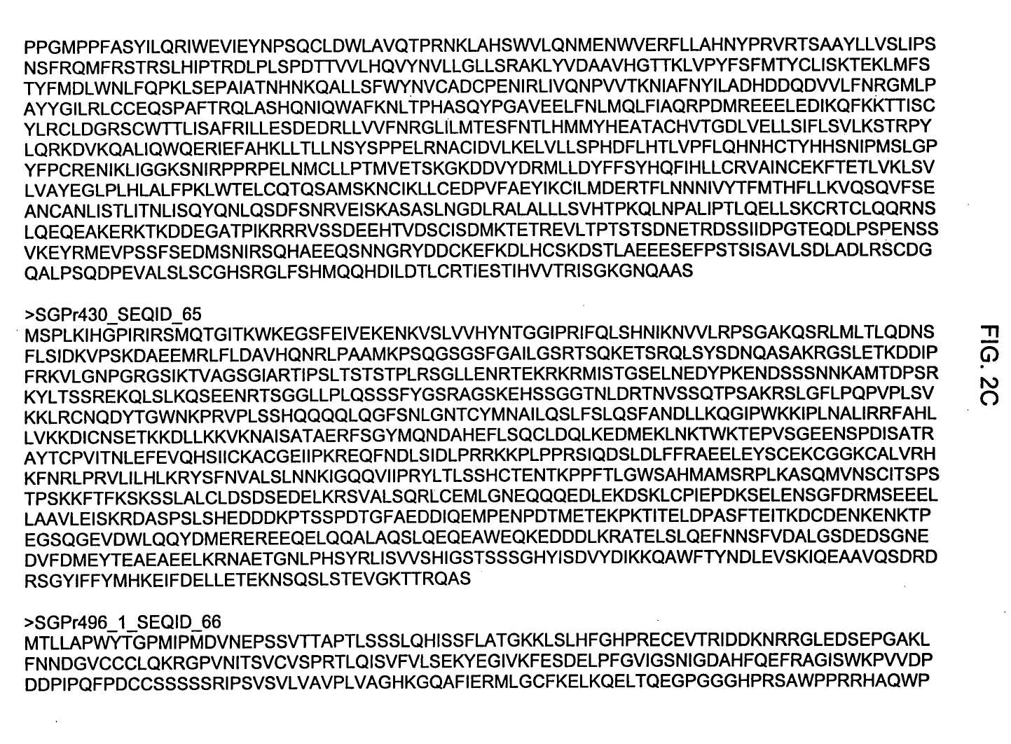

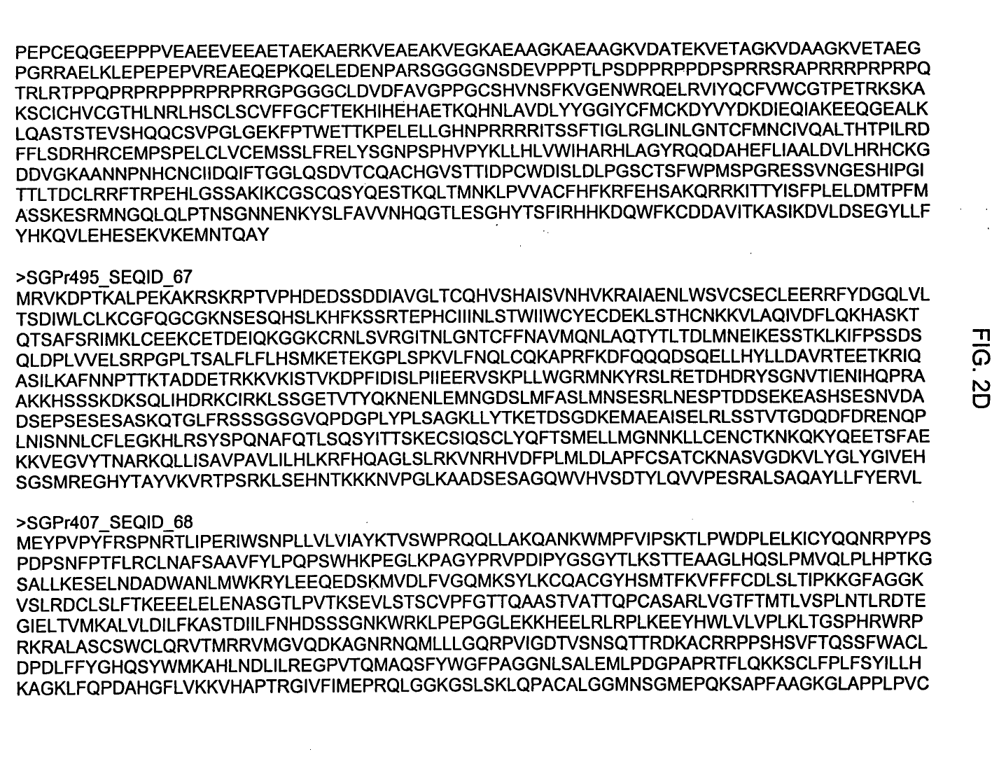

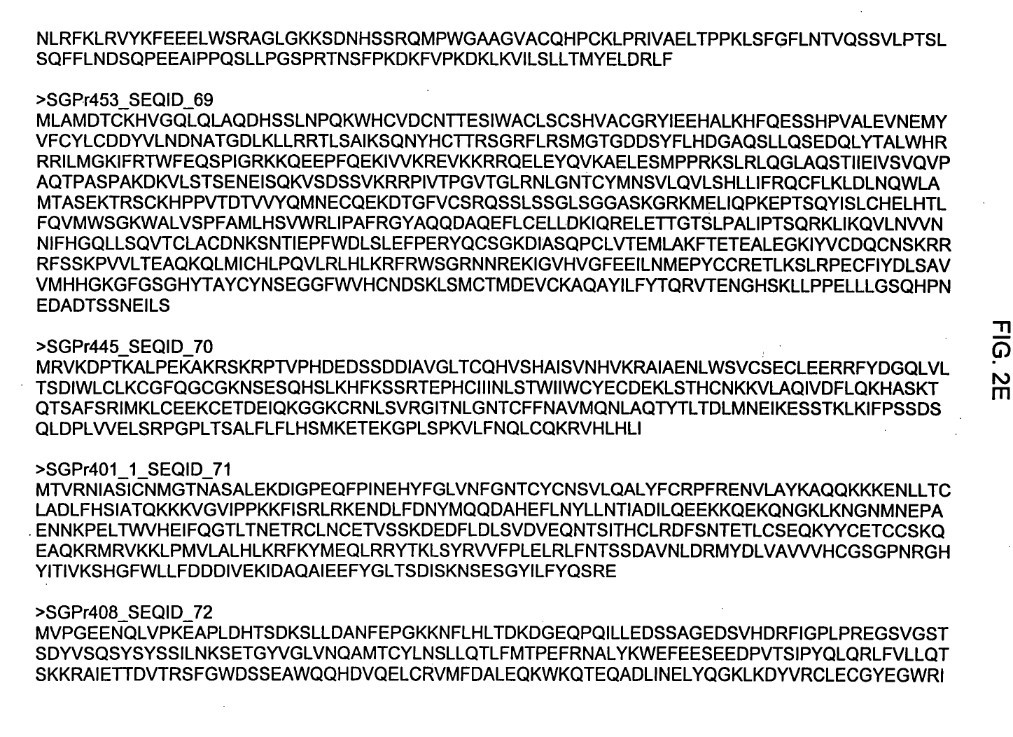

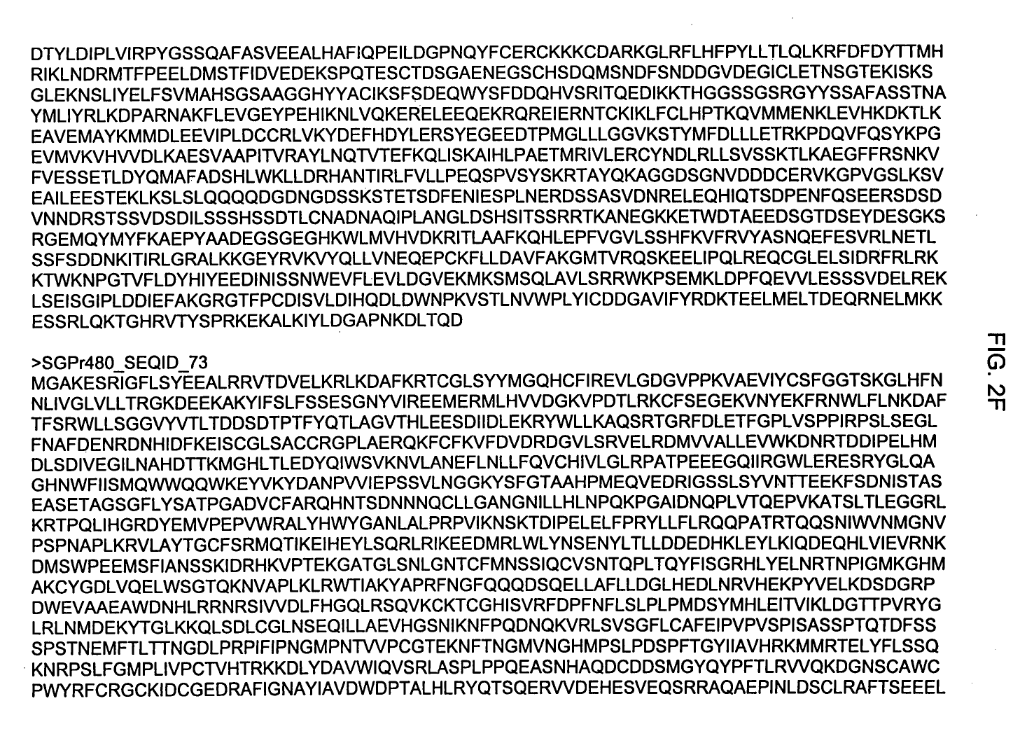

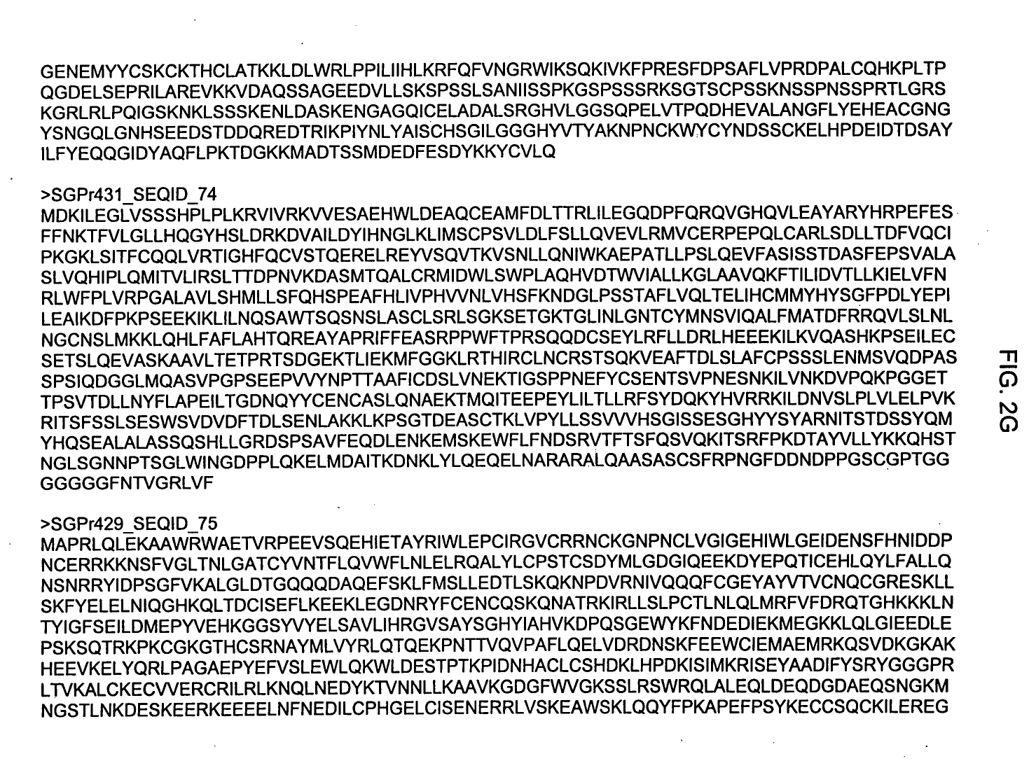

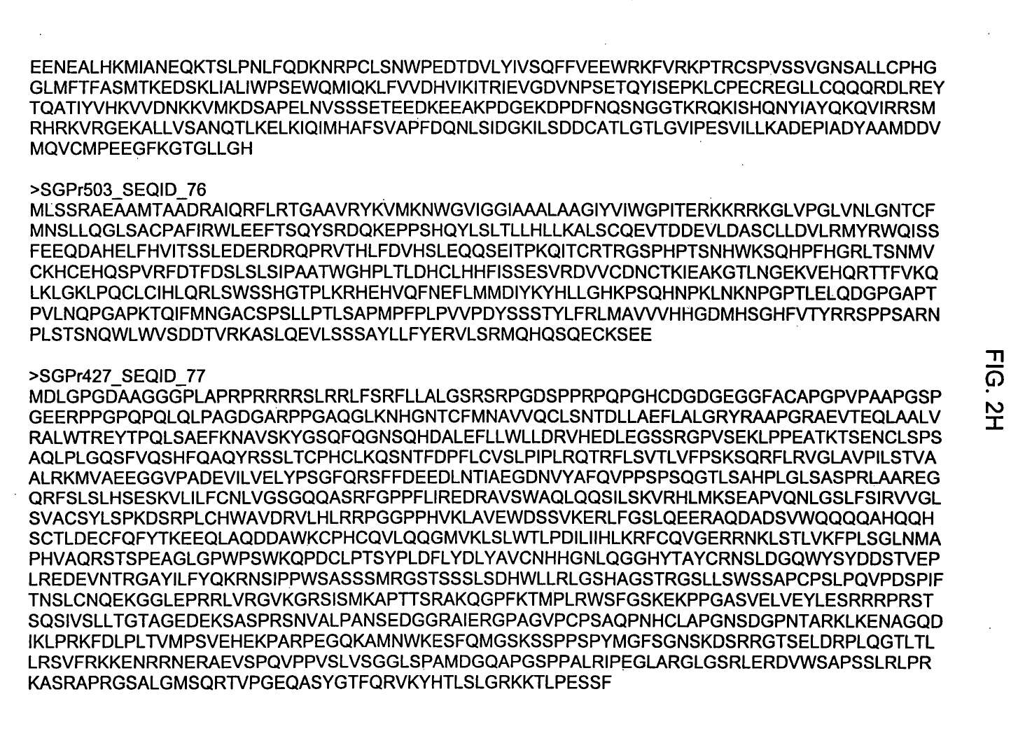

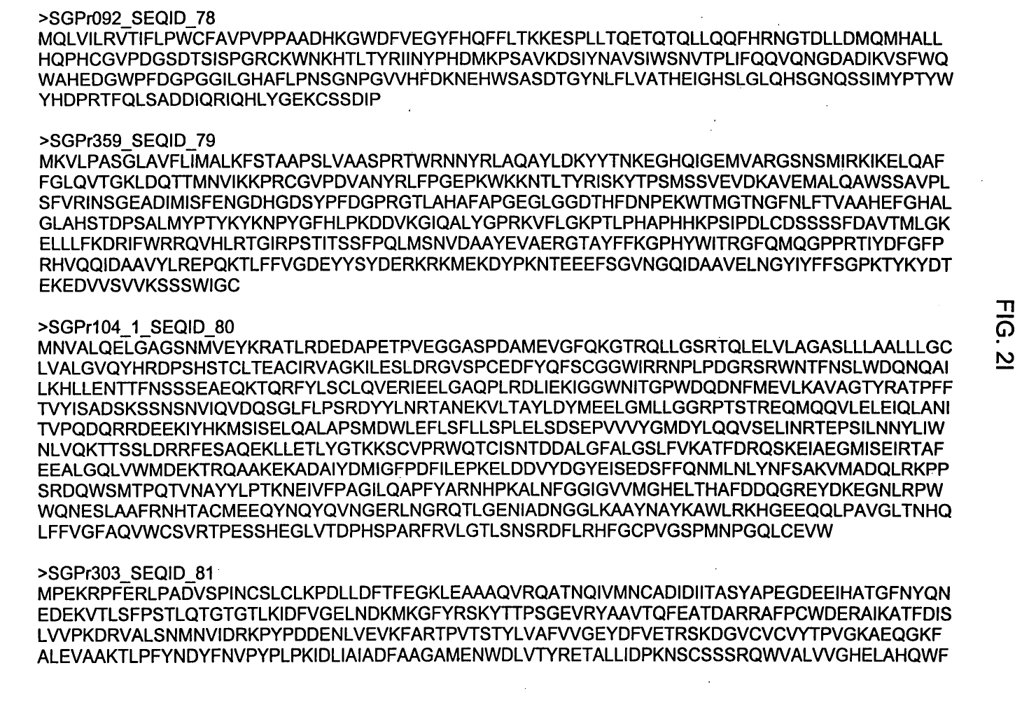

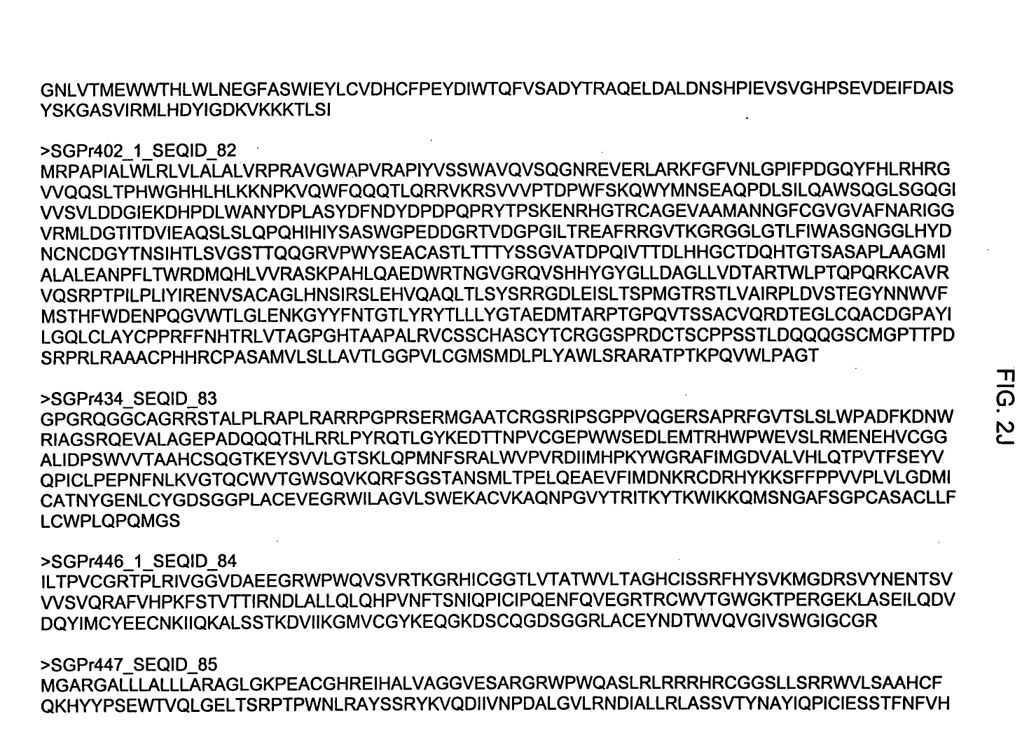

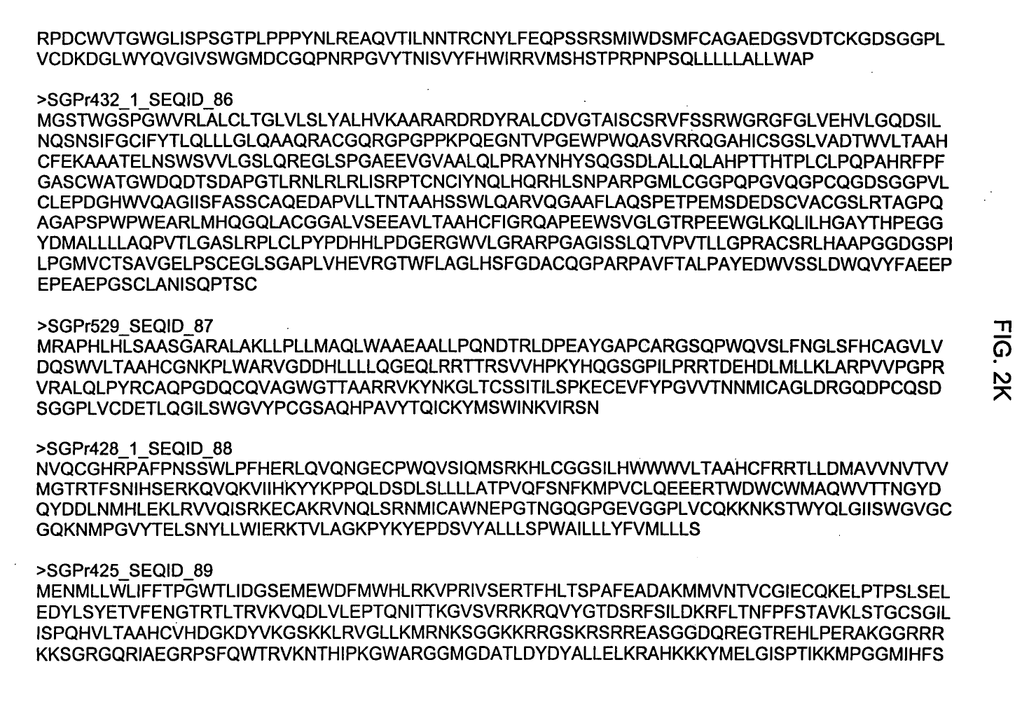

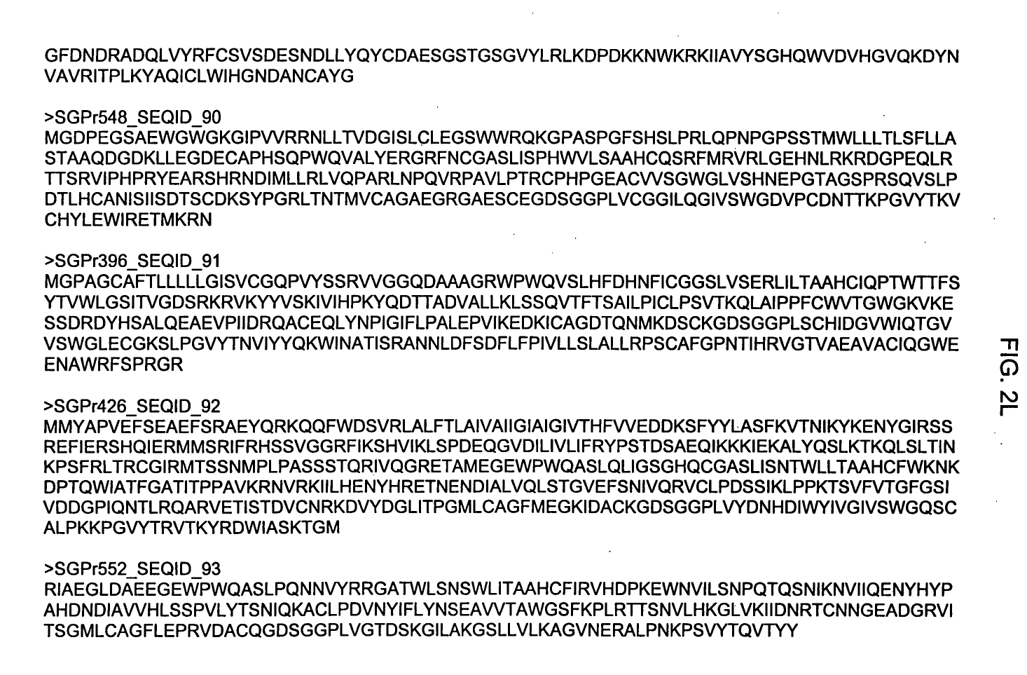

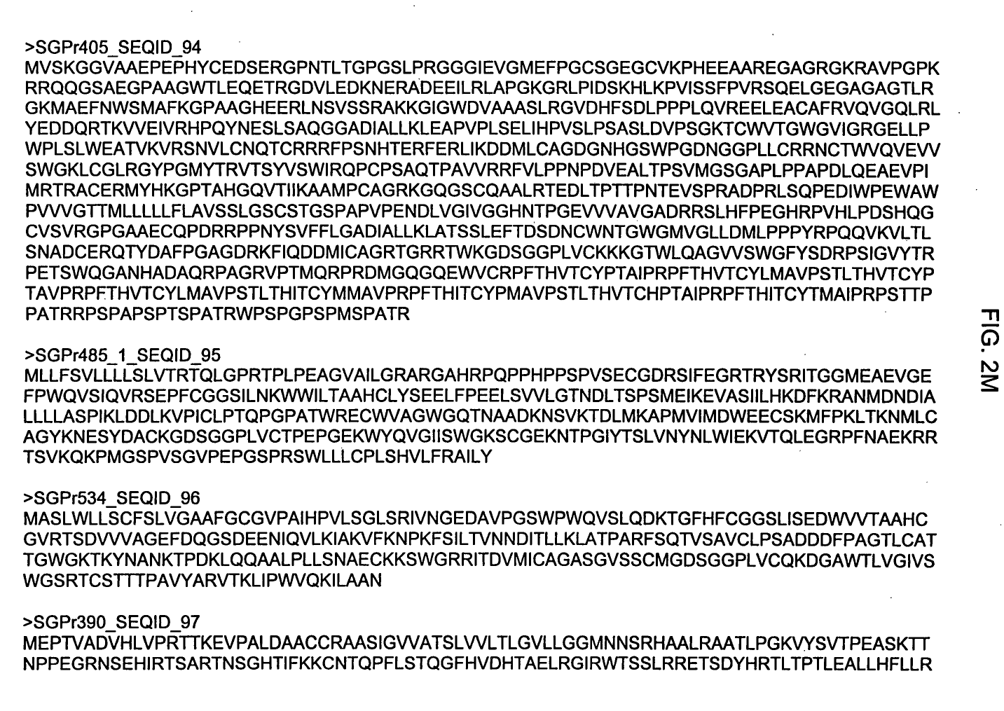

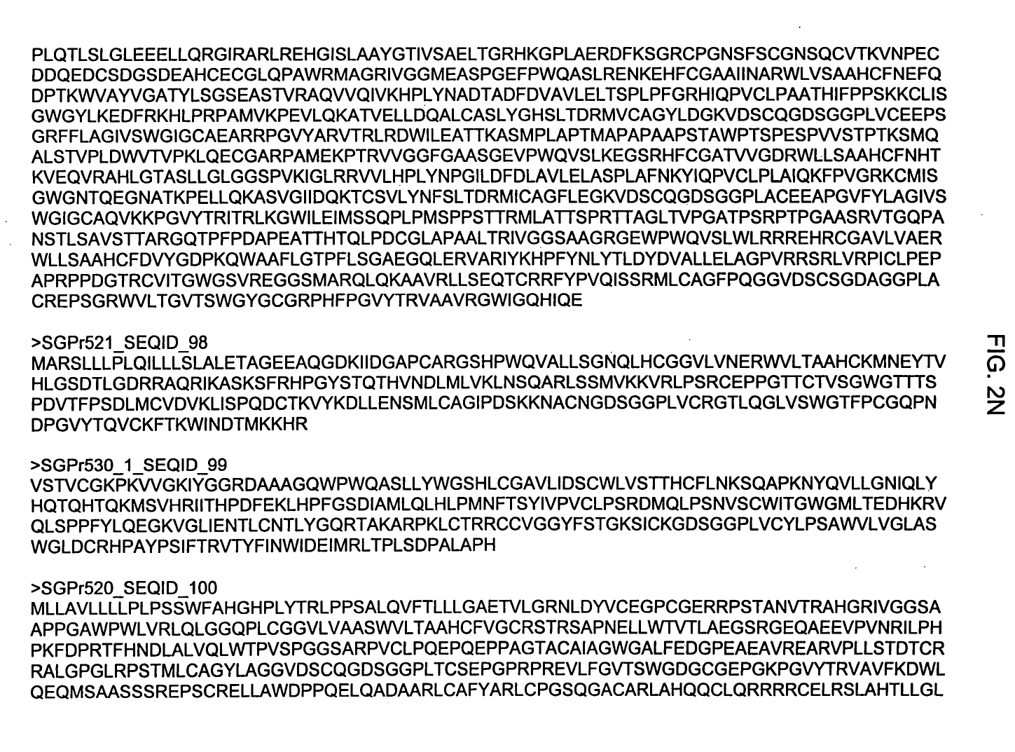

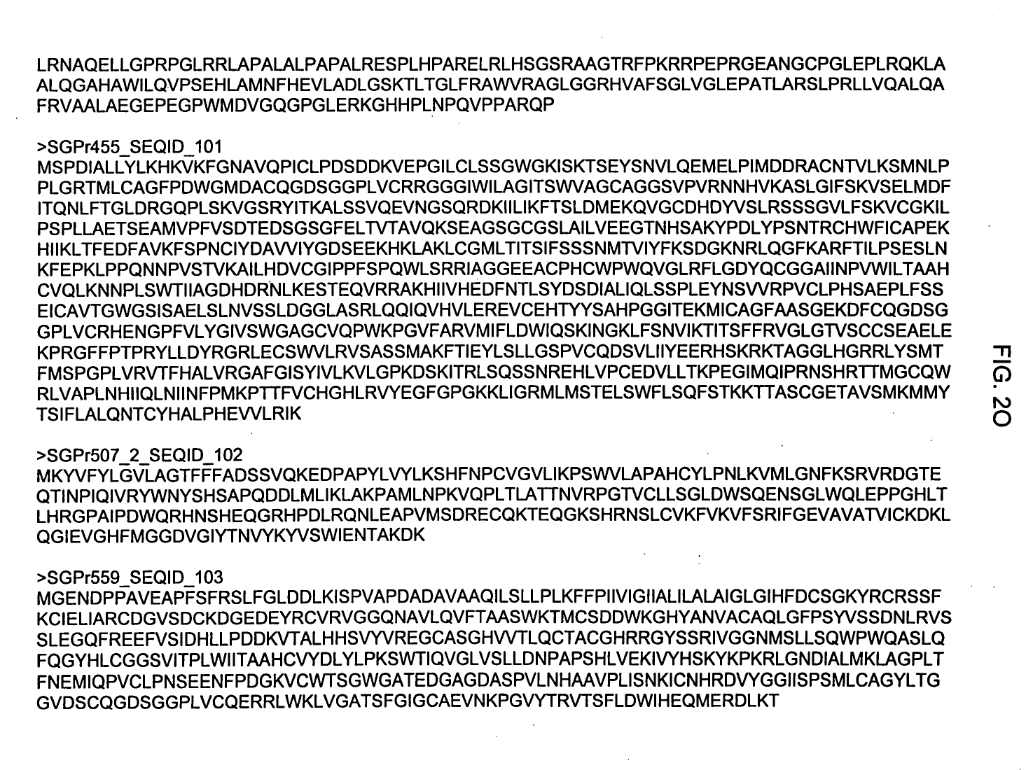

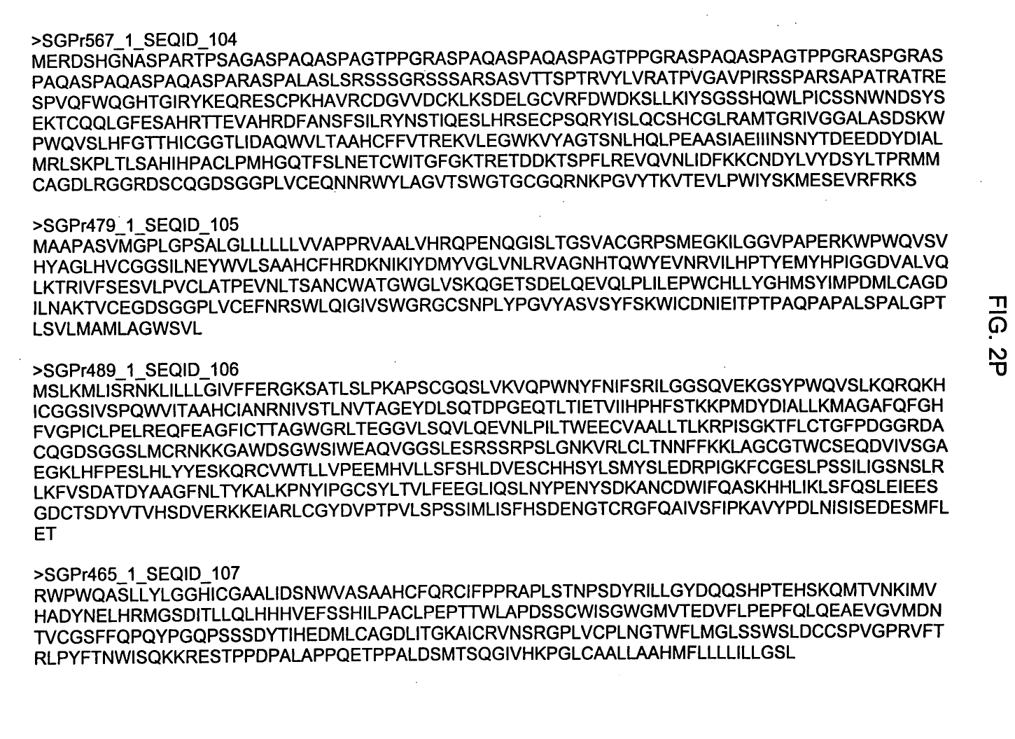

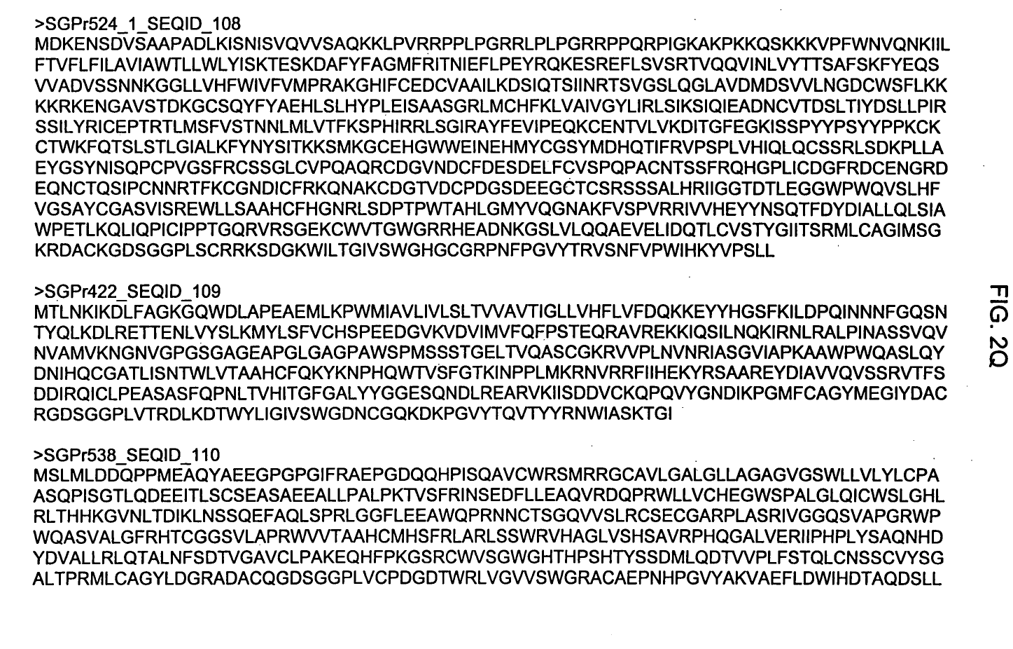

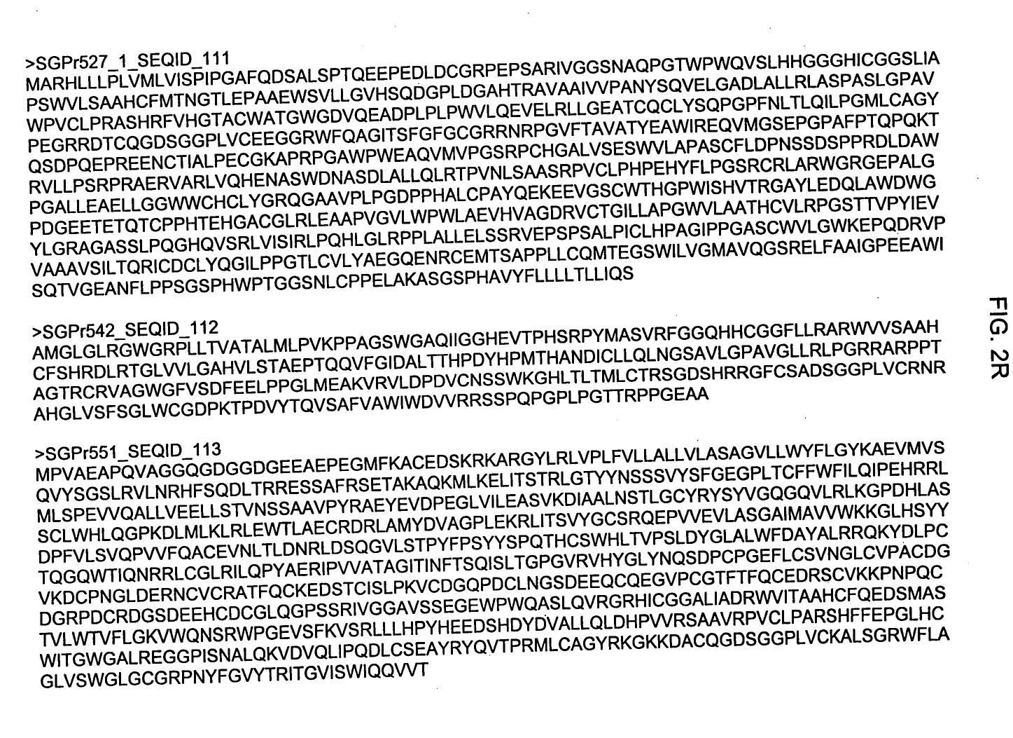

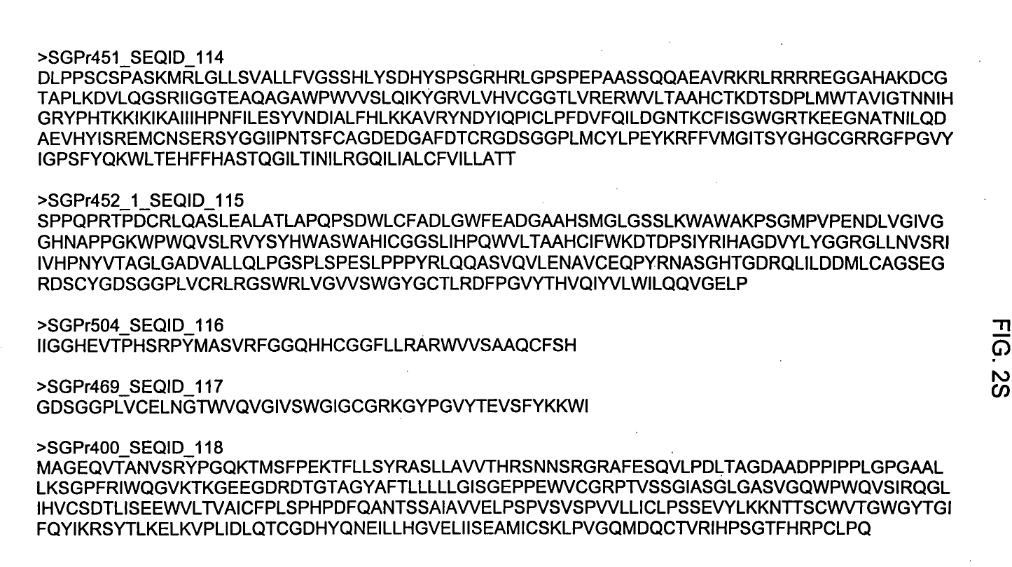

### DETAILED DESCRIPTION OF THE INVENTION

The following description of the background of the invention is provided to aid in understanding the invention, but is not admitted to be or to describe prior art to the invention.

Proteases are enzymes capable of severing the amino acid backbone of other proteins, and are involved in a large number of diverse processes within the body. Their normal functions include modulation of apoptosis (caspases) (Salvesen and Dixon, Cell, 1997, 91:44346), control of blood pressure (renin, angiotensin-converting enzymes) (van Hooft et al., 1991, N Engl J Med. 324(19):1305-11, and chapters 254 and 359 in Barrett et al., Handbook of Proteolytic Enzymes, 1998, Academic Press, San Diego), tissue remodeling and tumor invasion (collagenase) (Vu et al., 1998, Cell 93:411-22, Werb, 1997, Cell, 91:439-442), development of Alzheimer's Disease (β-secretase) (De Strooper et al., 1999, Nature 398:518-22), protein turnover and cell-cycle regulation (proteosome) (Bastians et al., 1999, Mol. Biol. Cell. 10:3927-41, Gottesman, et al., 1997, Cell, 91:435-38, Larsen et al., 1997, Cell, 91:431-34), inflammation (TNF-α convertase) (Black et al., Nature, 1997, 385:729-33), and protein turnover (Bochtler et al., 1999, Annu. Rev. Biophys Biomol Struct. 28:295-317). Proteases may be classified into several major groups including serine proteases, cysteine proteases, aspartyl proteases, metalloproteases, threonine proteases, and other proteases.

1. Aspartyl Proteases (A1: Prosite Number PS00141):

Aspartyl proteases, also known as acid proteases, are a widely distributed family of proteolytic enzymes in vertebrates, fungi, plants, retroviruses and some plant viruses. Aspartate proteases of eukaryotes are monomeric enzymes which consist of two domains. Each domain contains an active site centered on a catalytic aspartyl residue. The two domains most probably evolved from the duplication of an ancestral gene encoding a primordial domain. Enzymes in this class include cathepsin E, renin, presenilin (PS1), and the APP secretases.

Cathepsin E

Cathepsin E is an immunologically discrete aspartic protease found in the gastrointestinal tract (Azuma et al., 1992 J. Biol. Chem., 267:1609-1614). Cathepsin E is an intracellular proteinase that does not appear to be involved in the digestion of dietary protein. It is found in highest concentration in the surface of epithelial mucus-producing cells of the stomach. It is the first aspartic proteinase expressed in the fetal stomach and is found in more than half of gastric cancers. It appears, therefore, to be an ‘oncofetal’ antigen. Its association with stomach cancers suggests it may play a role in the development of this disease.

Renin

Released by the juxtaglomerular cells of the kidney, renin catalyzes the first step in the activation pathway of angiotensinogen—a cascade that can result in aldosterone release, vasoconstriction, and increase in blood pressure. Renin cleaves angiotensinogen to form angiotensin I, which is converted to angiotensin II by angiotensin I converting enzyme, an important regulator of blood pressure and electrolyte balance. Renin occurs in other organs than the kidney, e.g., in the brain, where it is implicated in the regulation of numerous activities.

Presenilin Proteins

Alzheimer's disease (AD) patients with an inherited form of the disease carry mutations in the presenilin proteins (PSEN1; PSEN2) or the amyloid precursor protein (APP). These disease-linked mutations result in increased production of the longer form of amyloid-beta (main component of amyloid deposits found in AD brains) (Saftig et al., Eur. Arch, Psychiatry Clin. Neurosci, 1999, 249:271-79). Presenilins are postulated to regulate APP processing through their effects on gamma-secretase, an enzyme that cleaves APP (Cruts et al., 1998, Hum. Mutat., 11:183-190, Haass et al., Science, 1999, 286:916-19). Also, it is thought that the presenilins are involved in the cleavage of the Notch receptor, such that that they either directly regulate gamma-secretase activity or themselves are protease enzymes (De Strooper et al., Nature, 1999, 398:518-22). Two alternative transcripts of PSEN2 have been identified (Sato et al., 1999, J. Neurochem. 72(6):2498-505). Point mutations in the PS1 gene result in a selective increase in the production of the amyloidogenic peptide amyloid-beta (1-42) by proteolytic processing of the amyloid precursor protein (APP) (Lemere et al., 1996, Nat Med 2(10):1146-50). The possible role of PS1 in normal APP processing was studied by De Strooper et al. (Nature 391: 387-390, 1998) in neuronal cultures derived from PS1-deficient mouse embryos. They found that cleavage by alpha- and beta-secretase of the extracellular domain of APP was not affected by the absence of PS1, whereas cleavage by gamma-secretase of the transmembrane domain of APP was prevented, causing C-terminal fragments of APP to accumulate and a 5-fold drop in the production of amyloid peptide. Pulse-chase experiments indicated that PSI deficiency specifically decreased the turnover of the membrane-associated fragments of APP. Thus, PS1 appears to facilitate a proteolytic activity that cleaves the integral membrane domain of APP. The results indicated to the authors that mutations in PS1 that manifest clinically cause a gain of function, and that inhibition of PS1 activity is a potential target for anti-amyloidogenic therapy in Alzheimer disease.

Beta-Secretase

Beta-secretase, expressed specifically in the brain, is responsible for the proteolytic processing of the amyloid precursor protein (APP) associated with Alzheimer's disease (Potter et al., 2000, Nat. Biotechnol 18(2):125-26). It cleaves at the amino terminus of the beta peptide sequence, between residues 671 and 672 of APP, leading to the generation and extracellular release of beta-cleaved soluble APP, and a carboxyterminal fragment that is later released by gamma-secretase (Kinberly et al., 2000 J. Biol. Chem. 275(5):3173-78). Yan et al.(Nature, 1999, 402:533-37) identified a new membrane-bound aspartyl protease (Asp2) with beta-secretase activity. The Asp2 gene is expressed widely in brain and other tissues. Decreasing the expression of Asp2 in cells reduces amyloid beta-peptide production and blocks the accumulation of the carboxy-terminal APP fragment that is created by beta-secretase cleavage. Asp2 is a new protein target for drugs that are designed to block the production of amyloid beta-peptide peptide and the consequent formation of amyloid plaque in Alzheimer's disease.

Two aspartyl proteases involved in human placentation have recently been isolated:decidual aspartyl protease (DAP-1) and DAP-2 (Moses et al., Mol. Hum Reprod. 1999, 5:983-89).

Another member of the aspartyl peptidase family is HIV-1 retropepsin, from the human immunodeficiency virus type 1. This enzyme is vital for processing of the viral polyprotein and maturation of the mature virion.

2. Cysteine Proteases

Another class of proteases which perform a wide variety of functions within the body are the cysteine proteases. Among their roles are the processing of precursor proteins, and intracelluar degradation of proteins marked for disposal via the ubiquitin pathway. Eukaryotic cysteine proteases are a family of proteolytic enzymes which contain an active site cysteine. Catalysis proceeds through a thioester intermediate and is facilitated by a nearby histidine side chain; an asparagine completes the essential catalytic triad. Peptidases in this family with important roles in disease include the caspases, calpain, hedgehog, and Ubiquitin hydolases.

Cysteine proteases are produced by a large number of cells including those of the immune system (macrophages, monocytes, etc.). These immune cells exercise their protective role in the body, in part, by migrating to sites of inflammation and secreting molecules, among the secreted molecules are cysteine proteases.

Under some conditions, the inappropriate regulation of cysteine proteases of the immune system can lead to autoimmune diseases such as rheumatoid arthritis. For example, the over-secretion of the cysteine protease cathepsin C causes the degradation of elastin, collagen, laminin, and other structural proteins found in bones. Bone subjected to this inappropriate digestion is more susceptible to metastasis.

Caspase (C14)—Anopotosis

A cascade of protease reactions is believed to be responsible for the apoptotic changes observed in mammalian cells undergoing programmed cell death. This cascade involves many members of the aspartate-specific cysteine proteases of the caspase family, including caspases 2, 3, 6, 7, 8 and 10 (Salvesen and Dixit, Cell 1997, 91:443-446). Cancer cells that escape apoptotic signals, generated by cytotoxic chemotherapeutics or loss of normal cellular survival signals (as in metastatic cells), can go on to develop palpable tumors.

Other caspases are also involved in the activation of pro-inflammatory cytokines. Caspase 1 specifically processes the precursors of IL-1 p, and IL-18 (interferon-γ-inducing factor)(Salvesen and Dixit Cell 1997).

Calpain (C2)—Axonal Death, Dystrophies

Calcium-dependent cysteine proteases, collectively called calpain, are widely distributed in mammalian cells (Wang, 2000, Trends Neurosci. 23(1):20-26). The calpains are nonlysosomal intracellular cysteine proteases. The mammalian calpains include 2 ubiquitous proteins, CAPN1 and CAPN2, as well as 2 stomach-specific proteins, and CAPN3, which is muscle-specific (Herasse et al., 1999, Mol. Cell. Biol. 19(6):4047-55). The ubiquitous enzymes consist of heterodimers with distinct large subunits associated with a common small subunit, all of which are encoded by different genes. The large subunits of calpains can be subdivided into 4 domains; domains I and III, whose functions remain unknown, show no homology with known proteins. The former, however, may be important for the regulation of the proteolytic activity. Domain II shows similarity with other cysteine proteases, which share histidine, cysteine, and asparagine residues at their active sites. Domain IV is calmodulin-like. CAPN5 and CAPN6 differ from previously identified vertebrate calpains in that they lack a calmodulin-like domain IV (Ohno et al., 1990, Cytogenet. Cell Genet. 53(4):225-29).

Mutations in the CAPN3 gene have been associated with limb-girdle muscular dystrophy, type 2A (LGMD2A) (Allamand et al., 1995, Hum. Molec. Genet. 4:459-463). The slowly progressive muscle weakness associated with this disease is usually first evident in the pelvic girdle and then spreads to the upper limbs while sparing facial muscles. Calpain has also been implicated in the development of hyperactive Cdk5 leading to neuronal cell death associated with Alzheimer's disease (Patrick et al., 1999, Nature 402:615-622).

Hedgehog (C46)—Cancer

The organization and morphology of the developing embryo are established through a series of inductive interactions. One family of vertebrate genes has been described related to the Drosophila gene ‘hedgehog’ (hh) that encodes inductive signals during embryogenesis (Johnson and Tabin, 1997, Cell 90:979-990). ‘Hedgehog’ encodes a secreted protein that is involved in establishing cell fates at several points during Drosophila development (Marigo et al., 1995, Genomics 28:44-51). There are 3 known mammalian homologs of hh: Sonic hedgehog (Shh), Indian hedgehog (Ihh), and desert hedgehog (Dhh) (Johnson and Tabin, 1997, Cell 90:979-990). Like its Drosophila cognate, Shh encodes a signal that is instrumental in patterning the early embryo. It is expressed in Hensen's node, the floorplate of the neural tube, the early gut endoderm, the posterior of the limb buds, and throughout the notochord (Chiang et al., 1996, Nature 383:407-413). It has been implicated as the key inductive signal in patterning of the ventral neural tube, the anterior-posterior limb axis, and the ventral somites. Oro et al. (“Basal cell carcinomas in mice overexpressing sonic hedgehog.” Science 276: 817-821, 1997) showed that transgenic mice overexpressing SHH in the skin developed many features of the basal cell nevus syndrome, demonstrating that SHH is sufficient to induce basal cell carcinomas (BCCs) in mice. The data suggested that SHH may have a role in human tumorigenesis. Activating mutations of SHH or another ‘hedgehog’ gene may be an alternative pathway for BCC formation in humans. The human mutation his133tyr (his134tyr in mouse) is a candidate. It is distinct from loss-of-function mutations reported for individuals with holoprosencephaly (Oro et al., 1997, Science 276:817-821). His133 lies adjacent in the catalytic site to his134, one of the conserved residues thought to be necessary for catalysis. SHH may be a dominant oncogene in multiple human tumors, a mirror of the tumor suppressor activity of the opposing ‘patched’ (PTCH) gene (Aszterbaum et al., 1998, J. Invest. Derm. 110:885-888). The rapid and frequent appearance of Shh-induced tumors in the mice suggested that disruption of the SHH-PTC pathway is sufficient to create BCCs.

Members of the vertebrate hedgehog family (Sonic, Indian, and Desert) have been shown to be essential for the development of various organ systems, including neural, somite, limb, skeletal, and for male gonad morphogenesis. Desert hedgehog is expressed in the developing retina, whereas Indian hedgehog (Ihh) is expressed in the developing and mature retinal pigmented epithelium beginning at embryonic day 13 (Levine et al., J.Neurosci., 1997, 17(16):6277-88). Dhh has also been implicated in having a role in the regulation of spermatogenesis. Sertoli cell precursors express Sry, sex determining gene, which leads to testis development in mammals. Dhh expression is initiated in Sertoli cell precursors shortly after the activation of Sry and persists in the testis into the adult. Bitgood et al. (Curr. Biol., 1996, 6(3):298-304) disclose that female mice homozygous for a Dhh-null mutation show no obvious phenotype, whereas males are viable but infertile having a complete absence of mature sperm, demonstrating that Dhh signaling plays an essential role in the regulation of mammalian spermatogenesis. Dhh has also been found to have a role in the and maintenance of protective nerve sheaths endo-, peri- and epineurium. In Dhh knockout mice, the connective tissue sheaths in adult nerves appear highly abnormal by electron microscopy. Mirsky et al., (Ann. N.Y Acad. Sci., 1999, 883:196-202) demonstrate that Dhh signaling from Schwann cells to the mesenchyme is involved in the formation of a morphologically and functionally normal perineurium.

Recent advances in developmental and molecular biology during embryogenesis and organogenesis have provided new insights into the mechanism of bone formation. Iwasaki et al., (J. Bone Joint Surg. Br., 1999, 81(6):1076-82) demonstrate that Indian Hedgehog (Ihh) is expressed in cartilage cell precursors and later in mature and hypertrophic chondrocytes. Ihh plays a critical role in the morphogenesis of the vertebrate skeleton. Becker et al. (Dev. Biol., 1997, 187(2):298-310) provide data which suggests that Ihh is also involved in mediating differentiation of extraembryonic endoderm during early mouse embryogenesis. Short limbed dwarfism, with decreased chondrocyte proliferation and extensive hypertrophy are the results of targeted deletion of Ihh (Karp et al., 2000, Development 127(3):543-48). The expression of Ihh mRNA and protein is unregulated dramatically as F9 cells differentiate in response to retinoic acid, into either parietal endoderm or embryoid bodies, containing an outer visceral endoderm layer. RT-PCR analysis of blastocyst outgrowth cultures demonstrates that whereas little or no Ihh message is present in blastocysts, significant levels appear upon subsequent days of culture, coincident with the emergence of parietal endoderm cells.

Ubiguitin Hydrolases (C12)—Apoptosis, Checkpoint Integrity

14 genes in this patent belong to the ubiquitin hydrolase family, SEQID:5, SEQID:6, SEQID:7, SEQID:8, SEQID:9, SEQID:10, SEQID:11, SEQID:12, SEQID:13, SEQID:14, SEQID:15, SEQID:16, SEQID:17, SEQID:18. The polypeptides encoded by these genes may have one or more of the following activities.

Ubiquitin carboxyl-terminal hydrolases (3.1.2.15) (deubiquitinating enzymes) are thiol proteases that recognize and hydrolyze the peptide bond at the C-terminal glycine of ubiquitin. These enzymes are involved in the processing of poly-ubiquitin precursors as well as that of ubiquinated proteins. In eukaryotic cells, the covalent attachment of ubiquitin to proteins plays a role in a variety of cellular processes. In many cases, ubiquitination leads to protein degradation by the 26S proteasome. Protein ubiquitination is reversible, and the removal of ubiquitin is catalyzed by deubiquitinating enzymes, or DUBs. A defect in these enzymes, catalyzing the removal of ubiquitin from ubiquinated proteins, may be characteristic of neurodegenerative diseases such as Alzheimer's, Parkinson's, progressive supranuclear palsy, and Pick's and Kuf's disease.

Panain (C1)—Cathepsins K, S and B,—Bone Resorbtion, Ag Processing (Prosite PS00139).

One gene in this patent belongs to the Papain family, SEQID:4. The polypeptide encoded by this gene may have one or more of the following activities.

Cathepsin K, a member of the papain family of peptidases, is involved in osteoclastic resorption. It plays an important role in extracellular degradation and may have a role in disorders of bone remodeling, such as pyncodysostosis, an autosomal recessive osteochondrodysplasia characterized by osteosclerosis and short stature. Antigen presentation by major histocompatibility complex (MHC) class II molecules requires the participation of different proteases in the endocytic route to degrade endocytosed antigens as well as the MHC class II-associated invariant chain. Only cathepsin S, a member of the papain family, appears to be essential for complete destruction of the invariant chain. Cathepsin B is overexpressed in tumors of the lung, prostate, colon, breast, and stomach. Hughes et al. (Proc. Nat. Acad. Sci. 95: 12410-12415, 1998) found an amplicon at 8p23-p22 that resulted in cathepsin B overexpression in esophageal adenocarcinoma Abundant extracellular expression of CTSB protein was found in 29 of 40 (72.5%) of esophageal adenocarcinoma specimens by use of immunohistochemical analysis. The findings were thought to support an important role for CTSB in esophageal adenocarcinoma and possibly in other tumors.

Cathepsin B, a lysomal protease, is being studied as a prognostic marker in various cancers (breast, pulmonary adenocarcinomas).

Cysteine Protease AEP

The cysteine protease AEP plays another role in the immune functions. It has been implicated in the protease step required for antigen processing in B cells. (Manoury et al. Nature 396:695-699 (1998))

Hepatitis A Viral Protease (C3E)

The Hepatitis A genome encodes a cysteine protease required for enzymatic cleavages in vivo to yield mature proteins (Wang, 1999, Prog. Drug Res. 52:197-219). This enzyme and its homologs in other viruses (such as hepatitis E virus) are potential targets for chemotherapeutic intervention.

3. Metalloproteases

Collagenase (M10)—Invasion

Two genes in this patent are members of the M10 family, SEQID:19 and SEQID:20. The polypeptides encoded by these genes may have one or more of the following activities.

Matrix degradation is an essential step in the spread of cancer. The 72- and 92-kD type IV collagenases are members of a group of secreted zinc metalloproteases which, in mammals, degrade the collagens of the extracellular matrix. Other members of this group include interstitial collagenase and stromelysin (Nagase et al., 1992, Matrix Suppl. 1:421-424). By targeted disruption in embryonic stem cells, Vu et al. (Cell, 1998, 934:11-22) created homozygous mice with a null mutation in the MMP9/gelatinase B gene. These mice exhibited an abnormal pattern of skeletal growth plate vascularization and ossification. Growth plates from MMP9-null mice in culture showed a delayed release of an angiogenic activator, establishing a role for this proteinase in controlling angiogenesis.

MMP2 (gelatinase A) have been associated with the aggressiveness of human cancers (Chenard et al., 1999, Int. J. Cancer, 82:208-12). In a study comparing basal cell carcinomas (BCC) with the more aggressive squamous cell carcinomas (SCC), both MMP2 and MMP9 were expressed at a higher level in SCC (Dumas et al., 1999, Anticancer Res., 19(4B):2929-38). Additionally, expression of MMP2 and MMP9 in T lymphocytes has recently been shown to be modulated by the Ras/MAP kinase signaling pathways (Esparza et al., 1999, Blood, 94:2754-66) (see also, Li et al., 1998, Biochim. Biophys. Acta, 1405:110-20).

ADAMs (M12)—TNF, Inflammation, Growth Factor Processing

The ADAM peptidases are a family of proteins containing a disintegrin and metalloproteinase (ADAM) domain (Werb and Yan, Science, 1998, 282:1279-1280). Members of this family are cell surface proteins with a unique structure possessing both potential adhesion and protease domains (Primakoff and Myles, Trends in Genet., 2000, 16:83-87). Activity of these proteases can be linked to TNF, inflammation, and/or growth factor processing.

ADAM proteases have also been characterized as having a pro- and metalloproteinase domain, a disintegrin domain, a cysteine-rich region and an EGF repeat (Blobel, 1997, Cell, 90:589-592 which is hereby incorporated herein by reference in its entirety including any figures, tables, or drawings). They have been associated with the release from the plasma membrane of numerous proteins including Tumor Necrosis Factor-α (TNF-α), kit-ligand, TGFα, Fas-ligand, cytokine receptors such as the Il-6 receptor and the NGF receptor, as well as adhesion proteins such as L-selectin, and the b amyloid precursor proteins (Blobel, 1997, Cell, 90:589-592).

Tumor necrosis factor-α is synthesized as a proinflammatory cytokine from a 233-amino acid precursor. Conversion of the membrane-bound precursor to a secreted mature protein is mediated by a protease termed TNF-α convertase. TNF-α is involved in a variety of diseases. ADAM17, which contains a disintegrin and metalloproteinase domains, is also called ‘tumor necrosis factor-α converting enzyme’ (TACE) (Black et al., Nature, 1997, 385:729-33). The gene encodes an 824-amino acid polypeptide containing the features of the ADAM family: a secretory signal sequence, a disintegrin domain, and a metalloprotease domain. Expression studies showed that the encoded protein cleaves precursor tumor necrosis factor-α to its mature form. This enzyme may also play a role in the processing of Transforming Growth Factor-α (TGF-α), as mice which lack the gene are similar in phenotype to those that lack TGF-α (Peschon et al., Science, 282:1281-1284).

Neprylisin (M13)—Endothelin-Converting Enzyme

One gene in this patent, SEQID:21, is a member of this family. The polypeptide encoded by this gene may have one or more of the following activities.

Neprylisin, a metallopeptidase active in degradation of enkephalins and other bioactive peptides, is a drug target in hypertension and renal disease (Oefner, et al., J. Mol. Biol., 2000, 296:341-49).

Carboxypeptidase (M14)—Neurotransmitter Processing

Three genes in this application are Zn carboxypeptidases, SEQID:1, SEQID:2, and SEQID:3. The polypeptides encoded by these genes may have one or more of the following activites.

Carboxypeptidases specifically remove COOH-terminal basic amino acids (arginine or lysine). They have important functions in many biologic processes, including activation, inactivation, or modulation of peptide hormone activity, neurotransmitter processing, and alteration of physical properties of proteins and enzymes.

Dipeptidase (M2)—ACE

One protease in this patent is a member of the M2 family: SEQID:22. The polypeptide encoded by this gene may have one or more of the following activities.

Angiotensin I converting enzyme (EC 3.4.15.1 ), or kininase II, is adipeptidyl carboxypeptidase that plays an important role in blood pressure regulation and electrolyte balance by hydrolyzing angiotensin I into angiotensin II, a potent vasopressor, andaldosterone-stimulating peptide. The enzyme is also able to inactivate bradykinin, a potent vasodilator. Although angiotensin-converting enzyme has been studied primarily in the context of its role in blood pressure regulation, this widely distributed enzyme has many other physiologic functions. There are two forms of ACE: a testis-specific isozyme and a somatic isozyme which has two active centers.

Matrix Metalloproteases (M10B)—Tissue Remodeling and Inflammation

The matrix metalloproteases (MMPs) are a family of related matrix-degrading enzymes that are important in tissue remodeling and repair during development and inflammation. Abnormal expression is associated with various diseases such as tumor invasiveness, arthritis, and atherosclerosis. MMP activity may also be related to tobacco-induced pulmonary emphysema.

The matrix metalloproteases (MMPs) are a family of related matrix-degrading enzymes that are important in tissue remodeling and repair during development and inflammation (Belotti et al., 1999, Int. J. Biol. Markers 14(4):232-38). Abnormal expression is associated with various diseases such as tumor invasiveness (Johansson and Kahari, 2000, Histol. Histopathol. 15(l):225-37), arthritis (Malemud et al., 1999, Front. Biosci. 4:D762-71), and atherosclerosis (Nagase, 1997, Biol. Chem. 378(3-4):151-60). MMP activity may also be related to tobacco-induced pulmonary emphysema (Dhami et al., Am. J. Respir. Cell Mol. Biol., 2000, 22:244-52).

SREBP Protease (M50)

The sterol regulatory element-binding proteins protease functions in the intra-membrane proteolysis and release of sterol-regulatory binding proteins (SREBPs) (Duncan et al., 1997, J. Biol. Chem. 272:12778-85). SREBPs activate genes of cholesterol and fatty acid metabolism, making the SREBP protease an attractive target for therapeutic modulation (Brown et al., 1997, Cell 89:331-340).

Metalloprotease Processing of Growth Factors

In addition to the processing of TGF-α described above, metalloproteases have been directly demonstrated to be active in the processing of the precursor of other growth factors such as heparin-binding EGF (proHB-EFG) (Izumi et al., EMBO J, 1998,17:7260-72), and amphiregulin (Brown et al., 1998, J. Biol. Chem., 27:17258-68).

Additionally, metalloproteases have recently been shown to be instrumental in the communication whereby stimulation of a GPCR pathway results in stimulation of the MAP kinase pathway (Prenzel et al., 1999, Nature, 402:884-888). The growth factor intermediate in the pathway, HB-EGF is released by the cell in a proteolytic step regulated by the GPCR pathway involving an uncharacterized metalloprotease. After release, the HB-EGF is bound by the extracellular matrix and then presented to the EGF receptors on the surface, resulting in the activation of the MAP kinase pathway (Prenzel et al., 1999, Nature, 402:884-888).

A recent study by Gallea-Robache et al. (1997) has also implicated a metalloprotease family displaying different substrate specificites in the shedding of other growth factors including macrophage colony-stimulating factor (M-CSF) and stem cell factor (SCF) (Gallea-Robache et al., 1997, Cytokine 9:340-46). The shedding of M-CSF (also known as CSF-1) has been linked to activation of Protein Kinase C by phorbol esters (Stein et al., 1991, Oncogene, 6:601-05).

4. Serine Proteases

The serine proteases are a class which includes trypsin, kallikrein, chymotrypsin, elastase, thrombin, tissue plasminogen activator (tPA), urokinase plasminogen activator (uPA), plasrnin (Werb, Cell, 1997, 91:439-442), kallikrein (Clements, Biol. Res., 1998, 31151-59), and cathepsin G (Shamamian et al., Surgery, 2000, 127:14247). These proteases have in common a well-conserved catalytic triad of amino acid residues in their active site consisting of histidine-57, aspartic acid-102, and serine-195 (using the chymotrypsin numbering system). Serine protease activity has been linked to coagulation and they may have use as tumor markers.

Serine proteases can be further subclassified by their specificity in substrates. The elastases prefer to cleave substrates adjacent to small aliphatic residues such as valine, chymases prefer to cleave near large aromatic hydrophobic residures, and tryptases prefer positively charged residues. One additional class of serine protease has been described recently which prefers to cleave adjacent to a proline. This prolyl endopeptidase has been implicated in the progression of memory loss, in Alzheimer's patients (Toide et al., 1998, Rev. Neurosci. 9(1):17-29).

A partial list of proteases known to belong to this large and important family include: blood coagulation factors VII, IX, X, XI and XII; thrombin; plasminogen; complement components C1r, C1s, C2; complement factors B, D and I; complement-activating component of RA-reactive factor; elastases 1, 2, 3A, 3B (protease E); hepatocyte growth factor activator; glandular (tissue) kallikreins including EGF-binding protein types A, B, and C; NGF-γ chain, γ-renin, and prostate specific antigen (PSA); plasma kallikrein; mast cell proteases; myeloblastin (proteinase 3) (Wegener's autoantigen); plasminogen activators (urokinase-type, and tissue-type); and the trypsins I, II, III, and IV. These peptidases play key roles in coagulation, tumorigenesis, control of blood pressure, release of growth factors, and other roles. (http://www.babraham.co.uk/Merops/Merops.htm).

Proteases of the trypsin family in this patent include SGPr434, SEQID:24; SGPr446_{—}1, SEQID:25; SGPr447, SEQID:26; SGPr432_{—}1, SEQID:27; SGPr529, SEQID:28; SGPr428_{—}1, SEQID:29; SGPr425, SEQID:30; SGPr548, SEQID:31; SGPr396, SEQID:32; SGPr426, SEQID:33; SGPr552, SEQID:34; SGPr405, SEQID:35; SGPr485_{—}1, SEQID:36; SGPr534, SEQID:37; SGPr390, SEQID:38; SGPr521, SEQID:39; SGPr530_{—}1, SEQID:40; SGPr520, SEQID:41; SGPr455, SEQID:42; SGPr507_{—}2, SEQID:43; SGPr559, SEQID:44; SGPr567_{—}1, SEQID:45; SGPr479_{—}1, SEQID:46; SGPr489_{—}1, SEQID:47; SGPr465_{—}1, SEQID:48; SGPr524_{—}1, SEQID:49; SGPr422, SEQID:50; SGPr538, SEQID:51; SGPr527_{—}1, SEQID:52; SGPr542, SEQID:53; SGPr551, SEQID:54; SGPr451, SEQID:55; SGPr452_{—}1, SEQID:56; SGPr504, SEQID:57; SGPr469, SEQID:58; SGPr400, SEQID:59. SEQID:23 is a serine protease of the subtilase sub-family. Limited proteolysis of most large protein precursors is carried out in vivo by the subtilisin-like pro-protein convertases. Many important biological processes such as peptide hormone synthesis, viral protein processing and receptor maturation involve proteolytic processing by these enzymes, making them potential targets for the development of novel therapeutic agents (Bergeron F, J Mol Endocrinol February 2000; 24(1):1-22)

5. Threonine Peptidases (T1)—(Prosite PDOC00326/PDOC00668)

Proteasomal Subunits (TIA)

The proteasome is a multicatalytic threonine proteinase complex involved in ATP/ubiquitin dependent non-lysosomal proteolysis of cellular substrates. It is responsible for selective elimination of proteins with aberrant structures, as well as naturally occurring short-lived proteins related to metabolic regulation and cell-cycle progression (Momand et al., 2000, Gene 242(1-2):15-29, Bochtler et al., 1999, Annu. Rev. Biophys Biomol Struct. 28:295-317). The proteasome inhibitor lactacystin reversibly inhibits proliferation of human endothelial cells, suggesting a role for proteasomes in angiogenesis (Kumeda, et al., Anticancer Res. September-October 1999; 19(5B):3961-8). Another important function of the proteasome in higher vertebrates is to generate the peptides presented on MHC-class 1 molecules to circulating lymphocytes (Castelli et al., 1997, Int. J. Clin. Lab. Res. 27(2):103-10). The proteasome has a sedimentation coefficient of 26S and is composed of a 20S catalytic core and a 22S regulatory complex. Eukaryotic 20S proteasomes have a molecular mass of 700 to 800 kD and consist of a set of over 15 kinds of polypeptides of 21 to 32 kD. All eukaryotic 20S proteasome subunits can be classified grossly into 2 subfamilies, α and β, by their high similarity with either the α or β subunits of the archaebacterium Thermoplasma acidophilum (Mayr et al., 1999, Biol. Chem. 380(10):1183-92). Several of the components have been identified as threonine peptidases, suggesting that this class of peptidases plays a key role in regulating metabolic pathways and cell-cycle progression, among other functions (Yorgin et al., 2000, J. Immunol. 164(6):2915-23).

6. Peptidases of Unknown Catalytic Mechanism

The prenyl-protein specific protease responsible for post-translational processing of the Ras proto-oncogene and other prenylated proteins falls into this class. This class also includes several viral peptidases that may play a role in mammalian infection, including cardiovirus endopeptidase 2A (encephalomyocarditis virus) (Molla et al., 1993, J. Virol. 67(8):4688-95), NS2-3 protease (hepatitis C virus) (Blight et al., 1998, Antivir. Ther. 3(Suppl 3):71-81), endopeptidase (infectious pancreatic necrosis virus) (Lejal et al., J. Gen. Virol., 2000, 81:983-992), and the Npro endopeptidase (hog cholera virus) (Tratschin et al., 1998, J. Virol. 72(9):7681-84).

Nucleic Acid Probes, Methods, and Kits for Detection of Proteases

A nucleic acid probe of the present invention may be used to probe an appropriate chromosomal or cDNA library by usual hybridization methods to obtain other nucleic acid molecules of the present invention. A chromosomal DNA or cDNA library may be prepared from appropriate cells according to recognized methods in the art (cf. “Molecular Cloning: A Laboratory Manual”, second edition, Cold Spring Harbor Laboratory, Sambrook, Fritsch, & Maniatis, eds., 1989).

In the alternative, chemical synthesis can be carried out in order to obtain nucleic acid probes having nucleotide sequences which correspond to N-terminal and C-terminal portions of the amino acid sequence of the polypeptide of interest. The synthesized nucleic acid probes may be used as primers in a polymerase chain reaction (PCR) carried out in accordance with recognized PCR techniques, essentially according to PCR Protocols, “A Guide to Methods and Applications”, Academic Press, Michael, et al., eds., 1990, utilizing the appropriate chromosomal or cDNA library to obtain the fragment of the present invention.

One skilled in the art can readily design such probes based on the sequence disclosed herein using methods of computer alignment and sequence analysis known in the art (“Molecular Cloning: A Laboratory Manual”, 1989, supra). The hybridization probes of the present invention can be labeled by standard labeling techniques such as with a radiolabel, enzyme label, fluorescent label, biotin-avidin label, chemiluminescence, and the like. After hybridization, the probes may be visualized using known methods.

The nucleic acid probes of the present invention include RNA, as well as DNA probes, such probes being generated using techniques known in the art. The nucleic acid probe may be immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling nucleic acid probes to such solid supports are well known in the art.

The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample which is compatible with the method utilized.

One method of detecting the presence of nucleic acids of the invention in a sample comprises (a) contacting said sample with the above-described nucleic acid probe under conditions such that hybridization occurs, and (b) detecting the presence of said probe bound to said nucleic acid molecule. One skilled in the art would select the nucleic acid probe according to techniques known in the art as described above. Samples to be tested include but should not be limited to RNA samples of human tissue.

A kit for detecting the presence of nucleic acids of the invention in a sample comprises at least one container means having disposed therein the above-described nucleic acid probe. The kit may further comprise other containers comprising one or more of the following: wash reagents and reagents capable of detecting the presence of bound nucleic acid probe. Examples of detection reagents include, but are not limited to radiolabelled probes, enzymatic labeled probes (horseradish peroxidase, alkaline phosphatase), and affinity labeled probes (biotin, avidin, or steptavidin). Preferably, the kit further comprises instructions for use.

In detail, a compartmentalized kit includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample, a container which contains the probe or primers used in the assay, containers which contain wash reagents (such as phosphate buffered saline, Tris-buffers, and the like), and containers which contain the reagents used to detect the hybridized probe, bound antibody, amplified product, or the like. One skilled in the art will readily recognize that the nucleic acid probes described in the present invention can readily be incorporated into one of the established kit formats which are well known in the art.

DNA Constructs Comprising a Protease Nucleic Acid Molecule and Cells Containing These Constructs.

The present invention also relates to a recombinant DNA molecule comprising, 5′ to 3′, a promoter effective to initiate transcription in a host cell and the above-described nucleic acid molecules. In addition, the present invention relates to a recombinant DNA molecule comprising a vector and an above-described nucleic acid molecule. The present invention also relates to a nucleic acid molecule comprising a transcriptional region functional in a cell, a sequence complementary to an RNA sequence encoding an amino acid sequence corresponding to the above-described polypeptide, and a transcriptional termination region functional in said cell. The above-described molecules may be isolated and/or purified DNA molecules.

The present invention also relates to a cell or organism that contains an above-described nucleic acid molecule and thereby is capable of expressing a polypeptide. The polypeptide may be purified from cells which have been altered to express the polypeptide. A cell is said to be “altered to express a desired polypeptide” when the cell, through genetic manipulation, is made to produce a protein which it normally does not produce or which the cell normally produces at lower levels. One skilled in the art can readily adapt procedures for introducing and expressing either genomic, cDNA, or synthetic sequences into either eukaryotic or prokaryotic cells.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

If desired, the non-coding region 3′ to the sequence encoding a protease of the invention may be obtained by the above-described methods. This region may be retained for its transcriptional termination regulatory sequences, such as termination and polyadenylation. Thus, by retaining the 3′-region naturally contiguous to the DNA sequence encoding a protease of the invention, the transcriptional termination signals may be provided. Where the transcriptional termination signals are not satisfactorily functional in the expression host cell, then a 3′ region functional in the host cell may be substituted.

Two DNA sequences (such as a promoter region sequence and a sequence encoding a protease of the invention) are said to be operably linked if the nature of the linkage between the two DNA sequences allows the protease sequence to be transcribed, i.e., where the linkage does not (1) result in the introduction of a frame-shift mutation, (2) interfere with the ability of the promoter region sequence to direct the transcription of a gene sequence encoding a protease of the invention, or (3) interfere with the ability of the gene sequence of a protease of the invention to be transcribed by the promoter region sequence. Thus, a promoter region would be operably linked to a DNA sequence if the promoter were capable of effecting transcription of that DNA sequence. Thus, to express a gene encoding a protease of the invention, transcriptional and translational signals recognized by an appropriate host are necessary.

The present invention encompasses the expression of a gene encoding a protease of the invention (or a functional derivative thereof) in either prokaryotic or eukaryotic cells. Prokaryotic hosts are, generally, very efficient and convenient for the production of recombinant proteins and are, therefore, one type of preferred expression system for proteases of the invention. Prokaryotes most frequently are represented by various strains of E. coli. However, other microbial strains may also be used, including other bacterial strains.

In prokaryotic systems, plasmid vectors that contain replication sites and control sequences derived from a species compatible with the host may be used. Examples of suitable plasmid vectors may include pBR322, pUC 118, pUC 119 and the like; suitable phage or bacteriophage vectors may include λgt10, λgt11 and the like; and suitable virus vectors may include pMAM-neo, pKRC and the like. Preferably, the selected vector of the present invention has the capacity to replicate in the selected host cell.

Recognized prokaryotic hosts include bacteria such as E. coli, Bacillus, Streptomyces, Pseudomonas, Salmonella, Serratia, and the like. However, under such conditions, the polypeptide will not be glycosylated. The prokaryotic host must be compatible with the replicon and control sequences in the expression plasmid.

To express a protease of the invention (or a functional derivative thereof) in a prokaryotic cell, it is necessary to operably link the sequence encoding the protease of the invention to a functional prokaryotic promoter. Such promoters may be either constitutive or, more preferably, regulatable (i.e., inducible or derepressible). Examples of constitutive promoters include the int promoter of bacteriophage %, the bla promoter of the β-lactamase gene sequence of pBR322, and the cat promoter of the chloramphenicol acetyl transferase gene sequence of pPR325, and the like. Examples of inducible prokaryotic promoters include the major right and left promoters of bacteriophage λ (P_{L} and P_{R}), the trp, recA, λacZ, λacI, and gal promoters of E. coli, the α-amylase (Ulmanen et al., J. Bacteriol. 162:176-182, 1985) and the z,900 -28-specific promoters of B. subtilis (Gilman et al., Gene Sequence 32:11-20, 1984), the promoters of the bacteriophages of Bacillus (Gryczan, in: The Molecular Biology of the Bacilli, Academic Press, Inc., NY, 1982), and Streptomyces promoters (Ward et al., Mol. Gen. Genet. 203:468-478, 1986). Prokaryotic promoters are reviewed by Glick (Ind. Microbiot. 1:277-282, 1987), Cenatiempo (Biochimie 68:505-516, 1986), and Gottesman (Ann. Rev. Genet. 18:415-442, 1984).

Proper expression in a prokaryotic cell may also require the presence of a ribosome-binding site upstream of the gene sequence-encoding sequence. Such ribosome-binding sites are disclosed, for example, by Gold et al. (Ann. Rev. Microbiol. 35:365-404, 1981). The selection of control sequences, expression vectors, transformation methods, and the like, are dependent on the type of host cell used to express the gene. As used herein, “cell”, “cell line”, and “cell culture” may be used interchangeably and all such designations include progeny. Thus, the words “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the-originally transformed cell.

Host cells which may be used in the expression systems of the present invention are not strictly limited, provided that they are suitable for use in the expression of the protease polypeptide of interest. Suitable hosts may often include eukaryotic cells. Preferred eukaryotic hosts include, for example, yeast, fungi, insect cells, mammalian cells either in vivo, or in tissue culture. Mammalian cells which may be useful as hosts include HeLa cells, cells of fibroblast origin such as VERO or CHO-KI, or cells of lymphoid origin and their derivatives. Preferred mammalian host cells include SP2/0 and J558L, as well as neuroblastoma cell lines such as IMR 332, which may provide better capacities for correct post-translational processing.

In addition, plant cells are also available as hosts, and control sequences compatible with plant cells are available, such as the cauliflower mosaic virus 35S and 19S, and nopaline synthase promoter and polyadenylation signal sequences. Another preferred host is an insect cell, for example the Drosophila larvae. Using insect cells as hosts, the Drosophila alcohol dehydrogenase promoter can be used (Rubin, Science 240:1453-1459, 1988). Alternatively, baculovirus vectors can be engineered to express large amounts of proteases of the invention in insect cells (Jasny, Science 238:1653, 1987; Miller et al., in: Genetic Engineering, Vol. 8, Plenum, Setlow et al., eds., pp. 277-297, 1986).

Any of a series of yeast expression systems can be utilized which incorporate promoter and termination elements from the actively expressed sequences coding for glycolytic enzymes that are produced in large quantities when yeast are grown in mediums rich in glucose. Known glycolytic gene sequences can also provide very efficient transcriptional control signals. Yeast provides substantial advantages in that it can also carry out post-translational modifications. A number of recombinant DNA strategies exist utilizing strong promoter sequences and high copy number plasmids which can be utilized for production of the desired proteins in yeast. Yeast recognizes leader sequences on cloned mammalian genes and secretes peptides bearing leader sequences (i.e., pre-peptides). Several possible vector systems are available for the expression of proteases of the invention in a mammalian host.

A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has-a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation.

Expression of proteases of the invention in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-31, 1981); and the yeast gal4 gene sequence promoter (Johnston et al., Proc. Natl. Acad. Sci. (USA) 79:6971-6975, 1982; Silver et al., Proc. Natl. Acad. Sci. (USA) 81:5951-5955, 1984).

Translation of eukaryotic mRNA is initiated at the codon which encodes the first methionine. For this reason, it is preferable to ensure that the linkage between a eukaryotic promoter and a DNA sequence which encodes a protease of the invention (or a functional derivative thereof) does not contain any intervening codons which are capable of encoding a methionine (i.e., AUG). The presence of such codons results either in the formation of a fusion protein (if the AUG codon is in the same reading frame as the protease of the invention coding sequence) or a frame-shift mutation (if the AUG codon is not in the same reading frame as the protease of the invention coding sequence).

A nucleic acid molecule encoding a protease of the invention and an operably linked promoter may be introduced into a recipient prokaryotic or eukaryotic cell either as a nonreplicating DNA or RNA molecule, which may either be a linear molecule or, more preferably, a closed covalent circular molecule. Since such molecules are incapable of autonomous replication, the expression of the gene may occur through the transient expression of the introduced sequence. Alternatively, permanent expression may occur through the integration of the introduced DNA sequence into the host chromosome.

A vector may be employed which is capable of integrating the desired gene sequences into the host cell chromosome. Cells which have stably integrated the introduced DNA into their chromosomes can be selected by also introducing one or more markers which allow for selection of host cells which contain the expression vector. The marker may provide for prototrophy to an auxotrophic host, biocide resistance, e.g., antibiotics, or heavy metals, such as copper, or the like. The selectable marker gene sequence can either be directly linked to the DNA gene sequences to be expressed, or introduced into the same cell by co-transfection. Additional elements may also be needed for optimal synthesis of mRNA. These elements may include splice signals, as well as transcription promoters, enhancers, and termination signals. cDNA expression vectors incorporating such elements include those described by Okayama (Mol. Cell. Biol. 3:280-289, 1983).

The introduced nucleic acid molecule can be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Preferred prokaryotic vectors include plasmids such as those capable of replication in E. coli (such as, for example, pBR322, ColE1, pSC101, pACYC 184, πcVX; “Molecular Cloning: A Laboratory Manual”, 1989, supra). Bacillus plasmids include pC194, pC221, pT127, and the like (Gryczan, In: The Molecular Biology of the Bacilli, Academic Press, NY, pp. 307-329, 1982). Suitable Streptomyces plasmids include p1J101 (Kendall et al., J. Bacteriol. 169:4177-4183, 1987), and streptomyces bacteriophages such as φC31 (Chater et al., In: Sixth International Symposium on Actinomycetales Biology, Akademiai Kaido, Budapest, Hungary, pp. 45-54, 1986). Pseudomonas plasmids are reviewed by John et al. (Rev. Infect. Dis. 8:693-704, 1986), and Izaki (Jpn. J. Bacteriol. 33:729-742, 1978).

Preferred eukaryotic plasmids include, for example, BPV, vaccinia, SV40, 2-micron circle, and the like, or their derivatives. Such plasmids are well known in the art (Botstein et al., Miami Wntr. Symp. 19:265-274, 1982; Broach, In: The Molecular Biology of the Yeast Saccharomyces: Life Cycle and Inheritance, Cold Spring Harbor Laboratory, Cold Spring Harbor, N.Y., p. 445-470, 1981; Broach, Cell 28:203-204, 1982; Bollon et al., J. Clin. Hematol. Oncol. 10:39-48, 1980; Maniatis, In: Cell Biology: A Comprehensive Treatise, Vol. 3, Gene Sequence Expression, Academic Press, NY, pp. 563-608, 1980).

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene(s) results in the production of a protease of the invention, or fragments thereof. This can take place in the transformed cells as such, or following the induction of these cells to differentiate (for example, by administration of bromodeoxyuracil to neuroblastoma cells or the like). A variety of incubation conditions can be used to form the peptide of the present invention. The most preferred conditions are those which mimic physiological conditions.

Antibodies, Hybridomas, Methods of Use and Kits for Detection of Proteases

The present invention relates to an antibody having binding affinity to a protease of the invention. The protease polypeptide may have the amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118, or a functional derivative thereof, or at least 9 contiguous amino acids thereof (preferably, at least 20, 30, 35, or 40 contiguous amino acids thereof).

The present invention also relates to an antibody having specific binding affinity to a protease of the invention. Such an antibody may be isolated by comparing its binding affinity to a protease of the invention with its binding affinity to other polypeptides. Those which bind selectively to a protease of the invention would be chosen for use in methods requiring a distinction between a protease of the invention and other polypeptides. Such methods could include, but should not be limited to, the analysis of altered protease expression in tissue containing other polypeptides.

The proteases of the present invention can be used in a variety of procedures and methods, such as for the generation of antibodies, for use in identifying pharmaceutical compositions, and for studying DNA/protein interaction.

The proteases of the present invention can be used to produce antibodies or hybridomas. One skilled in the art will recognize that if an antibody is desired, such a peptide could be generated as described herein and used as an immunogen. The antibodies of the present invention include monoclonal and polyclonal antibodies, as well fragments of these antibodies, and humanized forms. Humanized forms of the antibodies of the present invention may be generated using one of the procedures known in the art such as chimerization or CDR grafting.

The present invention also relates to a hybridoma which produces the above-described monoclonal antibody, or binding fragment thereof. A hybridoma is an immortalized cell line which is capable of secreting a specific monoclonal antibody.

In general, techniques for preparing monoclonal antibodies and hybridomas are well known in the art (Campbell, Monoclonal Antibody Technology: Laboratory Techniques in Biochemistry and Molecular Biology, Elsevier Science Publishers, Amsterdam, The Netherlands, 1984; St. Groth et al., J. Immunol. Methods 35:1-21, 1980). Any animal (mouse, rabbit, and the like) which is known to produce antibodies can be immunized with the selected polypeptide. Methods for immunization are well known in the art. Such methods include subcutaneous or intraperitoneal injection of the polypeptide. One skilled in the art will recognize that the amount of polypeptide used for immunization will vary based on the animal which is immunized, the antigenicity of the polypeptide and the site of injection.

The polypeptide may be modified or administered in an adjuvant in order to increase the peptide antigenicity. Methods of increasing the antigenicity of a polypeptide are well known in the art. Such procedures include coupling the antigen with a heterologous protein (such as globulin or β-galactosidase) or through the inclusion of an adjuvant during immunization.

For monoclonal antibodies, spleen cells from the immunized animals are removed, fused with myeloma cells, such as SP2/0-Agl4 myeloma cells, and allowed to become monoclonal antibody producing hybridoma cells. Any one of a number of methods well known in the art can be used to identify the hybridoma cell which produces an antibody with the desired characteristics. These include screening the hybridomas with an ELISA assay, western blot analysis, or radioimmunoassay (Lutz et al., Exp. Cell Res. 175:109-124, 1988). Hybridomas secreting the desired antibodies are cloned and the class and subclass are determined using procedures known in the art (Campbell, “Monoclonal Antibody Technology: Laboratory Techniques in Biochemistry and Molecular Biology”, supra, 1984).

For polyclonal antibodies, antibody-containing antisera is isolated from the immunized animal and is screened for the presence of antibodies with the desired specificity using one of the above-described procedures. The above-described antibodies may be detectably labeled. Antibodies can be detectably labeled through the use of radioisotopes, affinity labels (such as biotin, avidin, and the like), enzymatic labels (such as horseradish peroxidase, alkaline phosphatase, and the like) fluorescent labels (such as FITC or rhodamine, and the like), paramagnetic atoms, and the like. Procedures for accomplishing such labeling are well-known in the art, for example, see Stemberger et al., J. Histochem. Cytochem. 18:315, 1970; Bayer et al., Meth. Enzym. 62:308, 1979; Engval et al., Immunol. 109:129, 1972; Goding, J. Immunol. Meth. 13:215, 1976. The antibodies of the present invention may be indirectly labelled by the use of secondary labelled antibodies, such as labelled anti-rabbit antibodies. The labeled antibodies of the present invention can be used for in vitro, in vivo, and in situ assays to identify cells or tissues which express a specific peptide.

The above-described antibodies may also be immobilized on a solid support. Examples of such solid supports include plastics such as polycarbonate, complex carbohydrates such as agarose and sepharose, acrylic resins such as polyacrylamide and latex beads. Techniques for coupling antibodies to such solid supports are well known in the art (Weir et al., “Handbook of Experimental Immunology” 4th Ed., Blackwell Scientific Publications, Oxford, England, Chapter 10, 1986; Jacoby et al., Meth. Enzym. 34, Academic Press, N.Y., 1974). The immobilized antibodies of the present invention can be used for in vitro, in vivo, and in situ assays as well as in immunochromotography.

Furthermore, one skilled in the art can readily adapt currently available procedures, as well as the techniques, methods and kits disclosed herein with regard to antibodies, to generate peptides capable of binding to a specific peptide sequence in order to generate rationally designed antipeptide peptides (Hurby et al., “Application of Synthetic Peptides: Antisense Peptides”, In Synthetic Peptides, A User's Guide, W. H. Freeman, NY, pp. 289-307, 1992; Kaspczak et al., Biochemistry 28:9230-9238, 1989).

Anti-peptide peptides can be generated by replacing the basic amino acid residues found in the peptide sequences of the proteases of the invention with acidic residues, while maintaining hydrophobic and uncharged polar groups. For example, lysine, arginine, and/or histidine residues are replaced with aspartic acid or glutamic acid and glutamic acid residues are replaced by lysine, arginine or histidine.

The present invention also encompasses a method of detecting a protease polypeptide in a sample, comprising: (a) contacting the sample with an above-described antibody, under conditions such that immunocomplexes form, and (b) detecting the presence of said antibody bound to the polypeptide. In detail, the methods comprise incubating a test sample with one or more of the antibodies of the present invention and assaying whether the antibody binds to the test sample. Altered levels of a protease of the invention in a sample as compared to normal levels may indicate disease.

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats (such as radioimmunoassays, enzyme-linked immunosorbent assays, diffusion-based Ouchterlony, or rocket immunofluorescent assays) can readily be adapted to employ the antibodies of the present invention. Examples of such assays can be found in Chard (“An Introduction to Radioimmunoassay and Related Techniques” Elsevier Science Publishers, Amsterdam, The Netherlands, 1986), Bullock et al. (“Techniques in Immunocytochemistry” Academic Press, Orlando, Fla. Vol. 1, 1982; Vol. 2, 1983; Vol. 3, 1985), Tijssen (“Practice and Theory of Enzyme Immunoassays: Laboratory Techniques in Biochemistry and Molecular Biology” Elsevier Science Publishers, Amsterdam, The Netherlands, 1985).

The immunological assay test samples of the present invention include cells, protein or membrane extracts of cells, or biological fluids such as blood, serum, plasma, or urine. The test samples used in the above-described method will vary based on the assay format, nature of the detection method and the tissues, cells or extracts used as the sample to be assayed. Methods for preparing protein extracts or membrane extracts of cells are well known in the art and can readily be adapted in order to obtain a sample which is testable with the system utilized.

A kit contains all the necessary reagents to carry out the previously described methods of detection. The kit may comprise: (i) a first container means containing an above-described antibody, and (ii) second container means containing a conjugate comprising a binding partner of the antibody and a label. In another preferred embodiment, the kit further comprises one or more other containers comprising one or more of the following: wash reagents and reagents capable of detecting the presence of bound antibodies.

Examples of detection reagents include, but are not limited to, labeled secondary antibodies, or in the alternative, if the primary antibody is labeled, the chromophoric, enzymatic, or antibody binding reagents which are capable of reacting with the labeled antibody. The compartmentalized kit may be as described above for nucleic acid probe kits. One skilled in the art will readily recognize that the antibodies described in the present invention can readily be incorporated into one of the established kit formats which are well known in the art.

Isolation of Compounds Which Interact with Proteases

The present invention also relates to a method of detecting a compound capable of binding to a protease of the invention comprising incubating the compound with a protease of the invention and detecting the presence of the compound bound to the protease. The compound may be present within a complex mixture, for example, serum, body fluid, or cell extracts.

The present invention also relates to a method of detecting an agonist or antagonist of protease activity or protease binding partner activity comprising incubating cells that produce a protease of the invention in the presence of a compound and detecting changes in the level of protease activity or protease binding partner activity. The compounds thus identified would produce a change in activity indicative of the presence of the compound. The compound may be present within a complex mixture, for example, serum, body fluid, or cell extracts. Once the compound is identified it can be isolated using techniques well known in the art.

The present invention also encompasses a method of modulating protease associated activity in a mammal comprising administering to said mammal an agonist or antagonist to a protease of the invention in an amount sufficient to effect said modulation. A method of treating diseases in a mammal with an agonist or antagonist of the activity of one of the proteases of the invention comprising administering the agonist or antagonist to a mammal in an amount sufficient to agonize or antagonize protease-associated functions is also encompassed in the present application.

In an effort to discover novel treatments for diseases, biomedical researchers and chemists have designed, synthesized, and tested molecules that inhibit the function of proteases. Some small organic molecules form a class of compounds that modulate the function of protein proteases.

Examples of molecules that have been reported to inhibit the function of protein proteases include, but are not limited to, phenylmethylsulfonyl fluoride (PMSF), diisopropylfluorophosphate (DFP) (chapter 3, Barrett et al., Handbook of Proteolytic Enzymes, 1998, Academic Press, San Diego), 3,4-dichloroisocoumarin (DCI) (Id., chapter 16), serpins (Id., chapter 37), E-64 (trans-epoxysuccinyl L-leucylamido-(4-guanidino) butane) (Id., chapter 188), peptidyl-diazomethanes, peptidyl-O-acyl-hydroxamates, epoxysuccinyl-peptides (Id., chapter 210), DAN, EPNP (1,2-epoxy-3(p-nitrophenoxy)propane) (Id., chapter 298), thiorphan (dl-3-Mercapto-2-benzylpropanoyl-glycine) (Id., chapter 362), CGS 26303, PD 069185 (Id., chapter 363), and COT989-00 (N-4-hydroxy-N1-[1-(s)-(4-aminosulfonyl)phenylethyl-aminocarboxyl-2-cyclohexylethyl)-2R-[4-methyl)phenylpropyl]succinamide) (Id., chapter 401). Other protease inhibitors include, but are not limited to, aprotinin, amastatin, antipain, calcineurin autoinhibitory fragment, and histatin 5 (Id.). Preferably, these inhibitors will have molecular weights from 100 to 200 daltons, from 200 to 300 daltons, from 300 to 400 daltons, from 400 to 600 daltons, from 600 to 1000 daltons, from 1000 to 2000 daltons, from 2000 to 4000 daltons, and from 4000 to 8000 daltons.

Compounds that can traverse cell membranes and are resistant to acid hydrolysis are potentially advantageous as therapeutics as they can become highly bioavailable after being administered orally to patients. However, many of these protease inhibitors only weakly inhibit the function of proteases. In addition, many inhibit a variety of proteases and will therefore cause multiple side-effects as therapeutics for diseases.

Transgenic Animals.

A variety of methods are available for the production of transgenic animals associated with this invention. DNA can be injected into the pronucleus of a fertilized egg before fusion of the male and female pronuclei, or injected into the nucleus of an embryonic cell (e.g., the nucleus of a two-cell embryo) following the initiation of cell division (Brinster et al., Proc. Nat. Acad. Sci. USA 82:4438-4442, 1985). Embryos can be infected with viruses, especially retroviruses, modified to carry inorganic-ion receptor nucleotide sequences of the invention.

Pluripotent stem cells derived from the inner cell mass of the embryo and stabilized in culture can be manipulated in culture to incorporate nucleotide sequences of the invention. A transgenic animal can be produced from such cells through implantation into a blastocyst that is implanted into a foster mother and allowed to come to term. Animals suitable for transgenic experiments can be obtained from standard commercial sources such as Charles River (Wilmington, Mass.), Taconic (Germantown, N.Y.), Harlan Sprague Dawley (Indianapolis, Ind.), etc.

The procedures for manipulation of the rodent embryo and for microinjection of DNA into the pronucleus of the zygote are well known to those of ordinary skill in the art (Hogan et al., supra). Microinjection procedures for fish, amphibian eggs and birds are detailed in Houdebine and Chourrout (Experientia 47:897-905, 1991). Other procedures for introduction of DNA into tissues of animals are described in U.S. Pat. No. 4,945,050 (Sanford et al., Jul. 30, 1990).

By way of example only, to prepare a transgenic mouse, female mice are induced to superovulate. Females are placed with males, and the mated females are sacrificed by CO₂ asphyxiation or cervical dislocation and embryos are recovered from excised oviducts. Surrounding cumulus cells are removed. Pronuclear embryos are then washed and stored until the time of injection. Randomly cycling adult female mice are paired with vasectomized males. Recipient females are mated at the same time as donor females. Embryos then are transferred surgically. The procedure for generating transgenic rats is similar to that of mice (Hammer et al., Cell 63:1099-1112, 1990).

Methods for the culturing of embryonic stem (ES) cells and the subsequent production of transgenic animals by the introduction of DNA into ES cells using methods such as electroporation, calcium phosphate/DNA precipitation and direct injection also are well known to those of ordinary skill in the art (Teratocarcinomas and Embryonic Stem Cells, A Practical Approach, E. J. Robertson, ed., IRL Press, 1987).

In cases involving random gene integration, a clone containing the sequence(s) of the invention is co-transfected with a gene encoding resistance. Alternatively, the gene encoding neomycin resistance is physically linked to the sequence(s) of the invention. Transfection and isolation of desired clones are carried out by any one of several methods well known to those of ordinary skill in the art (E. J. Robertson, supra).

DNA molecules introduced into ES cells can also be integrated into the chromosome through the process of homologous recombination (Capecchi, Science 244:1288-1292, 1989). Methods for positive selection of the recombination event (i.e., neo resistance) and dual positive-negative selection (i.e., neo resistance and gancyclovir resistance) and the subsequent identification of the desired clones by PCR have been described by Capecchi, supra and Joyner et al. (Nature 338:153-156, 1989), the teachings of which are incorporated herein in their entirety including any drawings. The final phase of the procedure is to inject targeted ES cells into blastocysts and to transfer the blastocysts into pseudopregnant females. The resulting chimeric animals are bred and the offspring are analyzed by Southern blotting to identify individuals that carry the transgene. Procedures for the production of non-rodent mammals and other animals have been discussed by others (Houdebine and Chourrout, supra; Pursel et al., Science 244:1281-1288, 1989; and Simms et al., Bio/Technology 6:179-183, 1988).

Thus, the invention provides transgenic, nonhuman mammals containing a transgene encoding a protease of the invention or a gene affecting the expression of the protease. Such transgenic nonhuman mammals are particularly useful as an in vivo test system for studying the effects of introduction of a protease, or regulating the expression of a protease (i.e., through the introduction of additional genes, antisense nucleic acids, or ribozymes).

A “transgenic animal” is an animal having cells that contain DNA which has been artificially inserted into a cell, which DNA becomes part of the genome of the animal which develops from that cell. Preferred transgenic animals are primates, mice, rats, cows, pigs, horses, goats, sheep, dogs and cats. The transgenic DNA may encode human proteases. Native expression in an animal may be reduced by providing an amount of antisense RNA or DNA effective to reduce expression of the receptor.

Gene Therapy

Proteases or their genetic sequences will also be useful in gene therapy (reviewed in Miller, Nature 357:455-460, 1992). Miller states that advances have resulted in practical approaches to human gene therapy that have demonstrated positive initial results. The basic science of gene therapy is described in Mulligan (Science 260:926-931, 1993).

In one preferred embodiment, an expression vector containing a protease coding sequence is inserted into cells, the cells are grown in vitro and then infused in large numbers into patients. In another preferred embodiment, a DNA segment containing a promoter of choice (for example a strong promoter) is transferred into cells containing an endogenous gene encoding proteases of the invention in such a manner that the promoter segment enhances expression of the endogenous protease gene (for example, the promoter segment is transferred to the cell such that it becomes directly linked to the endogenous protease gene).

The gene therapy may involve the use of an adenovirus containing protease cDNA targeted to a tumor, systemic protease increase by implantation of engineered cells, injection with protease-encoding virus, or injection of naked protease DNA into appropriate tissues.

Target cell populations may be modified by introducing altered forms of one or more components of the protein complexes in order to modulate the activity of such complexes. For example, by reducing or inhibiting a complex component activity within target cells, an abnormal signal transduction event(s) leading to a condition may be decreased, inhibited, or reversed. Deletion or missense mutants of a component, that retain the ability to interact with other components of the protein complexes but cannot function in signal transduction, may be used to inhibit an abnormal, deleterious signal transduction event.

Expression vectors derived from viruses such as retroviruses, vaccinia virus, adenovirus, adeno-associated virus, herpes viruses, several RNA viruses, or bovine papilloma virus, may be used for delivery of nucleotide sequences (e.g., cDNA) encod-ing recombinant protease of the invention protein into the targeted cell population (e.g., tumor cells). Methods which are well known to those skilled in the art can be used to construct recombinant viral vectors containing coding sequences (Maniatis et al., Molecular Cloning: A Laboratory Manual, Cold Spring Harbor Laboratory, N.Y., 1989; Ausubel et al., Current Protocols in Molecular Biolog. Greene Publishing Associates and Wiley Interscience, N.Y., 1989). Alternatively, recombinant nucleic acid molecules encoding protein sequences can be used as naked DNA or in a reconstituted system e.g., liposomes or other lipid systems for delivery to target cells (e.g., Felgner et al., Nature 337:387-8, 1989). Several other methods for the direct transfer of plasmid DNA into cells exist for use in human gene therapy and involve targeting the DNA to receptors on cells by complexing the plasmid DNA to proteins (Miller, supra).

In its simplest form, gene transfer can be performed by simply injecting minute amounts of DNA into the nucleus of a cell, through a process of microinjection (Capecchi, Cell 22:479-88, 1980). Once recombinant genes are introduced into a cell, they can be recognized by the cell's normal mechanisms for transcription and translation, and a gene product will be expressed. Other methods have also been attempted for introducing DNA into larger numbers of cells. These methods include: transfection, wherein DNA is precipitated with calcium phosphate and taken into cells by pinocytosis (Chen et al., Mol. Cell Biol. 7:2745-52, 1987); electroporation, wherein cells are exposed to large voltage pulses to introduce holes into the membrane (Chu et al., Nucleic Acids Res. 15:1311-26, 1987); lipofection/liposome fusion, wherein DNA is packaged into lipophilic vesicles which fuse with a target cell (Felgner et al., Proc. Natl. Acad. Sci. USA. 84:7413-7417, 1987); and particle bombardment using DNA bound to small projectiles (Yang et al., Proc. Natl. Acad. Sci. 87:9568-9572, 1990). Another method for introducing DNA into cells is to couple the DNA to chemically modified proteins.

It has also been shown that adenovirus proteins are capable of destabilizing endosomes and enhancing the uptake of DNA into cells. The admixture of adenovirus to solutions containing DNA complexes, or the binding of DNA to polylysine covalently attached to adenovirus using protein crosslinking agents substantially improves the uptake and expression of the recombinant gene (Curiel et al., Am. J. Respir. Cell. Mol. Biol., 6:247-52, 1992).

As used herein “gene transfer” means the process of introducing a foreign nucleic acid molecule into a cell. Gene transfer is commonly performed to enable the expression of a particular product encoded by the gene. The product may include a protein, polypeptide, anti-sense DNA or RNA, or enzymatically active RNA. Gene transfer can be performed in cultured cells or by direct administration into animals. Generally gene transfer involves the process of nucleic acid contact with a target cell by non-specific or receptor mediated interactions, uptake of nucleic acid into the cell through the membrane or by endocytosis, and release of nucleic acid into the cyto-plasm from the plasma membrane or endosome. Expression may require, in addition, movement of the nucleic acid into the nucleus of the cell and binding to appropriate nuclear factors for transcription.

As used herein “gene therapy” is a form of gene transfer and is included within the definition of gene transfer as used herein and specifically refers to gene transfer to express a therapeutic product from a cell in vivo or in vitro. Gene transfer can be performed ex vivo on cells which are then transplanted into a patient, or can be performed by direct administration of the nucleic acid or nucleic acid-protein complex into the patient.

In another preferred embodiment, a vector having nucleic acid sequences encoding a protease polypeptide is provided in which the nucleic acid sequence is expressed only in specific tissue. Methods of achieving tissue-specific gene expression are set forth in International Publication No. WO 93/09236, filed Nov. 3, 1992 and published May 13, 1993.

In all of the preceding vectors set forth above, a further aspect of the invention is that the nucleic acid sequence contained in the vector may include additions, deletions or modifications to some or all of the sequence of the nucleic acid, as defined above.

Expression, including over-expression, of a protease polypeptide of the invention can be inhibited by administration of an antisense molecule that binds to and inhibits expression of the mRNA encoding the polypeptide. Alternatively, expression can be inhibited in an analogous manner using a ribozyme that cleaves the mRNA. General methods of using antisense and ribozyme technology to control gene expression, or of gene therapy methods for expression of an exogenous gene in this manner are well known in the art. Each of these methods utilizes a system, such as a vector, encoding either an antisense or ribozyme transcript of a protease polypeptide of the invention.

The term “ribozyme” refers to an RNA structure of one or more RNAs having catalytic properties. Ribozymes generally exhibit endonuclease, ligase or polymerase activity. Ribozymes are structural RNA molecules which mediate a number of RNA self-cleavage reactions. Various types of trans-acting ribozymes, including “hammerhead” and “hairpin” types, which have different secondary structures, have been identified. A variety of ribozymes have been characterized. See, for example, U.S. Pat. Nos. 5,246,921, 5,225,347, 5,225,337 and 5,149,796. Mixed ribozymes comprising deoxyribo and ribooligonucleotides with catalytic activity have been described. Perreault, et al., Nature, 344:565-567 (1990).

As used herein, “antisense” refers of nucleic acid molecules or their derivatives which specifically hybridize, e.g., bind, under cellular conditions, with the genomic DNA and/or cellular mRNA encoding a protease polypeptide of the invention, so as to inhibit expression of that protein, for example, by inhibiting transcription and/or translation. The binding may be by conventional base pair complementarity, or, for example, in the case of binding to DNA duplexes, through specific interactions in the major groove of the double helix.

In one aspect, the antisense construct is an nucleic acid which is generated ex vivo and that, when introduced into the cell, can inhibit gene expression by, without limitation, hybridizing with the mRNA and/or genomic sequences of a protease polynucleotide of the invention.

Antisense approaches can involve the design of oligonucleotides (either DNA or RNA) that are complementary to protease polypeptide mRNA and are based on the protease polynucleotides of the invention, including SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, and SEQ ID NO:59. The antisense oligonucleotides will bind to the protease polypeptide mRNA transcripts and prevent translation.

Although absolute complementarity is preferred, it is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In general, oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. (Wagner, R. (1994) Nature 372:333). Antisense oligonucleotides complementary to mRNA coding regions are less efficient inhibitors of translation but could be used in accordance with the invention. Whether designed to hybridize to the 5′, 3′ or coding region of the protease polypeptide mRNA, antisense nucleic acids should be at least six nucleotides in length, and are preferably less than about 100 and more preferably less than about 50 or 30 nucleotides in length. Typically they should be between 10 and 25 nucleotides in length. Such principles will inform the practitioner in selecting the appropriate oligonucleotides In preferred embodiments, the antisense sequence is selected from an oligonucleotide sequence that comprises, consists of, or consists essentially of about 10-30, and more preferably 15-25, contiguous nucleotide bases of a nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:1, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQID NO:47, SEQID NO:48, SEQID NO:49, SEQ ID NO:50, SEQID NO:51, SEQ ID NO:52, SEQ ID NO:53, SEQ ID NO:54, SEQ ID NO:55, SEQ ID NO:56, SEQ ID NO:57, SEQ ID NO:58, and SEQ ID NO:59 or domains thereof.

In another preferred embodiment, the invention includes an isolated, enriched or purified nucleic acid molecule comprising, consisting of or consisting essentially of about 10-30, and more preferably 15-25 contiguous nucleotide bases of a nucleic acid sequence that encodes a polypeptide that selected from the group consisting of SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118.

Using the sequences of the present invention, antisense oligonucleotides can be designed. Such antisense oligonucleotides would be administered to cells expressing the target protease and the levels of the target RNA or protein with that of an internal control RNA or protein would be compared. Results obtained using the antisense oligonucleotide would also be compared with those obtained using a suitable control oligonucleotide. A preferred control oligonucleotide is an oligonucleotide of approximately the same length as the test oligonucleotide. Those antisense oligonucleotides resulting in a reduction in levels of target RNA or protein would be selected.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. USA 84:648-652; PCT Publication No. WO 88/09810, published Dec. 15, 1988) or the blood-brain barrier (see, e.g., PCT Publication No. WO 89/10134, published Apr. 25, 1988), hybridization-triggered cleavage agents. (See, e.g., Krol et al. (1988) BioTechniques 6:958-976) or intercalating agents. (See, e.g, Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from moieties such as 5-fluorouracil, 5-bromouracil, 5-chlorouracil, 5-iodouracil, hypoxanthine, xanthine, 4-acetylcytosine, and 5-(carboxyhydroxyethyl)uracil. The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof. (see also U.S. Pat. Nos. 5,176,996; 5,264,564; and 5,256,775)

In yet a further embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al. (1987) Nucl. Acids Res. 15:6625-6641). The oligonucleotide is a 2′-0-methylribonucleotide (Inoue et al. (1987) Nucl. Acids Res. 15:6131-6148), or a chimeric RNA-DNA analogue (Inoue et al. (1987) FEBS Lett. 215:327-330).

Also suitable are peptidyl nucleic acids, which are polypeptides such as polyserine, polythreonine, etc. including copolymers containing various amino acids, which are substituted at side-chain positions with nucleic acids (T,A,G,C,U). Chains of such polymers are able to hybridize through complementary bases in the same manner as natural DNA/RNA. Alternatively, an antisense construct of the present invention can be delivered, for example, as an expression plasmid or vector that, when transcribed in the cell, produces RNA complementary to at least a unique portion of the cellular mRNA which encodes a protease polypeptide of the invention.

While antisense nucleotides complementary to the protease polypeptide coding region sequence can be used, those complementary to the transcribed untranslated region are most preferred.

In another preferred embodiment, a method of gene replacement is set forth. “Gene replacement” as used herein means supplying a nucleic acid sequence which is capable of being expressed in vivo in an animal and thereby providing or augmenting the function of an endogenous gene which is missing or defective in the animal.

Pharmaceutical Formulations and Routes of Administration

The compounds described herein, including protease polypeptides of the invention, antisense molecules, ribozymes, and any other compound that modulates the activity of a protease polypeptide of the invention, can be administered to a human patient per se, or in pharmaceutical compositions where it is mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., latest edition.

A. Routes of Administration

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into a solid tumor, often in a depot or sustained release formulation.

Furthermore, one may administer the drug in a targeted drug delivery system, for example, in a liposome coated with tumor-specific antibody. The liposomes will be targeted to and taken up selectively by the tumor.

B. Composition/Formulation

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Suitable carriers include excipients such as, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:D5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the protease modulating compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

C. Effective Dosage

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

For any compound used in the methods of the invention, the therapeutically effective dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of the protease activity). Such information can be used to more accurately determine useful doses in humans.

Toxicity and therapeutic efficacy of the compounds described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g., Fingl et al., 1975, in The Pharmacological Basis of Therapeutics, Ch. 1 p. 1).

Dosage amount and interval may be adjusted individually to provide plasma levels of the active moiety which are sufficient to maintain the protease modulating effects, or minimal effective concentration (MEC). The MEC will vary for each compound but can be estimated from in vitro-data; e.g., the concentration necessary to achieve 50-90% inhibition of the protease using the assays described herein. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

D. Packaging

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the polynucleotide for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition. Suitable conditions indicated on the label may include treatment of a tumor, inhibition of angiogenesis, treatment of fibrosis, diabetes, and the like.

Functional Derivatives

Also provided herein are functional derivatives of a polypeptide or nucleic acid of the invention. By “functional derivative” is meant a “chemical derivative,” “fragment,” or “variant,” of the polypeptide or nucleic acid of the invention, which terms are defined below. A functional derivative retains at least a portion of the function of the protein, for example reactivity with an antibody specific for the protein, enzymatic activity or binding activity mediated through noncatalytic domains, which permits its utility in accordance with the present invention. It is well known in the art that due to the degeneracy of the genetic code numerous different nucleic acid sequences can code for the same amino acid sequence. Equally, it is also well known in the art that conservative changes in amino acid can be made to arrive at a protein or polypeptide that retains the functionality of the original. In both cases, all permutations are intended to be covered by this disclosure.

Included within the scope of this invention are the functional equivalents of the herein-described isolated nucleic acid molecules. The degeneracy of the genetic code permits substitution of certain codons by other codons that specify the same amino acid and hence would give rise to the same protein. The nucleic acid sequence can vary substantially since, with the exception of methionine and tryptophan, the known amino acids can be coded for by more than one codon. Thus, portions or all of the genes of the invention could be synthesized to give a nucleic acid sequence significantly different from one selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, and SEQ ID NO:59. The encoded amino acid sequence thereof would, however, be preserved.

In addition, the nucleic acid sequence may comprise a nucleotide sequence which results from the addition, deletion or substitution of at least one nucleotide to the 5′-end and/or the 3′-end of the nucleic acid formula selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:l 1, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, and SEQ ID NO:59, or a derivative thereof. Any nucleotide or polynucleotide may be used in this regard, provided that its addition, deletion or substitution does not alter the amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118 which is encoded by the nucleotide sequence. For example, the present invention is intended to include any nucleic acid sequence resulting from the addition of ATG as an initiation codon at the 5′-end of the inventive nucleic acid sequence or its derivative, or from the addition of TTA, TAG or TGA as a termination codon at the 3′-end of the inventive nucleotide sequence or its derivative. Moreover, the nucleic acid molecule of the present invention may, as necessary, have restriction endonuclease recognition sites added to its 5′-end and/or 3′-end.

Such functional alterations of a given nucleic acid sequence afford an opportunity to promote secretion and/or processing of heterologous proteins encoded by foreign nucleic acid sequences fused thereto. All variations of the nucleotide sequence of the protease genes of the invention and fragments thereof permitted by the genetic code are, therefore, included in this invention.

Further, it is possible to delete codons or to substitute one or more codons with codons other than degenerate codons to produce a structurally modified polypeptide, but one which has substantially the same utility or activity as the polypeptide produced by the unmodified nucleic acid molecule. As recognized in the art, the two polypeptides are functionally equivalent, as are the two nucleic acid molecules that give rise to their production, even though the differences between the nucleic acid molecules ate not related to the degeneracy of the genetic code.

A “chemical derivative” of the complex contains additional chemical moieties not normally a part of the protein. Covalent modifications of the protein or peptides are included within the scope of this invention. Such modifications may be introduced into the molecule by reacting targeted amino acid residues of the peptide with an organic derivatizing agent that is capable of reacting with selected side chains or terminal residues, as described below.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing primary amine containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine α-amino group.

Tyrosyl residues are well-known targets of modification for introduction of spectral labels by reaction with aromatic diazonium compounds or tetranitromethane. Most commonly, N-acetylimidizol and tetranitromethane are used to form O-acetyl tyrosyl species and 3-nitro derivatives, respectively.

Carboxyl side groups (aspartyl or glutamyl) are selectively modified by reaction with carbodiimide (R′—N—C—N—R′) such as 1-cyclohexyl-3-(2-morpholinyl(4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Derivatization with bifunctional agents is useful, for example, for cross-linking the component peptides of the protein to each other or to other proteins in a complex to a water-insoluble support matrix or to other macromolecular carriers. Commonly used cross-linking agents include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[p-azidophenyl)dithiolpropioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other modifications include hydroxylation of proline and lysine, phosphorylation of hydroxyl groups of seryl or threonyl residues, methylation of the α-amino groups of lysine, arginine, and histidine side chains (Creighton, T. E., Proteins: Structure and Molecular Properties, W.H. Freeman & Co., San Francisco, pp. 79-86 (1983)), acetylation of the N-terminal amine, and, in some instances, amidation of the C-terminal carboxyl groups.

Such derivatized moieties may improve the stability, solubility, absorption, biological half life, and the like. The moieties may alternatively eliminate or attenuate any undesirable side effect of the protein complex and the like. Moieties capable of mediating such effects are disclosed, for example, in Remington's Pharmaceutical Sciences, 18th ed., Mack Publishing Co., Easton, Pa. (1990).

The term “fragment” is used to indicate a polypeptide derived from the amino acid sequence of the proteins, of the complexes having a length less than the full-length polypeptide from which it has been derived. Such a fragment may, for example, be produced by proteolytic cleavage of the full-length protein. Preferably, the fragment is obtained recombinantly by appropriately modifying the DNA sequence encoding the proteins to delete one or more amino acids at one or more sites of the C-terminus, N-terminus, and/or within the native sequence. Fragments of a protein are useful for screening for substances that act to modulate signal transduction, as described herein. It is understood that such fragments may retain one or more characterizing portions of the native complex. Examples of such retained characteristics include: catalytic activity; substrate specificity; interaction with other molecules in the intact cell; regulatory functions; or binding with an antibody specific for the native complex, or an epitope thereof.

Another functional derivative intended to be within the scope of the present invention is a “variant” polypeptide which either lacks one or more amino acids or contains additional or substituted amino acids relative to the native polypeptide. The variant may be derived from a naturally occurring complex component by appropriately modifying the protein DNA coding sequence to add, remove, and/or to modify codons for one or more amino acids at one or more sites of the C-terminus, N-terminus, and/or within the native sequence. It is understood that such variants having added, substituted and/or additional amino acids retain one or more characterizing portions of the native protein, as described above.

A functional derivative of a protein with deleted, inserted and/or substituted amino acid residues may be prepared using standard techniques well-known to those of ordinary skill in the art. For example, the modified components of the functional derivatives may be produced using site-directed mutagenesis techniques (as exemplified by Adelman et al., 1983, DNA 2:183) wherein nucleotides in the DNA coding the sequence are modified such that a modified coding sequence is modified, and thereafter expressing this recombinant DNA in a prokaryotic or eukaryotic host cell, using techniques such as those described above. Alternatively, proteins with amino acid deletions, insertions and/or substitutions may be conveniently prepared by direct chemical synthesis, using methods well-known in the art. The functional derivatives of the proteins typically exhibit the same qualitative biological activity as the native proteins.

### TABLES AND DESCRIPTION THEROF

This patent describes novel protease identified in databases of genomic sequence. The results are summarized in four tables, which are described below.

Table 1 documents the name of each gene, the classification of each gene, the positions of the open reading frames within the sequence, and the length of the corresponding peptide. From left to right the data presented is as follows: “Gene Name”, “ID#na”, “ID#aa”, “FL/Cat”, “Superfamily”, “Group”, “Family”, “NA_length”, “ORF Start”, “ORF End”, “ORF Length”, and “AA_length”. “Gene name” refers to name given the sequence encoding the protease enzyme. Each gene is represented by “SGPr” designation followed by an arbitrary number. The SGPr name usually represents multiple overlapping sequences built into a single contiguous sequence (a “contig”). The “ID#na” and “ID#aa” refer to the identification numbers given each nucleic acid and amino acid sequence in this patent application. “FL/Cat” refers to the length of the gene, with FL indicating full length, and “Cat” indicating that only the catalytic domain is presented. “Partial” in this column indicates that the sequence encodes a partial catalytic domain. “Superfamily” identifies whether the gene is a protease. “Group” and “Family” refer to the protease classification defined by sequence homology. “NA_length” refers to the length in nucleotides of the corresponding nucleic acid sequence. “ORF start” refers to the beginning nucleotide of the open reading frame. “ORF end” refers to the last nucleotide of the open reading frame, including the stop codon. “ORF length” refers to the length in nucleotides of the open reading frame (including the stop codon). “AA length” refers to the length in amino acids of the peptide encoded in the corresponding nucleic acid sequence.

TABLE 1
Proteases

| | | | | | | | | ORF | ORF | ORF | |
|---|---|---|---|---|---|---|---|---|---|---|---|
| Gene Name | ID#na | ID#aa | FL/Cat | Superfamily | Group | Family | NA_length | Start | End | Length | AA_length |
| SGPr397 | 1 | 60 | FL | Protease | Carboxy- | Zn carboxy- | 948 | 1 | 948 | 948 | 315 |
| | | | | | peptidase | peptidase | | | | | |
| SGPr413 | 2 | 61 | FL | Protease | Carboxy- | Zn carboxy- | 1125 | 1 | 1125 | 1125 | 374 |
| | | | | | peptidase | peptidase | | | | | |
| SGPr404 | 3 | 62 | FL | Protease | Carboxy- | Zn carboxy- | 1590 | 1 | 1590 | 1590 | 529 |
| | | | | | peptidase | pepyidase | | | | | |
| SGPr536_1 | 4 | 63 | FL | Protease | Cysteine | papain | 1404 | 1 | 1404 | 1404 | 467 |
| SGPr414 | 5 | 64 | FL | Protease | Cysteine | UCH2b | 10062 | 1 | 10062 | 10062 | 3353 |
| SGPr430 | 6 | 65 | FL | Protease | Cysteine | UCH2b | 2943 | 1 | 2943 | 2943 | 980 |
| SGPr496_1 | 7 | 66 | FL | Protease | Cysteine | UCH2b | 2862 | 1 | 2862 | 2862 | 953 |
| SGPr495 | 8 | 67 | FL | Protease | Cysteine | UCH2b | 2352 | 1 | 2352 | 2352 | 783 |
| SGPr407 | 9 | 68 | FL | Protease | Cysteine | UCH2b | 2259 | 1 | 2259 | 2259 | 752 |
| SGPr453 | 10 | 69 | FL | Protease | Cysteine | UCH2b | 2139 | 1 | 2139 | 2139 | 712 |
| SGPr445 | 11 | 70 | FL | Protease | Cysteine | UCH2b | 870 | 1 | 870 | 870 | 289 |
| SGPr401_1 | 12 | 71 | FL | Protease | Cysteine | UCH2b | 1101 | 1 | 1101 | 1101 | 366 |
| SGPr408 | 13 | 72 | FL | Protease | Cysteine | UCH2b | 3864 | 1 | 3864 | 3864 | 1287 |
| SGPr480 | 14 | 73 | FL | Protease | Cysteine | UCH2b | 4815 | 1 | 4815 | 4815 | 1604 |
| SGPr431 | 15 | 74 | FL | Protease | Cysteine | UCH2b | 3129 | 1 | 3129 | 3129 | 1042 |
| SGPr429 | 16 | 75 | FL | Protease | Cysteine | UCH2b | 3102 | 1 | 3102 | 3102 | 1033 |
| SGPr503 | 17 | 76 | FL | Protease | Cysteine | UCH2b | 1554 | 1 | 1554 | 1554 | 517 |
| SGPr427 | 18 | 77 | FL | Protease | Cysteine | UCH2b | 3372 | 1 | 3372 | 3372 | 1123 |
| SGPr092 | 19 | 78 | FL | Protease | Metallo- | PepM10 | 786 | 1 | 786 | 786 | 261 |
| | | | | | protease | | | | | | |
| SGPr359 | 20 | 79 | FL | Protease | Metallo- | PepM10 | 1452 | 1 | 1452 | 1452 | 483 |
| | | | | | protease | | | | | | |
| SGPr104_1 | 21 | 80 | FL | Protease | Metallo- | PepM13 | 2298 | 1 | 2298 | 2298 | 765 |
| | | | | | protease | | | | | | |
| SGPr303 | 22 | 81 | CAT | Protease | Metallo- | PepM2 | 1257 | 1 | 1257 | 1257 | 418 |
| | | | | | protease | | | | | | |
| SGPr402_1 | 23 | 82 | FL | Protease | Serine | subtilase | 2268 | 1 | 2268 | 2268 | 755 |
| SGPr434 | 24 | 83 | FL | Protease | Serine | trypsin | 1176 | 1 | 1176 | 1176 | 391 |
| SGPr446_1 | 25 | 84 | CAT | Protease | Serine | trypsin | 681 | 1 | 681 | 681 | 226 |
| SGPr447 | 26 | 85 | FL | Protease | Serine | trypsin | 888 | 1 | 888 | 888 | 295 |
| SGPr432_1 | 27 | 88 | FL | Protease | Serine | trypsin | 1887 | 1 | 1887 | 1887 | 628 |
| SGPr529 | 28 | 87 | FL | Protease | Serine | trypsin | 831 | 1 | 831 | 831 | 276 |
| SGPr428_1 | 29 | 88 | CAT | Protease | Serine | trypsin | 858 | 1 | 858 | 858 | 285 |
| SGPr425 | 30 | 89 | FL | Protease | Serine | trypsin | 1242 | 1 | 1242 | 1242 | 413 |
| SGPr548 | 31 | 90 | FL | Protease | Serine | trypsin | 963 | 1 | 963 | 963 | 320 |
| SGPr396 | 32 | 91 | FL | Protease | Serine | trypsin | 987 | 1 | 987 | 987 | 328 |
| SGPr426 | 33 | 92 | FL | Protease | Serine | trypsin | 1278 | 1 | 1278 | 1278 | 425 |
| SGPr552 | 34 | 93 | CAT | Protease | Serine | trypsin | 666 | 1 | 666 | 666 | 221 |
| SGPr405 | 35 | 94 | FL | Protease | Serine | trypsin | 2847 | 1 | 2847 | 2847 | 948 |
| SGPr485_1 | 36 | 95 | FL | Protease | Serine | trypsin | 1059 | 1 | 1059 | 1059 | 352 |
| SGPr534 | 37 | 96 | FL | Protease | Serine | trypsin | 792 | 1 | 792 | 792 | 263 |
| SGPr390 | 38 | 97 | FL | Protease | Serine | trypsin | 3387 | 1 | 3387 | 3387 | 1128 |
| SGPr521 | 39 | 98 | FL | Protease | Serine | trypsin | 762 | 1 | 762 | 762 | 253 |
| SGPr530_1 | 40 | 99 | CAT | Protease | Serine | trypsin | 816 | 1 | 816 | 816 | 271 |
| SGPr520 | 41 | 100 | FL | Protease | Serine | trypsin | 1737 | 1 | 1737 | 1737 | 578 |
| SGPr455 | 42 | 101 | FL | Protease | Serine | trypsin | 2913 | 1 | 2913 | 2913 | 970 |
| SGPr507_2 | 43 | 102 | FL | Protease | Serine | trypsin | 798 | 1 | 798 | 798 | 265 |
| SGPr559 | 44 | 103 | FL | Protease | Serine | trypsin | 1365 | 1 | 1365 | 1365 | 454 |
| SGPr567_1 | 45 | 104 | FL | Protease | Serine | trypsin | 1614 | 1 | 1614 | 1614 | 537 |
| SGPr479_1 | 46 | 105 | FL | Protease | Serine | trypsin | 981 | 1 | 981 | 981 | 326 |
| SGPr489_1 | 47 | 106 | CAT | Protease | Serine | trypsin | 1671 | 1 | 1671 | 1671 | 556 |
| SGPr465_1 | 48 | 107 | CAT | Protease | Serine | trypsin | 894 | 1 | 884 | 894 | 297 |
| SGPr524_1 | 49 | 108 | FL | Protease | Serine | trypsin | 2553 | 1 | 2553 | 2553 | 850 |
| SGPr422 | 50 | 109 | FL | Protease | Serine | trypsin | 1344 | 1 | 1344 | 1344 | 447 |
| SGPr538 | 51 | 110 | FL | Protease | Serine | trypsin | 1374 | 1 | 1374 | 1374 | 457 |
| SGPr527_1 | 52 | 111 | FL | Protease | Serine | trypsin | 2457 | 1 | 2457 | 2457 | 818 |
| SGPr542 | 53 | 112 | FL | Protease | Serine | trypsin | 855 | 1 | 855 | 855 | 284 |
| SGPr551 | 54 | 113 | FL | Protease | Serine | trypsin | 2409 | 1 | 2409 | 2409 | 802 |
| SGPr451 | 55 | 114 | FL | Protease | Serine | trypsin | 1080 | 1 | 1080 | 1080 | 359 |
| SGPr452_1 | 56 | 115 | FL | Protease | Serine | trypsin | 867 | 1 | 867 | 867 | 288 |
| SGPr504 | 57 | 116 | Partial | Protease | Serine | trypsin | 135 | 1 | 135 | 135 | 44 |
| SGPr469 | 58 | 117 | Partial | Protease | Serine | trypsin | 138 | 1 | 138 | 138 | 45 |
| SGPr400 | 59 | 118 | Partial | Protease | Serine | trypsin | 930 | 1 | 930 | 930 | 309 |

Table 2 lists the following features of the genes described in this patent application: chromosomal localization, single nucleotide polymorphisms (SNPs), representation in dbEST, and repeat regions. From left to right the data presented is as follows: “Gene Name”, “ID#na”, “ID#aa”, “FL/Cat”, “Superfamily”, “Group”, “Family”, “Chromosome”, “SNPs”, “dbEST_hits”, & “Repeats”. The contents of the first 7 columns (i.e.,. “Gene Name”, “ID#na”, “ID#aa”, “FL/Cat”, “Superfamily”, “Group”, “Family”) are as described above for Table 1. “Chromosome” refers to the cytogenetic localization of the gene. Information in the “SNPs” column describes the nucleic acid position and degenerate nature of candidate single nucleotide polymorphisms (SNPs; please see table of polymorphism below). These SNPs were identified by blastn of the DNA sequence against the database of single nucleotide polymorphisms maintained at NCBI (http://www.ncbi.nlm.nih.gov/SNP/sn blastByChr.html). “dbEST hits” lists accession numbers of entries in the public database of ESTs (dbEST, http://www.ncbi.nlm.nih.gov/dbEST/index.html) that contain at least 150 bp of 100% identity to the corresponding gene. These ESTs were identified by blastn of dbEST. “Repeats” contains information about the location of short sequences, approximately 20 bp in length, that are of low complexity and that are present in several distinct genes.

TABLE 2
CHR, SNPs, dbEST, Repeats

| Gene Name | ID#na | ID#aa | FL/Cat | Superfamily | Group | Family | Chromosome |
|---|---|---|---|---|---|---|---|
| SGPr397 | 1 | 60 | FL | Protease | Carboxypeptidase | Zn carboxypeptidase | 6q12 |
| SGPr413 | 2 | 61 | FL | Protease | Carboxypeptidase | Zn carboxypeptidase | 2q35 |
| SGPr404 | 3 | 62 | FL | | Carboxypeptidase | Zn carboxypeptidase | 10q26 |
| SGPr536_1 | 4 | 63 | FL | Protease | Cysteine | papain | 1p35 |
| SGPr414 | 5 | 64 | FL | Protease | Cysteine | UCH2b | 2p14 |
| SGPr430 | 6 | 65 | FL | Protease | Cysteine | UCH2b | 2q37 |
| SGPr496_1 | 7 | 66 | FL | Protease | Cysteine | UCH2b | Xp11.4 |
| SGPr495 | 8 | 67 | FL | Protease | Cysteine | UCH2b | 6q16 |
| SGPr407 | 9 | 68 | FL | Protease | Cysteine | UCH2b | 2q37 |
| SGPr453 | 10 | 69 | FL | Protease | Cysteine | UCH2b | 12q23 |
| SGPr445 | 11 | 70 | FL | | Cysteine | UCH2b | 6q16 |
| SGPr401_1 | 12 | 71 | FL | | Cysteine | UCH2b | 4q11 |
| SGPr406 | 13 | 72 | FL | Protease | Cysteine | UCH2b | 11p15 |
| SGPr480 | 14 | 73 | FL | Protease | Cysteine | UCH2b | 17q24 |
| SGPr431 | 15 | 74 | FL | Protease | Cysteine | UCH2b | 4q31.3 |
| SGPr429 | 16 | 75 | FL | Protease | Cysteine | UCH2b | 1p36.2 |
| SGPr503 | 17 | 76 | FL | Protease | Cysteine | UCH2b | 12q24.3 |
| SGPr427 | 18 | 77 | FL | Protease | Cysteine | UCH2b | 17p13 |
| SGPr092 | 19 | 78 | FL | Protease | Metalloprotease | PepM10 | 11p15 |
| SGPr359 | 20 | 79 | FL | Protease | Metalloprotease | PepM10 | 11q22 |
| SGPr104_1 | 21 | 80 | FL | Protease | Metalloprotease | PepM13 | 3q27 |
| SGPr303 | 22 | 81 | CAT | Protease | Metalloprotease | PepM2 | 17q11.1 |
| SGPr402_1 | 23 | 82 | FL | Protease | Serine | subtilase | 19q11 |
| SGPr434 | 24 | 83 | FL | Protease | Serine | | 3p21 |
| SGPr446_1 | 25 | 84 | CAT | Protease | Serine | trypsin | 3p21 |
| SGPr447 | 26 | 85 | FL | Protease | Serine | trypsin | 16p13.3 |
| SGPr432_1 | 27 | 86 | FL | Protease | Serine | trypsin | Unknown |
| SGPr529 | 28 | 87 | FL | Protease | Serine | trypsin | 19q13.4 |
| SGPr426_1 | 29 | 88 | CAT | Protease | Serine | trypsin | 8p23 |
| SGPr425 | 30 | 89 | FL | Protease | Serine | trypsin | 6q14 |
| SGPr548 | 31 | 90 | FL | | Serine | trypsin | 19q13.4 |
| SGPr396 | 32 | 91 | FL | | Serine | trypsin | 4q32 |
| SGPr426 | 33 | 92 | FL | Protease | Serine | trypsin | 4q13 |
| SGPr552 | 34 | 93 | CAT | Protease | Serine | trypsin | 4q13 |
| SGPr405 | 35 | 94 | FL | Protease | Serine | trypsin | 16p13.3 |
| SGPr485_1 | 36 | 95 | FL | Protease | Serine | trypsin | 8p23 |
| SGPr534 | 37 | 96 | FL | Protease | Serine | trypsin | 16q23 |
| SGPr390 | 38 | 97 | FL | Protease | Serine | trypsin | 19q11 |
| SGPr521 | 39 | 98 | FL | | Serine | trypsin | 19q13.4 |
| SGPr530_1 | 40 | 99 | CAT | Protease | Serine | trypsin | 9q22 |
| SGPr520 | 41 | 100 | FL | Protease | Serine | trypsin | 2q37 |
| SGPr455 | 42 | 101 | FL | Protease | Serine | trypsin | 12p11.2 |
| SGPr507_2 | 43 | 102 | FL | Protease | Serine | trypsin | 7q36 |
| SGPr559 | 44 | 103 | FL | Protease | Serine | trypsin | 21q22 |
| SGPr567_1 | 45 | 104 | FL | Protease | Serine | trypsin | 11q23 |
| SGPr479_1 | 46 | 105 | FL | | Serine | trypsin | 1q42 |
| SGPr489_1 | 47 | 106 | CAT | | | trypsin | 11p15 |
| SGPr465_1 | 48 | 107 | CAT | Protease | | trypsin | Unknown |
| SGPr524_1 | 49 | 108 | FL | Protease | Serine | trypsin | Unknown |
| SGPr422 | 50 | 109 | FL | Protease | Serine | trypsin | 4q13 |
| SGPr538 | 51 | 110 | FL | | Serine | trypsin | 11q23 |
| SGPr527_1 | 52 | 111 | FL | Protease | Serine | trypsin | Unknown |
| SGPr542 | 53 | 112 | FL | Protease | Serine | trypsin | 19q13.1 |
| SGPr551 | 54 | 113 | FL | Protease | Serine | trypsin | 22q13 |
| SGPr451 | 55 | 114 | FL | | Serine | trypsin | 12q23 |
| SGPr452_1 | 56 | 115 | FL | | Serine | trypsin | 16p13.3 |
| SGPr504 | 57 | 116 | Partial | Protease | Serine | trypsin | Unknown |
| SGPr469 | 58 | 117 | Partial | Protease | Serine | trypsin | Unknown |
| SGPr400 | 59 | 118 | Partial | Protease | Serine | trypsin | 4q32 |

| Gene Name | SNPs | dbEST_hits | Repeats |
|---|---|---|---|
| SGPr397 | | AV763490 | |
| SGPr413 | | none | |
| SGPr404 | ss1782198_aflelePos = 201, | AA045746, | 477 ggagctgctgctgctgctggtg 498 |
| | agaaggcctaygaagggg | AA148684, | |
| | | AA047483 | |
| SGPr536_1 | | AL542213, | 480 gctgctgctgctgctggtgcag 501 |
| | | AL547246, | |
| | | AL552037 | |
| SGPr414 | ss16542 allelePos = 101, | AU118237, | 2249 accaccaccaccaccaccatcaccaccaccac 2280 |
| | ctaccctagcygaggaaga | AU131420, | |
| | | AU125083 | |
| SGPr430 | ss1534585_allelePos = 51, | W87666, | |
| | tggaatarctcggac, | AI076108, | |
| | rs1055687_allelePos = 51, | BG612664 | |
| | tggtaatccgkgtagagg | | |
| SGPr495_1 | ss1029756_allelePos = 101, | AW851066, | |
| | agagaaataygagggtatt | AW851065, | |
| | | AW851076 | |
| SGPr495 | | AL559960, | |
| | | AL530470, | |
| | | AL516184 | |
| SGPr407 | | none | |
| SGPr453 | | BG722436, | 553 gtagtaaaaagagaagtaaa 572 |
| | | AI927881, | |
| | | BG771888 | |
| SGPr445 | | AL559960, | |
| | | AL530470, | |
| | | AL516184 | |
| SGPr401_1 | | AU124898, | |
| | | AU134553, | |
| | | AI269069 | |
| SGPr406 | | BG741190, | |
| | | BF575498, | |
| | | BG170829 | |
| SGPr480 | | AU131748, | |
| | | AU120381, | |
| | | BG420766 | |
| SGPr431 | | BG575871, | |
| | | BG113469, | |
| | | BG112979 | |
| SGPr429 | | AL518266, | |
| | | BG681225, | |
| | | BG217186 | |
| SGPr503 | | BG678894, | 1534 gagtgcaagtctgaagaatg 1553 |
| | | BG476418, | |
| | | BE264732 | |
| SGPr427 | | BG831111, | |
| | | AW996553, | |
| | | BE614914 | |
| SGPr092 | | BG189720, | |
| | | AW966183, | |
| | | BG198356 | |
| SGPr359 | | BG187290 | |
| SGPr104_1 | | BF511209, | |
| | | AW341249, | |
| | | AL119270 | |
| SGPr303 | | AU138954, | |
| | | BG251083, | |
| | | AW161660 | |
| SGPr402_1 | | AL041695, | |
| | | AA454137, | |
| | | BG719638 | |
| SGPr434 | | AW137088, | |
| | | BF593342 | |
| SGPr446_1 | | AW243584 | 708 ggtgggcatcatcagctgggg 618 |
| SGPr447 | | none | |
| SGPr432_1 | | BE264142, | |
| | | BG474605, | |
| | | BF304202 | |
| SGPr529 | ss1550333_allelePos = 51, | BE898352, | |
| | taggggatgaycacctgct; | BG469321 | |
| | ss1546197_allelePos = 51, | | |
| | gccggacsactcgc | | |
| SGPr426_1 | | none | 473 catgcacctggaaaagctg 491 |
| SGPr425 | ss674620_allelePos = | AL551286, | 1111 tcagggcaccagtgggtgga 1130 |
| | 201, gagcatctgcrggagagag | AA445948, | |
| | | AA424073 | |
| SGPr548 | | none | |
| SGPr396 | | none | |
| SGPr426 | | none | |
| SGPr552 | | none | |
| SGPr405 | | none | |
| SGPr465_1 | ss1532791_allelePos = 51, | AA781356 | |
| | tggagakaagaacac | | |
| SGPr534 | ss1522946_allelePos = 51, | AW583018, | 172 cacttctgcgggggctccctcatc 195 |
| | gctctaccwccacgccc; | AW582942, | |
| | ss1522943_allelePos = 51, | AW960025 | |
| | cgcacctgctcyaccaccac; | | |
| | ss1522933_allelePos = 51, | | |
| | ctgccagaaggayggagcctgg; | | |
| | ss1522931_allelePos = 51 | | |
| | total len = 101, | | |
| | gtctgccaraaggacg; | | |
| | ss1522930_allelePos = 51, | | |
| | gggtgactctggmggccccct; | | |
| | ss1522928 allelePos = 51, | | |
| | tgcatgggygactctgg | | |
| SGPr390 | ss82431_allelPos = 99, | C16607 | |
| | gccgtgarcaccactg; | | |
| | ss1320361_allelePos = 225, | | |
| | agcggccascattggcgt | | |
| SGPr521 | | AA542994, | 646 caaggtctggtgtcctgggg 685 |
| | | BE713379, | |
| | | W58737 | |
| SGPr530_1 | | none | |
| SGPr520 | | none | |
| SGPr455 | | AW450155, | |
| | | AW995496 | |
| SGPr507_2 | | BG217724, | |
| | | BG219738, | |
| | | BG192709 | |
| SGPr559 | | AI978874, | |
| | | AI469095, | |
| | | BF435670 | |
| SGPr567_1 | | BE732381, | |
| | | R78581, | |
| | | AW845106 | |
| SGPr479_1 | | BG718703, | 780 tggaattgtgagctggggccg 800 |
| | | AA401705, | |
| | | AA398170 | |
| SGPr489_1 | | AW271430, | |
| | | AW237893, | |
| SGPr465_1 | | none | |
| SGPr524_1 | ss2013558_allelePos = 201, | none | 711 aaaaaaaaagaaaagaaaggaaaa 734 |
| | gacatggawgtggacgac; | | |
| | ss2014128_allelePos = 356, | | |
| | acaatttttygagtgccca; | | |
| | ss895409_allelePos = 101, | | |
| | aatttttygagtgcc | | |
| SGPr422 | ss1091793_allelePos = 101, | none | |
| | acatacgccrgatttgtttg; | | |
| | ss448607_allelePos = 101, | | |
| | tgggagcrggtcctgcct | | |
| SGPr538 | | AL538140, | 545 tgggaggcttcctggaggag 564 |
| | | BF934870, | |
| SGPr527_1 | | AW450407, | |
| | | AI190509, | |
| | | AI864473 | |
| SGPr542 | | none | |
| SGPr551 | rs881144_allelePos = 200, | AV693114, | |
| | ctgcagccctaygccgagagg; | N70418, | |
| | rs855791_allelePos = 101, | AA609068 | |
| | agcgaggyctatcgcta | | |
| SGPr451 | ss1881349_allelePos = 201, | BG722131, | |
| | gggcgcatgcaragg; | BG722203 | |
| | ss1266911_allelePos = 101, | | |
| | ccactgcactaaagacrctag | | |
| SGPr452_1 | | none | |
| SGPr504 | | none | |
| SGPr469 | | AW753029, | 55 gggattgtgagctggggc 72 |
| | | Z19070 | |
| SGPr400 | | none | |

Table 3 lists the extent and the boundaries of the protease catalytic domains, and other protein domains. The column headings are: “Gene Name”, “ID#na”, “ID#aa”, “FL/Cat”, “Profile_start”, “Profile_end”, “Domain_start”, “Domain_end”, and “Profile”. The contents of the first 7 columns (i.e.,. “Gene Name”, “ID#na”, “ID#aa”, “FL/Cat”, “Superfamily”, “Group”, “Family”) are as described above for Table 1. “Profile Start”, “Profile End”, “Domain Start” and “Domain End” refer to data obtained using a Hidden-Markov Model to define catalytic range boundaries. The boundaries of the catalytic domain(s) within the overall protein are noted in the “Domain Start” and “Domain End” columns. “Profile” indicates the identity of the Hidden Markov Model used to identify catalytic and other types of domains within the protein sequence. Whether the HMMR search was done with a complete (“Global”) or Smith Waterman (“Local”) model, is described below. Starting from a multiple sequence alignment of catalytic domains, two hidden Markov models were built. One of them allows for partial matches to the catalytic domain; this is a “local” HMM, similar to Smith-Waterman alignments in sequence matching. The other model allows matches only to the complete catalytic domain; this is a “global” HMM similar to Needleman-Wunsch alignments in sequence matching. The Smith Waterman local model is more specific, allowing for fragmentary matches to the catalytic domain whereas the global “complete” model is more sensitive, allowing for remote homologue identification. These domains were identified using PFAM (http://tfam.wustl.edu/hmmsearch.shtml) models, a large collection of multiple sequence alignments and hidden Markov models covering many common protein domains. Version 5.5 of Pfam (September 2000) contains alignments and models for 2478 protein families (http://pfam.wustl.edu/faq.shtml). The PFAM alignments were downloaded from http://pfam.wustl.edu/mmsearch.shtml and the HMMr searches were run locally on a Timelogic computer (TimeLogic Corporation, Incline Village, Nev.). A number of proteins have more than one domain recognized by the HMM searches. For these proteins, the domains have been listed in separate rows.

TABLE 3
Protease Domains, Other Domains

| Gene Name | ID#na | ID#aa | FL/Cat | Profile_start | Profile_end | Domain_start | Domain_end | Profile |
|---|---|---|---|---|---|---|---|---|
| SGPr397 | 1 | 60 | FL | 1 | 146 | 139 | 280 | Zn carboxypeptidase (PF00246) |
| SGPr397 | 1 | 60 | FL | 1 | 82 | 41 | 120 | Carboxypeptidase activation peptide |
| SGPr413 | 2 | 61 | FL | 1 | 248 | 50 | 291 | Zn carboxypeptidase (PF00246) |
| SGPr404 | 3 | 62 | FL | 1 | 248 | 91 | 466 | Zn carboxypeptidase (PF00246) |
| SGPr536_1 | 4 | 63 | FL | 1 | 337 | 203 | 456 | papain (PF00112) |
| SGPr414 | 5 | 64 | FL | 1 | 72 | 1951 | 2045 | Ubiquitin carboxyl-terminal |
| | | | | | | | | hydrolase family 2b (PF00443) |
| SGPr414 | 5 | 64 | FL | 1 | 32 | 1701 | 1732 | UCH2b (PF00442) |
| SGPr430 | 6 | 65 | FL | 1 | 72 | 886 | 951 | Ubiquitin carboxyl-terminal |
| | | | | | | | | hydrolase family 2b (PF00443) |
| SGPr430 | 6 | 65 | FL | 1 | 32 | 342 | 373 | UCH2b (PF00442) |
| SGPr496_1 | 7 | 66 | FL | 1 | 72 | 875 | 935 | Ubiquitin carboxyl-terminal |
| | | | | | | | | hydrolase family 2b (PF00443) |
| SGPr496_1 | 7 | 66 | FL | 1 | 32 | 593 | 624 | UCH2b (PF00442) |
| SGPr496_1 | 7 | 66 | FL | 1 | 82 | 485 | 534 | Zn-finger in ubiquitin- |
| | | | | | | | | hydrolases (PF02148) |
| SGPr495 | 8 | 67 | FL | 1 | 72 | 695 | 781 | Ubiquitin carboxyl-terminal |
| | | | | | | | | hydrotase family 2b (PF00443) |
| SGPr495 | 8 | 67 | FL | 1 | 32 | 190 | 221 | UCH2b (PF00442) |
| SGPr495 | 8 | 67 | FL | 7 | 82 | 78 | 148 | Zn-finger in ubiquitin-hydrolases (PF02148) |
| SGPr407 | 9 | 68 | FL | 80 | 90 | 481 | 491 | Ubiquitin carboxyl-terminal |
| | | | | | | | | hydrolase family 2b (PF00443) |
| SGPr453 | 10 | 69 | FL | 1 | 72 | 615 | 677 | Ubiquitin carboxyl-terminal |
| | | | | | | | | hydrolase family 2b (PF00443) |
| SGPr453 | 10 | 69 | FL | 1 | 32 | 273 | 304 | UCH2b (PF00442) |
| SGPr453 | 10 | 69 | FL | 1 | 82 | 29 | 99 | Zn-finger in ubiquitin-hydrolases (PF02148) |
| SGPr445 | 11 | 70 | FL | 1 | 32 | 190 | 221 | Ubiquitin carboxyl-terminal |
| | | | | | | | | hydrolase family 2b (PF00443) |
| SGPr445 | 11 | 70 | FL | 7 | 82 | 78 | 148 | Zn-finger in ubiquitin-hydrolases (PF02148) |
| SGPr401_1 | 12 | 71 | FL | 1 | 72 | 292 | 384 | Ubiquitin carboxyl-terminal |
| | | | | | | | | hydrolase family 2b (PF00443) |
| SGPr401_1 | 12 | 71 | FL | 1 | 32 | 35 | 66 | UCH2b (PF00442) |
| SGPr408 | 13 | 72 | FL | 1 | 72 | 395 | 475 | Ubiquitin carboxyl-termimal |
| | | | | | | | | hydrolase family 2b (PF00443) |
| SGPr408 | 13 | 72 | FL | 1 | 32 | 100 | 131 | UCH2b (PF00442) |
| SGPr480 | 14 | 73 | FL | 1 | 72 | 1506 | 1566 | Ubiquitin carboxyl-terminal |
| | | | | | | | | hydrolase family 2b (PF00443) |
| SGPr480 | 14 | 73 | FL | 1 | 32 | 734 | 765 | UCH2b (PF00442) |
| SGPr480 | 14 | 73 | FL | 1 | 29 | 268 | 296 | EF hand (PF00036) |
| SGPr480 | 14 | 73 | FL | 1 | 29 | 232 | 260 | EF hand (PF00036) |
| SGPr431 | 15 | 74 | FL | 1 | 72 | 838 | 948 | Ubiquitin carboxyl-terminal |
| | | | | | | | | hydrolase family 2b (PF00443) |
| SGPr431 | 15 | 74 | FL | 1 | 32 | 445 | 476 | UCH2b (PF00442) |
| SGPr429 | 16 | 75 | FL | 1 | 72 | 332 | 419 | Ubiquitin carboxyl-terminal |
| | | | | | | | | hydrolase family 2b (PF00443) |
| SGPr429 | 16 | 75 | FL | 1 | 32 | 89 | 120 | UCH2b (PF00442) |
| SGPr503 | 17 | 76 | FL | 1 | 72 | 432 | 501 | Ubiquitin carboxyl-terminal |
| | | | | | | | | hydrolase family 2b (PF00443) |
| SGPr503 | 17 | 76 | FL | 1 | 32 | 68 | 99 | UCH2b (PF00442) |
| SGPr427 | 18 | 77 | FL | 1 | 72 | 648 | 709 | Ubiquitin carboxy-terminal |
| | | | | | | | | hydrolase family 2b (PF00443) |
| SGPr427 | 18 | 77 | FL | 1 | 29 | 101 | 129 | UCH2b (PF00442) |
| SGPr092 | 19 | 78 | FL | 49 | 168 | 75 | 194 | Peptidase_M10 (PF00413) |
| SGPr092 | 19 | 78 | FL | 168 | 179 | 207 | 218 | ADAM (PF00413) |
| SGPr359 | 20 | 79 | FL | 1 | 168 | 44 | 212 | Peptidase_M10 (PF00413) |
| SGPr359 | 20 | 79 | FL | 1 | 50 | 302 | 443 | 3 × Hemopexin (PF00045) |
| SGPr104_1 | 21 | 80 | FL | 1 | 222 | 561 | 764 | Peptidase_M13 (PF01431) |
| SGPr303 | 22 | 81 | CAT | 1 | 416 | 10 | 397 | Peptidase_M1 (PF01433) |
| SGPr402_1 | 23 | 82 | FL | 1 | 360 | 118 | 437 | subtilase (PF00082) |
| SGPr434 | 24 | 83 | FL | 129 | 136 | 39 | 46 | p20-ICE (PF00656) |
| SGPr446_1 | 25 | 84 | CAT | 1 | 242 | 13 | 227 | Trypsin (PF00089) |
| SGPr447 | 26 | 85 | FL | 1 | 259 | 33 | 270 | Trypsin (PF00089) |
| SGPr432_1 | 27 | 86 | FL | 6 | 259 | 117 | 343 | Trypsin (PF00089) |
| SGPr529 | 28 | 87 | FL | 413 | 416 | 184 | 187 | Trypsin (PF00089) |
| SGPr428_1 | 29 | 88 | CAT | 7 | 259 | 24 | 246 | Trypsin (PF00089) |
| SGPr425 | 30 | 89 | FL | 387 | 406 | 287 | 306 | Trypsin (PF00089) |
| SGPr548 | 31 | 90 | FL | 1 | 259 | 88 | 313 | Trypsin (PF00089) |
| SGPr396 | 32 | 91 | FL | 1 | 259 | 28 | 262 | Trypsin (PF00089) |
| SGPr426 | 33 | 92 | FL | 1 | 259 | 194 | 419 | Trypsin (PF00089) |
| SGPr552 | 34 | 93 | CAT | 1 | 255 | 2 | 222 | Trypsin (PF00089) |
| SGPr405 | 35 | 94 | FL | 60 | 259 | 218 | 406 | Trypsin (PF00089) |
| SGPr405 | 35 | 94 | FL | 126 | 209 | 419 | 496 | Trypsin (PF00089) |
| SGPr405 | 35 | 94 | FL | 122 | 251 | 636 | 761 | Trypsin (PF00089) |
| SGPr485_1 | 36 | 95 | FL | 1 | 259 | 68 | 295 | Trypsin (PF00089) |
| SGPr534 | 37 | 96 | FL | 1 | 259 | 34 | 256 | Trypsin (PF00089) |
| SGPr390 | 38 | 97 | FL | 1 | 259 | 896 | 1122 | Trypsin (PF00089) |
| SGPr390 | 38 | 97 | FL | 1 | 259 | 264 | 500 | Trypsin (PF00089) |
| SGPr390 | 38 | 97 | FL | 1 | 259 | 573 | 800 | Trypsin (PF00089) |
| SGPr521 | 39 | 98 | FL | 1 | 259 | 30 | 245 | Trypsin (PF00089) |
| SGPr530_1 | 40 | 99 | CAT | 1 | 259 | 14 | 255 | Trypsin (PF00089) |
| SGPr520 | 41 | 100 | FL | 1 | 259 | 73 | 306 | Trypsin (PF00089) |
| SGPr455 | 42 | 101 | FL | 1 | 259 | | 674 | Trypsin (PF00089) |
| SGPr455 | 42 | 101 | FL | 109 | 259 | 4 | 156 | Trypsin (PF00089) |
| SGPr455 | 42 | 101 | FL | 2 | 116 | 175 | 812 | 3 × CUB domains (PF00431) |
| SGPr507_2 | 43 | 102 | FL | 35 | 148 | 42 | 135 | Trypsin (PF00089) |
| SGPr507_2 | 43 | 102 | FL | 247 | 259 | 246 | 258 | Trypsin (PF00089) |
| SGPr559 | 44 | 103 | FL | 1 | 259 | 217 | 444 | Trypsin (PF00089) |
| SGPr559 | 44 | 103 | FL | 1 | 43 | 71 | 109 | Low-density lipoprotein |
| | | | | | | | | receptor domain class A (PF00057) |
| SGPr567_1 | 45 | 104 | FL | 1 | 259 | 296 | 524 | Trypsin (PF00089) |
| SGPr479_1 | 46 | 105 | FL | 1 | 259 | 60 | 288 | Trypsin (PF00089) |
| SGPr489_1 | 47 | 106 | CAT | 1 | 227 | 56 | 257 | Trypsin (PF00089) |
| SGPr489_1 | 47 | 106 | CAT | 1 | 116 | 304 | 533 | 2 × CUB domains (PF00431_{—} |
| SGPr465_1 | 48 | 107 | CAT | 12 | 259 | 2 | 240 | Trypsin (PF00089) |
| SGPr524_1 | 49 | 108 | FL | 1 | 259 | 613 | 842 | Trypsin (PF00089) |
| SGPr524_1 | 49 | 108 | FL | 1 | 43 | 489 | 603 | 3 × Low-density lipoprotein |
| | | | | | | | | receptor domain class A (PF00057) |
| SGPr422 | 50 | 109 | FL | 1 | 259 | 216 | 441 | Trypsin (PF00089) |
| SGPr538 | 51 | 110 | FL | 1 | 259 | 216 | 448 | Trypsin (PF00089) |
| SGPr527_1 | 52 | 111 | FL | 1 | 259 | 47 | 286 | Trypsin (PF00089) |
| SGPr527_1 | 52 | 111 | FL | 1 | 156 | 323 | 454 | Trypsin (PF00089) |
| SGPr527_1 | 52 | 111 | FL | 12 | 149 | 564 | 679 | Trypsin (PF00089) |
| SGPr542 | 53 | 112 | FL | 1 | 259 | 35 | 259 | Trypsin (PF00089) |
| SGPr551 | 54 | 113 | FL | 1 | 259 | 568 | 797 | Trypsin (PF00089) |
| SGPr551 | 54 | 113 | FL | 1 | 43 | 447 | 559 | 3 × Low-denslty lipoprotein |
| | | | | | | | | receptor domain class A (PF00057) |
| SGPr451 | 55 | 114 | FL | 1 | 259 | 89 | 324 | Trypsin (PF00089) |
| SGPr452_1 | 56 | 115 | FL | 1 | 259 | 73 | 280 | Trypsin (PF00089) |
| SGPr504 | 57 | 116 | Partial | 1 | 52 | 1 | 45 | Trypsin (PF00089) |
| SGPr469 | 58 | 117 | Partial | 210 | 259 | 1 | 46 | Trypsin (PF00089) |
| SGPr400 | 59 | 118 | Partial | 1 | 198 | 133 | 261 | Trypsin (PF00089) |

Table 4 describes the results of Smith Waterman similarity searches (Matrix: Pam100; gap open/extension penalties 12/2) of the amino acid sequences against the NCBI database of non-redundant protein sequences (http://www.ncbi.nlm.nih.gov/Entrez/Drotein.html). The column headings are: “Gene Name”, “ID#na”, “ID#aa”, “FL/Cat”, “Superfamily”, “Group”, “Family”, “Pscore”, “aa_length”, “aa_ID_match”, “% Identity”, “% Similar”, “ACC#_nraa_match”, and “Description”. The contents of the first 7 columns (i.e., “Gene Name”, “ID#na”, “ID#aa”, “FL/Cat”, “Superfamily”, “Group”, “Family”) are as described above for Table 1. “Pscore” refers to the Smith Waterman probability score. This number approximates the chance that the alignment occurred by chance. Thus, a very low number, such as 2.10E-64, indicates that there is a very significant match between the query and the database target. “aa_length” refers to the length of the protein in amino acids. “aa_ID_match” indicates the number of amino acids that were identical in the alignment. “% Identity” lists the percent of amino acids that were identical over the aligned region. “% Similarity” lists the percent of amino acids that were similar over the alignment. “ACC#nraa_match” lists the accession number of the most similar protein in the NCBI database of non-redundant proteins. “Description” contains the name of the most similar protein in the NCBI database of non-redundant proteins.

TABLE 4
Smith Waterman

| Gene Name | ID#na | ID#aa | FL/Cat | Superfamily | Group | Family | Pacore | aa_length | aa_ID_match |
|---|---|---|---|---|---|---|---|---|---|
| SGPr397 | 1 | 60 | FL | Protease | Carboxy- | Zn carboxy- | 3.10E−220 | 315 | 315 |
| | | | | | peptidase | peptidase | | | |
| SGPr413 | 2 | 61 | FL | Protease | Carboxy- | Zn carboxy- | 5.90E−93 | 374 | 146 |
| | | | | | peptidase | peptidase | | | |
| SGPr404 | 3 | 62 | FL | Protease | Carboxy- | Zn carboxy- | 0 | 529 | 502 |
| | | | | | peptidase | peptidase | | | |
| SGPr536_1 | 4 | 63 | FL | Protease | Cysteine | papain | 1.10E−276 | 487 | 487 |
| SGPr414 | 5 | 64 | FL | Protease | Cysteine | UCH2b | 0 | 3353 | 1259 |
| SGPr430 | 6 | 65 | FL | Protease | Cysyeine | UCH2b | 0 | 960 | 930 |
| SGPr496_1 | 7 | 66 | FL | Protease | Cvsleine | UCH2b | 2.00E−190 | 953 | 496 |
| SGPr495 | 8 | 67 | FL | Protease | Cysteine | UCH2b | 2.40E−176 | 783 | 262 |
| SGPr407 | 9 | 68 | FL | Protease | Cysteine | UCH2b | 2.60E−40 | 753 | 60 |
| SGPr453 | 10 | 69 | FL | Protease | Cysteine | UCH2b | 0 | 712 | 712 |
| SGPr445 | 11 | 70 | FL | Protease | Cysteine | UCH2b | 3.60E−185 | 289 | 269 |
| SGPr401_1 | 12 | 71 | FL | Protease | Cysteine | UCH2b | 7.30E−254 | 366 | 366 |
| SGPr408 | 13 | 72 | FL | Protease | Cysteine | UCH2b | 0 | 1267 | 1287 |
| SGPr480 | 14 | 73 | FL | Protease | Cvsteine | UCH2b | 0 | 1604 | 1272 |
| SGPr431 | 15 | 74 | FL | Protease | Cysteine | UCH2b | 2.40E−251 | 1042 | 397 |
| SGPr429 | 16 | 75 | FL | Protease | Cvsteine | UCH2b | 1.50E−250 | 1033 | 368 |
| SGPr503 | 17 | 76 | FL | Protease | Cysteine | UCH2b | 0 | 517 | 506 |
| SGPr427 | 18 | 77 | FL | Protease | Cysteine | UCH2b | 1.60E−92 | 1123 | 269 |
| SGPr092 | 19 | 78 | FL | Protease | Metallo- | PepM10 | 4.70E−171 | 261 | 261 |
| | | | | | protease | | | | |
| SGPr359 | 20 | 79 | FL | Protease | Metallo- | PepM10 | 0 | 483 | 483 |
| | | | | | protease | | | | |
| SGPr104_1 | 21 | 80 | FL | Protease | Metallo- | PepM13 | 0 | 765 | 785 |
| | | | | | protease | | | | |
| SGPr303 | 22 | 81 | CAT | Protease | Metallo- | PepM2 | 2.20E−284 | 419 | 407 |
| | | | | | protease | | | | |
| SGPr402_1 | 23 | 82 | FL | Protease | Serine | subtilase | 0 | 755 | 513 |
| SGPr434 | 24 | 83 | FL | Protease | Serine | trypsin | 8.20E−43 | 391 | 104 |
| SGPr448_1 | 25 | 84 | CAT | Protease | Serine | trypsin | 2.50E−40 | 227 | 107 |
| SGPr447 | 26 | 85 | FL | Protease | Serine | trypsin | 1.00E−97 | 296 | 167 |
| SGPr432_1 | 27 | 86 | FL | Protease | Serine | trypsin | 3.70E−56 | 626 | 95 |
| SGPr529 | 28 | 87 | FL | Protease | Serine | trypsin | 1.70E−164 | 276 | 276 |
| SGPr428_1 | 29 | 88 | CAT | | Serine | trypsin | 1.90E−56 | 265 | 92 |
| SGPr425 | 30 | 89 | FL | Protease | Serine | trypsin | 5.80E−268 | 413 | 412 |
| SGPr548 | 31 | 90 | FL | Protease | Serine | trypsin | 2.60E−168 | 320 | 250 |
| SGPr396 | 32 | 91 | FL | Protease | Serine | trypsin | 1.60E−56 | 326 | 111 |
| SGPr426 | 33 | 92 | FL | Protease | Serine | trypsin | 7.70E−93 | 425 | 181 |
| SGPr552 | 34 | 93 | CAT | Protease | Serine | trypsin | 1.20E−45 | 222 | 96 |
| SGPr405 | 35 | 94 | FL | Protease | Serine | trypsin | 1.10E−30 | 948 | 111 |
| SGPr465_1 | 36 | 95 | FL | Protease | Serine | trypsin | 7.20E−133 | 352 | 223 |
| SGPr534 | 37 | 96 | FL | Protease | Serine | trypsin | 3.60E−165 | 283 | 253 |
| SGPr390 | 38 | 97 | FL | Protease | Serine | trypsin | 2.60E−53 | 1128 | 135 |
| SgPr521 | 39 | 98 | FL | Protease | Serine | trypsin | 2.30E−155 | 253 | 253 |
| SGPr530_1 | 40 | 99 | CAT | Protease | Serine | trypsin | 1.10E−95 | 272 | 142 |
| SGPr520 | 41 | 100 | FL | Protease | Serine | trypsin | 1.50E−83 | 570 | 150 |
| SGPr455 | 42 | 101 | FL | Protease | Serine | trypsin | 5.90E−179 | 970 | 399 |
| SGPr507_2 | 43 | 102 | FL | Protease | Serine | trypsin | 2.40E−121 | 205 | 195 |
| SGPr559 | 44 | 103 | FL | Protease | Serine | trypsin | 1.40E−265 | 454 | 454 |
| SGPr587_1 | 45 | 104 | FL | Protease | Serine | trypsin | 1.70E−135 | 537 | 534 |
| SGPr479_1 | 46 | 105 | FL | Protease | Serine | trypsin | 1.70E−39 | 326 | 107 |
| SGPr489_1 | 47 | 106 | CAT | Protease | Serine | trypsin | 2.70E−90 | 550 | 194 |
| SGPr465_1 | 48 | 107 | CAT | Protease | Serine | trypsin | 2.70E−76 | 290 | 144 |
| SGPr524_1 | 49 | 108 | FL | Protease | Serine | trypsin | 1.30E−79 | 850 | 193 |
| SGPr422 | 50 | 109 | FL | Proteese | Serine | trypsin | 4.90E−80 | 447 | 173 |
| SGPr538 | 51 | 110 | FL | Protease | Serine | trypsin | 9.1e−315 | 457 | 457 |
| SGPr527_1 | 52 | 111 | FL | Protease | Serine | trypsin | 1.30E−52 | 816 | 114 |
| SGPr542 | 53 | 112 | FL | Protease | Serine | trypsin | 2.70E−41 | 284 | 110 |
| SGPr551 | 54 | 113 | FL | Protease | Serine | trypsin | 0 | 802 | 675 |
| SGPr451 | 55 | 114 | FL | Protease | Serine | trypsin | 9.90E−41 | 359 | 101 |
| SGPr452_1 | 56 | 115 | FL | Protease | Serine | trypsin | 1.40E−81 | 288 | 142 |
| SGPr504 | 57 | 116 | Partial | Protease | Serine | trypsin | 2.40E−13 | 45 | 26 |
| SGPr469 | 58 | 117 | Partial | Protease | Serine | trypsin | 2.20E−17 | 46 | 32 |
| SGPr400 | 59 | 118 | Partial | Protease | Serine | trypsin | 2.30E−16 | 309 | 72 |

| Gene Name | % Identity | % Similar | ACC#_nraa_match | Description |
|---|---|---|---|---|
| SGPr397 | 100 | 100 | NP_065094.1 | carboxypeptidase B |
| | | | | precursor [Homo sapiens] |
| SGPr413 | 49 | 68 | AAF01344.1 | (AF190274) carboxypeptidase |
| | | | | homolog [Bothrops jararaca] |
| SGPr404 | 94 | 96 | NP_061355.1 | carboxypeptidase X2 |
| | | | | [Mus Musculus] |
| SGPr536_1 | 100 | 100 | NP_071447.1 | P3ECSL [Homo sapiens] |
| SGPr414 | 99 | 100 | NP_055524.1 | KIAA0570 gene product |
| | | | | [Homo sapiens] |
| SGPr430 | 99 | 99 | BAB13420.1 | (AB045814) KIAA1594 protein |
| | | | | [Homo sapiens] |
| SGPr496_1 | 95 | 98 | AAF66953.1 | (AF229643) ubiquitin specific |
| | | | | protease [Mus musculus] |
| SGPr495 | 100 | 100 | AAH05991.1 | (BC005991) Unknown (protein |
| | | | | for MGC:14793) [Homo sapiens] |
| SGPr407 | 70 | 84 | NP_036607.1 | ubiquitin specific protease 23: |
| | | | | NEDOS-specific protease |
| | | | | [Homo sapiens] |
| SGPr453 | 100 | 100 | NP_115523.1 | [symbol] |
| SGPr445 | 100 | 100 | AAH05991.1 | (BC005991) Unknown (protein for |
| | | | | MGC:14793) [Homo sapiens] |
| SGPr401_1 | 100 | 100 | NP_073743.1 | hypothetical protein FLJ12552 |
| | | | | [Homo sapiens] |
| SGPr408 | 100 | 100 | BAB55083.1 | (AK027382) unnamed protein |
| | | | | product [Homo sapiens] |
| SGPr480 | 99 | 99 | NP_115971.1 | ubiquitin specific protease |
| | | | | [Homo sapiens] |
| SGPr431 | 100 | 100 | NP_115946.1 | HP43 8KD protein |
| | | | | [Homo sapiens] |
| SGPr429 | 100 | 100 | NP_115812.1 | hypothetical protein FLJ23277 |
| | | | | [Homo sapiens] |
| SGPr503 | 100 | 100 | AAH04868.1 | (BC004868) Unknown (protein |
| | | | | for MGC:10703) |
| | | | | [Homo sapiens] |
| SGPr427 | 36 | 53 | AAF47260.1 | (AE003465) CG3872 gene product |
| | | | | [Drosophila melanogaster] |
| SGPr092 | 100 | 100 | XP_011971.1 | [symbol] |
| SGPr359 | 100 | 100 | NP_004762.1 | [symbol] |
| SGPr104_1 | 100 | 100 | NP_055508.1 | KIAA0604 gene product |
| | | | | [Homo sapiens] |
| SGPr303 | 97 | 96 | CAA10709.1 | [symbol] |
| SGPr402_1 | 82 | 89 | P29121 | NEUROENDOCRINE CONVERTASE 3 |
| | | | | PRECURSOR [Mus musculus] |
| SGPr434 | 42 | 59 | NP_036164.1 | transmembrane tryptase |
| | | | | [Mus musculus] |
| SGPr446_1 | 45 | 57 | NP_038949.4 | [symbol] protease |
| | | | | [Mus musculus] |
| SGPr447 | 60 | 77 | BAB30277.1 | (AK016509) putative |
| | | | | [Mus musculus] |
| SGPr432_1 | 100 | 100 | NP_076869.1 | hypothetical protein |
| | | | | IMAGE3455200 |
| | | | | [Homo sapiens] |
| SGPr529 | 100 | 100 | NP_002767.1 | [symbol] 10; protease, |
| | | | | serine-like, t [Homo sapiens] |
| SGPr426_1 | 53 | 73 | BAB24215.1 | (AK005740) putative |
| | | | | [Mus musculus] |
| SGPr425 | 99 | 99 | CAC35071.1 | [AL121939] dJ223E3.1 |
| | | | | (putative secreted |
| | | | | protein Z51G13) |
| | | | | [Homo sapiens] |
| SGPr548 | 100 | 100 | AAG09459.1 | (AF242195) KLK15 |
| | | | | [Homo sapiens] |
| SGPr396 | 44 | 61 | BAA84941.1 | (AB016694) epidermis specific serine |
| | | | | protease [ [symbol] ] |
| SGPr426 | 43 | 61 | NP_054777.1 | DESC1 protein [Homo sapiens] |
| SGPr552 | 42 | 59 | NP_054777.1 | DESC1 protein [Homo sapiens] |
| SGPr405 | 54 | 65 | P19236 | MASTOCYTOMA PROTEASE |
| | | | | PRECURSOR [ [symbol] ] |
| SGPr485_1 | 94 | 90 | BAB03589.1 | (AB046651) hypothetical |
| | | | | protein [ [symbol] ] |
| SGPr534 | 96 | 96 | NP_001897.1 | [symbol] [Homo sapiens] |
| SGPr390 | 40 | 69 | BAB23684.1 | (AK004939) putative |
| | | | | [Mus musculus] |
| SGPr521 | 100 | 100 | NP_005037.1 | [symbol] |
| SGPr530_1 | 100 | 100 | CAC12709.1 | [symbol] |
| SGPr520 | 73 | 63 | BAB24587.1 | (AKO06434) putative |
| | | | | [Mus musculus] |
| SGPr455 | 41 | 58 | T30337 | polyprotein - African |
| | | | | clawed frog |
| SGPr507_2 | 73 | 81 | NP_080593.1 | RIXEN cDNA 17000 16005 |
| | | | | gene [Mus musculus] |
| SGPr559 | 100 | 100 | NP_078927.1 | transmembrane protease, |
| | | | | serine 3 [Homo sapiens] |
| SGPr587_1 | 99 | 99 | NP_114435.1 | mosaic serine protease |
| | | | | [Homo sapiens] |
| SGPr479_1 | 42 | 57 | NP_114154.1 | [symbol] [Homo sapiens] |
| SGPr489_1 | 37 | 54 | T30338 | oviductin - [ [symbol] ] |
| SGPr465_1 | 48 | 68 | NP_033381.1 | testicular serine protease |
| | | | | 1 [Mus musculus] |
| SGPr524_1 | 41 | 55 | BAB23684.1 | (AK004839) putative |
| | | | | [Mus musculus] |
| SGPr422 | 39 | 59 | NP_054777.1 | DESC1 protein |
| | | | | [Homo sapiens] |
| SGPr538 | 100 | 100 | NP_110397.1 | [symbol] [Homo sapiens] |
| SGPr527_1 | 42 | 59 | AAH03651.1 | (BC003851) Similar to |
| | | | | protease, serine, [symbol] |
| | | | | [Mus Musculus] |
| SGPr542 | 43 | 58 | NP_005308 1 | [symbol] [Homo sapiens] |
| SGPr551 | 84 | 90 | BAB23684.1 | (AKD04939) putative |
| | | | | [Mus musculus] |
| SGPr451 | 39 | 59 | NP_072152.1 | adrenal secretory serine |
| | | | | protease precursor |
| | | | | [Rattus norvegicus] |
| SGPr452_1 | 57 | 72 | AAK15264.1 | [symbol] [Mus musculus] |
| SGPr504 | 81 | 88 | NP_002095.1 | [symbol] K precursor, [symbol] 3; |
| | | | | [symbol] [Homo sapiens] |
| SGPr469 | 69 | 84 | BAB30277.1 | (AK016509) putative |
| | | | | [Mus musculus] |
| SGPr400 | 38 | 45 | NP_038164.1 | transmembrane tryptase |
| | | | | [Mus musculus] |

### EXAMPLES

The examples below are not limiting and are merely representative of various aspects and features of the present invention. The examples below demonstrate the isolation and characterization of the proteases of the invention.

### Example 1

### Identification of Genomic Fragments Encoding Proteases

Novel proteases were identified from the Celera human genomic sequence databases, and from the public Human Genome Sequencing project (http://www.ncbi.nlm.nih.gov/) using hidden Markov models (HMMR). The genomic database entries were translated in six open reading frames and searched against the model using a Timelogic Decypher box with a Field programmable array (FPGA) accelerated version of HMMR2. 1. The DNA sequences encoding the predicted protein sequences aligning to the HMMR profile were extracted from the original genomic database. The nucleic acid sequences were then clustered using the Pangea Clustering tool to eliminate repetitive entries. The putative protease sequences were then sequentially run through a series of queries and filters to identify novel protease sequences. Specifically, the HMMR identified sequences were searched using BLASTN and BLASTX against a nucleotide and amino acid repository containing known human proteases and all subsequent new protease sequences as they are identified. The output was parsed into a spreadsheet to facilitate elimination of known genes by manual inspection. Two models were used, a “complete” model and a “partial” or Smith Waterman model. The partial model was used to identify sub-catalytic domains, whereas the complete model was used to identify complete catalytic domains. The selected hits were then queried using BLASTN against the public mRNA and EST databases to confirm they are indeed unique.

Extension of partial DNA sequences to encompass the longer sequences, including full-length open-reading frame, was carried out by several methods. Iterative blastn searching of the cDNA databases listed in Table 5 was used to find cDNAs that extended the genomic sequences. “LifeGold” databases are from Incyte Genomics, Inc (http://www.incyte.com/). NCBI databases are from the National Center for Biotechnology Information (http://www.ncbi.nlm.nih.gov/ ). All blastn searches were conducted using a penalty for a nucleotide mismatch of −3 and reward for a nucleotide match of 1. The gapped blast algorithm is described in: Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs“, Nucleic Acids Res. 25:3389-3402).

Extension of partial DNA sequences to encompass the full-length open-reading frame was also carried out by iterative searches of genomic databases. The first method made use of the Smith-Waterman algorithm to carry out protein-protein searches of the closest homologue or orthologue to the partial. The target databases consisted of Genscan [Chris Burge and Sam Karlin “Prediction of Complete Gene Structures in Human Genomic DNA”, JMB (1997) 268(1):78-94)] and open-reading frame (ORF) predictions of all human genomic sequence derived from the human genome project (HGP) as well as from Celera. The complete set of genomic databases searched is shown in Table 6 below. Genomic sequences encoding potential extensions were further assessed by blastp analysis against the NCBI nonredundant to confirm the novelty of the hit. The extending genomic sequences were incorporated into the cDNA sequence after removal of potential introns using the Seqman program from DNAStar. The default parameters used for Smith-Waterman searches were Matrix: PAM100; gap-opening penalty: 12; gap extension penalty: 2. Genscan predictions were made using the Genscan program as detailed in Chris Burge and Sam Karlin “Prediction of Complete Gene Structures in Human Genomic DNA”, JMB (1997) 268(l):78-94). ORF predictions from genomic DNA were made using a standard 6-frame translation.

Another method for defining DNA extensions from genomic sequence used iterative searches of genomic databases through the Genscan program to predict exon splicing [Burge and Karlin, JMB (1997) 268(1):78-94)]. These predicted genes were then assessed to see if they represented “real” extensions of the partial genes based on homology to related proteases.

Another method involved using the Genewise program (http://www.sanger.ac.uk/Software/Wise2/) to predict potential ORFs based on homology to the closest orthologue/homologue. Genewise requires two inputs, the homologous protein, and genomic DNA containing the gene of interest. The genomic DNA was identified by blastn searches of Celera and Human Genome Project databases. The orthologs were identified by blastp searches of the NCBI non-redundant protein database (NRAA). Genewise compares the protein sequence to a genomic DNA sequence, allowing for introns and frameshifting errors.

TABLE 5
Databases used for cDNA-based sequence extensions

| Database | Database Date |
|---|---|
| LifeGold templates | May 2001 |
| LifeGold compseqs | May 2001 |
| LifeGold compseqs | May 2001 |
| LifeGold compseqs | May 2001 |
| LifeGold fl | May 2001 |
| LifeGold flft | May 2001 |
| NCBI human Ests | May 2001 |
| NCBI murine Ests | May 2001 |
| NCBI nonredundant | May 2001 |

TABLE 6
DATABASES USED FOR GENOMIC-BASED
SEQUENCE EXTENSIONS

| Database | Number of entries | Database Date |
|---|---|---|
| Celera v. 1-5 | 5,306,158 | January 2000 |
| Celera v. 6-10 | 4,209,980 | May 2000 |
| Celera v. 11-14 | 7,222,425 | April 2000 |
| Celera v. 15 | 243,044 | April 2000 |
| Celera v. 16-17 | 25,885 | April 2000 |
| Celera Assembly 5 (release 25 h) | 479,986 | May 2001 |
| HGP Phase 0 | 3,189 | Nov 1/00 |
| HGP Phase 1 | 20,447 | Jan 1/01 |
| HGP Phase 2 | 1,619 | Jan 1/01 |
| HGP Phase 3 | 9,224 | May 2001 |
| HGP Chromosomal assemblies | 2759 | May 2001 |

Results:

The sources for the sequence information used to extend the genes in the provisional patents are listed below. For genes that were extended using Genewise, the accession numbers of the protein ortholog and the genomic DNA are given. (Genewise uses the ortholog to assemble the coding sequence of the target gene from the genomic sequence). The amino acid sequences for the orthologs were obtained from the NCBI non-redundant database of proteins.(http://www.ncbi.nlm.nih.gov/Entrez/protein.html). The genomic DNA came from two sources: Celera and NCBI-NRNA, as indicated below. cDNA sources are also listed below. All of the genomic sequences were used as input for Genscan predictions to predict splice sites [Burge and Karlin, JMB (1997) 268(1):78-94)]. Abbreviations: HGP: Human Genome Project; NCBI, National Center for Biotechnology Information.

SGPr397, SEQ ID NO:1, SEQ ID NO:60

Genewise orthologs: BAB25826.1, XP_{—}005284.2, NP_{—}065094.1.

Genomic DNA sources: Celera_asm5h 181000001172043

cDNA Sources: Public (gi|9966830|ref|NM_{—}020361.1).

SGPr413, SEQ ID NO:2, SEQ ID NO:61

Genewise orthologs: gi|6013463, XP_{—}003009.1, P15086.

Genomic DNA sources: Celera_asm5h 300475633

SGPr404, SEQ ID NO:3, SEQ ID NO:62

Genewise orthologs: BAB31768.1, NP_{—}061355.1, AAH03713.

Genomic DNA sources: Celera_asm5h 90000641768196

SGPr536_{—}1, SEQ ID NO:4, SEQ ID NO:63

Genewise orthologs: BAB18637.

Genomic DNA sources: 90000642234172

SGPr414, SEQ ID NO:5, SEQ ID NO:64

Genewise orthologs: AAF50752. 1.

Genomic DNA sources: 90000628114448

cDNA Sources: AK023845.1|AK023845 Homo sapiens cDNA FLJ13783 fis; Incyte 399773.5.

SGPr430, SEQ ID NO:6, SEQ ID NO:65

Genewise orthologs: NP_{—}065954.

Genomic DNA sources: 301015601

cDNA Sources: AB046814 Homo sapiens mRNA for KIAA1594.

SGPr496_{—}1, SEQ ID NO:7, SEQ ID NO:66

Genewise orthologs: AAH07196, AAF66953.

Genomic DNA sources: 90000627702299

SGPr495, SEQ ID NO:8, SEQ ID NO:67

Genewise orthologs: NP_{—}006438.

Genomic DNA sources: 90000627041101

SGPr407, SEQ ID NO:9, SEQ ID NO:68

Genewise orthologs: BAB27431, AAH03130, NP_{—}057656.

Genomic DNA sources: 92000003986525

SGPr453, SEQ ID NO:10, SEQ ID NO:69

Genewise orthologs: NP_{—}006528.

Genomic DNA sources: 90000640175777

cDNA Sources: AL136825.1|HSM801793 Homo sapiens mRNA; Incyte 428428.1

SGPr445, SEQ ID NO:11, SEQ ID NO:70

Genewise orthologs: NP_{—}006438.

Genomic DNA sources: 90000627041101

cDNA Sources: 9863487 328 bp ubiquitin carboxyl-terminal hydrolase; Incyte 4802789CA2

SGPr401_{—}1, SEQ ID NO:12, SEQ ID NO:71

Genewise orthologs: BAB14881, NP_{—}073743, BAB24720.

Genomic DNA sources: 92000004473288

cDNA Sources: NM_{—}022832.1|Homo sapiens hypothetical protein FLJ12552 (FLJ12552)

SGPr408, SEQ ID NO:13, SEQ ID NO:72

Genewise orthologs: Q24574, AAF50752.

Genomic DNA sources: 90000628565543

cDNA Sources: AK027362.

SGPr480, SEQ ID NO:14, SEQ ID NO:73

Genewise orthologs: AAF49100, T29010.

Genomic DNA sources: 90000640697688

cDNA Sources: EF_hand; CAAX: NP_{—}115971.

SGPr431, SEQ ID NO:15, SEQ ID NO:74

Genewise orthologs: AAK26248, BAA92610, Q92353.

Genomic DNA sources: 90000642340202

SGPr429, SEQ ID NO:16, SEQ ID NO:75

Genewise orthologs: BAB15591, AAG42764, gi_{—}11358453.

Genomic DNA sources: 90000642540891

cDNA Sources: AK026930.1|AK026930 Homo sapiens cDNA: FLJ23277.

SGPr503, SEQ ID NO:17, SEQ ID NO:76

Genewise orthologs: AAF40451, AAF46096, AAH04868.

Genomic DNA sources: 90000642658172

cDNA Sources: BC004868.1|BC004868 Homo sapiens, clone MGC:10702; Incyte 5432879CB1.

SGPr427, SEQ ID NO:18, SEQ ID NO:77

Genewise orthologs: XP_{—}003288, AAC27356, BAA86517.

Genomic DNA sources: 181000001646773

cDNA Sources: Incyte 7485896CB1

SGPr092, SEQ ID NO:19, SEQ ID NO:78

Genewise orthologs: XP_{—}011971.1, NP_{—}068573.1, AAF80180.1.

Genomic DNA sources: Celera_asm5h 300261795

cDNA Sources: gi|12736016.

SGPr359, SEQ ID NO:20, SEQ ID NO:79

Genewise orthologs: 1.) gi|115458451ref 2.) gi|12006364|gb 3.) gi|3511149|gb|A.

Genomic DNA sources: Celera_asm5h 90000642045264

cDNA Sources: gi|13639688.

SGPr104_{—}1, SEQ ID NO:21, SEQ ID NO:80

Genewise orthologs: 1.) gi|7662200|ref.

Genomic DNA sources: HGP_s gi|1203907814

cDNA Sources: NP_{—}055508.1.

SGPr303, SEQ ID NO:22, SEQ ID NO:81

Genewise orthologs: CAA10709.1.

Genomic DNA sources: HGP_s gi|8082389_{—}31

SGPr402_{—}1, SEQ ID NO:23, SEQ ID NO:82

Genewise orthologs: A54306, I77530, A45357

Genomic DNA sources: Celera_asm5h 92000004018126

SGPr434, SEQ ID NO:24, SEQ ID NO:83

Genewise orthologs: gi|6755819, gi|6912728, gi|8570164.

Genomic DNA sources: 90000628646128, 160000117588372, 165000100269164, 90000628646080

cDNA Sources: gi|6141221, gi|3754092, Incyte 1856589CB1.

SGPr446_{—}1, SEQ ID NO:25, SEQ ID NO:84

Genewise orthologs: gi_{—}11055972, gi_{—}12839280, gi_{—}13633971.

Genomic DNA sources: 90000628646080

SGPr447, SEQ ID NO:26, SEQ ID NO:85

Genewise orthologs: gi|12855280, gi|1055972, gi|8777606.

Genomic DNA sources: 90000628729589

SGPr432_{—}1, SEQ ID NO:27, SEQ ID NO:86

Genewise orthologs: gi_{—}11181573, gi_{—}12832944, gi_{—}13124769, gi_{—}13277969, gi_{—}13632973.

Genomic DNA sources: 90000631961624

cDNA Sources: Incyte EST 474674.1.

SGPr529, SEQ ID NO:28, SEQ ID NO:87

Genewise orthologs: NP_{—}002767, AAH02100

Genomic DNA sources: Celera_asm5h 92000003497776

cDNA Sources: gi|4506157.

SGPr428_{—}1, SEQ ID NO:29, SEQ ID NO:88

Genewise orthologs: gi|12838473, gi|12839985, gi|9651113, gi|4165315.

Genomic DNA sources: 90000627342893

SGPr425, SEQ ID NO:30, SEQ ID NO:89

Genewise orthologs: gi_{—}12844896, gi_{—}6005882.

Genomic DNA sources: 181000004221955

cDNA Sources: Incyte Sequence 400833.1.

SGPr548, SEQ ID NO:31, SEQ ID NO:90

Genewise orthologs: gi|9957760, gi|5803199, gi|6681654.

Genomic DNA sources: 92000003497776, gi|11178143

cDNA Sources: gi|9957759.

SGPr396, SEQ ID NO:32, SEQ ID NO:91

Genewise orthologs: gi_{—}11055972, gi_{—}12839280, gi_{—}6680267, gi_{—}8393560, gi_{—}9757698.

Genomic DNA sources: 90000632590917

cDNA Sources: Incyte Sequence 7480224CB1.

SGPr426, SEQ ID NO:33, SEQ ID NO:92

Genewise orthologs: gi_{—}13640890, gi_{—}13646365, gi_{—}7661558.

Genomic DNA sources: 90000641479138

cDNA Sources: Incyte Sequence 7481056CB1.

SGPr552, SEQ ID NO:34, SEQ ID NO:93

Genewise orthologs: gi|7661558, gi|4758508.

Genomic DNA sources: 90000641479138

SGPr405, SEQ ID NO:35, SEQ ID NO:94

Genewise orthologs: gi_{—}7415931, gi_{—}126839, gi_{—}136423, gi_{—}13183572.

Genomic DNA sources: gi|13509126

cDNA Sources: Incyte seqs 7474351CB1 and 134360.1.

SGPr485_{—}1, SEQ ID NO:36, SEQ ID NO:95

Genewise orthologs: gi|9651113.

Genomic DNA sources: 90000627342893

cDNA Sources: Incyte Sequence 6026494CA2.

SGPr534, SEQ ID NO:37, SEQ ID NO:96

Genewise orthologs: gi|4503135.

Genomic DNA sources: 92000004436076, 165000101932709, 92000004433469

cDNA Sources: Incyte ESTs: 1383391.20, 1383391.10, 1383391.13, 7691434H1, 2070278CB1, 741522CA2; NCBI ESTs: gi|7260671, gi|7260006, gi|7260642, gi|7259962, gi|2018619, gi|7260655, gi|2019751.

SGPr390, SEQ ID NO:38, SEQ ID NO:97

Genewise orthologs: BAB23684

Genomic DNA sources: hCG22693

SGPr521, SEQ ID NO:39, SEQ ID NO:98

Genewise orthologs: BAB55604, AAFO1139, AAF01139

Genomic DNA sources: HGP_s gi|1178143_{—}10

cDNA Sources: gi|4826949.

SGPr530_{—}1, SEQ ID NO:40, SEQ ID NO:99

Genewise orthologs: gi_{—}12314133, NP_{—}033381.1 3, NP_{—}033382.1

Genomic DNA sources: Celera_asm5h 181000001848433

SGPr520, SEQ ID NO:41, SEQ ID NO:100

Genewise orthologs: gi|12839535 gi|1352368, gi|4506151.

Genomic DNA sources: 90000640807190

cDNA Sources: ESTs gi|3745759, 7472044CB1, 7474338CB1, gi|3703426, gi|5392427, gi|2142177, gi|2103202, LIB4218-103-R1-K1-H5, LIB4218-085-Q1-K1-C6, LIB4752-019-R1-K1-H4

SGPr455, SEQ ID NO:42, SEQ ID NO:101

Genewise orthologs: gi|7512178, gi|7512176.

Genomic DNA sources: 90000641321557

cDNA Sources: Incyte template 987279.1.

SGPr507_{—}2, SEQ ID NO:43, SEQ ID NO:102

Genewise orthologs: gi|3385812, gi|12854692, gi|2499862.

Genomic DNA sources: 90000642611957

SGPr559, SEQ ID NO:44, SEQ ID NO:103

Genewise orthologs: XP_{—}016993, BAB20079

Genomic DNA sources: Celera_asm5h 335001064013332

cDNA Sources: gi|13173471.

SGPr567_{—}1, SEQ ID NO:45, SEQ ID NO:104

Genewise orthologs: NP_{—}114435, Q9JIQ8

Genomic DNA sources: Celera_asm5h 90000642045213

cDNA Sources: gi|14042983|ref|NM_{—}032046.1.

SGPr479_{—}1, SEQ ID NO:46, SEQ ID NO:105

Genewise orthologs: NP_{—}114154, NP_{—}038949, NP_{—}033382

Genomic DNA sources: 90000624931837

cDNA Sources: EST gi|3997890 and Incyte EST 7480124CB1.

SGPr489_{—}1, SEQ ID NO:47, SEQ ID NO:106

Genewise orthologs: gi|7512176, gi|7512178, gi|9757698.

Genomic DNA sources: 90000628565500

SGPr465_{—}1, SEQ ID NO:48, SEQ ID NO:107

Genewise orthologs: gi|6678293, gi|6678295.

Genomic DNA sources: gi|3431162

SGPr524_{—}1, SEQ ID NO:49, SEQ ID NO:108

Genewise orthologs: gi|12836503, gi|10257390, gi|11415040.

Genomic DNA sources: 90000626428259

SGPr422, SEQ ID NO:50, SEQ ID NO:109

Genewise orthologs: gi|7661558, gi|4758508.

Genomic DNA sources: 90000641479138

SGPr538, SEQ ID NO:51, SEQ ID NO:110

Genewise orthologs: NP_{—}110397, Q9ER04, NP_{—}109634

Genomic DNA sources: Celera_asm5h 90000642044035 and 90000642045412

cDNA Sources: gi|13540535.

SGPr527_{—}1, SEQ ID NO:52, SEQ ID NO:111

Genewise orthologs: gi|1181573, gi|13277969, gi|10441463.

Genomic DNA sources: 90000631961624

cDNA Sources: Incyte 2751509CB1.

SGPr542, SEQ ID NO:53, SEQ ID NO:112

Genewise orthologs: gi|705760, gi|4885369.

Genomic DNA sources: 92000004018116, gi|2896799, 92000004013323, 92000004013330, 165000100427031

SGPr551, SEQ ID NO:54, SEQ ID NO:113

Genewise orthologs: BAB23684.1, NP_{—}035306.2, BAB03502.1

Genomic DNA sources: Celera_{—asm}5h 90000643090998

SGPr451, SEQ ID NO:55, SEQ ID NO:114

Genewise orthologs: gi_{—}5002340, gi|2018322, gi|1480413.

Genomic DNA sources: 181000000828193

SGPr452_{—}1, SEQ ID NO:56, SEQ ID NO:115

Genewise orthologs: gi|3183572, gi|339983, gi|7415931.

Genomic DNA sources: 92000004034678

SGPr504, SEQ ID NO:57, SEQ ID NO:116

Genewise orthologs: gi|1633237

Genomic DNA sources: celera_asm5h 92000004018137

SGPr469, SEQ ID NO:58, SEQ ID NO:117

Genewise orthologs: BAB30277, CAB41988, XP_{—}016204

Genomic DNA sources: GA_x2HTBKPYW7D

SGPr400, SEQ ID NO:59, SEQ ID NO:118

Genewise orthologs: gi|6755819, gi|6912728.

Genomic DNA sources: 90000632590917

### DESCRIPTION OF NOVEL PROTEASE POLYNUCLEOTIDES

SGPr397, SEQ ID NO:1, SEQ ID NO:60 is 948 nucleotides long. The open reading frame starts at position 1 and ends at position 948, giving an ORF length of 948 nucleotides. The predicted protein is 315 amino acids long. This sequence codes for a full length protein. It is classified as (superfamily/group/family): Protease, Carboxypeptidase, Zn carboxypeptidase. The cytogenetic position of this gene is 8q12. This sequence is represented in the database of public ESTs (dbEST) by the following ESTs: AV763490.

SGPr413, SEQ ID NO:2, SEQ ID NO:61 is 1125 nucleotides long. The open reading frame starts at position 1 and ends at position 1125, giving an ORF length of 1125 nucleotides. The predicted protein is 374 amino acids long. This sequence codes for a full length protein. It is classified as (superfamily/group/family): Protease, Carboxypeptidase, Zn carboxypeptidase. The cytogenetic position of this gene is 2q35.This sequence is represented in the database of public ESTs (dbEST) by the following ESTs: none.

SGPr404, SEQ ID NO:3, SEQ ID NO:62 is 1590 nucleotides long. The open reading frame starts at position 1 and ends at position 1590, giving an ORF length of 1590 nucleotides. The predicted protein is 529 amino acids long. This sequence codes for a full length protein. It is classified as (superfamily/group/family): Protease, Carboxypeptidase, Zn carboxypeptidase. The cytogenetic position of this gene is 10q26. This nucleotide sequence contains the following single nucleotide polymorphisms (the accession number of SNP is given, with the allele position, followed by the sequence surrounding the SNP within the gene): ss1782198_allelePos=201, agaaggcctaygaagggg. SNP ss1782198 occurs at nucleotide 612 (aa 58) of the ORF (C or T=silent; AA 204=tyrosine with either nucleotide). This sequence is represented in the database of public ESTs (dbEST) by the following ESTs: AA045748, AA148684, AA047483. The nucleic acid contains short repetitive sequence (the position and sequence of the repeat): 477 ggagctgctgctgctgctggtg 498.

SGPr536_{—}1, SEQ ID NO:4, SEQ ID NO:63 is 1404 nucleotides long. The open reading frame starts at position 1 and ends at position 1404, giving an ORF length of 1404 nucleotides. The predicted protein is 467 amino acids long. This sequence codes for a full length protein. It is classified as (superfamily/group/family): Protease, Cysteine, papain. The cytogenetic position of this gene is 1p35. This sequence is represented in the database of public ESTs (dbEST) by the following ESTs: AL542213, AL547246, AL552037. The nucleic acid contains short repetitive sequence (the position and sequence of the repeat): 480 gctgctgctgctgctggtgcag 501.

SGPr414, SEQ ID NO:5, SEQ ID NO:64 is 10062 nucleotides long. The open reading frame starts at position 1 and ends at position 10062, giving an ORF length of 10062 nucleotides. The predicted protein is 3353 amino acids long. This sequence codes for a full length protein. It is classified as (superfamily/group/family): Protease, Cysteine, UCH2b. The cytogenetic position of this gene is 2p14. This nucleotide sequence contains the following single nucleotide polymorphisms (the accession number of SNP is given, followed by the sequence surrounding the SNP within the gene): ss16542_allelePos=101, ctaccctagcygaggaaga. SNP ss16542 occurs at nucleotide 9807 (aa 3269, alanine) of the ORF. The SNP is silent. This sequence is represented in the database of public ESTs (dbEST) by the following ESTs: AU118237, AU131420, AU125083. The nucleic acid contains short repetitive sequence (the position and sequence of the repeat): 2249 accaccaccaccaccaccatcaccaccaccac 2280.

SGPr430, SEQ ID NO:6, SEQ ID NO:65 is 2943 nucleotides long. The open reading frame starts at position 1 and ends at position 2943, giving an ORF length of 2943 nucleotides. The predicted protein is 980 amino acids long. This sequence codes for a full length protein. It is classified as (superfamily/group/family): Protease, Cysteine, UCH2b. The cytogenetic position of this gene is 2q37. This nucleotide sequence contains the following single nucleotide polymorphisms (the accession number of SNP is given, with the allele position, followed by the sequence surrounding the SNP within the gene): ss1534585_allelePos=51, tggaatarctcggac; rs1055687_allelePos=51, tggtaatccgkgtagagg. SNP ss1534585 occurs at nucleotide 538 (aa 180) of the ORF (A or G). The SNP ss1534585 changes amino acid 180. If nucleotide 538 is an adenine, amino acid 180 is a threonine; if it is a guanine, amino acid 180 is an alanine. A second SNP, rs1055687, codes for a G or T at nucleotide 499. rs1055687 changes the amino acid sequence of the gene. Amino acid 167 is a cysteine if nucleotide 499 is a thymidine; amino acid 167 is a glycine if nucleotide 499 is a guanine. This sequence is represented in the database of public ESTs (dbEST) by the following ESTs: W87666, AI076108, BG612864.

SGPr496_{—}1, SEQ ID NO:7, SEQ ID NO:66 is 2862 nucleotides long. The open reading frame starts at position 1 and ends at position 2862, giving an ORF length of 2862 nucleotides. The predicted protein is 953 amino acids long. This sequence codes for a full length protein. It is classified as (superfamily/group/family): Protease, Cysteine, UCH2b. The cytogenetic position of this gene is Xp11.4. This nucleotide sequence contains the following single nucleotide polymorphisms (the accession number of SNP is given, with the allele position, followed by the sequence surrounding the SNP within the gene): ss1029756_allelePos=101, agagaaataygagggtatt. SNP ss1029756 codes for a C or T at nucleotide 351. Amino acid 117 is a tyrosine with either nucleotide, so the SNP is silent. This sequence is represented in the database of public ESTs (dbEST) by the following ESTs: AW851066, AW851065, AW851076.

SGPr495, SEQ ID NO:8, SEQ ID NO:67 is 2352 nucleotides long. The open reading frame starts at position 1 and ends at position 2352, giving an ORF length of 2352 nucleotides. The predicted protein is 783 amino acids long. This sequence codes for a full length protein. It is classified as (superfamily/group/family): Protease, Cysteine, UCH2b. The cytogenetic position of this gene is 6q16 This sequence is represented in the database of public ESTs (dbEST) by the following ESTs: AL559960, AL530470, AL516184.

SGPr407, SEQ ID NO:9, SEQ ID NO:68 is 2259 nucleotides long. The open reading frame starts at position 1 and ends at position 2259, giving an ORF length of 2259 nucleotides. The predicted protein is 752 amino acids long. This sequence codes for a full length protein. It is classified as (superfamily/group/family): Protease, Cysteine, UCH2b. The cytogenetic position of this gene is 2q37. This sequence is represented in the database of public ESTs (dbEST) by the following ESTs: none.

SGPr453, SEQ ID NO:10, SEQ ID NO:69 is 2139 nucleotides long. The open reading frame starts at position 1 and ends at position 2139, giving an ORF length of 2139 nucleotides. The predicted protein is 712 amino acids long. This sequence codes for a fall length protein. It is classified as (superfamily/group/family): Protease, Cysteine, UCH2b. The cytogenetic position of this gene is 12q23. This sequence is represented in the database of public ESTs (dbEST) by the following ESTs: BG722436, AI927881, BG771888. The nucleic acid contains short repetitive sequence (the position and sequence of the repeat): 553 gtagtaaaaagagaagtaaa 572.

SGPr445, SEQ ID NO:11, SEQ ID NO:70 is 870 nucleotides long. The open reading frame starts at position 1 and ends at position 870, giving an ORF length of 870 nucleotides. The predicted protein is 289 amino acids long. This sequence codes for a full length protein. It is classified as (superfamily/group/family): Protease, Cysteine, UCH2b. The cytogenetic position of this gene is 6q16. This sequence is represented in the database of public ESTs (dbEST) by the following ESTs: AL559960, AL530470, AL516184.

SGPr401_{—}1, SEQ ID NO:12, SEQ ID NO:71 is 1101 nucleotides long. The open reading frame starts at position 1 and ends at position 1101, giving an ORF length of 1101 nucleotides. The predicted protein is 366 amino acids long. This sequence codes for a full length protein. It is classified as (superfamily/group/family): Protease, Cysteine, UCH2b. The cytogenetic position of this gene is 4q11. This sequence is represented in the database of public ESTs (dbEST) by the following ESTs: AU124898, AU134553, AI269069.

SGPr408, SEQ ID NO:13, SEQ ID NO:72 is 3864 nucleotides long. The open reading frame starts at position 1 and ends at position 3864, giving an ORF length of 3864 nucleotides. The predicted protein is 1287 amino acids long. This sequence codes for a full length protein. It is classified as (superfamily/group/family): Protease, Cysteine, UCH2b. The cytogenetic position of this gene is 11p15. This sequence is represented in the database of public ESTs (dbEST) by the following ESTs: BG741190, BF575498, BG170829.

SGPr480, SEQ ID NO:14, SEQ ID NO:73 is 4815 nucleotides long. The open reading frame starts at position 1 and ends at position 4815, giving an ORF length of 4815 nucleotides. The predicted protein is 1604 amino acids long. This sequence codes for a partial protein. It is classified as (superfamily/group/family): Protease, Cysteine, UCH2b. The cytogenetic position of this gene is 17q24.This sequence is represented in the database of public ESTs (dbEST) by the following ESTs: AU131748, AU120381, BG420766.

SGPr431, SEQ ID NO:15, SEQ ID NO:74 is 3129 nucleotides long. The open reading frame starts at position 1 and ends at position 3129, giving an ORF length of 3129 nucleotides. The predicted protein is 1042 amino acids long. This sequence codes for a full length protein. It is classified as (superfamily/group/family): Protease, Cysteine, UCH2b. The cytogenetic position of this gene is 4q31.3.This sequence is represented in the database of public ESTs (dbEST) by the following ESTs: BG575871, BG113469, BG112979.

SGPr429, SEQ ID NO:16, SEQ ID NO:75 is 3102 nucleotides long. The open reading frame starts at position 1 and ends at position 3102, giving an ORF length of 3102 nucleotides. The predicted protein is 1033 amino acids long. This sequence codes for a full length protein. It is classified as (superfamily/group/family): Protease, Cysteine, UCH2b. The cytogenetic position of this gene is 1p36.2. This sequence is represented in the database of public ESTs (dbEST) by the following ESTs: AL518266, BG681225, BG217186.

SGPr503, SEQ ID NO:17, SEQ ID NO:76 is 1554 nucleotides long. The open reading frame starts at position 1 and ends at position 1554, giving an ORF length of 1554 nucleotides. The predicted protein is 517 amino acids long. This sequence codes for a full length-protein. It is classified as (superfamily/group/family): Protease, Cysteine, UCH2b. The cytogenetic position of this gene is 12q24.3. This sequence is represented in the database of public ESTs (dbEST) by the following ESTs: BG678894, BG476418, BE264732. The nucleic acid contains short repetitive sequence (the position and sequence of the repeat): 1534 gagtgcaagtctgaagaatg 1553.

SGPr427, SEQ ID NO:18, SEQ ID NO:77 is 3372 nucleotides long. The open reading frame starts at position 1 and ends at position 3372, giving an ORF length of 3372 nucleotides. The predicted protein is 1123 amino acids long. This sequence codes for a full length protein. It is classified as (superfamily/group/family): Protease, Cysteine, UCH2b. The cytogenetic position of this gene is 17p13. This sequence is represented in the database of public ESTs (dbEST) by the following ESTs: BG831111, AW996553, BE614914.

SGPr092, SEQ ID NO:19, SEQ ID NO:78 is 786 nucleotides long. The open reading frame starts at position 1 and ends at position 786, giving an ORE length of 786 nucleotides. The predicted protein is 261 amino acids long. This sequence codes for a full length protein. It is classified as (superfamily/group/family): Protease, Metalloprotease, PepM10. The cytogenetic position of this gene is 11p15. This sequence is represented in the database of public ESTs (dbEST) by the following ESTs: BG189720, AW966183, BG198356.

SGPr359, SEQ ID NO:20, SEQ ID NO:79 is 1452 nucleotides long. The open reading frame starts at position 1 and ends at position 1452, giving an ORF length of 1452 nucleotides. The predicted protein is 483 amino acids long. This sequence codes for a full length protein. It is classified as (superfamily/group/family): Protease, Metalloprotease, PepM1). The cytogenetic position of this gene is 11q22. This sequence is represented in the database of public ESTs (dbEST) by the following ESTs: BG187290.

SGPr104_{—}1, SEQ ID NO:21, SEQ ID NO:80 is 2298 nucleotides long. The open reading frame starts at position 1 and ends at position 2298, giving an ORF length of 2298 nucleotides. The predicted protein is 765 amino acids long. This sequence codes for a full length protein. It is classified as (superfamily/group/family): Protease, Metalloprotease, PepM13. The cytogenetic position of this gene is 3q27. This sequence is represented in the database of public ESTs (dbEST) by the following ESTs: BF511209, AW341249, AL119270.

SGPr303, SEQ ID NO:22, SEQ ID NO:81 is 1257 nucleotides long. The open reading frame starts at position 1 and ends at position 1257, giving an ORF length of 1257 nucleotides. The predicted protein is 418 amino acids long. This sequence codes for a full length catalytic domain. It is classified as (superfamily/group/family): Protease, Metalloprotease, PepM2. The cytogenetic position of this gene is 17q11.1. This sequence is represented in the database of public ESTs (dbEST) by the following ESTs: AU138954, BG251083, AW161660.

SGPr402_{—}1, SEQ ID NO:23, SEQ ID NO:82 is 2268 nucleotides long. The open reading frame starts at position 1 and ends at position 2268, giving an ORF length of 2268 nucleotides. The predicted protein is 755 amino acids long. This sequence codes for a full length protein. It is classified as (superfamily/group/family): Protease, Serine, subtilase. The cytogenetic position of this gene is 19q11. This sequence is represented in the database of public ESTs (dbEST) by the following ESTs: AL041695, AA454137, BG719638.

SGPr434, SEQ ID NO:24, SEQ ID NO:83 is 1176 nucleotides long. The open reading frame starts at position 1 and ends at position 1176, giving an ORF length of 1176 nucleotides. The predicted protein is 391 amino acids long. This sequence codes for a full length-protein. It is classified as (superfamily/group/family): Protease, Serine, Trypsin. The cytogenetic position of this gene is 3p21. This sequence is represented in the database of public ESTs (dbEST) by the following ESTs: AW137088, BF593342.

SGPr446_{—}1, SEQ ID NO:25, SEQ ID NO:84 is 681 nucleotides long. The open reading frame starts at position 1 and ends at position 681, giving an ORF length of 681 nucleotides. The predicted protein is 226 amino acids long. This sequence codes for a full length catalyc domain. It is classified as (superfamily/group/family): Protease, Serine, Trypsin. The cytogenetic position of this gene is 3p21. This sequence is represented in the database of public ESTs (dbEST) by the following ESTs: AW243584. The nucleic acid contains short repetitive sequence (the position and sequence of the repeat): 798 ggtgggcatcatcagctgggg 818.

SGPr447, SEQ ID NO:26, SEQ ID NO:85 is 888 nucleotides long. The open reading frame starts at position 1 and ends at position 888, giving an ORF length of 888 nucleotides. The predicted protein is 295 amino acids long. This sequence codes for a partial protein. It is classified as (superfamily/group/family): Protease, Serine, Trypsin. The cytogenetic position of this gene is 16p13.3. This sequence is represented in the database of public ESTs (dbEST) by the following ESTs: none.

SGPr432_{—}1, SEQ ID NO:27, SEQ ID NO:86 is 1887 nucleotides long. The open reading frame starts at position 1 and ends at position 1887, giving an ORF length of 1887 nucleotides. The predicted protein is 628 amino acids long. This sequence codes for a full length protein. It is classified as (superfamily/group/family): Protease, Serine, Trypsin. The cytogenetic position of this gene is unknown. This sequence is represented in the database of public ESTs (dbEST) by the following ESTs: BE264142, BG474605, BF304202.

SGPr529, SEQ ID NO:28, SEQ ID NO:87 is 831 nucleotides long. The open reading frame starts at position 1 and ends at position 831, giving an ORF length of 831 nucleotides. The predicted protein is 276 amino acids long. This sequence codes for a full length protein. It is classified as (superfamily/group/family): Protease, Serine, Trypsin. The cytogenetic position of this gene is 19q13.4. This nucleotide sequence contains the following single nucleotide polymorphisms (the accession number of SNP is given, with the allele position, followed by the sequence surrounding the SNP within the gene): ss1550333_allelePos=51, taggggatgaycacctgct; ss1546197_allelePos=51, gccggacsactcgc. SNP ss1550333 codes for a C or T at nucleotide 297. Amino acid 99 is an aspartic acid with either nucleotide, so the SNP is silent. There is another SNP, ss1546197, that codes for a C or G at position 336; amino acid 112 is a threonine when either nucleotide is present, and so this SNP is silent. This sequence is represented in the database of public ESTs (dbEST) by the following ESTs: BE898352, BG469321.

SGPr428_{—}1, SEQ ID NO:29, SEQ ID NO:88 is 858 nucleotides long. The open reading frame starts at position 1 and ends at position 858, giving an ORF length of 858 nucleotides. The predicted protein is 285 amino acids long. This sequence codes for a full length catalytic domain. It is classified as (superfamily/group/family): Protease, Serine, Trypsin. The cytogenetic position of this gene is 8p23. This sequence is represented in the database of public ESTs (dbEST) by the following ESTs: none. The nucleic acid contains short repetitive sequence (the position and sequence of the repeat): 473 catgcacctggaaaagctg 491.

SGPr425, SEQ ID NO:30, SEQ ID NO:89 is 1242 nucleotides long. The open reading frame starts at position 1 and ends at position 1242, giving an ORF length of 1242 nucleotides. The predicted protein is 413 amino acids long. This sequence codes for a full length protein. It is classified as (superfamily/group/family): Protease, Serine, Trypsin. The cytogenetic position of this gene is 6q14. This nucleotide sequence contains the following single nucleotide polymorphisms (the accession number of SNP is given, with the allele position, followed by the sequence surrounding the SNP within the gene): ss674620_allelePos=201, gagcatctgcVggagagag, SNP ss674620 codes for a G or A or C at nucleotide 671. If the nucleotide is a guanine, amino acid 224 is an arginine; if it is an adenine, amino acid 224 is a glutamine; if the nucleotide is a cytosine, the amino acid at 224 is a proline. This sequence is represented in the database of public ESTs (dbEST) by the following ESTs: AL551286, AA445948, AA424073. The nucleic acid contains short repetitive sequence (the position and sequence of the repeat): 1111 tcagggcaccagtgggtgga 1130.

SGPr548, SEQ ID NO:31, SEQ ID NO:90 is 963 nucleotides long. The open reading frame starts at position 1 and ends at position 963, giving an ORF length of 963 nucleotides. The predicted protein is 320 amino acids long. This sequence codes for a full length protein. It is classified as (superfamily/group/family): Protease, Serine, Trypsin. The cytogenetic position of this gene is 19q13.4. This sequence is represented in the database of public ESTs (dbEST) by the following ESTs: none.

SGPr396, SEQ ID NO:32, SEQ ID NO:91 is 987 nucleotides long. The open reading frame starts at position 1 and ends at position 987, giving an ORF length of 987 nucleotides. The predicted protein is 328 amino acids long. This sequence codes for a full length protein. It is classified as (superfamily/group/family): Protease, Serine, Trypsin. The cytogenetic position of this gene is 4q32. This sequence is represented in the database of public ESTs (dbEST) by the following ESTs: none.

SGPr426, SEQ ID NO:33, SEQ ID NO:92 is 1278 nucleotides long. The open reading frame starts at position 1 and ends at position 1278, giving an ORF length of 1278 nucleotides. The predicted protein is 425 amino acids long. This sequence codes for a full length protein. It is classified as (superfamily/group/family): Protease, Serine, Trypsin. The cytogenetic position of this gene is 4q13. This sequence is represented in the database of public ESTs (dbEST) by the following ESTs: none.

SGPr552, SEQ ID NO:34, SEQ ID NO:93 is 666 nucleotides long. The open reading frame starts at position 1 and ends at position 666, giving an ORF length of 666 nucleotides. The predicted protein is 221 amino acids long. This sequence codes for a full length catalytic domain. It is classified as (superfamily/group/family): Protease, Serine, Trypsin. The cytogenetic position of this gene is 4q13. This sequence is represented in the database of public ESTs (dbEST) by the following ESTs: none.

SGPr405, SEQ ID NO:35, SEQ ID NO:94 is 2847 nucleotides long. The open reading frame starts at position 1 and ends at position 2847, giving an ORF length of 2847 nucleotides. The predicted protein is 948 amino acids long. This sequence codes for a full length protein. It is classified as (superfamily/group/family): Protease, Serine, Trypsin. The cytogenetic position of this gene is 16p13.3. This sequence is represented in the database of public ESTs (dbEST) by the following ESTs: none.

SGPr485_{—}1, SEQ ID NO:36, SEQ ID NO:95 is 1059 nucleotides long. The open reading frame starts at position 1 and ends at position 1059, giving an ORF length of 1059 nucleotides. The predicted protein is 352 amino acids long. This sequence codes for a full length protein. It is classified as (superfamily/group/family): Protease, Serine, Trypsin. The cytogenetic position of this gene is 8p23. This nucleotide sequence contains the following single nucleotide polymorphisms (the accession number of SNP is given, with the allele position, followed by the sequence surrounding the SNP within the gene): ss1532791_allelePos=51 , tggagakaagaacac. ss1532791 codes for a G or a T at position 834. This polymorphism changes amino acid 278. If the nucleotide at 834 is a guanine, amino acid 278 is a glutamic acid (E); if the nucleotide is a thymine, amino acid 278 is an aspartic acid (D). This sequence is represented in the database of public ESTs (dbEST) by the following ESTs: AA781356.

SGPr534, SEQ ID NO:37, SEQ ID NO:96 is 792 nucleotides long. The open reading frame starts at position 1 and ends at position 792, giving an ORF length of 792 nucleotides. The predicted protein is 263 amino acids long. This sequence codes for a full length protein. It is classified as (superfamily/group/family): Protease, Serine, Trypsin. The cytogenetic position of this gene is 16q23. This nucleotide sequence contains the following six single nucleotide polymorphisms (the accession number of SNP is given, with the allele position, followed by the sequence surrounding the SNP within the gene):

ss1522946_allelePos=51, gctctaccwccacgccc;
ss1522943_allelePos=51, cgcacctgctcyaccaccac;
ss1522933_allelePos=51, ctgccagaaggayggagcctgg;
ss1522931_allelePos=51 total len = 101, gtctgccaraaggacg;
ss1522930_allelePos=51, gggtgactctggmggccccct;
ss1522928_allelePos=51, tgcatgggygactctgg;

SNP ss1522946 codes for A or T at position 721. If 721 is adenine, amino acid 241 is threonine (T); if 721 is Thymine, amino acid 241 is serine (S).

SNP ss1522943 codes for C or T at position 717; this SNP is silent (239=serine).

SNP ss1522933 codes for C or T at 666; this SNP is silent (222=aspartic acid).

SNP ss1522931 codes for A or G at position 660; this SNP is silent (220=glutamine).

SNP ss1522930 codes for A or C at position 642; this SNP is silent (214=glycine).

SNP ss1522928 codes for a C or T at position 633; this SNP is silent (211=glycine).

This sequence is represented in the database of public ESTs (dbEST) by the following ESTs: AW583018, AW582942, AW960025. The nucleic acid contains short repetitive sequence (the position and sequence of the repeat): 172 cacttctgcgggggctccctcatc 195.

SGPr390, SEQ ID NO:38, SEQ ID NO:97 is 3387 nucleotides long. The open reading frame starts at position 1 and ends at position 3387, giving an ORF length of 3387 nucleotides. The predicted protein is 1128 amino acids long. This sequence codes for a full length protein. It is classified as (superfamily/group/family): Protease, Serine, Trypsin. The cytogenetic position of this gene is 19q11. This nucleotide sequence contains the following single nucleotide polymorphisms (the accession number of SNP is given, with the allele position, followed by the sequence surrounding the SNP within the gene): ss82431_allelePos=99 , gccgtgarcaccactg; ss1320361_allelePos=225,agcggccascattggcgt. ss82431 codes for an A or G at position 2585. If this nucleotide ia an adenine, amino acid 862 is an asparagine (N); if this nucleotide is a guanine, amino acid 862 is a serine. The SNP ss1320361 codes for C or G at position 89. If position 89 is a cytosine, amino acid 30 is a threonine (T). If position 89 is a guanine, amino acid 30 is a serine. This sequence is represented in the database of public ESTs (dbEST) by the following ESTs: C16607.

SGPr521, SEQ ID NO:39, SEQ ID NO:98 is 762 nucleotides long. The open reading frame starts at position 1 and ends at position 762, giving an ORF length of 762 nucleotides. The predicted protein is 253 amino acids long. This sequence codes for a full length protein. It is classified as (superfamily/group/family): Protease, Serine, Trypsin. The cytogenetic position of this gene is 19q13.4.This sequence is represented in the database of public ESTs (dbEST) by the following ESTs: AA542994, BE713379, W58737. The nucleic acid contains short repetitive sequence (the position and sequence of the repeat): 646 caaggtctggtgtcctgggg 665.

SGPr530_{—}1, SEQ ID NO:40, SEQ ID NO:99 is 816 nucleotides long. The open reading frame starts at position 1 and ends at position 816, giving an ORF length of 816 nucleotides. The predicted protein is 271 amino acids long. This sequence codes for a full length catalytic domain. It is classified as (superfamily/group/family): Protease, Serine, Trypsin. The cytogenetic position of this gene is 9q22. This sequence is represented in the database of public ESTs (dbEST) by the following ESTs: none.

SGPr520, SEQ ID NO:41, SEQ ID NO:100 is 1737 nucleotides long. The open reading frame starts at position 1 and ends at position 1737, giving an ORF length of 1737 nucleotides. The predicted protein is 578 amino acids long. This sequence codes for a full length protein. It is classified as (superfamily/group/family): Protease, Serine, Trypsin. The cytogenetic position of this gene is 2q37. This sequence is represented in the database of public ESTs (dbEST) by the following ESTs: none.

SGPr455, SEQ ID NO:42, SEQ ID NO:101 is 2913 nucleotides long. The open reading frame starts at position 1 and ends at position 2913, giving an ORF length of 2913 nucleotides. The predicted protein is 970 amino acids long. This sequence codes for a full length protein. It is classified as (superfamily/group/family): Protease, Serine, Trypsin. The cytogenetic position of this gene is 12p11.2. This sequence is represented in the database of public ESTs (dbEST) by the following ESTs: AW450155, AW995496.

SGPr507_{—}2, SEQ ID NO:43, SEQ ID NO:102 is 798 nucleotides long. The open reading frame starts at position 1 and ends at position 798, giving an ORF length of 798 nucleotides. The predicted protein is 265 amino acids long. This sequence codes for a full length protein. It is classified as (superfamily/group/family): Protease, Serine, Trypsin. The cytogenetic position of this gene is 7q36. This sequence is represented in the database of public ESTs (dbEST) by the following ESTs: BG217724, BG219738, BG192709.

SGPr559, SEQ ID NO:44, SEQ ID NO:103 is 1365 nucleotides long. The open reading frame starts at position 1 and ends at position 1365, giving an ORF length of 1365 nucleotides. The predicted protein is 454 amino acids long. This sequence codes for a full length protein. It is classified as (superfamily/group/family): Protease, Serine, Trypsin. The cytogenetic position of this gene is 21q22. This sequence is represented in the database of public ESTs (dbEST) by the following ESTs: AI978874, AI469095, BF435670

SGPr567_{—}1, SEQ ID NO:45, SEQ ID NO:104 is 1614 nucleotides long. The open reading frame starts at position 1 and ends at position 1614, giving an ORF length of 1614 nucleotides. The predicted protein is 537 amino acids long. This sequence codes for a full length protein. It is classified as (superfamily/group/family): Protease, Serine, Trypsin. The cytogenetic position of this gene is 11 q23. This sequence is represented in the database of public ESTs (dbEST) by the following ESTs: BE732381, R78581, AW845106.

SGPr479_{—}1, SEQ ID NO:46, SEQ ID NO:105 is 981 nucleotides long. The open reading frame starts at position 1 and ends at position 981, giving an ORF length of 981 nucleotides. The predicted protein is 326 amino acids long. This sequence codes for a full length protein. It is classified as (superfamily/group/family): Protease, Serine, Trypsin. The cytogenetic position of this gene is 1q42. This sequence is represented in the database of public ESTs (dbEST) by the following ESTs: BG718703, AA401705, AA398170. The nucleic acid contains short repetitive sequence (the position and sequence of the repeat): 780 tggaattgtgagctggggccg 800.

SGPr489_{—}1, SEQ ID NO:47, SEQ ID NO:106 is 1671 nucleotides long. The open reading frame starts at position 1 and ends at position 1671, giving an ORF length of 1671 nucleotides. The predicted protein is 556 amino acids long. This sequence codes for a full length catalytic domain. It is classified as (superfamily/group/family): Protease, Serine, Trypsin. The cytogenetic position of this gene is 11p15. This sequence is represented in the database of public ESTs (dbEST) by the following ESTs: AW271430, AW237893.

SGPr465_{—}1, SEQ ID NO:48, SEQ ID NO:107 is 894 nucleotides long. The open reading frame starts at position 1 and ends at position 894, giving an ORF length of 894 nucleotides. The predicted protein is 297 amino acids long. This sequence codes for a full length catalytic domain. It is classified as (superfamily/group/family): Protease, Serine, Trypsin. The cytogenetic position of this gene is unknown. This sequence is represented in the database of public ESTs (dbEST) by the following ESTs: none.

SGPr524_{—}1, SEQ ID NO:49, SEQ ID NO:108 is 2553 nucleotides long. The open reading frame starts at position 1 and ends at position 2553, giving an ORF length of 2553 nucleotides. The predicted protein is 850 amino acids long. This sequence codes for a full length protein. It is classified as (superfamily/group/family): Protease, Serine, Trypsin. The cytogenetic position of this gene is unknown. This nucleotide sequence contains the following single nucleotide polymorphisms (the accession number of SNP is given, with the allele position, followed by the sequence surrounding the SNP within the gene): ss2013558_allelePos=201, gacatggawgtggacgac; ss2014128_allelePos=35 8, acaatttttygagtgccca. ss2013558 codes for a T of C at position 675; this is a silent SNP. Ss20114128 codes for a C or T at 1369; if the nucleotide is a cytosine, amino acid 457 is an arginine; if the nucleotide 1369 is a thymine, a stop codon is introduced, truncating the protein to 456 amino acids. This sequence is represented in the database of public ESTs (dbEST) by the following ESTs: none. The nucleic acid contains short repetitive sequence (the position and sequence of the repeat): 711 aaaaaaaaagaaaagaaaggaaaa 734.

SGPr422, SEQ ID NO:50, SEQ ID NO:109 is 1344 nucleotides long. The open reading frame starts at position 1 and ends at position 1344, giving an ORF length of 1344 nucleotides. The predicted protein is 447 amino acids long. This sequence codes for a full length protein. It is classified as (superfamily/group/family): Protease, Serine, Trypsin. The cytogenetic position of this gene is 4q13. This nucleotide sequence contains the following single nucleotide polymorphisms (the accession number of SNP is given, with the allele position, followed by the sequence surrounding the SNP within the gene): ss1091793_allelePos=101, acatacgccrgatttgtttg; ss448607_allelePos=101, tgggagcrggtcctgcct. SNP ss1091793 codes for an adenine or guanine at position 956. If 956 is guanine, amino acid 319 is arginine (R); if nucleotide 956 is adenine, amino acid 319 is glutamine (Q). The SNP ss448607 codes for an A or G at position 552. This is silent (amino acid 184=alanine). This sequence is represented in the database of public ESTs (dbEST) by the following-ESTs: none.

SGPr538, SEQ ID NO:51, SEQ ID NO:110 is 1374 nucleotides long. The open reading frame starts at position 1 and ends at position 1374, giving an ORF length of 1374 nucleotides. The predicted protein is 457 amino acids long. This sequence codes for a full length protein. It is classified as (superfamily/group/family): Protease, Serine, Trypsin. The cytogenetic position of this gene is 11 q23. This sequence is represented in the database of public ESTs (dbEST) by the following ESTs: AL538140, BF934870. The nucleic acid contains short repetitive sequence (the position and sequence of the repeat): 545 tgggaggcttcctggaggag 564.

SGPr527_{—}1, SEQ ID NO:52, SEQ ID NO:111 is 2457 nucleotides long. The open reading frame starts at position 1 and ends at position 2457, giving an ORF length of 2457 nucleotides. The predicted protein is 818 amino acids long. This sequence codes for a full length protein. It is classified as (superfamily/group/family): Protease, Serine, Trypsin. The cytogenetic position of this gene is unknown. This sequence is represented in the database of public ESTs (dbEST) by the following ESTs: AW450407, AI190509, AI864473.

SGPr542, SEQ ID NO:53, SEQ ID NO:112 is 855 nucleotides long. The open reading frame starts at position 1 and ends at position 855, giving an ORF length of 855 nucleotides. The predicted protein is 284 amino acids long. This sequence codes for a full length protein. It is classified as (superfamily/group/family): Protease, Serine, Trypsin. The cytogenetic position of this gene is 19q13.1. This sequence is represented in the database of public ESTs (dbEST) by the following ESTs: none.

SGPr551, SEQ ID NO:54, SEQ ID NO:113 is 2409 nucleotides long. The open reading frame starts at position 1 and ends at position 2409, giving an ORF length of 2409 nucleotides. The predicted protein is 802 amino acids long. This sequence codes for a full length protein. It is classified as (superfamily/group/family): Protease, Serine, Trypsin. The cytogenetic position of this gene is 22q13. This nucleotide sequence contains the following single nucleotide polymorphisms (the accession number of SNP is given, with the allele position, followed by the sequence surrounding the SNP within the gene): rs881144_allelePos=200, ctgcagccctaygccgagagg; rs855791_allelePos=101, agcgaggyctatcgcta. SNP rs881144 codes for a C or T at position 1227; this a a silent SNP (409=tyrosine). SNP rs855791 codes for C or T at position 2180. If the nucleotide at 2180 is cytosine, the amino acid at 727 is alanine; if the nucleotide is thymine, amino acid 727 is valine. This sequence is represented in the database of public ESTs (dbEST) by the following ESTs: AV693114, N70418, AA609066

SGPr451, SEQ ID NO:55, SEQ ID NO:114 is 1080 nucleotides long. The open reading frame starts at position 1 and ends at position 1080, giving an ORF length of 1080 nucleotides. The predicted protein is 359 amino acids long. This sequence codes for a full length protein. It is classified as (superfamily/group/family): Protease, Serine, Trypsin. The cytogenetic position of this gene is 12q23. This nucleotide sequence contains the following single nucleotide polymorphisms (the accession number of SNP is given, with the allele position, followed by the sequence surrounding the SNP within the gene): ss1881349_allelePos=201, gggcgcatgcaragg; ss1266911_allelePos=101 , ccactgcactaaagacrctag. SNP ss1881349 codes for an A or G at position 217. If the nucleotide at 217 is adenine, amino acid 73 is lysine (K); if the nucleotide is guanine, amino acid 73 is glutamic acid (E). The SNP ss1266911 codes for an A or G at position 412. If 412 is guanine, amino acid 138 is alanine (A); if 412 is adenine, amino acid 138 is threonine (T). This sequence is represented in the database of public ESTs (dbEST) by the following ESTs: BG722131, BG722203,

SGPr452_{—}1, SEQ ID NO:56, SEQ ID NO:115 is 867 nucleotides long. The open reading frame starts at position 1 and ends at position 867, giving an ORF length of 867 nucleotides. The predicted protein is 288 amino acids long. This sequence codes for a full length protein. It is classified as (superfamily/group/family): Protease, Serine, Trypsin. The cytogenetic position of this gene is 16p13.3. This sequence is represented in the database of public ESTs (dbEST) by the following ESTs: none

SGPr504, SEQ ID NO:57, SEQ ID NO:116 is 135 nucleotides long. The open reading frame starts at position 1 and ends at position 135, giving an ORF length of 135 nucleotides. The predicted protein is 44 amino acids long. This sequence codes for a partial length protein. It is classified as (superfamily/group/family): Protease, Serine, Trypsin. The cytogenetic position of this gene is unknown. This sequence is represented in the database of public ESTs (dbEST) by the following ESTs: none

SGPr469, SEQ ID NO:58, SEQ ID NO:117 is 138 nucleotides long. The open reading frame starts at position 1 and ends at position 138, giving an ORF length of 138 nucleotides. The predicted protein is 45 amino acids long. This sequence codes for a partial length protein. It is classified as (superfamily/group/family): Protease, Serine, Trypsin. The cytogenetic position of this gene is unknown. This sequence is represented in the database of public ESTs (dbEST) by the following ESTs: AW753029, Z19070. The nucleic acid contains short repetitive sequence (the position and sequence of the repeat): 55 gggattgtgagctggggc 72.

SGPr400, SEQ ID NO:59, SEQ ID NO:118 is 930 nucleotides long. The open reading frame starts at position 1 and ends at position 930, giving an ORF length of 930 nucleotides. The predicted protein is 309 amino acids long. This sequence codes for a partial length protein. It is classified as (superfamily/group/family): Protease, Serine, Trypsin. The cytogenetic position of this gene is 4q32. This sequence is represented in the database of public ESTs (dbEST) by the following ESTs: none

### DESCRIPTION OF NOVEL PROTEASE POLYPEPTIDES

SGPr397, SEQ ID NO:1, SEQ ID NO:60 encodes a protein that is 315 amino acids long. It is classified as an Carboxypeptidase protease, of the Zn carboxypeptidase family. The protease domain(s) in this protein match the hidden Markov profile for a Zn carboxypeptidase (PF00246) domain, from amino acid 139 to amino acid 280. The positions within the HMMR profile that match the protein sequence are from profile position 1 to profile position 146. Other domains identified within this protein are: Carboxypeptidase activation peptide (PF02244) from amino acid 41 to 120. The pro-segment moiety (activation peptide) is responsible for modulation of folding and activity of the pro-enzyme (see http://pfam.wustl.edu/cgi-bin/getdesc?name=Propep_M14). The results of a Smith Waterman search (PAM100, gap open and extend penalties of 12 and 2) of the public database of amino acid sequences (NRAA) with this protein sequence yielded the following results: Pscore=3.10E-220; number of identical amino acids=315; percent identity=100%; percent similarity=100%; the accession number of the most similar entry in NRAA is NP_{—}065094.1; the name or description, and species, of the most similar protein in NRAA is: carboxypeptidase B precursor [Homo sapiens].

SGPr413, SEQ ID NO:2, SEQ ID NO:61 encodes a protein that is 374 amino acids long. It is classified as an Carboxypeptidase protease, of the Zn carboxypeptidase family. The protease domain(s) in this protein match the hidden Markov profile for a Zn carboxypeptidase (PF00246), from amino acid 50 to amino acid 291. The positions within the HMMR profile that match the protein sequence are from profile position 1 to profile position 248. The results of a Smith Waterman search (PAM100, gap open and extend penalties of 12 and 2) of the public database of amino acid sequences (NRAA) with this protein sequence yielded the following results: Pscore=5.90E-93; number of identical amino acids=146; percent identity=49%; percent similarity=68%; the accession number of the most similar entry in NRAA is AAF01344.1; the name or description, and species, of the most similar protein in NRAA is: (AF190274) carboxypeptidase homolog [Bothrops jararaca].

SGPr404, SEQ ID NO:3, SEQ ID NO:62 encodes a protein that is 529 amino acids long. It is classified as an Carboxypeptidase protease, of the Zn carboxypeptidase family. The protease domain(s) in this protein match the hidden Markov profile for a Zn carboxypeptidase (PF00246), from amino acid 91 to amino acid 466. The positions within the HMMR profile that match the protein sequence are from profile position 1 to profile position 248. The results of a Smith Waterman search (PAM100, gap open and extend penalties of 12 and 2) of the public database of amino acid sequences (NRAA) with this protein sequence yielded the following results: Pscore=0; number of identical amino acids=502; percent identity=94%; percent similarity=98%; the accession number of the most similar entry in NRAA is NP_{—}061355.1; the name or description, and species, of the most similar protein in NRAA is: carboxypeptidase X2 [Mus musculus].

SGPr536_{—}1, SEQ ID NO:4, SEQ ID NO:63 encodes a protein that is 467 amino acids long. It is classified as an Cysteine protease, of the papain family. The protease domain(s) in this protein match the hidden Markov profile for a papain (PF00112), from amino acid 203 to amino acid 456. The positions within the HMMR profile that match the protein sequence are from profile position 1 to profile position 337. The results of a Smith Waterman search (PAM100, gap open and extend penalties of 12 and 2) of the public database of amino acid sequences (NRAA) with this protein sequence yielded the following results: Pscore=1.10E-276; number of identical amino acids=467; percent identity=100%; percent similarity=100%; the accession number of the most similar entry in NRAA is NP_{—}071447.1; the name or description, and species, of the most similar protein in NRAA is: P3ECSL [Homo sapiens].

SGPr414, SEQ ID NO:5, SEQ ID NO:64 encodes a protein that is 3353 amino acids long. It is classified as a Cysteine protease, of the UCH2b family. The protease domain(s) in this protein match the hidden Markov profile for a Ubiquitin carboxyl-terminal hydrolase family 2b (PF00443), from amino acid 1951 to amino acid 2045. The positions within the HMMR profile that match the protein sequence are from profile position 1 to profile position 72. Other domains identified within this protein are: Ubiquitin carboxyl-terminal hydrolases family 2 (UCH2b, PF00442) from amino acid 1701 to 1731. Ubiquitin carboxyl-terminal hydrolases (EC 3.1.2.15) (UCH) (deubiquitinating enzymes) are thiol proteases that recognize and hydrolyze the peptide bond at the C-terminal glycine of ubiquitin. These enzymes are involved in the processing of poly-ubiquitin precursors as well as that of ubiquinated proteins. The results of a Smith Waterman search (PAM100, gap open and extend penalties of 12 and 2) of the public database of amino acid sequences (NRAA) with this protein sequence yielded the following results: Pscore=0; number of identical amino acids=1259; percent identity=99%; percent similarity=100%; the accession number of the most similar entry in NRAA is NP_{—}055524.1; the name or description, and species, of the most similar protein in NRAA is: KIAA0570 gene product [Homo sapiens].

SGPr430, SEQ ID NO:6, SEQ ID NO:65 encodes a protein that is 980 amino acids long. It is classified as a Cysteine protease, of the UCH2b family. The protease domain(s) in this protein match the hidden Markov profile for a Ubiquitin carboxyl-terminal hydrolase family 2b (PF00443), from amino acid 886 to amino acid 951. The positions within the HMMR profile that match the protein sequence are from profile position 1 to profile position 72. Other domains identified within this protein are: UCH2b (PF00442) from amino acids 342 to 373. The results of a Smith Waterman search (PAM100, gap open and extend penalties of 12 and 2) of the public database of amino acid sequences (NRAA) with this protein sequence yielded the following results: Pscore=0; number of identical amino acids=930; percent identity=99%; percent similarity=99%; the accession number of the most similar entry in NRAA is BAB13420.1; the name or description, and species, of the most similar protein in NRAA is: (AB046814) KIAA1594 protein [Homo sapiens].

SGPr496_{—}1, SEQ ID NO:7, SEQ ID NO:66 encodes a protein that is 953 amino acids long. It is classified as a Cysteine protease, of the UCH2b family. The protease domain(s) in this protein match the hidden Markov profile for a Ubiquitin carboxyl-terminal hydrolase family 2b (PF00443), from amino acid 875 to amino acid 935. The positions within the HMMR profile that match the protein sequence are from profile position 1 to profile position 72. Other domains identified within this protein are: UCH2b (PF00442) from 593 to 694; and a Zn-finger domain (PF02148), found in ubiquitin-hydrolases, from 465 to 534. The results of a Smith Waterman search (PAM100, gap open and extend penalties of 12 and 2) of the public database of amino acid sequences (NRAA) with this protein sequence yielded the following results: Pscore=2.00E-190; number of identical amino acids=496; percent identity=95%; percent similarity=98%; the accession number of the most similar entry in NRAA is AAF66953.1; the name or description, and species, of the most similar protein in NRAA is: (AF229643) ubiquitin specific protease [Mus musculus].

SGPr495, SEQ ID NO:8, SEQ ID NO:67 encodes a protein that is 783 amino acids long. It is classified as a Cysteine protease, of the UCH2b family. The protease domain(s) in this protein match the hidden Markov profile for a Ubiquitin carboxyl-terminal hydrolase family 2b (PF00443), from amino acid 695 to amino acid 781. The positions within the HMMR profile that match the protein sequence are from profile position 1 to profile position 72. Other domains identified within this protein are: UCH2b (PF00442) from 190 to 221; and Zn-finger in ubiquitin-hydrolases (PF02148) from 465 to 534. The results of a Smith Waterman search (PAM100, gap open and extend penalties of 12 and 2) of the public database of amino acid sequences (NRAA) with this protein sequence yielded the following results: Pscore=2.40E-176; number of identical amino acids=282; percent identity=100%; percent similarity=100%; the accession number of the most similar entry in NRAA is AAH05991.1; the name or description, and species, of the most similar protein in NRAA is: (BC005991) Unknown (protein for MGC: 14793) [Homo sapiens].

SGPr407, SEQ ID NO:9, SEQ ID NO:68 encodes a protein that is 752 amino acids long. It is classified as a Cysteine protease, of the UCH2b family. The protease domain(s) in this protein match the hidden Markov profile for a Ubiquitin carboxyl-terminal hydrolase family 2b (PF00443), from amino acid 481 to amino acid 491. The positions within the HMMR profile that match the protein sequence are from profile position 80 to profile position 90. The results of a Smith Waterman search (PAM100, gap open and extend penalties of 12 and 2) of the public database of amino acid sequences (NRAA) with this protein sequence yielded the following results: Pscore=2.60E-40; number of identical amino acids=80; percent identity=76%; percent similarity=84%; the accession number of the most similar entry in NRAA is NP_{—}036607.1; the name or description, and species, of the most similar protein in NRAA is: ubiquitin specific protease 23; NEDD8-specific protease [Homo sapiens].

SGPr453, SEQ ID NO:10, SEQ ID NO:69 encodes a protein that is 712 amino acids long. It is classified as a Cysteine protease, of the UCH2b family. The protease domain(s) in this protein match the hidden Markov profile for a Ubiquitin carboxyl-terminal hydrolase family 2b (PF00443), from amino acid 615 to amino acid 677. The positions within the HMMR profile that match the protein sequence are from profile position 1 to profile position 72. Other domains identified within this protein are: UCH2b (PF00442) from 273 to 304; and Zn-finger in ubiquitin-hydrolases (PF02148) from amino acids 29 to 99. The results of a Smith Waterman search (PAM100, gap open and extend penalties of 12 and 2) of the public database of amino acid sequences (NRAA) with this protein sequence yielded the following results: Pscore=0; number of identical amino acids=712; percent identity=100%; percent similarity=100%; the accession number of the most similar entry in NRAA is NP_{—}115523.1; the name or description, and species, of the most similar protein in NRAA is: hypothetical protein DKFZp434DO127 [Homo sapiens].

SGPr445, SEQ ID NO:11, SEQ ID NO:70 encodes a protein that is 289 amino acids long. It is classified as a Cysteine protease, of the UCH2b family. The protease domain(s) in this protein match the hidden Markov profile for a Ubiquitin carboxyl-terminal hydrolase family 2b (PF00443), from amino acid 190 to amino acid 221. The positions within the HMMR profile that match the protein sequence are from profile position 1 to profile position 32. The results of a Smith Waterman search (PAM100, gap open and extend penalties of 12 and 2) of the public database of amino acid sequences (NRAA) with this protein sequence yielded the following results: Pscore=3.60E-185; number of identical amino acids=289; percent identity=100%; percent similarity=100%; the accession number of the most similar entry in NRAA is AAH05991.1; the name or description, and species, of the most similar protein in NRAA is: (BC005991) Unknown (protein for MGC: 14793) [Homo sapiens].

SGPr401_{—}1, SEQ ID NO:12, SEQ ID NO:71 encodes a protein that is 366 amino acids long. It is classified as a Cysteine protease, of the UCH2b family. The protease domain(s) in this protein match the hidden Markov profile for a Ubiquitin carboxyl-terminal hydrolase family 2b (PF00443), from amino acid 292 to amino acid 364. The positions within the HMMR profile that match the protein sequence are from profile position 1 to profile position 72. Other domains identified within this protein are: UCH2b (PF00442) from amino acids 35 to 66. The results of a Smith Waterman search (PAM100, gap open and extend penalties of 12 and 2) of the public database of amino acid sequences (NRAA) with this protein sequence yielded the following results: Pscore=7.30E-254; number of identical amino acids=366; percent identity=100%; percent similarity=100%; the accession number of the most similar entry in NRAA is NP_{—}073743.1; the name or description, and. species, of the most similar protein in NRAA is: hypothetical protein FLJ12552 [Homo sapiens].

SGPr408, SEQ ID NO:13, SEQ ID NO:72 encodes a protein that is 1287 amino acids long. It is classified as a Cysteine protease, of the UCH2b family. The protease domain(s) in this protein match the hidden Markov profile for a Ubiquitin carboxyl-terminal hydrolase family 2b (PF00443), from amino acid 395 to amino acid 475. The positions within the HMMR profile that match the protein sequence are from profile position 1 to profile position 72. Other domains identified within this protein are: UCH2b (PF00442)from amino acids 100 to 131. The results of a Smith Waterman search (PAM100, gap open and extend penalties of 12 and 2) of the public database of amino acid sequences (NRAA) with this protein sequence yielded the following results: Pscore=0; number of identical amino acids=1287; percent identity=100%; percent similarity=100%; the accession number of the most similar entry in NRAA is BAB55063.1; the name or description, and species, of the most similar protein in NRAA is: (AK027362) unnamed protein product [Homo sapiens].

SGPr480, SEQ ID NO:14, SEQ ID NO:73 encodes a protein that is 1604 amino acids long. It is classified as-a Cysteine protease, of the UCH2b family. The protease domain(s) in this protein match the hidden Markov profile for a Ubiquitin carboxyl-terminal hydrolase family 2b (PF00443), from amino acid 1506 to amino acid 1566. The positions within the HMMR profile that match the protein sequence are from profile position 1 to profile position 72. Other domains identified within this protein are: UCH2b (PF00442) from 734 to 765; and two EF hands (PF00036) from 232 to 260, and from 268 to 296. Many calcium-binding proteins belong to the same evolutionary family and share a type of calcium-binding domain known as the EF-hand (see http://www.expasy.ch/cgi-bin/prosite-search-ac?PDOC00018). This type of domain consists of a twelve residue loop flanked on both side by a twelve residue alpha-helical domain. In an EF-hand loop the calcium ion is coordinated in a pentagonal bipyramidal configuration. This protein has a putative CAAX motif (CVLQ) which may direct it to the membrane fraction. The results of a Smith Waterman search (PAM100, gap open and extend penalties of 12 and 2) of the public database of amino acid sequences (NRAA) with this protein sequence yielded the following results: Pscore=0; number of identical amino acids=1272; percent identity=99%; percent similarity=99%; the accession number of the most similar entry in NRAA is NP_{—}115971.1; the name or description, and species, of the most similar protein in NRAA is: ubiquitin specific protease [Homo sapiens].

SGPr431, SEQ ID NO:15, SEQ ID NO:74 encodes a protein that is 1042 amino acids long. It is classified as a Cysteine protease, of the UCH2b family. The protease domain(s) in this protein match the hidden Markov profile for a Ubiquitin carboxyl-terminal hydrolase family 2b (PF00443), from amino acid 836 to amino acid 948. The positions within the HMMR profile that match the protein sequence are from profile position 1 to profile position 72. Other domains identified within this protein are: UCH2b (PF00442) from 445 to 476. The results of a Smith Waterman search (PAM100, gap open and extend penalties of 12 and 2) of the public database of amino acid sequences (NRAA) with this protein sequence yielded the following results: Pscore=2.40E-251; number of identical amino acids=397; percent identity=100%; percent similarity=100%; the accession number of the most similar entry in NRAA is NP_{—}115946.1; the name or description, and species, of the most similar protein in NRAA is: HP43.8KD protein [Homo sapiens].

SGPr429, SEQ ID NO:16, SEQ ID NO:75 encodes a protein that is 1033 amino acids long. It is classified as a Cysteine protease, of the UCH2b family. The protease domain(s) in this protein match the hidden Markov profile for a Ubiquitin carboxyl-terminal hydrolase family 2b (PF00443), from amino acid 332 to amino acid 419. The positions within the HMMR profile that match the protein sequence are from profile position 1 to profile position 72. Other domains identified within this protein are: UCH2b (PF00442) from 89 to 120. The results of a Smith Waterman search (PAM100, gap open and extend penalties of 12 and 2) of the public database of amino acid sequences (NRAA) with this protein sequence yielded the following results: Pscore=1.50E-250; number of identical amino acids=368; percent identity=100%; percent similarity=100%; the accession number of the most similar entry in NRAA is NP_{—}115612.1; the name or description, and species, of the most similar protein in NRAA is: hypothetical protein FLJ23277 [Homo sapiens]. This protein has a transmembrane domain from amino acid 87 to amino acid 109.

SGPr503, SEQ ID NO:17, SEQ ID NO:76 encodes a protein that is 517 amino acids long. It is classified as a Cysteine protease, of the UCH2b family. The protease domain(s) in this protein match the hidden Markov profile for a Ubiquitin carboxyl-terminal hydrolase family 2b (PF00443), from amino acid 432 to amino acid 501. The positions within the HMMR profile that match the protein sequence are from profile position 1 to profile position 72. Other domains identified within this protein are: UCH2b (PF00442) from 68 to 99. The results of a Smith Waterman search (PAM100, gap open and extend penalties of 12 and 2) of the public database of amino acid sequences (NRAA) with this protein sequence yielded the following results: Pscore=0; number of identical amino acids=508; percent identity=100%; percent similarity=100%; the accession number of the most similar entry in NRAA is AAH04868.1; the name or description, and species, of the most similar protein in NRAA is: (BC004868) Unknown (protein for MGC: 10702) [Homo sapiens]. This protein has a transmembrane domain from amino acid 35 to amino acid 57.

SGPr427, SEQ ID NO:18, SEQ ID NO:77 encodes a protein that is 1123 amino acids long. It is classified as a Cysteine protease, of the UCH2b family. The protease domain(s) in this protein match the hidden Markov profile for a Ubiquitin carboxyl-terminal hydrolase family 2b (PF00443), from amino acid 648 to amino acid 709. The positions within the HMMR profile that match the protein sequence are from profile position 1 to profile position 72. Other domains identified within this protein are: UCH2b (PF00442)from 101 to 129. The results of a Smith Waterman search (PAM100, gap open and extend penalties of 12 and 2) of the public database of amino acid sequences (NRAA) with this protein sequence yielded the following results: Pscore=1.80E-92; number of identical amino acids=269; percent identity=36%; percent similarity=53%; the accession number of the most similar entry in NRAA is AAF47260. 1; the name or description, and species, of the most similar protein in NRAA is: (AE003465) CG3872 gene product [Drosophila melanogaster]. long. It is classified as a Metalloprotease protease, of the PepM10 family. The protease domain(s) in this protein match the hidden Markov profile for a Peptidase_M10 (PF00413), from amino acid 75 to amino acid 194. The positions within the HMMR profile that match the protein sequence are from profile position 49 to profile position 168. Other domains identified within this protein are: ADAM domain, amino acid 207 to 218. The results of a Smith Waterman search (PAM100, gap open and extend penalties of 12 and 2) of the public database of amino acid sequences (NRAA) with this protein sequence yielded the following results: Pscore=4.70E-171; number of identical amino acids=261; percent identity=100%; percent similarity=100%; the accession number of the most similar entry in NRAA is XP_{—}011971.1; the-name or description, and species, of the most similar protein in NRAA is: matrix metalloproteinase 26 [Homo sapiens].

SGPr359, SEQ ID NO:20, SEQ-ID NO:79 encodes a protein that is 483 amino acids long. It is classified as a Metalloprotease protease, of the PepM10 family. The protease domain(s) in this protein match the hidden Markov profile for a Peptidase_M10 (PF00413), from amino acid 44 to amino acid 212. The positions within the HMMR profile that match the protein sequence are from profile position 1 to profile position 168. Other domains identified within this protein are: 3× Hemopexin (PF00045) domains from 302 to 403. Hemopexin is a serum glycoprotein that binds heme and transports it to the liver for breakdown and iron recovery, after which the free hemopexin returns to the circulation. Hemopexin-like domains have been found in two types of proteins: —in vitronectin, a cell adhesion and spreading factor found in plasma and tissues and in most members of the matrix metalloproteinases family (matrixins), including MMP-1, MMP-2, MMP-3, MMP-8, MMP-9, MMP-10, MMP-11, MMP-12, MMP-13, MMP-14, MMP-15, MMP-16, MMP-17, MMP-18, MMP-19, MMP-20, MMP-24, and MMP-25 (see htt-p://www.expasy.ch/cgi-bin/prosite-search-ac?PDOC00023). The results of a Smith Waterman search (PAM100, gap open and extend penalties of 12 and 2) of the public database of amino acid sequences (NRAA) with this protein sequence yielded the following results: Pscore=0; number of identical amino acids=483; percent identity=100%; percent similarity=100%; the accession number of the most similar entry in NRAA is NP_{—}004762.1; the name or description, and species, of the most similar protein in NRAA is: matrix metalloproteinase 20 preproprotein; enamelysin [Homo sapiens]. This protein has a transmembrane domain from amino acid 7 to amino acid 29. This may function as a signal peptide.

SGPr104_{—}1, SEQ ID NO:21, SEQ ID NO:80 encodes a protein that is 765 amino acids long. It is classified as a Metalloprotease protease, of the PepM 13 family. The protease domain(s) in this protein match the hidden Markov profile for a Peptidase_M13 (PF01431), from amino acid 561 to amino acid 764. The positions within the HMMR profile that match the protein sequence are from profile position 1 to profile position 222. The results of a Smith Waterman search (PAM100, gap open and extend penalties of 12 and 2) of the public database of amino acid sequences (NRAA) with this protein sequence yielded the following results: Pscore=0; number of identical amino acids=765; percent identity=100%; percent similarity=100%; the accession number of the most similar entry in NRAA is NP_{—}055508.1; the name or description, and species, of the most similar protein in NRAA is: KIAA0604 gene product [Homo sapiens]. This protein has a transmembrane domain from amino acid 61 to amino acid 83.

SGPr303, SEQ ID NO:22, SEQ ID NO:81 encodes a protein that is 418 amino acids long. It is classified as a Metalloprotease protease, of the PepM2 family. The protease domain(s) in this protein match the hidden Markov profile for a Peptidase_M1 (PF01433), from amino acid 10 to amino acid 397. The positions within the HMMR profile that match the protein sequence are from profile position 1 to profile position 416. The results of a Smith Waterman search (PAM100, gap open and extend penalties of 12 and 2) of the public database of amino acid sequences (NRAA) with this protein sequence yielded the following results: Pscore=2.20E-284; number of identical amino acids=407; percent identity=97%; percent similarity=98%; the accession number of the most similar entry in NRAA is CAA10709.1; the name or description, and species, of the most similar protein in NRAA is: (AJ132583) puromycin sensitive aminopeptidase [Homo sapiens].

SGPr402_{—}1, SEQ ID NO:23, SEQ ID NO:82 encodes a protein that is 755 amino acids long. It is classified as a Serine protease, of the subtilase family. The protease domain(s) in this protein match the hidden Markov profile for a subtilase (PF00082), from amino acid 118 to amino acid 437. The positions within the HMMR profile that match the protein sequence are from profile position 1 to profile position 360. The results of a Smith Waterman search (PAM100, gap open and extend penalties of 12 and 2) of the public database of amino acid sequences (NRAA) with this protein sequence yielded the following results: Pscore=0; number of identical amino acids=513; percent identity=82%; percent similarity=89%; the accession number of the most similar entry in NRAA is P29121; the name or description, and species, of the most similar protein in NRAA is: NEUROENDOCRINE CONVERTASE 3 PRECURSOR [Mus musculus].

SGPr434, SEQ ID NO:24, SEQ ID NO:83 encodes a protein that is 391 amino acids long. It is classified as a Serine protease, of the trypsin family. The protease domain(s) in this protein match the hidden Markov profile for a p20-ICE (PF00656), from amino acid 39 to amino acid 46. The positions within the HMMR profile that match the protein sequence are from profile position 129 to profile position 136. The results of a Smith Waterman search (PAM100, gap open and extend penalties of 12 and 2) of the public database of amino acid sequences (NRAA) with this protein sequence yielded the following results: Pscore=6.20E-43; number of identical amino acids=104; percent identity=42%; percent similarity=59%; the accession number of the most similar entry in NRAA is NP_{—}036164.1; the name or description, and species, of the most similar protein in NRAA is: transmembrane tryptase [Mus musculus].

SGPr446_{—}1, SEQ ID NO:25, SEQ ID NO:84 encodes a protein that is 226 amino acids long. It is classified as a Serine protease, of the trypsin family. The protease domain(s) in this protein match the hidden Markov profile for a trypsin (PF00089), from amino acid 13 to amino acid 227. The positions within the HMMR profile that match the protein sequence are from profile position 1 to profile position 242. The results of a Smith Waterman search (PAM100, gap open and extend penalties of 12 and 2) of the public database of amino acid sequences (NRAA) with this protein sequence yielded the following results: Pscore=2.50E-40; number of identical amino acids=107; percent identity=45%; percent similarity=57%; the accession number of the most similar entry in NRAA is NP_{—}038949.1; the name or description, and species, of the most similar protein in NRAA is: distal intestinal serine protease [Mus musculus].

SGPr447, SEQ ID NO:26, SEQ ID NO:85 encodes a protein that is 295 amino acids long. It is classified as a Serine protease, of the trypsin family. The protease domain(s) in this protein match the hidden Markov profile for a trypsin (PF00089), from amino acid 33 to amino acid 270. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=1.00E-97; number of identical amino acids=167; percent identity=60%; percent similarity=77%; the accession number of the most similar entry in NRAA is BAB30277.1; the name or description, and species, of the most similar protein in NRAA is: (AK016509) putative [Mus musculus].

SGPr432_{—}1, SEQ ID NO:27, SEQ ID NO:86 encodes a protein that is 628 amino acids long. It is classified as a Serine protease, of the trypsin family. The protease domain(s) in this protein match the hidden Markov profile for a trypsin (PF00089), from amino acid 117 to amino acid 343. The positions within the HMMR profile that match the protein sequence are from profile position 6 to profile position 259. The results of a Smith Waterman search (PAM100, gap open and extend penalties of 12 and 2) of the public database of amino acid sequences (NRAA) with this protein sequence yielded the following results: Pscore=3.70E-56; number of identical amino acids=95; percent identity=100%; percent similarity=100%; the accession number of the most similar entry in NRAA is NP_{—}076869.1; the name or description, and species, of the most similar protein in NRAA is: hypothetical protein IMAGE3455200 [Homo sapiens]. This protein has two transmembrane domains from amino acid 10 to amino acid 29, and from 82 to 99. The region from amino acid 10 to 29 may function as a signal peptide.

SGPr529, SEQ ID NO:28, SEQ ID NO:87 encodes a protein that is 276 amino acids long. It is classified as a Serine protease, of the trypsin family. The protease domain(s) in this protein match the hidden Markov profile for a trypsin (PF00089), from amino acid 184 to amino acid 187. The positions within the HMMR profile that match the protein sequence are from profile position 413 to profile position 416. The results of a Smith Waterman search (PAM100, gap open and extend penalties of 12 and 2) of the public database of amino acid sequences (NRAA) with this protein sequence yielded the following results: Pscore=1.70E-184; number of identical amino acids=276; percent identity=100%; percent similarity=100%; the accession number of the most similar entry in NRAA is NP_{—}002767.1; the name or description, and species, of the most similar protein in NRAA is: kallikrein 10; protease, serine-like, 1 [Homo sapiens].

SGPr428_{—}1, SEQ ID NO:29, SEQ ID NO:88 encodes a protein that is 285 amino acids long. It is classified as a Serine protease, of the trypsin family. The protease domain(s) in this protein match the hidden Markov profile for a trypsin (PF00089), from amino acid 24 to amino acid 246. The positions within the HMMR profile that match the protein sequence are from profile position 7 to profile position 259. The results of a Smith Waterman search (PAM100, gap open and extend penalties of 12 and 2) of the public database of amino acid sequences (NRAA) with this protein sequence yielded the following results: Pscore=1.90E-58; number of identical amino acids=92; percent identity=53%; percent similarity=73%; the accession number of the most similar entry in NRAA is BAB24215.1; the name or description, and species, of the most similar protein in NRAA is: (AK005740) putative [Mus musculus]. This protein has a transmembrane domain from amino acid 262 to amino acid 284.

SGPr425, SEQ ID NO:30, SEQ ID NO:89 encodes a protein that is 413 amino acids long. It is classified as a Serine protease, of the trypsin family. The protease domain(s) in this protein match the hidden Markov profile for a trypsin (PF00089), from amino acid 287 to amino acid 306. The positions within the HMMR profile that match the protein sequence are from profile position 387 to profile position 406. This protein has a putative CAAX motif (CAYG) which may direct it to the plasma membrane. The results of a Smith Waterman search (PAM100, gap open and extend penalties of 12 and 2) of the public database of amino acid sequences (NRAA) with this protein sequence yielded the following results: Pscore=5.80E-268; number of identical amino acids=412; percent identity=99%; percent similarity=99%; the accession number of the most similar entry in NRAA is CAC35071.1; the name or description, and species, of the most similar protein in NRAA is: (AL121939) dJ223E3.1 (putative secreted protein ZSIG13) [Homo sapiens].

SGPr548, SEQ ID NO:31, SEQ ID NO:90 encodes a protein that is 320 amino acids long. It is classified as a Serine protease, of the trypsin family. The protease domain(s) in this protein match the hidden Markov profile for a trypsin (PF00089), from amino acid 86 to amino acid 313. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=2.60E-168; number of identical amino acids=256; percent identity=100%; percent similarity=100%; the accession number of the most similar entry in NRAA is AAG09469.1; the name or description, and species, of the most similar protein in NRAA is: (AF242195) KLK15 [Homo sapiens].

SGPr396, SEQ ID NO:32, SEQ ID NO:91 encodes a protein that is 328 amino acids long. It is classified as a Serine protease, of the trypsin family. The protease domain(s) in this protein match the hidden Markov profile for a trypsin (PF00089), from amino acid 28 to amino acid 262. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=1.60E-56; number of identical amino acids=111; percent identity=44%; percent similarity=61%; the accession number of the most similar entry in NRAA is BAA84941.1; the name or description, and species, of the most similar protein in NRAA is: (AB018694) epidermis specific serine protease [Xenopus laevis].

SGPr426, SEQ ID NO:33, SEQ ID NO:92 encodes a protein that is 425 amino acids long. It is classified as a Serine protease, of the trypsin family. The protease domain(s) in this protein match the hidden Markov profile for a trypsin (PF00089), from amino acid 194 to amino acid 419. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=7.70E-93; number of identical amino acids=181; percent identity=43%; percent similarity=61%; the accession number of the most similar entry in NRAA is NP_{—}054777.1; the name or description, and species, of the most similar protein in NRAA is: DESC1 protein [Homo sapiens]. This protein has a transmembrane domain from amino acid 30 to amino acid 52. This region could function as a signal peptide.

SGPr552, SEQ ID NO:34, SEQ ID NO:93 encodes a protein that is 221 amino acids long. It is classified as a Serine protease, of the trypsin family. The protease domain(s) in this protein match the hidden Markov profile for a trypsin (PF00089), from amino acid 2 to amino acid 222. The positions within the HMMR profile that match the protein sequence are from profile position 1 to profile position 255. The results of a Smith Waterman search (PAM100, gap open and extend penalties of 12 and 2) of the public database of amino acid sequences (NRAA) with this protein sequence yielded the following results: Pscore=1.20E-45; number of identical amino acids=96; percent identity=42%; percent similarity=59%; the accession number of the most similar entry in NRAA is NP_{—}054777.1; the name or description, and species, of the most similar protein in NRAA is: DESC1 protein [Homo sapiens].

SGPr405, SEQ ID NO:35, SEQ ID NO:94 encodes a protein that is 948 amino acids long. It is classified as a Serine protease, of the trypsin family. The protease domain(s) in this protein match the hidden Markov profile for a trypsin (PF00089), from amino acid 218 to amino acid 406. The positions within the HMMR profile that match the protein sequence are from profile position 60 to profile position 259. Other domains identified within this protein are: two additional trypsin domains, from amino acids 419 to 496, and from amino acids 636 to 761. The results of a Smith Waterman search (PAM100, gap open and extend penalties of 12 and 2) of the public database of amino acid sequences (NRAA) with this protein sequence yielded the following results: Pscore=1.10E-30; number of identical amino acids=111; percent identity=54%; percent similarity=65%; the accession number of the most similar entry in NRAA is P19236; the name or description, and species, of the most similar protein in NRAA is: MASTOCYTOMA PROTEASE PRECURSOR [Canis familiaris].

SGPr485_{—}1, SEQ ID NO:36, SEQ ID NO:95 encodes a protein that is 352 amino acids long. It is classified as a Serine protease, of the trypsin family. The protease domain(s) in this protein match the hidden Markov profile for a trypsin (PF00089), from amino acid 68 to amino acid 295. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=7.20E-133; number of identical amino acids=223; percent identity=94%; percent similarity=96%; the accession number of the most similar entry in NRAA is BAB03569.1; the name or description, and species, of the most similar protein in NRAA is: (AB046651) hypothetical protein [Macaca fascicularis].

SGPr534, SEQ ID NO:37, SEQ ID NO:96 encodes a protein that is 263 amino acids long. It is classified as a Serine protease, of the trypsin family. The protease domain(s) in this protein match the hidden Markov profile for a trypsin (PF00089), from amino acid 34 to amino acid 256. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=3.60E-165; number of identical amino acids=253; percent identity=96%; percent similarity=98%; the accession number of the most similar entry in NRAA is NP_{—}001897.1; the name or description, and species, of the most similar protein in NRAA is: chymotrypsinogen B1 [Homo sapiens]. This protein has a transmembrane domain from amino acid 2 to amino acid 24. This region could function as a signal peptide.

SGPr390, SEQ ID NO:38, SEQ ID NO:97 encodes a protein that is 1128 amino acids long. It is classified as a Serine protease, of the trypsin family. The protease domain(s) in this protein match the hidden Markov profile for a trypsin (PF00089), from amino acid 896 to amino acid 1122. The positions within the HMMR profile that match the protein sequence are from profile position 1 to profile position 259. Other domains identified within this protein are: two trypsin domains, from amino acids 264 to 500, and from amino acids 573 to 800. The results of a Smith Waterman search (PAM100, gap open and extend penalties of 12 and 2) of the public database of amino acid sequences (NRAA) with this protein sequence yielded the following results: Pscore=2.60E-53; number of identical amino acids=135; percent identity=46%; percent similarity=59%; the accession number of the most similar entry in NRAA is BAB23684. 1; the name or description, and species, of the most similar protein in NRAA is: (AK004939) putative [Mus musculus]. This protein has a transmembrane domain from amino acid 28 to amino acid 50. This region could function as a signal peptide.

SGPr521, SEQ ID NO:39, SEQ ID NO:98 encodes a protein that is 253 amino acids long. It is classified as a Serine protease, of the trypsin family. The protease domain(s) in this protein match the hidden Markov profile for a trypsin (PF00089), from amino acid 30 to amino acid 245. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=2.30E-155; number of identical amino acids=253; percent identity=100%; percent similarity=100%; the accession number of the most similar entry in NRAA is NP_{—}005037.1; the name or description, and species, of the most similar protein in NRAA is: kallikrein 7 (chymotryptic, stratum corneum); protease, serine, 6 (chymotryptic, stratum corneum) [Homo sapiens].

SGPr5301, SEQ ID NO:40, SEQ ID NO:99 encodes a protein that is 271 amino acids long. It is classified as a Serine protease, of the trypsin family. The protease domain(s) in this protein match the hidden Markov profile for a trypsin (PF00089), from amino acid 14 to amino acid 255. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=1.10E-95; number of identical amino acids=142; percent identity=100%; percent similarity=100%; the accession number of the most similar entry in NRAA is CAC12709.1; the name or description, and species, of the most similar protein in NRAA is: (AL136097) bA62C3.1 (similar to testicular serine protease) [Homo sapiens].

SGPr520, SEQ ID NO:41, SEQ ID NO:100 encodes a protein that is 578 amino acids long. It is classified as a Serine protease, of the trypsin family. The protease domain(s) in this protein match the hidden Markov profile for a trypsin (PF00089), from amino acid 73 to amino acid 306. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=1.50E-83; number of identical amino acids=158; percent identity=73%; percent similarity=83%; the accession number of the most similar entry in NRAA is BAB24587.1; the name or description, and species, of the most similar protein in NRAA is: (AK006434) putative [Mus musculus].

SGPr455, SEQ ID NO:42, SEQ ID NO:101 encodes a protein that is 970 amino acids long. It is classified as a Serine protease, of the trypsin family. The protease domain(s) in this protein match the hidden Markov profile for a trypsin (PF00089), from amino acid 433 to amino acid 674. The positions within the HMMR profile that match the protein sequence are from profile position 1 to profile position 259. Other domains identified within this protein are: Trypsin, from amino acid 4 to 156; and three 3× CUB domains (PF00431) from amino acid 175 to 812. The results of a Smith Waterman search (PAM100, gap open and extend penalties of 12 and 2) of the public database of amino acid sequences (NRAA) with this protein sequence yielded the following results: Pscore=5.90E-179; number of identical amino acids=386; percent identity=41%; percent similarity=58%; the accession number of the most similar entry in NRAA is T30337; the name or description, and species, of the most similar protein in NRAA is: polyprotein—African clawed frog.

SGPr507_{—}2, SEQ ID NO:43, SEQ ID NO:102 encodes a protein that is 265 amino acids long. It is classified as a Serine protease, of the trypsin family. The protease domain(s) in this protein match the hidden Markov profile for a trypsin (PF00089), from amino acid 42 to amino acid 135. The positions within the HMMR profile that match the protein sequence are from profile position 35 to profile position 148. Other domains identified within this protein are: Trypsin domain from amino acid 247 to 258. The results of a Smith Waterman search (PAM100, gap open and extend penalties of 12 and 2) of the public database of amino acid sequences (NRAA) with this protein sequence yielded the following results: Pscore=2.40E-121; number of identical amino acids=195; percent identity=73%; percent similarity=81%; the accession number of the most similar entry in NRAA is NP_{—}080593.1; the name or description, and species, of the most similar protein in NRAA is: RIKEN cDNA 1700016G05 gene [Mus musculus).

SGPr559, SEQ ID NO:44, SEQ ID NO:103 encodes a protein that is 454 amino acids long. It is classified as a Serine protease, of the trypsin family. The protease domain(s) in this protein match the hidden Markov profile for a trypsin (PF00089), from amino acid 217 to amino acid 444. The positions within the HMMR profile that match the protein sequence are from profile position 1 to profile position 259. Other domains identified within this protein are: Low-density lipoprotein receptor domain class A (PF00057), from amino acid 71 to 109. LDL-receptors the class A domains form the binding site for LDL and calcium. The acidic residues between the fourth and sixth cysteines are important for high-affinity binding of positively charged sequences in LDLR's ligands. The repeat has been shown to consist of a beta-hairpin structure followed by a series of beta turns (see http://www.expasy.ch/cgi-bin/get-prodoc-entry?PDOC00929). The results of a Smith Waterman search (PAM100, gap open and extend penalties of 12 and 2) of the public database of amino acid sequences (NRAA) with this protein sequence yielded the following results: Pscore=1.40E-288; number of identical amino acids=454; percent identity=100%; percent similarity=100%; the accession number of the most similar entry in NRAA is NP_{—}076927.1; the name or description, and species, of the most similar protein in NRAA is: transmembrane protease, serine 3 [Homo sapiens]. This protein has a transmembrane domain from amino acid 49 to amino acid 71.

SGPr567_{—}1, SEQ ID NO:45, SEQ ID NO:104 encodes a protein that is 537 amino acids long. It is classified as a Serine protease, of the trypsin family. The protease domain(s) in this protein match the hidden Markov profile for a trypsin (PF00089), from amino acid 296 to amino acid 524. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=1.70E-135; number of identical amino acids=534; percent identity=99%; percent similarity=99%; the accession number of the most similar entry in NRAA is NP_{—}114435.1; the name or description, and species, of the most similar protein in NRAA is: mosaic serine protease [Homo sapiens].

SGPr479_{—}1, SEQ ID NO:46, SEQ ID NO:105 encodes a protein that is 326 amino acids long. It is classified as a Serine protease, of the trypsin family. The protease domain(s) in this protein match the hidden Markov profile for a trypsin (PF00089), from amino acid 60 to amino acid 288. The positions within the HMMR profile that match the protein sequence are from profile position 1 to profile position. The results of a Smith Waterman search (PAM100, gap open and extend penalties of 12 and 2) of the public database of amino acid sequences (NRAA) with this protein sequence yielded the following results: Pscore=1.70E-39; number of identical amino acids=107; percent identity=42%; percent similarity=57%; the accession number of the most similar entry in NRAA is NP_{—}114154.1; the name or description, and species, of the most similar protein in NRAA is: marapsin [Homo sapiens].

SGPr489_{—}1, SEQ ID NO:47, SEQ ID NO:106 encodes a protein that is 556 amino acids long. It is classified as a Serine protease, of the trypsin family. The protease domain(s) in this protein match the hidden Markov profile for a trypsin (PF00089), from amino acid 56 to amino acid 257. The positions within the HMMR profile that match the protein sequence are from profile position 1 to profile position 227. Other domains identified within this protein are: 2 × CUB domains (PF00431) from amino acids 304 to 503. The results of a Smith Waterman search (PAM100, gap open and extend penalties of 12 and 2) of the public database of amino acid sequences (NRAA) with this protein sequence yielded the following results: Pscore=2.70E-90; number of identical amino acids=194; percent identity=37%; percent similarity=54%; the accession number of the most similar entry in NRAA is T30338; the name or description, and species, of the most similar protein in NRAA is: oviductin—[Xenopus laevis].

SGPr465_{—}1, SEQ ID NO:48, SEQ ID NO:107 encodes a protein that is 297 amino acids long. It is classified as a Serine protease, of the trypsin family. The protease domain(s) in this protein match the hidden Markov profile for a trypsin (PF00089), from amino acid 2 to amino acid 240. The positions within the HMMR profile that match the protein sequence are from profile position 12 to profile position 259. The results of a Smith Waterman search (PAM100, gap open and extend penalties of 12 and 2) of the public database of amino acid sequences (NRAA) with this protein sequence yielded the following results: Pscore=2.70E-76; number of identical amino acids=144; percent identity=48%; percent similarity=66%; the accession number ofthe most similar entry in NRAA is NP_{—}033381.1; the name or description, and species, of the most similar protein in NRAA is: testicular serine protease 1 [Mus musculus].

SGPr524_{—}1, SEQ ID NO:49, SEQ ID NO:108 encodes a protein that is 850 amino acids long. It is classified as a Serine protease, of the trypsin family. The protease domain(s) in this protein match the hidden Markov profile for a trypsin (PF00089), from amino acid 613 to amino acid 842. The positions within the HMMR profile that match the protein sequence are from profile position 1 to profile position 259. Other domains identified within this protein are: three Low-density lipoprotein receptor domain class A domains (PF00057) from 489 to 603. LDL-receptors the class A domains form the binding site for LDL and calcium. The acidic residues between the fourth and sixth cysteines are important for high-affinity binding of positively charged sequences in LDLR's ligands. The repeat has been shown to consist of a beta-hairpin structure followed by a series of beta turns (see http://www.expasy.ch/cgi-bin/get-prodoc-entry?PDOC00929). The results of a Smith Waterman search (PAM100, gap open and extend penalties of 12 and 2) of the public database of amino acid sequences (NRAA) with this protein sequence yielded the following-results: Pscore=1.30E-79; number of identical amino acids=193; percent identity=41%; percent similarity=55%; the accession number of the most similar entry in NRAA is BAB23684.1; the name or description, and species, of the most similar protein in NRAA is: (AK004939) putative [Mus musculus]. This protein has a transmembrane domain from amino acid 77 to amino acid 99.

SGPr422, SEQ ID NO:50, SEQ ID NO:109 encodes a protein that is 447 amino acids long. It is classified as a Serine protease, of the trypsin family. The protease domain(s) in this protein match the hidden Markov profile for a trypsin (PF00089), from amino acid 216 to amino acid 441. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=4.90E-80; number of identical amino acids=173; percent identity=39%; percent similarity=59%; the accession number of the most similar entry in NRAA is NP_{—}054777.1; the name or description, and species, of the most similar protein in NRAA is: DESC1 protein [Homo sapiens]. This protein has a transmembrane domain from amino acid 32 to amino acid 54. This region could function as a signal peptide.

SGPr538, SEQ ID NO:51, SEQ ID NO:I 10 encodes a protein that is 457 amino acids long. It is classified as a Serine protease, of the trypsin family. The protease domain(s) in this protein match the hidden Markov profile for a trypsin (PF00089), from amino acid 218 to amino acid 448. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=9.1e-315; number of identical amino acids=457; percent identity=100%; percent similarity=100%; the accession number of the most similar entry in NRAA is NP_{—}110397.1; the name or description, and species, of the most similar protein in NRAA is: spinesin [Homo sapiens]. This protein has a transmembrane domain from amino acid 48 to amino acid 70. This region could function as a signal peptide.

SGPr527_{—}1, SEQ ID NO:52, SEQ ID NO:111 encodes a protein that is 818 amino acids long. It is classified as a Serine protease, of the trypsin family. The protease domain(s) in this protein match the hidden Markov profile for a trypsin (PF00089), from amino acid 47 to amino acid 286. The positions within the HMMR profile that match the protein sequence are from profile position 1 to profile position 259. Other domains identified within this protein are: two additional trypsin domains, from 323 to 454, and from 564 to 679. The results of a Smith Waterman search (PAM100, gap open and extend penalties of 12 and 2) of the public database of amino acid sequences (NRAA) with this protein sequence yielded the following results: Pscore=1.30E-52; number of identical amino acids=114; percent identity=42%; percent similarity=59%; the accession number of the most similar entry in NRAA is AAH03851.1; the name or description, and species, of the most similar protein in NRAA is: (BC003851) Similar to protease, serine, 8 (prostasin) [Mus musculus].

SGPr542, SEQ ID NO:53, SEQ ID NO:112 encodes a protein that is 284 amino acids long. It is classified as a Serine protease, of the trypsin family. The protease domain(s) in this protein match the hidden Markov profile for a trypsin (PF00089), from amino acid 35 to amino acid 259. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=2.70E-41; number of identical amino acids=110; percent identity=43%; percent similarity=58%; the accession number of the most similar entry in NRAA is NP_{—}005308.1; the name or description, and species, of the most similar protein in NRAA is: granzyme M precursor; lymphocyte met-ase 1 [Homo sapiens].

SGPr551, SEQ ID NO:54, SEQ ID NO:113 encodes a protein that is 802 amino acids long. It is classified as a Serine protease, of the trypsin family. The protease domain(s) in this protein match the hidden Markov profile for a trypsin (PF00089), from amino acid 568 to amino acid 797. The positions within the HMMR profile that match the protein sequence are from profile position 1 to profile position 259. Other domains identified within this protein are: three low-density lipoprotein receptor domain class A domains (PF00057) from 447 to 559. LDL-receptors the class A domains form the binding site for LDL and calcium. The acidic residues between the fourth and sixth cysteines are important for high-affinity binding of positively charged sequences in LDLR's ligands. The repeat has been shown to consist of a beta-hairpin structure followed by a series of beta turns (see http://www.expasy.ch/cgi-bin/get-prodoc-entry?PDOC00929). The results of a Smith Waterman search (PAM100, gap open and extend penalties of 12 and 2) of the public database of amino acid sequences (NRAA) with this protein sequence yielded the following results: Pscore=0; number of identical amino acids=675; percent identity=84%; percent similarity=90%; the accession number of the most similar entry in NRAA is BAB23684. 1; the name or description, and species, of the most similar protein in NRAA is: (AK004939) putative [Mus musculus]. This protein has a transmembrane domain from amino acid 44 to amino acid 66. This region could function as a signal peptide.

SGPr451, SEQ ID NO:55, SEQ ID NO:114 encodes a protein that is 359 amino acids long. It is classified as a Serine protease, of the trypsin family. The protease domain(s) in this protein match the hidden Markov profile for a trypsin (PF00089), from amino acid 89 to amino acid 324. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=9.90E-41; number of identical amino acids=101; percent identity=39%; percent similarity=59%; the accession number of the most similar entry in NRAA is NP_{—}072152.1; the name or description, and species, of the most similar protein in NRAA is: adrenal secretory serine protease precursor [Rattus norvegicus].

SGPr452_{—}1, SEQ ID NO:56, SEQ ID NO:115 encodes a protein that is 288 amino acids long. It is classified as a Serine protease, of the trypsin family. The protease domain(s) in this protein match the hidden Markov profile for a trypsin (PF00089), from amino acid 73 to amino acid 280. The positions within the HMMR profile that match the protein sequence are from profile position 1 to profile position 259. The results of a Smith Waterman search (PAM100, gap open and extend penalties of 12 and 2) of the public database of amino acid sequences (NRAA) with this protein sequence yielded the following results: Pscore=1.40E-81; number of identical amino acids=142; percent identity=57%; percent similarity=72%; the accession number of the most similar entry in NRAA is AAK15264.1; the name or description, and species, of the most similar protein in NRAA is: (AF305425) implantation serine proteinase 2 [Mus musculus].

SGPr504, SEQ ID NO:57, SEQ ID NO:116 encodes a protein that is 44 amino acids long. It is classified as a Serine protease, of the trypsin family. The protease domain(s) in this protein match the hidden Markov profile for a trypsin (PF00089), from amino acid 1 to amino acid 45. The positions within the HMMR profile that match the protein sequence are from profile position 1 to profile position 52. The results of a Smith Waterman search (PAM100, gap open and extend penalties of 12 and 2) of the public database of amino acid sequences (NRAA) with this protein sequence yielded the following results: Pscore=2.40E-13; number of identical amino acids=26; percent identity=61%; percent similarity=88%; the accession number of the most similar entry in NRAA is NP_{—}002095.1; the name or description, and species, of the most similar protein in NRAA is: granzyme K precursor; granzyme 3; granzyme K (serine protease, granzyme 3); tryptase II [Homo sapiens].

SGPr469, SEQ ID NO:58, SEQ ID NO:117 encodes a protein that is 45 amino acids long. It is classified as a Serine protease, of the trypsin family. The protease domain(s) in this protein match the hidden Markov profile for a trypsin (PF00089), from amino acid 1 to amino acid 46. The positions within the HMMR profile that match the protein sequence are from profile position 210 to profile position 259. The results of a Smith Waterman search (PAM100, gap open and extend penalties of 12 and 2) of the public database of amino acid sequences (NRAA) with this protein sequence yielded the following results: Pscore=2.20E-17; number of identical amino acids=32; percent identity=69%; percent similarity=84%; the accession number of the most similar entry in NRAA is BAB30277.1; the name or description, and species, of the most similar protein in NRAA is: (AK016509) putative [Mus musculus].

SGPr400, SEQ ID NO:59, SEQ ID NO:118 encodes a protein that is 309 amino acids long. It is classified as a Serine protease, of the trypsin family. The protease domain(s) in this protein match the hidden Markov profile for a trypsin (PF00089), from amino acid 133 to amino acid 281. The positions within the HM profile that match the protein sequence are from profile position 1 to profile position 198. The results of a Smith Waterman search (PAM100, gap open and extend penalties of 12 and 2) of the public database of amino acid sequences (NRAA) with this protein sequence yielded the following results: Pscore=2.30E-16; number of identical amino acids=72; percent identity=38%; percent similarity=48%; the accession number of the most similar entry in NRAA is NP_{—}036164.1; the name or description, and species, of the most similar protein in NRAA is: transmembrane tryptase [Mus musculus].

### Example 2

### Expression Analysis of Mammalian Proteases

Materials and Methods

Quantitative PCR Analysis

RNA is isolated from a variety of normal human tissues and cell lines. Single stranded cDNA is synthesized from 10 μg of each RNA as described above using the Superscript Preamplification System (GibcoBRL). These single strand templates are then linearly amplified with a pair of specific primers in a real time PCR reaction on a Light Cycler (Roche Molecular Biochemical). Graphical readout can provide quantitative analysis of the relative abundance of the targeted gene in the total RNA preparation.

DNA Array Based Expression Analysis

DNA-free RNA is isolated from a variety of normal human tissues, cryostat sections, and cell lines. Single stranded cDNA is synthesized from 10 μg RNA or 1 μg mRNA using a modification of the SMART PCR cDNA synthesis technique (Clontech). The procedure can be modified to allow asymmetric labeling of the 5′ and 3′ ends of each transcript with a unique oligonucleotide sequence. The resulting sscDNAs are then linearly amplified using Advantage long-range PCR (Clontech) on a Light Cycler PCR machine. Reactions are halted when the graphical real-time display demonstrates the products have begun to plateau. The double stranded cDNA products are purified using Millipore DNA purification matrix, dried, resuspended, quantified, and analyzed on an agarose gel. The resulting elements are referred to as “tissue cDNAs”.

Tissue cDNAs are spotted onto GAPS coated glass slides (Coming) using a Genetic Microsystems (GMS) arrayer at 500 ng/ul.

Fluorescent labeled oligonucleotides are synthesized to each novel exon, ensuring they contained internal mismatches with the closest known homologue. Typically oligos are 45 nucleotides long, labeled on the 5′ end with Cy5.

Exon-specific Cy5-labeled oligos are hybridized to the tissue cDNAs arrayed onto glass slides, and washed using standard buffers and conditions. Hybridizing signals are then quantified using a GMS Scanner.

Alternatively, tissue cDNAs are manually spotted onto Nylon membranes using a 384 pin replicator, and hybridized to ³²P-end labeled oligo probes.

Tissue cDNAs are generated from multiple RNA templates selected to provide information of relevance to the disease areas of interest and to reflect the biological mechanism of action for each protease. These templates include: human tumor cell lines, cryostat sections of primary human tumors and 32 normal human tissues to identify cancer-related genes; sections of normal, Alzheimer's, Parkinson's, and Schizophrenia brain regions for CNS-related genes; normal and diabetic or obese skeletal muscle, adipose, or liver for metabolic-related genes; and purified hematopoeitic cells, and lymphoid tissues for immune-related genes. To characterize gene mechanism of action, tissue cDNAs are generated to reflect angiogenesis (cultured endothelial cells treated with VEGF ligand, anti-angiogenic drugs, or hypoxia), motility (A549 cells stimulated with HGF ligand, orthotopic metastases, primary tumors with matched metastatic tumors), cell cycle (Hela, H1299, and other cell lines synchronized by drug block and harvested at various times in the cell cycle), checkpoint integrity and DNA repair (p53 normal or defective cells treated with γ-radiation, UV, cis-platinum, or oxidative stress), and cell survival (cells induced to differentiate or at various stages of apoptosis).

### Example 3

### Isolation of cDNAs Encoding Mammalian Proteases

Materials and Methods

Identification of Novel Clones

Total RNAs are isolated using the Guanidine Salts/Phenol extraction protocol of Chomczynski and Sacchi (P. Chomczynski and N. Sacchi, Anal. Biochem. 162:156 (1987)) from primary human tumors, normal and tumor cell lines, normal human tissues, and sorted human hematopoietic cells. These RNAs are used to generate single-stranded cDNA using the Superscript Preamplification System (GIBCO BRL, Gaithersburg, Md.; Gerard, G F et al. (1989), FOCUS 11, 66) under conditions recommended by the manufacturer. A typical reaction uses 10 μg total RNA with 1.5 μg oligo(dT)1₁₂₋₁₈ in a reaction volume of 60 μL. The product is treated with RNaseH and diluted to 100 μL with H₂O. For subsequent PCR amplification, 1-4 μL of this sscDNA is used in each reaction.

Degenerate oligonucleotides are synthesized on an Applied Biosystems 3948 DNA synthesizer using established phosphoramidite chemistry, precipitated with ethanol and used unpurified for PCR. These primers are derived from the sense and antisense strands of conserved motifs within the catalytic domain of several proteases. Degenerate nucleotide residue designations are: N=A, C, G, or T; R=A or G; Y═C or T;H=A, C or T not G; D=A, G or T not C; S═C or G; and W=A or T.

PCR reactions are performed using degenerate primers applied to multiple single-stranded cDNAs. The primers are added at a final concentration of 5 μM each to a mixture containing 10 mM TrisHCl, pH 8.3, 50 mM KCl, 1.5 mM MgCl₂, 200 μM each deoxynucleoside triphosphate, 0.001% gelatin, 1.5 U AmpliTaq DNA Polymerase (Perkin-Elmer/Cetus), and 1-4 μL cDNA. Following 3 min denaturation at 95° C., the cycling conditions are 94° C. for 30 s, 50° C. for 1 min, and 72° C. for 1 min 45 s for 35 cycles. PCR fragments migrating between 300-350 bp are isolated from 2% agarose gels using the GeneClean Kit (Bio101), and T-A cloned into the pCRII vector (Invitrogen Corp. U.S.A.) according to the manufacturer's protocol.

Colonies are selected for mini plasmid DNA-preparations using Qiagen columns and the plasmid DNA is sequenced using a cycle sequencing dye-terminator kit with AmpliTaq DNA Polymerase, FS (ABI, Foster City, Calif.). Sequencing reaction products are run on an ABI Prism 377 DNA Sequencer, and analyzed using the BLAST alignment algorithm (Altschul, S.F. et al., J. Mol. Biol. 215: 403-10).

Additional PCR strategies are employed to connect various PCR fragments or ESTs using exact or near exact oligonucleotide primers. PCR conditions are as described above except the annealing temperatures are calculated for each oligo pair using the formula: Tm=4(G+C)+2(A+T).

Isolation of cDNA Clones

Human cDNA libraries are probed with PCR or EST fragments corresponding to protease-related genes. Probes are ³²P-labeled by random priming and used at 2×10⁶ cpm/mL following standard techniques for library screening. Pre-hybridization (3 h) and hybridization (overnight) are conducted at 42° C. in 5×SSC, 5× Denhart's solution, 2.5% dextran sulfate, 50 mM Na₂PO₄/NaHPO₄, pH 7.0, 50% formamide with 100 mg/mL denatured salmon sperm DNA. Stringent washes are performed at 65° C. in 0.1×SSC and 0.1% SDS. DNA sequencing is carried out on both strands using a cycle sequencing dye-terminator kit with AmpliTaq DNA Polymerase, FS (ABI, Foster City, Calif.). Sequencing reaction products are run on an ABI Prism 377 DNA Sequencer.

### Example 4

### Expression Analysis of Mammalian Proteases

Materials and Methods

Northern Blot Analysis

Northern blots are prepared by running 10 μg total RNA isolated from 60 human tumor cell lines (such as HOP-92, EKVX, NCI-H23, NCI-H226, NCI-H322M, NCI-H460, NCI-H522, A549, HOP-62, OVCAR-3, OVCAR-4, OVCAR-5, OVCAR-8, IGROV1, SK-OV-3, SNB-19, SNB-75, U251, SF-268, SF-295, SF-539, CCRF-CEM, K-562, MOLT-4, HL-60, RPMI 8226, SR, DU-145, PC-3, HT-29, HCC-2998, HCT-116, SW620, Colo 205, HTC15, KM-12, UO-31, SN12C, A498, CaKi1, RXF-393, ACHN, 786-0, TK-10, LOX IMVI, Malme-3M, SK-MEL-2, SK-MEL-5, SK-MEL-28, UACC-62, UACC-257, M14, MCF-7, MCF-7/ADR RES; Hs578T, MDA-MB-231, MDA-MB-435, MDA-N, BT-549, T47D), from human adult tissues (such as thymus, lung, duodenum, colon, testis, brain, cerebellum, cortex, salivary gland, liver, pancreas, kidney, spleen, stomach, uterus, prostate, skeletal muscle, placenta, mammary gland, bladder, lymph node, adipose tissue), and 2 human fetal normal tissues (fetal liver, fetal brain), on a denaturing formaldehyde 1.2% agarose gel and transferring to nylon membranes.

Filters are hybridized with random primed [α³²P]dCTP-labeled probes synthesized from the inserts of several of the protease genes. Hybridization is performed at 42° C. overnight in 6×SSC, 0.1% SDS, 1× Denhardt's solution, 100 μg/mL denatured herring sperm DNA with 1-2×10⁶ cpm/mL of ³²P-labeled DNA probes. The filters are washed in 0.1×SSC/0.1% SDS, 65° C., and exposed on a Molecular Dynamics phosphorimager.

Quantitative PCR Analysis

RNA is isolated from a variety of normal human tissues and cell lines. Single stranded cDNA is synthesized from 10 μg of each RNA as described above using the Superscript Preamplification System (GibcoBRL). These single strand templates are then used in a 25 cycle PCR reaction with primers specific to each clone. Reaction products are electrophoresed on 2% agarose gels, stained with ethidium bromide and photographed on a UV light box. The relative intensity of the STK-specific bands were estimated for each sample.

DNA Array Based Expression Analysis

Plasmid DNA array blots are prepared by loading 0.5 μg denatured plasmid for each protease on a nylon membrane. The [γ³²P]dCTP labeled single stranded DNA probes are synthesized from the total RNA isolated from several human immune tissue sources or tumor cells (such as thymus, dendrocytes, mast cells, monocytes, B cells (primary, Jurkat, RPMI8226, SR), T cells (CD8/CD4+, TH1, TH2, CEM, MOLT4), K562 (megakaryocytes). Hybridization is performed at 42° C. for 16 hours in 6×SSC, 0.1% SDS, 1× Denhardt's solution, 100 μg/mL denatured herring sperm DNA with 10⁶ cpm/mL of [γ³²P]dCTP labeled single stranded probe. The filters are washed in 0.1×SSC/0.1% SDS, 65° C., and exposed for quantitative analysis on a Molecular Dynamics phosphorimager.

### Example 5

Protease Gene Expression

Vector Construction

Materials and Methods

Expression Vector Construction

Expression constructs are generated for some of the human cDNAs including: a) full-length clones in a pCDNA expression vector; and b) a GST-fusion construct containing the catalytic domain of the novel protease fused to the C-terminal end of a GST expression cassette; and c) a full-length clone containing a mutation within the predicted polypeptide cleaving site within the protease domain, inserted in the pCDNA vector.

These mutants of the protease might function as dominant negative constructs, and will be used to elucidate the function of these novel proteases.

### Example 6

Generation of Specific Immunoreagents to Proteases

Materials and Methods

Specific immunoreagents are raised in rabbits against KLH- or MAP-conjugated synthetic peptides corresponding to isolated protease polypeptides. C-terminal peptides were conjugated to KLH with glutaraldehyde, leaving a free C-terminus. Internal peptides were MAP-conjugated with a blocked N-terminus. Additional immunoreagents can also be generated by immunizing rabbits with the bacterially expressed GST-fusion proteins containing the cytoplasmic domains of each novel PTK or STK.

The various immune sera are first tested for reactivity and selectivity to recombinant protein, prior to testing for endogenous sources.

Western Blots

Proteins in SDS PAGE are transferred to immobilon membrane. The washing buffer is PBST (standard phosphate-buffered saline pH 7.4+0.1% Triton X-100). Blocking and antibody incubation buffer is PBST+5% milk. Antibody dilutions are varied from 1:1000 to 1:2000.

### Example 7

Recombinant Expression and Biological Assays for Proteases

Materials and Methods

Transient Expression of Proteases in Mammalian Cells

The pcDNA expression plasmids (10 μg DNA/100 mm plate) containing the protease constructs are introduced into 293 cells with lipofectamine (Gibco BRL). After 72 hours, the cells are harvested in 0.5 mL solubilization buffer (20 mM HEPES, pH 7.35, 150 mM NaCl, 10% glycerol, 1% Triton X-100, 1.5 mM MgCl₂, mM EGTA, 2 mM phenylmethylsulfonyl fluoride, 1 μg/mL aprotinin). Sample aliquots are resolved by SDS polyacrylamide gel electrophoresis (PAGE) on 6% acrylamide/0.5% bis-acrylamide gels and electrophoretically transferred to nitrocellulose. Non-specific binding is blocked by preincubating blots in Blotto (phosphate buffered saline containing 5% w/v non-fat dried milk and 0.2% v/v nonidet P-40 (Sigma)), and recombinant protein is detected using the various anti-peptide or anti-GST-fusion specific antisera.

In Vitro Protease Assays

In Vitro Protease Assay Using Fluorogenic Peptides

Assays are carried out using a spectrofluorometer, such as Perkin-Elmer 204S. The standard reaction mixtures (100 μl) contains 200 mM Tris-HCl, pH8.5, and 200 μM fluorogenic peptide substrate. After enzyme addition, reaction mixtures are incubated at 37° C. for 30 min and terminated by addition of 1.9 ml of 125 mM ZnSO4 (Brenner, C., and Fuller, R. S., 1992, Proc. Natl. Acad. Sci. U. S. A. 89:922-926). The precipitate is removed by centrifugation for 1 min in a microcentrifuge (15,000×g), and the rate of product (7-amino-4-methyl-coumarin) released into the supernatant solution is determined fluorometrically [(excitation)=385 nm, (emission)=465 nm]. Examples of substrates used in the literature include: Boc-Gly-Arg-Arg-4-methylcoumaryl-7-amide (MCA), Boc-Gln-Arg-Arg-MCA, Z-Arg-Arg-MCA, and pGlu-Arg-Thr-Lys-Arg-MCA. Stock solutions (100 mM) are prepared by dissolving peptides in dimethyl sulfoxide that are then diluted in water to 1 mM working stock before use. (Details of this assay can be found in: R. Yosuf, et al. J. Biol. Chem., Vol. 275, Issue 14, 9963-9969, Apr. 7, 2000 which is incorporated herein by reference in its entirety including any figures, tables, or drawings.)

Protease Assay in Intact Cells Using Fluorogenic Peptides

Calpain activity is measured by the rate of generation of the fluorescent product, AMC, from intracellular thiol-conjugated Boc-Leu-Met-CMAC (Rosser, B. G., Powers, S. P., and Gores, G. J. (1993) J. Biol. Chem. 268, 23593-23600). Cells are dispersed, grown on glass coverslips, continuously superfused with physiologic saline solution at 37° C., and sequentially imaged with a quantitative fluorescence imaging system. At t=0, Boc-Leu-Met-CMAC (10 μM, Molecular Probes) is introduced into the superfusion solution, and mean fluorescence intensity (excitation 350 nm, emission 470 nm) of individual cells is measured at 60-s intervals. At 10 min, TNF-alpha (30 ng/ml) is added to the superfusion solution with 10μM Boc-Leu-Met-CMAC. The slope of the fluorescence change with respect to time represents the intracellular calpain activity (Rosser, et al., 1993, J. Biol. Chem. 268:23593-23600). For calpain assays in whole cell populations, suspension cultures of cells are loaded with 10 μM Boc-Leu-Met-CMAC, and changes in intracellular fluorescence are measured prior to and after TNF-alpha addition at 37° C. using a FACS Vantage system. Cellular fluorescence of AMC is measured using a 360-nm excitation filter and a 405-nm long-pass emission filter. (Details of this assay can be found in: Han, et al., 1999, J Biol Chem, 274:787-794 which is incorporated herein by reference in its entirety including any figures, tables, or drawings)

Protease Assay Using Chromogenic Substrates

The proteolytic activity of enzymes is measured using a commercially available assay system (Athena Environmental Sciences, Inc.). The assay employs a universal substrate of a dye-protein conjugate cross linked to a matrix. Protease activity is determined spectrophotometrically by measuring the absorbance of the dye released from the matrix to the supernatant. Reaction vials containing the enzyme and substrate are incubated for 3 h at 37° C. The activity is measured at different incubation times, and reactions are terminated by adding 500 μl of 0.2 N NaOH to each vial. The absorbance of the supernatant in each reaction vial is measured at 450 nm. The proteolytic activity is monitored using 10 μl (approximately 10 μg) of purified protein incubated with 5 μg of -casein (Sigma) in 50 mM Tris-HCl (pH 7.5) for 30 min, 1 h or 2 h at 37° C. The reaction products are resolved by SDS-polyacrylamide gel electrophoresis and proteins visualized by staining with Coomassie Blue (Details of this assay can be found in: Faccio, et al., 2000, J Biol Chem, 275:2581-2588 which is incorporated herein by reference in its entirety including any figures, tables, or drawings).

Protease Assay Using Radiolabeled Substrate Bound to Membranes

Unlabeled protease is mixed with radiolabeled substrate-containing membranes in buffer (100 mM HEPES, 100 mM NaCl, 125 μM magnesium acetate, 125 μM zinc acetate, pH 7.5) and incubated at 30° C. Typically, each reaction had a final volume of 80-100 μl. Each reaction is normalized to the same final concentration of lysis buffer components (25 mM Tris, 0.1 M sorbitol, 0.5 mM EDTA, 0.01 % NaN₃, pH 7.5) because the amount of membranes added to each reaction is varied. To examine metal ion specificity, reactions are assembled without substrate and pretreated with 1.125 mM 1,10-orthophenanthroline for 20 min on ice. Subsequently, metal ions and substrate-containing membranes are added, and reactions are initiated by incubation at 30° C.; the additions result in dilution of the 1,10-orthophenanthroline to a final concentration of 1 mM. The metal ions are added in the form of acetate salts from 25-100 mM stock solutions (Zn²⁺, Mg²⁺, Cu²⁺, Co²⁺, or Ca²⁺) that are first acidified with 2 mM concentrated HCl and then neutralized with 1 mM HEPES, pH 7.5; this step is necessary to achieve full solubilization of zinc acetate. For analysis by immunoprecipitation, samples are diluted 10-20× with immunoprecipitation buffer (Berkower, C., and Michaelis, S. (1991)EMBO J. 10:3777-3785) containing 0.1% SDS, cleared of insoluble material (13,000×g for 5-10 min at 4° C.), and immunoprecipitated with substrate-specific antibody. Alternatively, samples are solubilized by SDS (final concentration, 0.5%), boiled for 3 min, and directly immunoprecipitated after dilution with immunoprecipitation buffer. Immunoprecipitates are subjected to SDS-polyacrylamide gel electrophoresis as described, fixed for 7 min with 20% trichloroacetic acid, dried, and exposed to a PhosphorImager screen for detection and quantitation (Molecular Dynamics, Sunnyvale, Calif.). All of the above reagents can be purchased from Sigma (Details of this assay can be found in: Schmidt, et al., 2000, J Biol Chem, 275:6227-6233 which is incorporated herein by reference in its entirety including any figures, tables, or drawings). Variation of this assay to apply to substrate not bound to membrane is straightforward.

A comprehensive discussion of various protease assays can be found in: The Handbook of Proteolytic Enzymes by Alan J. Barrett (Editor), Neil D. Rawlings (Editor), J. Fred Woessner (Editor) (February 1998) Academic Press, San Diego; ISBN: 0-12-079370-9 (Which is incorporated herein by reference in its entirety including any figures, tables, or drawings).

Similar assays are performed on bacterially expressed GST-fusion constructs of the proteases.

### Example 8a

Chromosomal Localization of Proteases

Materials And Methods

Several sources were used to find information about the chromosomal localization of each of the genes described in this patent. First, the Celera Browser was used to map the genes. Alternatively, the accession number of a genomic contig (identified by BLAST against NRNA) was used to query the Entrez Genome Browser (http://www.ncbi.nlm.nih.gov/PMGifs/Genomes/MapviewerHelp.html), and the cytogenetic localization was read from the NCBI data. References for association of the mapped sites with chromosomal amplifications found in human cancer can be found in: Knuutila, et al., Am J Pathol, 1998, 152:1107-1123. Information on mapped positions was also obtained by searching published literature (at NCBI, http://www.ncbi.nlm.nih.zov/entrez/query.fcgi) for documented association of the mapped position with human disease.

Results

The chromosomal regions for mapped genes are listed in Table 2.

The following section describes various diseases that map to chromosomal locations established for proteases included in this patent application. The protease polynucleotides of the present invention can be used to identify individuals who have, or are at risk for developing, relevant diseases. As discussed elsewhere in this application, the polypeptides and polynucleotides of the present invention are useful in identifying compounds that modulate protease activity, and in turn ameliorate various diseases.

SGPr397, SEQ ID_{—}1, maps to human chromosomal position 8q12. Chromosomal aberrations in this region are associated with breast cancer: Rummukainen J, et al. Cancer Genet Cytogenet. Apr. 1, 2001;126(1):1-7.

SGPr413, SEQ ID NO:2, maps to human chromosomal position 2q35. This region is highly implicated in osteoarthritis (Loughlin J, et al., Linkage analysis of chromosome 2q in osteoarthritis. Rheumatology. 2000 April;39(4): 377-81).

SGPr404, SEQ ID NO:3, maps to human chromosomal position 10q26. Genomic amplification of this region has been associated with the following cancers (Knuutila): Malignant fibrous histiocytoma of soft tissue.

SGPr536_{—}1, SEQ ID NO:4, maps to human chromosomal position 1p35.

SGPr414, SEQ ID NO:5, maps to human chromosomal position 2p14.

SGPr430, SEQ ID NO:6, maps to human chromosomal position 2q37 This region is highly implicated in osteoarthritis (Loughlin J, et al. Linkage analysis of chromosome 2q in osteoarthritis. Rheumatology. 2000 April;39(4): 377-81).

SGPr496_{—}1, SEQ ID NO:7, maps to human chromosomal position Xp11.4. (Knuutila): small cell lung cancer and prostate cancer.

SGPr495, SEQ ID NO:8, maps to human chromosomal position 6q16.

SGPr407, SEQ ID NO:9, maps to human chromosomal position 2q37. This region is highly implicated in osteoarthritis (Loughlin J, et al., Linkage analysis of chromosome 2q in osteoarthritis. Rheumatology. 2000 April;39(4): 377-81).

SGPr453, SEQ ID NO:10, maps to human chromosomal position 12q23.

SGPr445, SEQ ID NO:11, maps to human chromosomal position 6q16.

SGPr401_{—}1, SEQ ID NO:12, maps to human chromosomal position 4q11. Genomic amplification of this region has been associated with the following cancers (Knuutila): Follicular carcinoma.

SGPr408, SEQ ID NO:13, maps to human chromosomal position 11p15.

SGPr480, SEQ ID NO:14, maps to human chromosomal position 17q24. Genomic amplification of this region has been associated with the following cancers (Knuutila): Non-small cell lung cancer, and testicular cancer.

SGPr431, SEQ ID NO:15, maps to human chromosomal position 4q31.3. Genomic amplification of this region has been associated with the following cancers (Knuutila): Osteosarcoma.

SGPr429, SEQ ID NO:16, maps to human chromosomal position 1p36. 2. Genomic amplification of this region has been associated with the following cancers (Knuutila): alveolar cancer.

SGPr503, SEQ ID NO:17, maps to human chromosomal position 12q24.3. Genomic amplification of this region has been associated with the following cancers (Knuutila): Non-small cell lung cancer.

SGPr427, SEQ ID NO:18, maps to human chromosomal position 17p13.

SGPr092, SEQ ID NO:19, maps to human chromosomal position 11p15.

SGPr359, SEQ ID NO:20, maps to human chromosomal position 11q22. Genomic amplification of this region has been associated with the following cancers (Knuutila): Uterine cervix cancer.

SGPr104_{—}1, SEQ ID NO:21, maps to human chromosomal position 3q27. Genomic amplification of this region has been associated with the following cancers (Knuutila): Squamous cell carcinomas of the head and neck; Malignant fibrous histiocytoma of soft tissue.

SGPr303, SEQ ID NO:22, maps to human chromosomal position 17q11.1. Genomic amplification of this region has been associated with the following cancers (Knuutila): Breast carcinoma and Hepatocellular carcinoma.

SGPr402_{—}1, SEQ ID NO:23, maps to human chromosomal position 19q11. Genomic amplification of this region has been associated with the following cancers (Knuutila): Leiomyosarcoma.

SGPr434, SEQ ID NO:24, maps to human chromosomal position 3p21. Genomic amplification of this region has been associated with the following cancers (Knuutila): Bladder carcinoma.

SGPr446_{—}1, SEQ ID NO:25, maps to human chromosomal position 3p21. Genomic amplification of this region has been associated with the following cancers (Knuutila): Bladder carcinoma.

SGPr447, SEQ ID NO:26, maps to human chromosomal position 16p13.3.

SGPr432_{—}1, SEQ ID NO:27, has not been assigned a chromosomal location.

SGPr529, SEQ ID NO:28, maps to human chromosomal position 19q13.4. Genomic amplification of this region has been associated with the following cancers (Knuutila): Breast carcinoma.

SGPr428_{—}1, SEQ ID NO:29, maps to human chromosomal position 8p23.

SGPr425, SEQ ID NO:30, maps to human chromosomal position 6q14.

SGPr548, SEQ ID NO:31, maps to human chromosomal position 19q13.4. Genomic amplification of this region has been associated with the following cancers Knuutila): Breast carcinoma.

SGPr396, SEQ ID NO:32, maps to human chromosomal position 4q32. Genomic amplification of this region has been associated with the following cancers (Knuutila): Non-small cell lung cancer.

SGPr426, SEQ ID NO:33, maps to human chromosomal position 4q13. Genomic amplification of this region has been associated with the following cancers (Knuutila): Non-small cell lung cancer.

SGPr552, SEQ ID NO:34, maps to human chromosomal position 4q13. Genomic amplification of this region has been associated with the following cancers (Knuutila): Non-small cell lung cancer.

SGPr405, SEQ ID NO:35, maps to human chromosomal position 16p13.3.

SGPr485_{—}1, SEQ ID NO:36, maps to human chromosomal position 8p23.

SGPr534, SEQ ID NO:37, maps to human chromosomal position 16q23. Genomic amplification of this region has been associated with the following cancers (Knuutila): Diffuse large cell lymphoma of stomach.

SGPr390, SEQ ID NO:38, maps to human chromosomal position 19q11. Genomic amplification of this region has been associated with the following cancers (Knuutila): Leiomyosarcoma.

SGPr521, SEQ ID NO:39, maps to human chromosomal position 19q13.4. Genomic amplification of this region has been associated with the following cancers (Knuutila): Breast carcinoma.

SGPr530_{—}1, SEQ ID NO:40, maps to human chromosomal position 9q22. Genomic amplification of this region has been associated with the following cancers (Knuutila): Non-small cell lung cancer.

SGPr520, SEQ ID NO:41, maps to human chromosomal position 2q37. This region is highly implicated in osteoarthritis (Loughlin J, et al., Linkage analysis of chromosome 2q in osteoarthritis. Rheumatology. 2000 April;39(4): 377-81).

SGPr455, SEQ ID NO:42, maps to human chromosomal position 12p11.2. Genomic amplification of this region has been associated with the following cancers (Knuutila): ovarian germ cell tumor, testicular cancer and non-small cell lung cancer.

SGPr507_{—}2, SEQ ID NO:43, maps to human chromosomal position 7q36. Genomic amplification of this region has been associated with the following cancers (Knuutila): Ovarian cancer.

SGPr559, SEQ ID NO:44, maps to human chromosomal position 21q22.

SGPr567_{—}1, SEQ ID NO:45, maps to human chromosomal position 11q23. Genomic amplification of this region has been associated with the following cancers (Knuutila): Pleural mesothelioma.

SGPr479_{—}1, SEQ ID NO:46, maps to human chromosomal position 1q42.

SGPr489_{—}1, SEQ ID NO:47, maps to human chromosomal position 11p15.

SGPr465_{—}1, SEQ ID NO:48, has not been assigned a chromosomal location.

SGPr524_{—}1, SEQ ID NO:49, has not been assigned a chromosomal location.

SGPr422, SEQ ID NO:50, maps to human chromosomal position 4q13. Genomic amplification of this region has been associated with the following cancers (Knuutila): Non-small cell lung cancer.

SGPr538, SEQ ID NO:51, maps to human chromosomal position 11q23. Genomic amplification of this region has been associated with the following cancers (Knuutila): Pleural mesothelioma.

SGPr527_{—}1, SEQ ID NO:52, has not been assigned a chromosomal location.

SGPr542, SEQ ID NO:53, maps to human chromosomal position 19q13.1. Genomic amplification of this region has been associated with the following cancers (Knuutila): Small cell lung cancer (highly associated, with 10 of 35 patients tested showing amplification).

SGPr551, SEQ ID NO:54, maps to human chromosomal position 22q13. Genomic amplification of this region has been associated with the following cancers (Knuutila): Osteosarcoma.

SGPr451, SEQ ID NO:55, maps to human chromosomal position 12q23.

SGPr452_{—}1, SEQ ID NO:56, maps to human chromosomal position 16p13.3.

SGPr504, SEQ ID NO:57, has not been assigned a chromosomal location.

SGPr469, SEQ ID NO:58, has not been assigned a chromosomal location.

SGPr400, SEQ ID NO:59, maps to human chromosomal position 4q32. Genomic amplification of this region has been associated with the following cancers (Knuutila): Non-small cell lung cancer.

### Example 8b

Candidate Single Nucleotide Polymorphisms (SNPs)

Materials and Methods

The most common variations in human DNA are single nucleotide polymorphisms (SNPs), which occur approximately once every 100 to 300 bases.

Because SNPs are expected to facilitate large-scale association genetics studies, there has recently been great interest in SNP discovery and detection. Candidate SNPs for the genes in this patent were identified by blastn searching the nucleic acid sequences against the public database of sequences containing documented SNPs (dbSNP, at NCBI, http://www.ncbi.nlm.nih.gov/SNP/snpblastpretty.html). dbSNP accession numbers for the SNP-containing sequences are given. SNPs were also identified by comparing several databases of expressed genes (dbEST, NRNA) and genomic sequence (i.e., NRNA) for single basepair mismatches. The results are shown in Table 1, in the column labeled “SNPs”. These are candidate SNPs—their actual frequency in the human population was not determined. The code below is standard for representing DNA sequence:

G = Guanosine
A = Adenosine
T = Thymidine
C = Cytidine
R = G or A, puRine
Y = C or T, pYrimidine
K = G or T, Keto
W = A or T, Weak (2 H-bonds)
S = C or G, Strong (3 H-bonds)
M = A or C, aMino
B = C, G or T (i.e., not A)
D = A, G or T (i.e., not C)
H = A, C or T (i.e., not G)
V = A, C or G (i.e., not T)
N = A, C, G or T, aNy
X = A, C, G or T

| | |
|---|---|
| complementary | G A T C R Y W S K M B V D H N X |
| DNA | +−+−+−+−+−+−+−+−+−+−+−+−+−+−+−+−+ |
| strands | C T A G Y R S W M K V B H D N X |

For example, if two versions of a gene exist, one with a “C” at a given position, and a second one with a “T: at the same position, then that position is represented as a Y, which means C or T. SNPs may be important in identifying heritable traits associated with a gene.

Results

The results of SNP identification are contained in Table 2 above, and in Example 1, under the section entitled DESCRIPTION OF NOVEL PROTEASE POLYNUCLEOTIDES. As discussed above, a variety of SNPs were identified in the protease polynucleotides of the present invention.

### Example 9

Demonstration of Gene Amplification by Southern Blotting

Materials and Methods

Nylon membranes are purchased from Boehringer Mannheim. Denaturing solution contains 0.4 M NaOH and 0.6 M NaCl. Neutralization solution contains 0.5 M Tris-HCL, pH 7.5 and 1.5 M NaCl. Hybridization solution contains 50% formamide, 6×SSPE, 2.5× Denhardt's solution, 0.2 mg/mL denatured salmon DNA, 0.1 mg/mL yeast tRNA, and 0.2% sodium dodecyl sulfate. Restriction enzymes are purchased from Boehringer Mannheim. Radiolabeled probes are prepared using the Prime-it II kit by Stratagene. The β-actin DNA fragment used for a probe template is purchased from Clontech.

Genomic DNA is isolated from a variety of tumor cell lines (such as MCF-7, MDA-MB-231, Calu-6, A549, HCT-15, HT-29, Colo 205, LS-180, DLD-1, HCT-116, PC3, CAPAN-2, MIA-PaCa-2, PANC-1, AsPc-1, BxPC-3, OVCAR-3, SKOV3, SW 626 and PA-1, and from two normal cell lines.

A 10 μg aliquot of each genomic DNA sample is digested with EcoR I restriction enzyme and a separate 10 μg sample is digested with Hind III restriction enzyme. The restriction-digested DNA samples are loaded onto a 0.7% agarose gel and, following electrophoretic separation, the DNA is capillary-transferred to a nylon membrane by standard methods (Sambrook, J. et al. (1989) Molecular Cloning: A Laboratory Manual, Cold Spring Harbor Laboratory).

### Example 10

Detection of Protein-Protein Interaction Through Phage Display

Materials and Methods

Phage display provides a method for isolating molecular interactions based on affinity for a desired bait. cDNA fragments cloned as fusions to phage coat proteins are displayed on the surface of the phage. Phage(s) interacting with a bait are enriched by affinity purification and the insert DNA from individual clones is analyzed.

T7 Phage Display Libraries

All libraries were constructed in the T7Select1-1b vector (Novagen) according to the manufacturer's directions.

Bait Presentation

Protein domains to be used as baits are generated as C-terminal fusions to GST and expressed in E. coli. Peptides are chemically synthesized and biotinylated at the N-terminus using a long chain spacer biotin reagent.

Selection

Aliquots of refreshed libraries (10¹⁰-10¹² pfu) supplemented with PanMix and a cocktail of E. coli inhibitors (Sigma P-8465) are incubated for 1-2 hrs at room temperature with the immobilized baits. Unbound phage is extensively washed (at least 4 times) with wash buffer.

After 3-4 rounds of selection, bound phage is eluted in 100 μL of 1% SDS and plated on agarose plates to obtain single plaques.

Identification of Insert DNAs

Individual plaques are picked into 25 μL of 10 mM EDTA and the phage is disrupted by heating at 70° C. for 10 min. 2 μL of the disrupted phage are added to 50 μL PCR reaction mix. The insert DNA is amplified by 35 rounds of thermal cycling (94° C., 50 sec; 50° C., 1 min; 72° C., 1 min).

Composition of Buffer

10× PanMix

5% Triton X-100

10% non-fat dry milk (Carnation)

10 mM EGTA

250 mM NaF

250 μg/mL Heparin (sigma)

250 μg/mL sheared, boiled salmon sperm DNA (sigma)

0.05% Na azide

Prepared in PBS

Wash Buffer

PBS supplemented with:

0.5% NP-40

25 μg/mL heparin

PCR reaction mix

| 1.0 mL | 10x PCR buffer (Perkin-Elnier, with 15 mM Mg) |
|---|---|
| 0.2 mL each dNTPs (10 mM stock) | |
| 0.1 mL | T7UP primer (15 pmol/84 L) GGAGCTGTCGTATTCCAGTC |
| 0.1 mL | T7DN primer (15 pmol/νL) |

AACCCCTCAAGACCCGTTTAG

0.2 mL25 mM MgCl₂ or MgSO₄ to compensate for EDTA

Q.S. to 10 mL with distilled water

Add 1 unit of Taq polymerase per 50 μL reaction

LIBRARY: T7 Select1-H441

### Example 11

Gene Expression Based on Incyte and Public ESTs

Materials and Methods

The nucleic acid sequences for the proteases were used as queries in a BLASTN search of the Incyte and public dbEST databases of expressed sequences. The tissue sources of the libraries in which the protease was represented are listed below, along with the frequency the gene occurred in specific tissues. The frequency is determined by the number of clones representing the gene within a given tissue source. The Incyte gene identification number or public NCBI accession number is given, followed by the tissue source. A brief summary of the tissue specificity is then given for each gene.

Results

SGPr397, SEQID:1,

| Incyte 366783.1 | Clones: 2 prostate, 3 colon, retina and small intestine |
|---|---|
| 366783.3 | 1 prostate clone |

Selective expression in prostate (3/8 clones) and colon (3/8 clones)

SGPr413, SEQID:2,

| Incyte 475365.6 | 5 clones, 3 in small intestine, plus |
|---|---|
| | prostate, breast tumor |
| 475365.5 | 10 clones: 8 in small intestine, plus brain (2) |

Highly selective expression in small intestine (11/15 clones)

SGPr404, SEQID:3,

| Incyte 1129157.1 | 213 clones, highest in brain (18), m/f genitalia |
|---|---|
| | (21/23), breast (14), and digestive (25) |
| 1129157.2 | 1: mixed |

Broad expression, some elevation in brain (18/214 clones), digestive tissues (25/118) and male/female genitalia (21, 23 clones) and breast (14 clones)

SGPr536_{—}1, SEQID:4,

| Incyte 233762.17 | 149 clones: no tissue >21 hits |
|---|---|
| 233762.15 | 15 clones, mixed |

Broad expression seen in 164 clones

SGPr414, SEQID:5,

| | |
|---|---|
| Incyte 399773.5 | 669 clones, 404 libraries, broadly distributed |

Expressed broadly and strongly (669 clones)

SGPr430, SEQID:6,

| Incyte 407823.1 | 21 clones (4 testis, 3 brain, 3 prostate) |
|---|---|
| 1136483.1 | 1 Prostate |
| 411246.1 | 2 ea lung tumor, sm intestine, |
| | fetal liver, and 1 heart |
| 322700.1 | T cells |
| 407823.2 | Fetal liver/spleen |

Mixed expression, hightest in testis (4/31 clones), brain (3/31) and prostate (4/31) and fetal liver/spleen (4/31)

SGPr496_{—}1, SEQID:7,

| | |
|---|---|
| Incyte 986031.1 | 12 clones: 2 ea lung, brain, adrenal tumor |

Selective expression in lung (2/12 clones), adrenal tumor (2/12) and brain (2/12)

SGPr495, SEQID:8,

| Incyte 350921.2 | 16 clones: 2 thymus, 3 colon, 3 brain |
|---|---|
| 350921.7 | Adrenal tumor |
| 350921.10 | 14 clones: 2 colon, 1 adrenal tumor |
| 350921.6 | 10 clones: 2 adrenal (1 tumor), 2 brain |
| 350921.1 | Adrenal |
| 350921.9 | Colon (2) Sm intestine, lung tumor |

Selectively expressed in adrenal gland (5/46 clones) and colon (7/46)

SGPr407, SEQID:9,

No ESTs

SGPr453, SEQID:10,

| Incyte 428428.1 | 17 clones: 3 lung (2 tumors), 2 prostate, 4 |
|---|---|
| | testis, 2 teratoma (hNT2) |
| 428428.5 | brain, lung, teratoma |
| 428428.6 | teratoma (2), lung, kidney |

Highly expressed in hNT2 teratoma cell line (5/24 clones), and selective for lung (5 clones) and testis (4 clones)

SGPr445, SEQID:11,

| Incyte 350921.7 | 1 adrenal tumor |
|---|---|
| 350921.10 | 14 incl 1 adrenal tumor |
| 350921.6 | 10 clones: normal and tumor adrenal (1 ea) |
| | colon tumor (2) |
| 350921.1 | Adrenal gland |
| 350921.8 | 2 prostate tumor, 1 retina |

Highest in adrenal gland (5/28 clones), indicates a possible involvement in adrenal hormone processing

SGPr401_{—}1, SEQID:12,

| Incyte 232414.1 | 169 clones: 69 NS, 18 male genitalia, (10 prostate), |
|---|---|
| | 11 female genitalia, 11 respiratory system, 5 kidney, |
| | 9 in one glioblastoma library. |
| 232414.2 | testis |

Selective for nervous system (69/170 clones), especially glioblastoma

SGPr408, SEQID:13,

| Incyte 233660.2 | 357 clones, 248 libraries: 54 brain, 26 hemic/immune |
|---|---|
| | 24/21 f/m genitalia, 21 digestive, 20 cardiovascular |
| 233660.11 | 13 clones, broad expression. |
| 233660.10 | 7 clones, broad expression |

Expressed broadly and strongly (377 clones)

SGPr480, SEQID:14,

| Incyte 1326256.3 | 274 clones, broad but highest in NS (59), hemic |
|---|---|
| | (35) genitalia (24/10, m/f). 6 clones in one |
| | pituitary gland library |
| 1326256.8 | 4 mixed clones |
| 1326256.1 | 26 clones, 13 in male genitalia |
| 1326256.10 | 10 clones mixed |

Broad, strong expression (over 300 clones)

SGPr431, SEQID:15,

| Incyte 236368.1 | 151 clones, 110 libraries, highest in divestive, |
|---|---|
| | nervous, hemic (18, 17, 18). 5, 4 hits each in |
| | two fetal liver/spleen libraries |
| 236368.2 | 1 fetal heart |
| 236368.14 | 7: mixed |

Broad and moderately strong expression (159 clones total)

SGPr429, SEQID:16,

| | | |
|---|---|---|
| Incyte 890540.9 | 41 | clones, broad |
| 890540.1 | 125 | clones, broad |
| 890540.8 | 15 | clones, broad |

Broad and moderately strong expression (181 clones)

SGPr503, SEQID:17,

| Incyte 1447357.3 | 107 clones highest in NS (15), male genitalia |
|---|---|
| | (11) and digestive tissue (15) |
| 1447357.1 | Dendritic cells |
| 245045.1 | 16 mixed |

Broad expression (124 clones), highest in nervous sytem (16), male genitalia (11) and digestive tissue (15)

SGPr427, SEQID:18,

| Incyte 903092.31 | 41 clones, 35 libraries; 9 clones in prostate, |
|---|---|
| | otherwise very broad |
| 903092.23 | 1 brain |

Expression elevated in prostate (9/42 clones)

SGPr092, SEQID:19,

| | |
|---|---|
| Incyte 339251.1 | 4/5 uterus, 1 mixed tissue |
| 339251.2 | 1/1 uterus; Highly selective expression in |
| | uterus (5/6 clones) |

SGPr359, SEQID:20,

| | |
|---|---|
| Incyte 391133.1 | mixed tissue (fetal lung, testis, B-cell) |

gi|7280399=same as Incyte

Mixed tissues, one EST only

SGPr104_{—}1, SEQID:21,

Incyte 12/23 clones in brain

| | |
|---|---|
| 232015.5 | 6/7 clones in brain |
| 232015.2 | 2/4 clones in brain |
| 232015.1 | 1/1 brain |
| 232015.6 | 1/1 brain |

Brain secific

SGPr303, SEQID:22,

| Incyte 323846.15 | 38 samples, highest in brain(8) breast(4), |
|---|---|
| | uterus and ovary(7) |
| 323846.1 | 304 clones, high in nervous sys(72) and |
| | genitalia (28 f, 24 m), other tissue |
| 414048.34 | 45 clones, highest in NS |
| 323846.11 | 8 clones |

Broad expression

SGPr402_{—}1, SEQID:23,

| Incyte 244407.4 | 25 clones, highest in testis (8) brain |
|---|---|
| | (4), uterus (2; 1 tumor) |
| 244407.2 | uterus |
| 244407.1 | testis |
| 244407.6 | uterus tumor |
| 244407.5 | fallopian tube tumor |
| 244407.9 | mixed tissue incl tumor, nasal tumor |

Enriched in genital samples.

SGPr434, SEQID:24,

| Incyte 110154.4 | no clone origin |
|---|---|
| 110154.6 | 2 prostate |
| 110154.12 | 1 prostate, 1 pituitary |
| 110154.11 | 1 pituitary |
| 110154.8 | fallopian tube tumor (2), mixed (1) |
| 110154.7 | Pituitary |
| 110154.5 | Thigh muscle (2) - tissue-specific splicing |
| 110154.10 | 3 heart, 2 brain, pituitary (61/62 match) |

Selective expression in prostate (3/17 clones), 4 pituitary gland (4/17 clones) and faloptian tube tumor (2/17 clones). May indicate a role in hormone processing in pituitary and prostate.-hormone processing.

SGPr446_{—}1, SEQID:25,

| Incyte 1040641.1 | Heart, Muscle |
|---|---|
| 1388371.1 | 2 Heart |

Specific for muscle (3/3 clones) especially heart muscle (2/3 clones)

SGPr447, SEQID:26,

| | |
|---|---|
| Incyte 1352932.1 | pancreas tumor |

Single clone from pancreas tumor

SGPr432_{—}1, SEQID:27,

| Incyte 474674.15 | 29 clones, mixed |
|---|---|
| 474674.30 | 90 clones, mixed |
| 474674.1 | 82 clones, mixed |

Broad and strong expression (201 clones total)

SGPr529, SEQID:28,

| Incyte 988019.3 | 71 clones, 23 in f genitalia. 9 from 1 ovary tumor |
|---|---|
| | library, 2 from another, 2 from another, and one |
| | from yet another (no normal ovaries). 5 from |
| | one pancreatic tumor line, 4 from pancreas tumor |
| | library |
| 988019.1 | breast skin |

Selective expression in pancreas (4/72 clones from one pancreatic tumor library and 5 from a pancreatic tumor line) and ovary (14 from ovary tumors, none from normal ovary).

SGPr428_{—}1, SEQID:29,

| | |
|---|---|
| Incyte 891146.1 | 4: brain, pituitary, blood, thymus |

Broad, low-level expression (4 clones all from differnet tissues)

SGPr425, SEQID:30,

| | |
|---|---|
| Incyte 400833.1 | 25 clones, mixed (<4 from any tissue, |
| | except 5 from ‘fetus’) |

Expressed broadly but not strongly (25 clones total)

SGPr548, SEQID:31,

| | |
|---|---|
| Incyte 971236.1 | 2 clones from mixed testis, fetal lung, B cells |

Rare transcript, just two clones from a mixed library of testis, fetal lung and B cells

SGPr396, SEQID:32,

| Incyte 209051.1 | Lung (1) |
|---|---|
| 889126.1 | Brain (1) |

Only 2 ESTs—lung and brain

SGPr426, SEQID:33, Incyte No ESTs

SGPr552, SEQID:34,

| Incyte 1510512.1 | tonsil, spinal cord |
|---|---|
| 1511222.1 | tonsil |
| 406221.1 | 83 clones: 16 in NS, 10 in hemic/immune, 9 in |
| | male genitalia, and several other tissues. 1 tonsil, |
| 981355.3 | 8 clones, 2 ovary tumor, 1 tonsil, varied |

Of 94 clones, see some selectivity in tonsil (3/94, but tonsil not
usually seen as an expression source), and nervous system (17 clones)

SGPr405, SEQID:35,

| | |
|---|---|
| Incyte 134360.1 | 1 kidney |

One clone, from kidney

SGPr485_{—}1, SEQID:36,

| | |
|---|---|
| Incyte 180576.2 | 5/5 clones in testis |

Testis specific (5/5 clones)

SGPr534, SEQID:37,

Incyte 1383391.20 112/114, matches well at start (103-165=perfect match) but maybe template artefact

| | |
|---|---|
| 1450812.1 | 1/1 pancreas, few mismatches are N's |
| 1383391.13 | 5/5 pancreas |
| 1045834.1 | 1/1 pancreas |

Almost completely pancreas-specific (118/120 clones from pancreas)

SGPr390, SEQID:38,

| | |
|---|---|
| Incyte 199428.9 | Bone tumor, small intestine |
| 199428.3 | 382 clones: 41 brain, 34/23 genitalia (m/f), |
| | 22 hemic/immune, 27 digestive |

Broad tissue distribution, highest in brain (41/382 clones), male and female genitalia (34 and 23 clones, respecively) and digestive system (27 clones)

SGPr521, SEQID:39,

| | |
|---|---|
| Incyte 427826.1 | 28 clones, most in sm intestine tumor (5, 1 |
| | library), neonatal keratinocytes (3 ea from 2 |
| | libraries), 8 ovary tumors, 5 breast skin |

Selective expression in ovarian tumors (8/28 clones), neonatal keratinocytes (6/28), breast keratinocytes (5) and in a small intestine tumor library (5 clones from one library)

SGPr530_{—}1, SEQID:40, No ESTs

SGPr520, SEQID:41,

| | |
|---|---|
| Incyte 405947.1 | 4/4 clones adrenal tumor (pheochromocytoma) |
| | (3 from one library, 1 from another) |
| 1338652.1 | 1/1 clones from adrenal tumor (pheochromocytoma) |
| 1477189.1 | 1/1 clones from adrenal (mixed normal and |
| | pheochromocytoma) |

Specific to pheochromocytoma (adrenal gland tumor): 4/5 clones from pheochromocytoma and 1/5 from mixed normal adrenal gland and pheochromocytoma.

SGPr455, SEQID:42,

| Incyte 1115833.1 | mixed fetal lung/testis/Bcell |
|---|---|
| 987279.1 | Brain (1), mixed tissues incl tumor (1) |

Three clones, only one (brain) with a specific source

SGPr507_{—}2, SEQID:43,

| Incyte 403891.1 | 10 clones: 6 in testis and 6 in mixed |
|---|---|
| | (testis, lung, Bcell) |
| 403891.2 | 1 brain |

Testis-selective: 6/11 clones from tesis and 5/11 from mixed libraries including testis samples

SGPr559, SEQID:44,

| Incyte 475100.1 | 35 clones, 11 in f genitalia, 8 in digestive: 7 |
|---|---|
| | uterus tumors (none normal), 4 in breast, 2 |
| | ovary tumors, 1 HeLa cervial tumor |
| 475100.6 | Th1 cells, HeLa cells |

Selective expression in tumors of the uterus (7/37 clones), ovary (2/37) cervix (2/37 from HeLa cervical tumor cell line), as well as breast (4)

SGPr567_{—}1, SEQID:45,

| | |
|---|---|
| Incyte 981355.3 | Mixed (2/8 clones from ovary tumor library, 1 ea |
| | from tonsil, brain, lung tumor, heart, placenta, |
| | dorsal root ganglion) |

Rare broad expression (8 clones from 7 different tissues).

SGPr479_{—}1, SEQID:46,

| | |
|---|---|
| Incyte 219214.1 | 1 testis |

Single EST, expressed in testis

SGPr489_{—}1, SEQID:47,

| Incyte 338956.1 | 2 kidney, 1 placenta |
|---|---|
| 1042306.1 | 1 mouth tumor, 2 fallopian tube tumor |
| 1384824.1 | Sm intestine, kidney |

Rare but broad expression, selective to kidney (3/8 clones) and fallopian tube tumor (2/8 from one library)

SGPr465_{—}1, SEQID:48, No ESTs

SGPr524_{—}1, SEQID:49,

| Incyte 952182.3 | 1 testis |
|---|---|
| 952182.2 | 1 testis |
| 952182.4 | 1 prostate |

Specific to male genitalia (2/3 clones in testis, 1/3 in prostate)

SGPr422, SEQID:50,

| Incyte 1511284.1 | 1 tonsil |
|---|---|
| 1351259.1 | 1 brain |

Rare transcript seen only in tonsil (1/2 clones) and brain (1/2 clones)

SGPr538, SEQID:51,

| Incyte 903092.29 | 4 clones, 3 brain, 1 breast |
|---|---|
| 903092.19 | 1 brain |
| 903092.22 | 2 brain |
| 903092.28 | 38 clones, 24 in brain |
| 903092.24 | 2 brain, 1 sm intestine |

Selective expression in nervous system (32/48 clones)

SGPr527_{—}1, SEQID:52,

| Incyte 65450.1 | 1 prostate tumor |
|---|---|
| 103554.2 | mixed tissues incl tumor |
| 228456.2 | 11 clones: mixed (2 brain, 2 blood) |
| 103554.1 | Mixed (3) |

Broad low-level expression (16 clones)

SGPr542, SEQID:53,

| | |
|---|---|
| Incyte 244085.1 | Expression is selective to hemopoetic cells: All |
| | 11 clones are from hemopoetic tissues: 6 from |
| | fetal liver/spleen, 4 of which are mast cells, 2 |
| | from umbilical cord blood, 1 from CD34+ bone |
| | marrow, and two clones from leukemias: 1 from AML |
| | blast cells and one from CML |

SGPr551, SEQID:54,

| | |
|---|---|
| Incyte 319529.1 | 22 clones: 7 liver, 1 fetal liver/spleen, 3 lung |
| 319529.2 | 1 liver |
| 319529.3 | 2 mixed tissues incl testis, 1 testis (tissue- |
| | specific splice) |

Selectively expressed in liver (9/26 clones), may have a testis-specific splice form (3/3 clones of one template)

SGPr451, SEQID:55,

| | |
|---|---|
| Incyte 1471541.1 | 1 mixed) |

SGPr452_{—}1, SEQID:56,

| | |
|---|---|
| Incyte 446374.1 | Mixed (melanocytes, uterus, fetal heart) |

No expression data (single EST from mixed tissues)

SGPr504, SEQID:57, Incyte 244085.1 Expression is selective to hemopoetic cells: All 11 clones are from hemopoetic tissues: 6 from fetal liver/spleen, 4 of which are mast cells, 2 from umbilical cord blood, 1 from CD34+ bone marrow, and two clones from leukemias: 1 from AML blast cells and one from CML

SGPr469, SEQID:58,

| Incyte 110154.10 | 7 clones: 3 heart, 2 brain, 1 pituitary |
|---|---|
| 110154.3 | heart, muscle, testis |

Selective expression in heart (4/10 clones) SGPr400, SEQID:59,

| | |
|---|---|
| Incyte 889126.1 | Brain |

Only one EST, in brain

### CONCLUSION

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

In view of the degeneracy of the genetic code, other combinations of nucleic acids also encode the claimed peptides and proteins of the invention. For example, all four nucleic acid sequences GCT, GCC, GCA, and GCG encode the amino acid alanine. Therefore, if for an amino acid there exists an average of three codons, a polypeptide of 100 amino acids in length will, on average, be encoded by 3100, or 5×1047, nucleic acid sequences. Thus, a nucleic acid sequence can be modified to form a second nucleic acid sequence, encoding the same polypeptide as encoded by the first nucleic acid sequences, using routine procedures and without undue experimentation. Thus, all possible nucleic acids that encode the claimed peptides and proteins are also fully described herein, as if all were written out in full taking into account the codon usage, especially that preferred in humans. Furthermore, changes in the amino acid sequences of polypeptides, or in the corresponding nucleic acid sequence encoding such polypeptide, may be designed or selected to take place in an area of the sequence where the significant activity of the polypeptide remains unchanged. For example, an amino acid change may take place within a β-turn, away from the active site of the polypeptide. Also changes such as deletions (e.g. removal of a segment of the polypeptide, or in the corresponding nucleic acid sequence encoding such polypeptide, which does not affect the active site) and additions (e.g. addition of more amino acids to the polypeptide sequence without affecting the function of the active site, such as the formation of GST-fusion proteins, or additions in the corresponding nucleic acid sequence encoding such polypeptide without affecting the function of the active site) are also within the scope of the present invention. Such changes to the polypeptides can be performed by those with ordinary skill in the art using routine procedures and without undue experimentation. Thus, all possible nucleic and/or amino acid sequences that can readily be determined not to affect a significant activity of the peptide or protein of the invention are also fully described herein.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims.

1. An isolated, enriched or purified nucleic acid molecule, wherein said nucleic acid molecule comprises a nucleotide sequence that:
(a) encodes a polypeptide having an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118 and biological domains thereof;
(b) is the complement of the nucleotide sequence of (a); or
(c) hybridizes under stringent conditions to the nucleotide molecule of (a) and encodes a protease polypeptide.

2. The nucleic acid molecule of claim 1, further comprising a vector or promoter operatively linked to the nucleotide sequence.

3. The nucleic acid molecule of claim 1, wherein said nucleic acid molecule is isolated, enriched, or purified from a mammal.

4. The nucleic acid molecule of claim 3, wherein said mammal is a human.

5. The nucleic acid molecule of claim 1 comprising a nucleic acid comprising a nucleotide sequence which hybridizes under stringent conditions to a nucleotide sequence encoding a protease polypeptide having an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118.

6. An isolated, enriched, or purified protease polypeptide, wherein said polypeptide comprises an amino acid sequence at least about 90% identical to a sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:1110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118 and biological domains thereof.

7. The protease polypeptide of claim 6, wherein said polypeptide is isolated, purified, or enriched from a mammal.

8. The protease polypeptide of claim 7, wherein said mammal is a human.

9. An antibody or antibody fragment having specific binding affinity to a protease polypeptide or to a domain of said polypeptide, wherein said polypeptide comprises an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118.

10. A hybridoma which produces the antibody of claim 9.

11. A kit comprising an antibody which binds to a polypeptide of claim 6 and a negative control antibody.

12. A method for identifying a substance that modulates the activity of a protease polypeptide comprising the steps of:
(a) contacting a protease polypeptide substantially identical to an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111SEQ ID NO:112,SEQ ID NO:113,SEQ ID NO:114, SEQ ID NO:115, SEQ ID NO:116, SEQ ID NO:117 and SEQ ID NO:118 with a test substance;
(b) measuring the activity of said polypeptide; and
(c) determining whether said substance modulates the activity of said polypeptide.

13. A method for identifying a substance that modulates the activity of a protease polypeptide in a cell comprising the steps of:
(a) expressing a protease polypeptide in a cell, wherein said polypeptide comprises a sequence substantially identical to an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ED NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118;
(b) adding a test substance to said cell; and
(c) monitoring a change in cell phenotype, cell proliferation, cell differentiation or the interaction between said polypeptide and a natural binding partner.

14. A method for treating a disease or disorder by administering to a patient in need of such treatment a substance that modulates the activity of a protease substantially identical to an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118.

15. The method of claim 14, wherein said disease or disorder is selected from the group consisting of cancers, immune-related diseases and disorders, cardiovascular disease, brain or neuronal-associated diseases, metabolic disorders and inflammatory disorders.

16. The method of claim 15, wherein said disease or disorder is selected from the group consisting of cancers of tissues; cancers of blood or hematopoietic origin; cancers of the breast, colon, lung, prostrate, cervical, brain, ovarian, bladder or kidney.

17. The method of claim 15, wherein said disease or disorder is selected from the group consisting of central or peripheral nervous system diseases, migraines; pain; sexual dysfunction; mood disorders; attention disorders; cognition disorders; hypotension; hypertension; psychotic disorders; neurological disorders and dyskinesias.

18. The method of claim 15, wherein said substance modulates protease activity in vitro.

19. The method of claim 18, wherein said substance is a protease inhibitor.

20. A method for detection of a protease polypeptide in a sample as a diagnostic tool for a disease or disorder, wherein said method comprises:
(a) contacting said sample with a nucleic acid probe which hybridizes under hybridization assay conditions to a nucleic acid target region of a protease polypeptide having an amino acid sequence selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118, said probe comprising the nucleic acid sequence encoding the polypeptide, fragments thereof, and the complements of the sequences and fragments; and
(b) detecting the presence or amount of the probe:target region hybrid as an indication of the disease.

21. The method of claim 20, wherein said disease or disorder is selected from the group consisting of cancers, immune-related diseases and disorders, cardiovascular disease, brain or neuronal-associated diseases, metabolic disorders and inflammatory disorders.

22. The method of claim 21, wherein said disease or disorder is selected from the group consisting of cancers of tissues; cancers of hematopoietic cancers of blood or hematopoietic origin; cancers of the breast, colon, lung, prostrate, cervical, brain, ovarian, bladder or kidney.

23. The method of claim 21, wherein said disease or disorder is selected from the group consisting of central or peripheral nervous systems disease, migraines, pain; sexual dysfunction; mood disorders; attention disorders; cognition disorders; hypotension; hypertension; psychotic disorders; neurological disorders; and dyskinesias.

24. The isolated, enriched or purified nucleic acid molecule of claim 1 comprising a nucleic molecule encoding a biological domain of a protease polypeptide having a sequence selected from the group consisting of SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ'ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118.

25. The nucleic acid molecule of claim 1 comprising a nucleic acid sequence encoding a protease polypeptide having an amino acid sequence that has least 90% identity to a polypeptide selected from the group consisting of those set forth in SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117 and SEQ ID NO:118.

26. The nucleic acid molecule of claim 1 wherein the molecule comprises a nucleotide sequence substantially identical to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, and SEQ ID NO:59.

27. An isolated, enriched or purified nucleic acid molecule consisting essentially of about 10-30 contiguous nucleotide bases of a nucleic acid sequence that encodes a polypeptide that is selected from the group consisting of SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO: 112, SEQ ID NO: 113, SEQ ID NO:114, SEQ ID NO:115, SEQ ID NO:116, SEQ ID NO:117 and SEQ ID NO:118.

28. The isolated, enriched or purified nucleic acid molecule of claim 27 consisting essentially of about 10-30 contiguous nucleotide bases of a nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, and SEQ ID NO:59.

29. A recombinant cell comprising the nucleic acid molecule of claim 1.

30. A method for detecting the presence or amount of protease polypeptide in a sample comprising
(a) contacting the sample with the antibody of claim 9 under conditions suitable for protease-antibody immunocomplex formation; and
(b) detecting the presence or amount of the antibody conjugated to the protease polypeptide.
